SEQUENCE LISTING (42 sequences)

SEQ ID NO: 1 - DNA, 2043 bp, Homo sapiens
  source 1..2043: mol_type = other DNA
atgccggggttcctggttcgcatcctccctctgttgctggttctgctgcttctgggccctacgcgcggcttgcgcaatgccacccagaggatgtttgaaattgactatagccgggactccttcctcaaggatggccagccatttcgctacatctcaggaagcattcactactcccgtgtgccccgcttctactggaaggaccggctgctgaagatgaagatggctgggctgaacgccatccagacgtatgtgccctggaactttcatgagccctggccaggacagtaccagttttctgaggaccatgatgtggaatattttcttcggctggctcatgagctgggactgctggttatcctgaggcccgggccctacatctgtgcagagtgggaaatgggaggattacctgcttggctgctagagaaagagtctattcttctccgctcctccgacccagattacctggcagctgtggacaagtggttgggagtccttctgcccaagatgaagcctctcctctatcagaatggagggccagttataacagtgcaggttgaaaatgaatatggcagctactttgcctgtgattttgactacctgcgcttcctgcagaagcgctttcgccaccatctgggggatgatgtggttctgtttaccactgatggagcacataaaacattcctgaaatgtggggccctgcagggcctctacaccacggtggactttggaacaggcagcaacatcacagatgctttcctaagccagaggaagtgtgagcccaaaggacccttgatcaattctgaattctatactggctggctagatcactggggccaacctcactccacaatcaagaccgaagcagtggcttcctccctctatgatatacttgcccgtggggcgagtgtgaacttgtacatgtttataggtgggaccaattttgcctattggaatggggccaactcaccctatgcagcacagcccaccagctacgactatgatgccccactgagtgaggctggggacctcactgagaagtattttgctctgcgaaacatcatccagaagtttgaaaaagtaccagaaggtcctatccctccatctacaccaaagtttgcatatggaaaggtcactttggaaaagttaaagacagtgggagcagctctggacattctgtgtccctctgggcccatcaaaagcctttatcccttgacatttatccaggtgaaacagcattatgggtttgtgctgtaccggacaacacttcctcaagattgcagcaacccagcacctctctcttcacccctcaatggagtccacgatcgagcatatgttgctgtggatgggatcccccagggagtccttgagcgaaacaatgtgatcactctgaacataacagggaaagctggagccactctggaccttctggtagagaacatgggacgtgtgaactatggtgcatatatcaacgattttaagggtttggtttctaacctgactctcagttccaatatcctcacggactggacgatctttccactggacactgaggatgcagtgcgcagccacctggggggctggggacaccgtgacagtggccaccatgatgaagcctgggcccacaactcatccaactacacgctcccggccttttatatggggaacttctccattcccagtgggatcccagacttgccccaggacacctttatccagtttcctggatggaccaagggccaggtctggattaatggctttaaccttggccgctattggccagcccggggccctcagttgaccttgtttgtgccccagcacatcctgatgacctcggccccaaacaccatcaccgtgctggaactggagtgggcaccctgcagcagtgatgatccagaactatgtgctgtgacgttcgtggacaggccagttattggctcatctgtgacctacgatcatccctccaaacctgttgaaaaaagactcatgcccccacccccgcaaaaaaacaaagattcatggctggaccatgtatgatgatgaaag

SEQ ID NO: 2 - DNA, 2043 bp, synthetic construct
  source 1..2043: mol_type = other DNA
atgccgggtttcctggtgcgtatcctgccgctgctgctggttctgctgctgctgggtccgacccgtggcctgcgtaacgcgacccagcgtatgtttgagattgactacagccgtgatagcttcctgaaggatggtcaaccgtttcgttacatcagcggcagcattcactatagccgtgtgccgcgtttctactggaaagaccgtctgctgaagatgaaaatggcgggtctgaacgcgatccagacctatgttccgtggaactttcacgagccgtggccgggtcaatatcaattcagcgaggaccacgatgtggaatactttctgcgtctggcgcacgaactgggtctgctggttatcctgcgtccgggcccgtacatttgcgcggagtgggaaatgggtggcctgccggcgtggctgctggagaaggaaagcatcctgctgcgtagcagcgacccggattatctggcggcggtggataaatggctgggtgttctgctgccgaagatgaaaccgctgctgtaccagaacggtggcccggtgattaccgtgcaagttgagaacgaatacggcagctatttcgcgtgcgactttgattacctgcgtttcctgcagaagcgttttcgtcaccacctgggtgacgatgtggttctgttcaccaccgacggcgcgcacaagacctttctgaaatgcggtgcgctgcagggcctgtataccaccgttgacttcggtaccggcagcaacatcaccgatgcgtttctgagccagcgtaagtgcgagccgaaaggtccgctgattaacagcgaattctacaccggttggctggatcactggggccaaccgcacagcaccatcaagaccgaggcggtggcgagcagcctgtatgacattctggcgcgtggtgcgagcgttaacctgtacatgttcatcggtggcaccaactttgcgtattggaacggtgcgaacagcccgtatgcggcgcagccgaccagctacgactatgatgcgccgctgagcgaggcgggtgacctgaccgaaaaatatttcgcgctgcgtaacatcattcaaaagtttgagaaagttccggaaggtccgatcccgccgagcaccccgaagtttgcgtacggcaaagtgaccctggaaaagctgaaaaccgttggtgcggcgctggatatcctgtgcccgagcggtccgattaagagcctgtatccgctgaccttcattcaggtgaaacaacactacggttttgttctgtatcgtaccaccctgccgcaggactgcagcaacccggcgccgctgagcagcccgctgaacggcgttcacgaccgtgcgtacgtggcggttgatggtatcccgcaaggcgtgctggagcgtaacaacgttatcaccctgaacattaccggtaaagcgggtgcgaccctggacctgctggtggaaaacatgggtcgtgttaactacggcgcgtatattaacgatttcaaaggtctggtgagcaacctgaccctgagcagcaacatcctgaccgactggaccatttttccgctggacaccgaggatgcggttcgtagccacctgggtggctggggtcaccgtgacagcggtcaccacgatgaagcgtgggcgcacaacagcagcaactacaccctgccggcgttctatatgggtaactttagcattccgagcggcattccggacctgccgcaggataccttcatccaatttccgggttggaccaaaggccaagtgtggattaacggtttcaacctgggtcgttattggccggcgcgtggtccgcagctgaccctgtttgtgccgcaacacatcctgatgaccagcgcgccgaacaccattaccgttctggagctggaatgggcgccgtgcagcagcgatgatccggagctgtgcgcggtgaccttcgttgaccgtccggtgatcggcagcagcgttacctacgatcacccgagcaagccggtggaaaaacgtctgatgccgccgccgccgcagaagaacaaagacagctggctggatcacgtttaatgatgaaag

SEQ ID NO: 3 - DNA, 536 bp, synthetic construct
  source 1..536: mol_type = other DNA
atcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgc

SEQ ID NO: 4 - DNA, 2600 bp, synthetic construct
  source 1..2600: mol_type = other DNA
atcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatctgccgccaccatgccggggttcctggttcgcatcctccctctgttgctggttctgctgcttctgggccctacgcgcggcttgcgcaatgccacccagaggatgtttgaaattgactatagccgggactccttcctcaaggatggccagccatttcgctacatctcaggaagcattcactactcccgtgtgccccgcttctactggaaggaccggctgctgaagatgaagatggctgggctgaacgccatccagacgtatgtgccctggaactttcatgagccctggccaggacagtaccagttttctgaggaccatgatgtggaatattttcttcggctggctcatgagctgggactgctggttatcctgaggcccgggccctacatctgtgcagagtgggaaatgggaggattacctgcttggctgctagagaaagagtctattcttctccgctcctccgacccagattacctggcagctgtggacaagtggttgggagtccttctgcccaagatgaagcctctcctctatcagaatggagggccagttataacagtgcaggttgaaaatgaatatggcagctactttgcctgtgattttgactacctgcgcttcctgcagaagcgctttcgccaccatctgggggatgatgtggttctgtttaccactgatggagcacataaaacattcctgaaatgtggggccctgcagggcctctacaccacggtggactttggaacaggcagcaacatcacagatgctttcctaagccagaggaagtgtgagcccaaaggacccttgatcaattctgaattctatactggctggctagatcactggggccaacctcactccacaatcaagaccgaagcagtggcttcctccctctatgatatacttgcccgtggggcgagtgtgaacttgtacatgtttataggtgggaccaattttgcctattggaatggggccaactcaccctatgcagcacagcccaccagctacgactatgatgccccactgagtgaggctggggacctcactgagaagtattttgctctgcgaaacatcatccagaagtttgaaaaagtaccagaaggtcctatccctccatctacaccaaagtttgcatatggaaaggtcactttggaaaagttaaagacagtgggagcagctctggacattctgtgtccctctgggcccatcaaaagcctttatcccttgacatttatccaggtgaaacagcattatgggtttgtgctgtaccggacaacacttcctcaagattgcagcaacccagcacctctctcttcacccctcaatggagtccacgatcgagcatatgttgctgtggatgggatcccccagggagtccttgagcgaaacaatgtgatcactctgaacataacagggaaagctggagccactctggaccttctggtagagaacatgggacgtgtgaactatggtgcatatatcaacgattttaagggtttggtttctaacctgactctcagttccaatatcctcacggactggacgatctttccactggacactgaggatgcagtgcgcagccacctggggggctggggacaccgtgacagtggccaccatgatgaagcctgggcccacaactcatccaactacacgctcccggccttttatatggggaacttctccattcccagtgggatcccagacttgccccaggacacctttatccagtttcctggatggaccaagggccaggtctggattaatggctttaaccttggccgctattggccagcccggggccctcagttgaccttgtttgtgccccagcacatcctgatgacctcggccccaaacaccatcaccgtgctggaactggagtgggcaccctgcagcagtgatgatccagaactatgtgctgtgacgttcgtggacaggccagttattggctcatctgtgacctacgatcatccctccaaacctgttgaaaaaagactcatgcccccacccccgcaaaaaaacaaagattcatggctggaccatgtatgatgatgaaaggtcgac

SEQ ID NO: 5 - DNA, 2600 bp, synthetic construct
  source 1..2600: mol_type = other DNA
atcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatccgccgccaccatgccgggtttcctggtgcgtatcctgccgctgctgctggttctgctgctgctgggtccgacccgtggcctgcgtaacgcgacccagcgtatgtttgagattgactacagccgtgatagcttcctgaaggatggtcaaccgtttcgttacatcagcggcagcattcactatagccgtgtgccgcgtttctactggaaagaccgtctgctgaagatgaaaatggcgggtctgaacgcgatccagacctatgttccgtggaactttcacgagccgtggccgggtcaatatcaattcagcgaggaccacgatgtggaatactttctgcgtctggcgcacgaactgggtctgctggttatcctgcgtccgggcccgtacatttgcgcggagtgggaaatgggtggcctgccggcgtggctgctggagaaggaaagcatcctgctgcgtagcagcgacccggattatctggcggcggtggataaatggctgggtgttctgctgccgaagatgaaaccgctgctgtaccagaacggtggcccggtgattaccgtgcaagttgagaacgaatacggcagctatttcgcgtgcgactttgattacctgcgtttcctgcagaagcgttttcgtcaccacctgggtgacgatgtggttctgttcaccaccgacggcgcgcacaagacctttctgaaatgcggtgcgctgcagggcctgtataccaccgttgacttcggtaccggcagcaacatcaccgatgcgtttctgagccagcgtaagtgcgagccgaaaggtccgctgattaacagcgaattctacaccggttggctggatcactggggccaaccgcacagcaccatcaagaccgaggcggtggcgagcagcctgtatgacattctggcgcgtggtgcgagcgttaacctgtacatgttcatcggtggcaccaactttgcgtattggaacggtgcgaacagcccgtatgcggcgcagccgaccagctacgactatgatgcgccgctgagcgaggcgggtgacctgaccgaaaaatatttcgcgctgcgtaacatcattcaaaagtttgagaaagttccggaaggtccgatcccgccgagcaccccgaagtttgcgtacggcaaagtgaccctggaaaagctgaaaaccgttggtgcggcgctggatatcctgtgcccgagcggtccgattaagagcctgtatccgctgaccttcattcaggtgaaacaacactacggttttgttctgtatcgtaccaccctgccgcaggactgcagcaacccggcgccgctgagcagcccgctgaacggcgttcacgaccgtgcgtacgtggcggttgatggtatcccgcaaggcgtgctggagcgtaacaacgttatcaccctgaacattaccggtaaagcgggtgcgaccctggacctgctggtggaaaacatgggtcgtgttaactacggcgcgtatattaacgatttcaaaggtctggtgagcaacctgaccctgagcagcaacatcctgaccgactggaccatttttccgctggacaccgaggatgcggttcgtagccacctgggtggctggggtcaccgtgacagcggtcaccacgatgaagcgtgggcgcacaacagcagcaactacaccctgccggcgttctatatgggtaactttagcattccgagcggcattccggacctgccgcaggataccttcatccaatttccgggttggaccaaaggccaagtgtggattaacggtttcaacctgggtcgttattggccggcgcgtggtccgcagctgaccctgtttgtgccgcaacacatcctgatgaccagcgcgccgaacaccattaccgttctggagctggaatgggcgccgtgcagcagcgatgatccggagctgtgcgcggtgaccttcgttgaccgtccggtgatcggcagcagcgttacctacgatcacccgagcaagccggtggaaaaacgtctgatgccgccgccgccgcagaagaacaaagacagctggctggatcacgtttaatgatgaaaggtcgac

SEQ ID NO: 6 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 7 - DNA, 584 bp, synthetic construct
  source 1..584: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 8 - DNA, 584 bp, synthetic construct
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 9 - DNA, 181 bp, synthetic construct
  source 1..181: mol_type = other DNA
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 10 - DNA, 138 bp, synthetic construct
  source 1..138: mol_type = other DNA
tcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggag

SEQ ID NO: 11 - DNA, 205 bp, synthetic construct
  source 1..205: mol_type = other DNA
tccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacct

SEQ ID NO: 12 - DNA, 117 bp, synthetic construct
  source 1..117: mol_type = other DNA
ttttaaaagaaaaggggggattggggggtacagtgcaggggaaagaatagtagacataatagcaacagacatacaaactaaagaattacaaaaacaaattacaaaattcaaaatttt

SEQ ID NO: 13 - DNA, 592 bp, synthetic construct
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 14 - DNA, 235 bp, synthetic construct
  source 1..235: mol_type = other DNA
tggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcag

SEQ ID NO: 15 - DNA, 131 bp, synthetic construct
  source 1..131: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 16 - DNA, 84 bp, synthetic construct
  source 1..84: mol_type = other DNA
taattttttttatttatgcagaggccgaggccgcctcggcctctgagctattccagaagtagtgaggaggcttttttggaggcc

SEQ ID NO: 17 - DNA, 456 bp, synthetic construct
  source 1..456: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaattt

SEQ ID NO: 18 - skipped

SEQ ID NO: 19 - DNA, 7110 bp, synthetic construct
  source 1..7110: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatatcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatccgccgccacccacctgcgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 20 - DNA, 2084 bp, synthetic construct
  source 1..2084: mol_type = other DNA
atgctccgggtccccctgtgtacgccgctcccgctcctggcactgctgcaactgctgggcgctgcgcacggcatctataatgtcacccagaggacatttaagctcgactacagccgggaccgcttcctcaaggatggacagccattccgatacatctcgggaagcattcattacttccggataccccgcttctactgggaggaccggctgctgaagatgaagatggctgggctgaatgctatccagatgtacgtgccctggaacttccatgaaccccaaccaggacaatatgagttttctggggaccgtgatgtggagcatttcatccagctggctcatgagctgggactcctggtgatcctgaggcctgggccctacatctgtgcagagtgggacatggggggcttacctgcttggctactagagaaacaatctatcgttctccggtcttctgacccagactaccttgtagctgtggataaatggctggcagtccttctgcccaagatgaagcccctgctctaccagaacggaggaccgatcataaccgtgcaggttgagaatgagtacgggtcctactttgcctgcgattacgactacctacgcttcctggtgcaccgcttccgctaccatctgggtaatgacgtcattctcttcaccaccgacggagcaagtgaaaaaatgctgaagtgtgggaccctgcaggacctgtacgccacagtggattttggaacaggcaacaatatcacacaagctttcctggtccagaggaagtttgaacctaaaggacctttgatcaattctgagttctatactggctggctagaccactggggtaaaccccattccacggtgaaaactaaaacactggctacctccctctataacctgcttgcccgtggggccaacgtgaacttgtacatgtttataggtgggaccaattttgcctattggaatggtgccaacacgccctatgagccacagcccaccagctatgactacgacgccccactgagcgaggctggggacctcactaagaagtattttgctcttcgagaagtcattcagatgtttaaagaagtcccagaaggccctatccctccgtctacacccaaatttgcatatggaaaagttgctctgagaaagttcaagacagtggctgaagctctgggtatcctgtgtcccaatgggccagtgaaaagcctctatcccctgacattcactcaggtaaaacagtattttgggtatgtgctgtaccgaacaacgcttcctcaagattgcagtaacccgaaacccattttctcttcacccttcaatggtgtccgtgatcgggcttacgtctctgtggacggggtcccccaaggaatccttgatcgaaacctcatgacagctctgaacatacaggggaaggctggagccacgctggacatcctggtggagaacatggggcgtgtgaactatggcagattcatcaatgacttcaagggtttgatttccaacatgactatcaactccactgtcctcaccaactggacggtcttcccactggacactgaggccatggtacgcaaccatctctggggccgggaggccagtgatgggggtcaccttgacggacggtcgacctccaattcttcggacctcatactccccaccttttacgtgggcaacttctccatcccctcgggcatcccagacctgccacaggacaccttcatccagtttcctgggtggtccaagggtcaagtatggatcaatggctttaacctcggccgatactggcccacaatgggcccacaaaagaccttgttcgtgccaaggaacatcctgaccacttcagccccaaacaacatcacagtgttggagctagagtttgcaccctgcagcgaggggaccccagagctgtgtacagtagagtttgttgacactccggtcatttcctgacctgacttgaccatcggtggccattttccaagccagtctggtcaagactcatggctgaacctctgagactgagccttggggagcacagctctactctggttacacggatcacctttgttgtgctagaatggaagctacat

SEQ ID NO: 21 - DNA, 3045 bp, Homo sapiens
  source 1..3045: mol_type = other DNA
atgggcgcctacgcgcgggcttcgggggtctgcgctcgcggctgcctggactcagcaggcccctggaccatgtcccgcgccctgcggccaccgctcccgcctctctgctttttccttttgttgctggcggctgccggtgctcgggccgggggatacgagacatgccccacagtgcagccgaacatgctgaacgtgcacctgctgcctcacacacatgatgacgtgggctggctcaaaaccgtggaccagtacttttatggaatcaagaatgacatccagcacgccggtgtgcagtacatcctggactcggtcatctctgccttgctggcagatcccacccgtcgcttcatttacgtggagattgccttcttctcccgttggtggcaccagcagacaaatgccacacaggaagtcgtgcgagaccttgtgcgccaggggcgcctggagttcgccaatggtggctgggtgatgaacgatgaggcagccacccactacggtgccatcgtggaccagatgacacttgggctgcgctttctggaggacacatttggcaatgatgggcgaccccgtgtggcctggcacattgaccccttcggccactctcgggagcaggcctcgctgtttgcgcagatgggcttcgacggcttcttctttgggcgccttgattatcaagataagtgggtacggatgcagaagctggagatggagcaggtgtggcgggccagcaccagcctgaagcccccgaccgcggacctcttcactggtgtgcttcccaatggttacaacccgccaaggaatctgtgctgggatgtgctgtgtgtcgatcagccgctggtggaggaccctcgcagccccgagtacaacgccaaggagctggtcgattacttcctaaatgtggccactgcccagggccggtattaccgcaccaaccacactgtgatgaccatgggctcggacttccaatatgagaatgccaacatgtggttcaagaaccttgacaagctcatccggctggtaaatgcgcagcaggcaaaaggaagcagtgtccatgttctctactccacccccgcttgttacctctgggagctgaacaaggccaacctcacctggtcagtgaaacatgacgacttcttcccttacgcggatggcccccaccagttctggaccggttacttttccagtcggccggccctcaaacgctacgagcgcctcagctacaacttcctgcaggtgtgcaaccagctggaggcgctggtgggcctggcggccaacgtgggaccctatggctccggagacagtgcacccctcaatgaggcgatggctgtgctccagcatcacgacgccgtcagcggcacctcccgccagcacgtggccaacgactacgcgcgccagcttgcggcaggctgggggccttgcgaggttcttctgagcaacgcgctggcgcggctcagaggcttcaaagatcacttcaccttttgccaacagctaaacatcagcatctgcccgctcagccagacggcggcgcgcttccaggtcatcgtttataatcccctggggcggaaggtgaattggatggtacggctgccggtcagcgaaggcgttttcgttgtgaaggaccccaatggcaggacagtgcccagcgatgtggtaatatttcccagctcagacagccaggcgcaccctccggagctgctgttctcagcctcactgcccgccctgggcttcagcacctattcagtagcccaggtgcctcgctggaagccccaggcccgcgcaccacagcccatccccagaagatcctggtcccctgctttaaccatcgaaaatgagcacatccgggcaacgtttgatcctgacacagggctgttgatggagattatgaacatgaatcagcaactcctgctgcctgttcgccagaccttcttctggtacaacgccagtataggtgacaacgaaagtgaccaggcctcaggtgcctacatcttcagacccaaccaacagaaaccgctgcctgtgagccgctgggctcagatccacctggtgaagacacccttggtgcaggaggtgcaccagaacttctcagcttggtgttcccaggtggttcgcctgtacccaggacagcggcacctggagctagagtggtcggtggggccgatacctgtgggcgacacctgggggaaggaggtcatcagccgttttgacacaccgctggagacaaagggacgcttctacacagacagcaatggccgggagatcctggagaggaggcgggattatcgacccacctggaaactgaaccagacggagcccgtggcaggaaactactatccagtcaacacccggatttacatcacggatggaaacatgcagctgactgtgctgactgaccgctcccaggggggcagcagcctgagagatggctcgctggagctcatggtgcaccgaaggctgctgaaggacgatggacgcggagtatcggagccactaatggagaacgggtcgggggcgtgggtgcgagggcgccacctggtgctgctggacacagcccaggctgcagccgccggacaccggctcctggcggagcaggaggtcctggcccctcaggtggtgctggccccgggtggcggcgccgcctacaatctcggggctcctccgcgcacgcagttctcagggctgcgcagggacctgccgccctcggtgcacctgctcacgctggccagctggggccccgaaatggtgctgctgcgcttggagcaccagtttgccgtaggagaggattccggacgtaacctgagcgcccccgttaccttgaacttgagggacctgttctccaccttcaccatcacccgcctgcaggagaccacgctggtggccaaccagctccgcgaggcagcctccaggctcaagtggacaacaaacacaggccccacaccccaccaaactccgtaccagctggacccggccaacatcacgctggaacccatggaaatccgcactttcctggcctcagttcaatggaaggaggtggatggttaggtctgctgg

SEQ ID NO: 22 - DNA, 3045 bp, synthetic construct
  source 1..3045: mol_type = other DNA
atgggcgcctacgcaagggcatccggcgtgtgcgcaaggggctgtctggactctgccggaccatggaccatgagcagagccctgaggccacctctgccaccactgtgcttctttctgctgctgctggcagcagcaggagcaagggcaggaggctatgagacatgtcctaccgtgcagccaaacatgctgaatgtgcacctgctgcctcacacccacgacgatgtgggctggctgaagacagtggaccagtacttttatggcatcaagaatgatatccagcacgccggcgtgcagtacatcctggactctgtgatcagcgccctgctggccgatccaacccggagattcatctatgtggagatcgccttctttagcagatggtggcaccagcagacaaacgccactcaggaggtggtgagggatctggtgcgccagggccggctggagtttgcaaacggaggatgggtcatgaatgatgaggccgccacacactacggcgccatcgtggaccagatgaccctgggcctgcgctttctggaggacacattcggcaacgatggcagacccagggtggcatggcacatcgatcctttcggccactctcgggagcaggccagcctgtttgcccagatgggcttcgacggcttctttttcggccgcctggactaccaggataagtgggtgcggatgcagaagctggagatggagcaagtgtggagggcctctaccagcctgaagcctccaaccgccgacctgtttacaggcgtgctgcctaacggctataatccccctcgcaatctgtgctgggacgtgctgtgcgtggaccagccactggtggaggacccccggagcccagagtacaacgccaaggagctggtggactatttcctgaatgtggccaccgcccagggcagatactataggacaaaccacaccgtgatgacaatgggctccgattttcagtacgagaacgccaatatgtggttcaagaacctggacaagctgatccgcctggtgaatgcacagcaggcaaagggcagctccgtgcacgtgctgtacagcaccccagcctgctatctgtgggagctgaacaaggccaatctgacatggtccgtgaagcacgacgacttcttcccatacgccgatggcccccaccagttttggaccggctatttctctagccggcccgccctgaagcggtacgagcggctgtcctataacttcctgcaggtgtgcaatcagctggaggccctggtgggactggcagcaaacgtgggaccatacggctccggcgactctgcccccctgaatgaggccatggccgtgctgcagcaccacgatgccgtgagcggcacctccagacagcacgtggccaacgactatgcaaggcagctggcagcaggatggggaccatgcgaggtgctgctgtctaatgccctggccagactgaggggctttaaggatcactttaccttctgccagcagctgaacatctccatctgtcctctgtctcagacagccgccagattccaggtcatcgtgtacaaccctctgggcagaaaagtgaattggatggtgaggctgccagtgagcgagggcgtgtttgtggtgaaggaccccaatggcaggaccgtgccttctgacgtggtcatcttcccatcctctgatagccaggcccacccacccgagctgctgttttccgcctctctgcccgccctgggcttctctacctatagcgtggcccaggtgccaaggtggaagcctcaggcaagggcaccacagccaatccctaggcgcagctggtcccccgccctgacaatcgagaacgagcacatcagagccaccttcgaccctgatacaggcctgctgatggagatcatgaacatgaatcagcagctgctgctgcccgtgaggcagacctttttctggtacaacgcctccatcggcgacaatgagtccgatcaggcctctggcgcctatatctttagacccaatcagcagaagccactgcccgtgtctagatgggcccagatccacctggtgaagacacctctggtgcaggaggtgcaccagaacttcagcgcctggtgttcccaggtggtgagactgtaccctggccagaggcacctggagctggagtggagcgtgggacctatcccagtgggcgatacctggggcaaggaagtgatctccagatttgacacccctctggagacaaagggccgcttctacacagattctaacggccgggagatcctggagcggagaagggactatagaccaacctggaagctgaatcagacagagcctgtggccggcaactactatccagtgaataccaggatctatatcacagacggcaatatgcagctgaccgtgctgacagatagatcccagggaggcagctccctgagggacggctctctggagctgatggtgcaccgccggctgctgaaggacgatggaaggggcgtgtccgagccactgatggagaacggctctggagcatgggtgcgcggccggcacctggtgctgctggataccgcacaggcagcagcagcaggacacaggctgctggccgagcaggaggtgctggccccacaggtggtgctggcaccaggaggaggagcagcctacaatctgggagcacctccaaggacccagttttccggactgagaagggacctgccacctagcgtgcacctgctgacactggcatcctggggaccagagatggtgctgctgaggctggagcaccagttcgcagtgggagaggatagcggaaggaacctgtccgccccagtgaccctgaatctgagggacctgttttctaccttcacaatcacccggctgcaggagaccacactggtggccaaccagctgagagaggccgccagcaggctgaagtggaccacaaataccggccccacacctcaccagacaccttaccagctggaccccgccaacatcaccctggagcccatggagatcagaacattcctggccagcgtgcagtggaaggaggtggacggctgagtctgctgg

SEQ ID NO: 23 - DNA, 2670 bp, synthetic construct
  source 1..2670: mol_type = other DNA
atcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatccagagcgcccactgcctaacggagagaccccatcgtggcgcgatcatgctccgggtccccctgtgtacgccgctcccgctcctggcactgctgcaactgctgggcgctgcgcacggcatctataatgtcacccagaggacatttaagctcgactacagccgggaccgcttcctcaaggatggacagccattccgatacatctcgggaagcattcattacttccggataccccgcttctactgggaggaccggctgctgaagatgaagatggctgggctgaatgctatccagatgtacgtgccctggaacttccatgaaccccaaccaggacaatatgagttttctggggaccgtgatgtggagcatttcatccagctggctcatgagctgggactcctggtgatcctgaggcctgggccctacatctgtgcagagtgggacatggggggcttacctgcttggctactagagaaacaatctatcgttctccggtcttctgacccagactaccttgtagctgtggataaatggctggcagtccttctgcccaagatgaagcccctgctctaccagaacggaggaccgatcataaccgtgcaggttgagaatgagtacgggtcctactttgcctgcgattacgactacctacgcttcctggtgcaccgcttccgctaccatctgggtaatgacgtcattctcttcaccaccgacggagcaagtgaaaaaatgctgaagtgtgggaccctgcaggacctgtacgccacagtggattttggaacaggcaacaatatcacacaagctttcctggtccagaggaagtttgaacctaaaggacctttgatcaattctgagttctatactggctggctagaccactggggtaaaccccattccacggtgaaaactaaaacactggctacctccctctataacctgcttgcccgtggggccaacgtgaacttgtacatgtttataggtgggaccaattttgcctattggaatggtgccaacacgccctatgagccacagcccaccagctatgactacgacgccccactgagcgaggctggggacctcactaagaagtattttgctcttcgagaagtcattcagatgtttaaagaagtcccagaaggccctatccctccgtctacacccaaatttgcatatggaaaagttgctctgagaaagttcaagacagtggctgaagctctgggtatcctgtgtcccaatgggccagtgaaaagcctctatcccctgacattcactcaggtaaaacagtattttgggtatgtgctgtaccgaacaacgcttcctcaagattgcagtaacccgaaacccattttctcttcacccttcaatggtgtccgtgatcgggcttacgtctctgtggacggggtcccccaaggaatccttgatcgaaacctcatgacagctctgaacatacaggggaaggctggagccacgctggacatcctggtggagaacatggggcgtgtgaactatggcagattcatcaatgacttcaagggtttgatttccaacatgactatcaactccactgtcctcaccaactggacggtcttcccactggacactgaggccatggtacgcaaccatctctggggccgggaggccagtgatgggggtcaccttgacggacggtcgacctccaattcttcggacctcatactccccaccttttacgtgggcaacttctccatcccctcgggcatcccagacctgccacaggacaccttcatccagtttcctgggtggtccaagggtcaagtatggatcaatggctttaacctcggccgatactggcccacaatgggcccacaaaagaccttgttcgtgccaaggaacatcctgaccacttcagccccaaacaacatcacagtgttggagctagagtttgcaccctgcagcgaggggaccccagagctgtgtacagtagagtttgttgacactccggtcatttcctgacctgacttgaccatcggtggccattttccaagccagtctggtcaagactcatggctgaacctctgagactgagccttggggagcacagctctactctggttacacggatcacctttgttgtgctagaatggaagctacat

SEQ ID NO: 24 - DNA, 3602 bp, synthetic construct
  source 1..3602: mol_type = other DNA
atcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatccgccgccaccatgggcgcctacgcgcgggcttcgggggtctgcgctcgcggctgcctggactcagcaggcccctggaccatgtcccgcgccctgcggccaccgctcccgcctctctgctttttccttttgttgctggcggctgccggtgctcgggccgggggatacgagacatgccccacagtgcagccgaacatgctgaacgtgcacctgctgcctcacacacatgatgacgtgggctggctcaaaaccgtggaccagtacttttatggaatcaagaatgacatccagcacgccggtgtgcagtacatcctggactcggtcatctctgccttgctggcagatcccacccgtcgcttcatttacgtggagattgccttcttctcccgttggtggcaccagcagacaaatgccacacaggaagtcgtgcgagaccttgtgcgccaggggcgcctggagttcgccaatggtggctgggtgatgaacgatgaggcagccacccactacggtgccatcgtggaccagatgacacttgggctgcgctttctggaggacacatttggcaatgatgggcgaccccgtgtggcctggcacattgaccccttcggccactctcgggagcaggcctcgctgtttgcgcagatgggcttcgacggcttcttctttgggcgccttgattatcaagataagtgggtacggatgcagaagctggagatggagcaggtgtggcgggccagcaccagcctgaagcccccgaccgcggacctcttcactggtgtgcttcccaatggttacaacccgccaaggaatctgtgctgggatgtgctgtgtgtcgatcagccgctggtggaggaccctcgcagccccgagtacaacgccaaggagctggtcgattacttcctaaatgtggccactgcccagggccggtattaccgcaccaaccacactgtgatgaccatgggctcggacttccaatatgagaatgccaacatgtggttcaagaaccttgacaagctcatccggctggtaaatgcgcagcaggcaaaaggaagcagtgtccatgttctctactccacccccgcttgttacctctgggagctgaacaaggccaacctcacctggtcagtgaaacatgacgacttcttcccttacgcggatggcccccaccagttctggaccggttacttttccagtcggccggccctcaaacgctacgagcgcctcagctacaacttcctgcaggtgtgcaaccagctggaggcgctggtgggcctggcggccaacgtgggaccctatggctccggagacagtgcacccctcaatgaggcgatggctgtgctccagcatcacgacgccgtcagcggcacctcccgccagcacgtggccaacgactacgcgcgccagcttgcggcaggctgggggccttgcgaggttcttctgagcaacgcgctggcgcggctcagaggcttcaaagatcacttcaccttttgccaacagctaaacatcagcatctgcccgctcagccagacggcggcgcgcttccaggtcatcgtttataatcccctggggcggaaggtgaattggatggtacggctgccggtcagcgaaggcgttttcgttgtgaaggaccccaatggcaggacagtgcccagcgatgtggtaatatttcccagctcagacagccaggcgcaccctccggagctgctgttctcagcctcactgcccgccctgggcttcagcacctattcagtagcccaggtgcctcgctggaagccccaggcccgcgcaccacagcccatccccagaagatcctggtcccctgctttaaccatcgaaaatgagcacatccgggcaacgtttgatcctgacacagggctgttgatggagattatgaacatgaatcagcaactcctgctgcctgttcgccagaccttcttctggtacaacgccagtataggtgacaacgaaagtgaccaggcctcaggtgcctacatcttcagacccaaccaacagaaaccgctgcctgtgagccgctgggctcagatccacctggtgaagacacccttggtgcaggaggtgcaccagaacttctcagcttggtgttcccaggtggttcgcctgtacccaggacagcggcacctggagctagagtggtcggtggggccgatacctgtgggcgacacctgggggaaggaggtcatcagccgttttgacacaccgctggagacaaagggacgcttctacacagacagcaatggccgggagatcctggagaggaggcgggattatcgacccacctggaaactgaaccagacggagcccgtggcaggaaactactatccagtcaacacccggatttacatcacggatggaaacatgcagctgactgtgctgactgaccgctcccaggggggcagcagcctgagagatggctcgctggagctcatggtgcaccgaaggctgctgaaggacgatggacgcggagtatcggagccactaatggagaacgggtcgggggcgtgggtgcgagggcgccacctggtgctgctggacacagcccaggctgcagccgccggacaccggctcctggcggagcaggaggtcctggcccctcaggtggtgctggccccgggtggcggcgccgcctacaatctcggggctcctccgcgcacgcagttctcagggctgcgcagggacctgccgccctcggtgcacctgctcacgctggccagctggggccccgaaatggtgctgctgcgcttggagcaccagtttgccgtaggagaggattccggacgtaacctgagcgcccccgttaccttgaacttgagggacctgttctccaccttcaccatcacccgcctgcaggagaccacgctggtggccaaccagctccgcgaggcagcctccaggctcaagtggacaacaaacacaggccccacaccccaccaaactccgtaccagctggacccggccaacatcacgctggaacccatggaaatccgcactttcctggcctcagttcaatggaaggaggtggatggttaggtctgctgggtcgac

SEQ ID NO: 25 - DNA, 3602 bp, synthetic construct
  source 1..3602: mol_type = other DNA
atcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatccgccgccaccatgggcgcctacgcaagggcatccggcgtgtgcgcaaggggctgtctggactctgccggaccatggaccatgagcagagccctgaggccacctctgccaccactgtgcttctttctgctgctgctggcagcagcaggagcaagggcaggaggctatgagacatgtcctaccgtgcagccaaacatgctgaatgtgcacctgctgcctcacacccacgacgatgtgggctggctgaagacagtggaccagtacttttatggcatcaagaatgatatccagcacgccggcgtgcagtacatcctggactctgtgatcagcgccctgctggccgatccaacccggagattcatctatgtggagatcgccttctttagcagatggtggcaccagcagacaaacgccactcaggaggtggtgagggatctggtgcgccagggccggctggagtttgcaaacggaggatgggtcatgaatgatgaggccgccacacactacggcgccatcgtggaccagatgaccctgggcctgcgctttctggaggacacattcggcaacgatggcagacccagggtggcatggcacatcgatcctttcggccactctcgggagcaggccagcctgtttgcccagatgggcttcgacggcttctttttcggccgcctggactaccaggataagtgggtgcggatgcagaagctggagatggagcaagtgtggagggcctctaccagcctgaagcctccaaccgccgacctgtttacaggcgtgctgcctaacggctataatccccctcgcaatctgtgctgggacgtgctgtgcgtggaccagccactggtggaggacccccggagcccagagtacaacgccaaggagctggtggactatttcctgaatgtggccaccgcccagggcagatactataggacaaaccacaccgtgatgacaatgggctccgattttcagtacgagaacgccaatatgtggttcaagaacctggacaagctgatccgcctggtgaatgcacagcaggcaaagggcagctccgtgcacgtgctgtacagcaccccagcctgctatctgtgggagctgaacaaggccaatctgacatggtccgtgaagcacgacgacttcttcccatacgccgatggcccccaccagttttggaccggctatttctctagccggcccgccctgaagcggtacgagcggctgtcctataacttcctgcaggtgtgcaatcagctggaggccctggtgggactggcagcaaacgtgggaccatacggctccggcgactctgcccccctgaatgaggccatggccgtgctgcagcaccacgatgccgtgagcggcacctccagacagcacgtggccaacgactatgcaaggcagctggcagcaggatggggaccatgcgaggtgctgctgtctaatgccctggccagactgaggggctttaaggatcactttaccttctgccagcagctgaacatctccatctgtcctctgtctcagacagccgccagattccaggtcatcgtgtacaaccctctgggcagaaaagtgaattggatggtgaggctgccagtgagcgagggcgtgtttgtggtgaaggaccccaatggcaggaccgtgccttctgacgtggtcatcttcccatcctctgatagccaggcccacccacccgagctgctgttttccgcctctctgcccgccctgggcttctctacctatagcgtggcccaggtgccaaggtggaagcctcaggcaagggcaccacagccaatccctaggcgcagctggtcccccgccctgacaatcgagaacgagcacatcagagccaccttcgaccctgatacaggcctgctgatggagatcatgaacatgaatcagcagctgctgctgcccgtgaggcagacctttttctggtacaacgcctccatcggcgacaatgagtccgatcaggcctctggcgcctatatctttagacccaatcagcagaagccactgcccgtgtctagatgggcccagatccacctggtgaagacacctctggtgcaggaggtgcaccagaacttcagcgcctggtgttcccaggtggtgagactgtaccctggccagaggcacctggagctggagtggagcgtgggacctatcccagtgggcgatacctggggcaaggaagtgatctccagatttgacacccctctggagacaaagggccgcttctacacagattctaacggccgggagatcctggagcggagaagggactatagaccaacctggaagctgaatcagacagagcctgtggccggcaactactatccagtgaataccaggatctatatcacagacggcaatatgcagctgaccgtgctgacagatagatcccagggaggcagctccctgagggacggctctctggagctgatggtgcaccgccggctgctgaaggacgatggaaggggcgtgtccgagccactgatggagaacggctctggagcatgggtgcgcggccggcacctggtgctgctggataccgcacaggcagcagcagcaggacacaggctgctggccgagcaggaggtgctggccccacaggtggtgctggcaccaggaggaggagcagcctacaatctgggagcacctccaaggacccagttttccggactgagaagggacctgccacctagcgtgcacctgctgacactggcatcctggggaccagagatggtgctgctgaggctggagcaccagttcgcagtgggagaggatagcggaaggaacctgtccgccccagtgaccctgaatctgagggacctgttttctaccttcacaatcacccggctgcaggagaccacactggtggccaaccagctgagagaggccgccagcaggctgaagtggaccacaaataccggccccacacctcaccagacaccttaccagctggaccccgccaacatcaccctggagcccatggagatcagaacattcctggccagcgtgcagtggaaggaggtggacggctgagtctgctgggtcgac

SEQ ID NO: 26 - DNA, 40 bp, synthetic construct
  source 1..40: mol_type = other DNA
gactcactatttgttttcgcgcccagttgcaaaaagtgtc

SEQ ID NO: 27 - DNA, 2640 bp, synthetic construct
  source 1..2640: mol_type = other DNA
atcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatctgactcactatttgttttcgcgcccagttgcaaaaagtgtcgccgccaccatgccggggttcctggttcgcatcctccctctgttgctggttctgctgcttctgggccctacgcgcggcttgcgcaatgccacccagaggatgtttgaaattgactatagccgggactccttcctcaaggatggccagccatttcgctacatctcaggaagcattcactactcccgtgtgccccgcttctactggaaggaccggctgctgaagatgaagatggctgggctgaacgccatccagacgtatgtgccctggaactttcatgagccctggccaggacagtaccagttttctgaggaccatgatgtggaatattttcttcggctggctcatgagctgggactgctggttatcctgaggcccgggccctacatctgtgcagagtgggaaatgggaggattacctgcttggctgctagagaaagagtctattcttctccgctcctccgacccagattacctggcagctgtggacaagtggttgggagtccttctgcccaagatgaagcctctcctctatcagaatggagggccagttataacagtgcaggttgaaaatgaatatggcagctactttgcctgtgattttgactacctgcgcttcctgcagaagcgctttcgccaccatctgggggatgatgtggttctgtttaccactgatggagcacataaaacattcctgaaatgtggggccctgcagggcctctacaccacggtggactttggaacaggcagcaacatcacagatgctttcctaagccagaggaagtgtgagcccaaaggacccttgatcaattctgaattctatactggctggctagatcactggggccaacctcactccacaatcaagaccgaagcagtggcttcctccctctatgatatacttgcccgtggggcgagtgtgaacttgtacatgtttataggtgggaccaattttgcctattggaatggggccaactcaccctatgcagcacagcccaccagctacgactatgatgccccactgagtgaggctggggacctcactgagaagtattttgctctgcgaaacatcatccagaagtttgaaaaagtaccagaaggtcctatccctccatctacaccaaagtttgcatatggaaaggtcactttggaaaagttaaagacagtgggagcagctctggacattctgtgtccctctgggcccatcaaaagcctttatcccttgacatttatccaggtgaaacagcattatgggtttgtgctgtaccggacaacacttcctcaagattgcagcaacccagcacctctctcttcacccctcaatggagtccacgatcgagcatatgttgctgtggatgggatcccccagggagtccttgagcgaaacaatgtgatcactctgaacataacagggaaagctggagccactctggaccttctggtagagaacatgggacgtgtgaactatggtgcatatatcaacgattttaagggtttggtttctaacctgactctcagttccaatatcctcacggactggacgatctttccactggacactgaggatgcagtgcgcagccacctggggggctggggacaccgtgacagtggccaccatgatgaagcctgggcccacaactcatccaactacacgctcccggccttttatatggggaacttctccattcccagtgggatcccagacttgccccaggacacctttatccagtttcctggatggaccaagggccaggtctggattaatggctttaaccttggccgctattggccagcccggggccctcagttgaccttgtttgtgccccagcacatcctgatgacctcggccccaaacaccatcaccgtgctggaactggagtgggcaccctgcagcagtgatgatccagaactatgtgctgtgacgttcgtggacaggccagttattggctcatctgtgacctacgatcatccctccaaacctgttgaaaaaagactcatgcccccacccccgcaaaaaaacaaagattcatggctggaccatgtatgatgatgaaaggtcgac

SEQ ID NO: 28 - DNA, 9144 bp, synthetic construct
  source 1..9144: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatatcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatctgccgccaccatgccggggttcctggttcgcatcctccctctgttgctggttctgctgcttctgggccctacgcgcggcttgcgcaatgccacccagaggatgtttgaaattgactatagccgggactccttcctcaaggatggccagccatttcgctacatctcaggaagcattcactactcccgtgtgccccgcttctactggaaggaccggctgctgaagatgaagatggctgggctgaacgccatccagacgtatgtgccctggaactttcatgagccctggccaggacagtaccagttttctgaggaccatgatgtggaatattttcttcggctggctcatgagctgggactgctggttatcctgaggcccgggccctacatctgtgcagagtgggaaatgggaggattacctgcttggctgctagagaaagagtctattcttctccgctcctccgacccagattacctggcagctgtggacaagtggttgggagtccttctgcccaagatgaagcctctcctctatcagaatggagggccagttataacagtgcaggttgaaaatgaatatggcagctactttgcctgtgattttgactacctgcgcttcctgcagaagcgctttcgccaccatctgggggatgatgtggttctgtttaccactgatggagcacataaaacattcctgaaatgtggggccctgcagggcctctacaccacggtggactttggaacaggcagcaacatcacagatgctttcctaagccagaggaagtgtgagcccaaaggacccttgatcaattctgaattctatactggctggctagatcactggggccaacctcactccacaatcaagaccgaagcagtggcttcctccctctatgatatacttgcccgtggggcgagtgtgaacttgtacatgtttataggtgggaccaattttgcctattggaatggggccaactcaccctatgcagcacagcccaccagctacgactatgatgccccactgagtgaggctggggacctcactgagaagtattttgctctgcgaaacatcatccagaagtttgaaaaagtaccagaaggtcctatccctccatctacaccaaagtttgcatatggaaaggtcactttggaaaagttaaagacagtgggagcagctctggacattctgtgtccctctgggcccatcaaaagcctttatcccttgacatttatccaggtgaaacagcattatgggtttgtgctgtaccggacaacacttcctcaagattgcagcaacccagcacctctctcttcacccctcaatggagtccacgatcgagcatatgttgctgtggatgggatcccccagggagtccttgagcgaaacaatgtgatcactctgaacataacagggaaagctggagccactctggaccttctggtagagaacatgggacgtgtgaactatggtgcatatatcaacgattttaagggtttggtttctaacctgactctcagttccaatatcctcacggactggacgatctttccactggacactgaggatgcagtgcgcagccacctggggggctggggacaccgtgacagtggccaccatgatgaagcctgggcccacaactcatccaactacacgctcccggccttttatatggggaacttctccattcccagtgggatcccagacttgccccaggacacctttatccagtttcctggatggaccaagggccaggtctggattaatggctttaaccttggccgctattggccagcccggggccctcagttgaccttgtttgtgccccagcacatcctgatgacctcggccccaaacaccatcaccgtgctggaactggagtgggcaccctgcagcagtgatgatccagaactatgtgctgtgacgttcgtggacaggccagttattggctcatctgtgacctacgatcatccctccaaacctgttgaaaaaagactcatgcccccacccccgcaaaaaaacaaagattcatggctggaccatgtatgatgatgaaaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 29 - DNA, 9144 bp, synthetic construct
  source 1..9144: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatatcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatccgccgccaccatgccgggtttcctggtgcgtatcctgccgctgctgctggttctgctgctgctgggtccgacccgtggcctgcgtaacgcgacccagcgtatgtttgagattgactacagccgtgatagcttcctgaaggatggtcaaccgtttcgttacatcagcggcagcattcactatagccgtgtgccgcgtttctactggaaagaccgtctgctgaagatgaaaatggcgggtctgaacgcgatccagacctatgttccgtggaactttcacgagccgtggccgggtcaatatcaattcagcgaggaccacgatgtggaatactttctgcgtctggcgcacgaactgggtctgctggttatcctgcgtccgggcccgtacatttgcgcggagtgggaaatgggtggcctgccggcgtggctgctggagaaggaaagcatcctgctgcgtagcagcgacccggattatctggcggcggtggataaatggctgggtgttctgctgccgaagatgaaaccgctgctgtaccagaacggtggcccggtgattaccgtgcaagttgagaacgaatacggcagctatttcgcgtgcgactttgattacctgcgtttcctgcagaagcgttttcgtcaccacctgggtgacgatgtggttctgttcaccaccgacggcgcgcacaagacctttctgaaatgcggtgcgctgcagggcctgtataccaccgttgacttcggtaccggcagcaacatcaccgatgcgtttctgagccagcgtaagtgcgagccgaaaggtccgctgattaacagcgaattctacaccggttggctggatcactggggccaaccgcacagcaccatcaagaccgaggcggtggcgagcagcctgtatgacattctggcgcgtggtgcgagcgttaacctgtacatgttcatcggtggcaccaactttgcgtattggaacggtgcgaacagcccgtatgcggcgcagccgaccagctacgactatgatgcgccgctgagcgaggcgggtgacctgaccgaaaaatatttcgcgctgcgtaacatcattcaaaagtttgagaaagttccggaaggtccgatcccgccgagcaccccgaagtttgcgtacggcaaagtgaccctggaaaagctgaaaaccgttggtgcggcgctggatatcctgtgcccgagcggtccgattaagagcctgtatccgctgaccttcattcaggtgaaacaacactacggttttgttctgtatcgtaccaccctgccgcaggactgcagcaacccggcgccgctgagcagcccgctgaacggcgttcacgaccgtgcgtacgtggcggttgatggtatcccgcaaggcgtgctggagcgtaacaacgttatcaccctgaacattaccggtaaagcgggtgcgaccctggacctgctggtggaaaacatgggtcgtgttaactacggcgcgtatattaacgatttcaaaggtctggtgagcaacctgaccctgagcagcaacatcctgaccgactggaccatttttccgctggacaccgaggatgcggttcgtagccacctgggtggctggggtcaccgtgacagcggtcaccacgatgaagcgtgggcgcacaacagcagcaactacaccctgccggcgttctatatgggtaactttagcattccgagcggcattccggacctgccgcaggataccttcatccaatttccgggttggaccaaaggccaagtgtggattaacggtttcaacctgggtcgttattggccggcgcgtggtccgcagctgaccctgtttgtgccgcaacacatcctgatgaccagcgcgccgaacaccattaccgttctggagctggaatgggcgccgtgcagcagcgatgatccggagctgtgcgcggtgaccttcgttgaccgtccggtgatcggcagcagcgttacctacgatcacccgagcaagccggtggaaaaacgtctgatgccgccgccgccgcagaagaacaaagacagctggctggatcacgtttaatgatgaaaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 30 - DNA, 9226 bp, synthetic construct
  source 1..9226: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatatcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatccagagcgcccactgcctaacggagagaccccatcgtggcgcgatcatgctccgggtccccctgtgtacgccgctcccgctcctggcactgctgcaactgctgggcgctgcgcacggcatctataatgtcacccagaggacatttaagctcgactacagccgggaccgcttcctcaaggatggacagccattccgatacatctcgggaagcattcattacttccggataccccgcttctactgggaggaccggctgctgaagatgaagatggctgggctgaatgctatccagatgtacgtgccctggaacttccatgaaccccaaccaggacaatatgagttttctggggaccgtgatgtggagcatttcatccagctggctcatgagctgggactcctggtgatcctgaggcctgggccctacatctgtgcagagtgggacatggggggcttacctgcttggctactagagaaacaatctatcgttctccggtcttctgacccagactaccttgtagctgtggataaatggctggcagtccttctgcccaagatgaagcccctgctctaccagaacggaggaccgatcataaccgtgcaggttgagaatgagtacgggtcctactttgcctgcgattacgactacctacgcttcctggtgcaccgcttccgctaccatctgggtaatgacgtcattctcttcaccaccgacggagcaagtgaaaaaatgctgaagtgtgggaccctgcaggacctgtacgccacagtggattttggaacaggcaacaatatcacacaagctttcctggtccagaggaagtttgaacctaaaggacctttgatcaattctgagttctatactggctggctagaccactggggtaaaccccattccacggtgaaaactaaaacactggctacctccctctataacctgcttgcccgtggggccaacgtgaacttgtacatgtttataggtgggaccaattttgcctattggaatggtgccaacacgccctatgagccacagcccaccagctatgactacgacgccccactgagcgaggctggggacctcactaagaagtattttgctcttcgagaagtcattcagatgtttaaagaagtcccagaaggccctatccctccgtctacacccaaatttgcatatggaaaagttgctctgagaaagttcaagacagtggctgaagctctgggtatcctgtgtcccaatgggccagtgaaaagcctctatcccctgacattcactcaggtaaaacagtattttgggtatgtgctgtaccgaacaacgcttcctcaagattgcagtaacccgaaacccattttctcttcacccttcaatggtgtccgtgatcgggcttacgtctctgtggacggggtcccccaaggaatccttgatcgaaacctcatgacagctctgaacatacaggggaaggctggagccacgctggacatcctggtggagaacatggggcgtgtgaactatggcagattcatcaatgacttcaagggtttgatttccaacatgactatcaactccactgtcctcaccaactggacggtcttcccactggacactgaggccatggtacgcaaccatctctggggccgggaggccagtgatgggggtcaccttgacggacggtcgacctccaattcttcggacctcatactccccaccttttacgtgggcaacttctccatcccctcgggcatcccagacctgccacaggacaccttcatccagtttcctgggtggtccaagggtcaagtatggatcaatggctttaacctcggccgatactggcccacaatgggcccacaaaagaccttgttcgtgccaaggaacatcctgaccacttcagccccaaacaacatcacagtgttggagctagagtttgcaccctgcagcgaggggaccccagagctgtgtacagtagagtttgttgacactccggtcatttcctgacctgacttgaccatcggtggccattttccaagccagtctggtcaagactcatggctgaacctctgagactgagccttggggagcacagctctactctggttacacggatcacctttgttgtgctagaatggaagctacatctcgaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 31 - DNA, 10146 bp, synthetic construct
  source 1..10146: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatatcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatccgccgccaccatgggcgcctacgcgcgggcttcgggggtctgcgctcgcggctgcctggactcagcaggcccctggaccatgtcccgcgccctgcggccaccgctcccgcctctctgctttttccttttgttgctggcggctgccggtgctcgggccgggggatacgagacatgccccacagtgcagccgaacatgctgaacgtgcacctgctgcctcacacacatgatgacgtgggctggctcaaaaccgtggaccagtacttttatggaatcaagaatgacatccagcacgccggtgtgcagtacatcctggactcggtcatctctgccttgctggcagatcccacccgtcgcttcatttacgtggagattgccttcttctcccgttggtggcaccagcagacaaatgccacacaggaagtcgtgcgagaccttgtgcgccaggggcgcctggagttcgccaatggtggctgggtgatgaacgatgaggcagccacccactacggtgccatcgtggaccagatgacacttgggctgcgctttctggaggacacatttggcaatgatgggcgaccccgtgtggcctggcacattgaccccttcggccactctcgggagcaggcctcgctgtttgcgcagatgggcttcgacggcttcttctttgggcgccttgattatcaagataagtgggtacggatgcagaagctggagatggagcaggtgtggcgggccagcaccagcctgaagcccccgaccgcggacctcttcactggtgtgcttcccaatggttacaacccgccaaggaatctgtgctgggatgtgctgtgtgtcgatcagccgctggtggaggaccctcgcagccccgagtacaacgccaaggagctggtcgattacttcctaaatgtggccactgcccagggccggtattaccgcaccaaccacactgtgatgaccatgggctcggacttccaatatgagaatgccaacatgtggttcaagaaccttgacaagctcatccggctggtaaatgcgcagcaggcaaaaggaagcagtgtccatgttctctactccacccccgcttgttacctctgggagctgaacaaggccaacctcacctggtcagtgaaacatgacgacttcttcccttacgcggatggcccccaccagttctggaccggttacttttccagtcggccggccctcaaacgctacgagcgcctcagctacaacttcctgcaggtgtgcaaccagctggaggcgctggtgggcctggcggccaacgtgggaccctatggctccggagacagtgcacccctcaatgaggcgatggctgtgctccagcatcacgacgccgtcagcggcacctcccgccagcacgtggccaacgactacgcgcgccagcttgcggcaggctgggggccttgcgaggttcttctgagcaacgcgctggcgcggctcagaggcttcaaagatcacttcaccttttgccaacagctaaacatcagcatctgcccgctcagccagacggcggcgcgcttccaggtcatcgtttataatcccctggggcggaaggtgaattggatggtacggctgccggtcagcgaaggcgttttcgttgtgaaggaccccaatggcaggacagtgcccagcgatgtggtaatatttcccagctcagacagccaggcgcaccctccggagctgctgttctcagcctcactgcccgccctgggcttcagcacctattcagtagcccaggtgcctcgctggaagccccaggcccgcgcaccacagcccatccccagaagatcctggtcccctgctttaaccatcgaaaatgagcacatccgggcaacgtttgatcctgacacagggctgttgatggagattatgaacatgaatcagcaactcctgctgcctgttcgccagaccttcttctggtacaacgccagtataggtgacaacgaaagtgaccaggcctcaggtgcctacatcttcagacccaaccaacagaaaccgctgcctgtgagccgctgggctcagatccacctggtgaagacacccttggtgcaggaggtgcaccagaacttctcagcttggtgttcccaggtggttcgcctgtacccaggacagcggcacctggagctagagtggtcggtggggccgatacctgtgggcgacacctgggggaaggaggtcatcagccgttttgacacaccgctggagacaaagggacgcttctacacagacagcaatggccgggagatcctggagaggaggcgggattatcgacccacctggaaactgaaccagacggagcccgtggcaggaaactactatccagtcaacacccggatttacatcacggatggaaacatgcagctgactgtgctgactgaccgctcccaggggggcagcagcctgagagatggctcgctggagctcatggtgcaccgaaggctgctgaaggacgatggacgcggagtatcggagccactaatggagaacgggtcgggggcgtgggtgcgagggcgccacctggtgctgctggacacagcccaggctgcagccgccggacaccggctcctggcggagcaggaggtcctggcccctcaggtggtgctggccccgggtggcggcgccgcctacaatctcggggctcctccgcgcacgcagttctcagggctgcgcagggacctgccgccctcggtgcacctgctcacgctggccagctggggccccgaaatggtgctgctgcgcttggagcaccagtttgccgtaggagaggattccggacgtaacctgagcgcccccgttaccttgaacttgagggacctgttctccaccttcaccatcacccgcctgcaggagaccacgctggtggccaaccagctccgcgaggcagcctccaggctcaagtggacaacaaacacaggccccacaccccaccaaactccgtaccagctggacccggccaacatcacgctggaacccatggaaatccgcactttcctggcctcagttcaatggaaggaggtggatggttaggtctgctgggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 32 - DNA, 10146 bp, synthetic construct
  source 1..10146: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatatcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatccgccgccaccatgggcgcctacgcaagggcatccggcgtgtgcgcaaggggctgtctggactctgccggaccatggaccatgagcagagccctgaggccacctctgccaccactgtgcttctttctgctgctgctggcagcagcaggagcaagggcaggaggctatgagacatgtcctaccgtgcagccaaacatgctgaatgtgcacctgctgcctcacacccacgacgatgtgggctggctgaagacagtggaccagtacttttatggcatcaagaatgatatccagcacgccggcgtgcagtacatcctggactctgtgatcagcgccctgctggccgatccaacccggagattcatctatgtggagatcgccttctttagcagatggtggcaccagcagacaaacgccactcaggaggtggtgagggatctggtgcgccagggccggctggagtttgcaaacggaggatgggtcatgaatgatgaggccgccacacactacggcgccatcgtggaccagatgaccctgggcctgcgctttctggaggacacattcggcaacgatggcagacccagggtggcatggcacatcgatcctttcggccactctcgggagcaggccagcctgtttgcccagatgggcttcgacggcttctttttcggccgcctggactaccaggataagtgggtgcggatgcagaagctggagatggagcaagtgtggagggcctctaccagcctgaagcctccaaccgccgacctgtttacaggcgtgctgcctaacggctataatccccctcgcaatctgtgctgggacgtgctgtgcgtggaccagccactggtggaggacccccggagcccagagtacaacgccaaggagctggtggactatttcctgaatgtggccaccgcccagggcagatactataggacaaaccacaccgtgatgacaatgggctccgattttcagtacgagaacgccaatatgtggttcaagaacctggacaagctgatccgcctggtgaatgcacagcaggcaaagggcagctccgtgcacgtgctgtacagcaccccagcctgctatctgtgggagctgaacaaggccaatctgacatggtccgtgaagcacgacgacttcttcccatacgccgatggcccccaccagttttggaccggctatttctctagccggcccgccctgaagcggtacgagcggctgtcctataacttcctgcaggtgtgcaatcagctggaggccctggtgggactggcagcaaacgtgggaccatacggctccggcgactctgcccccctgaatgaggccatggccgtgctgcagcaccacgatgccgtgagcggcacctccagacagcacgtggccaacgactatgcaaggcagctggcagcaggatggggaccatgcgaggtgctgctgtctaatgccctggccagactgaggggctttaaggatcactttaccttctgccagcagctgaacatctccatctgtcctctgtctcagacagccgccagattccaggtcatcgtgtacaaccctctgggcagaaaagtgaattggatggtgaggctgccagtgagcgagggcgtgtttgtggtgaaggaccccaatggcaggaccgtgccttctgacgtggtcatcttcccatcctctgatagccaggcccacccacccgagctgctgttttccgcctctctgcccgccctgggcttctctacctatagcgtggcccaggtgccaaggtggaagcctcaggcaagggcaccacagccaatccctaggcgcagctggtcccccgccctgacaatcgagaacgagcacatcagagccaccttcgaccctgatacaggcctgctgatggagatcatgaacatgaatcagcagctgctgctgcccgtgaggcagacctttttctggtacaacgcctccatcggcgacaatgagtccgatcaggcctctggcgcctatatctttagacccaatcagcagaagccactgcccgtgtctagatgggcccagatccacctggtgaagacacctctggtgcaggaggtgcaccagaacttcagcgcctggtgttcccaggtggtgagactgtaccctggccagaggcacctggagctggagtggagcgtgggacctatcccagtgggcgatacctggggcaaggaagtgatctccagatttgacacccctctggagacaaagggccgcttctacacagattctaacggccgggagatcctggagcggagaagggactatagaccaacctggaagctgaatcagacagagcctgtggccggcaactactatccagtgaataccaggatctatatcacagacggcaatatgcagctgaccgtgctgacagatagatcccagggaggcagctccctgagggacggctctctggagctgatggtgcaccgccggctgctgaaggacgatggaaggggcgtgtccgagccactgatggagaacggctctggagcatgggtgcgcggccggcacctggtgctgctggataccgcacaggcagcagcagcaggacacaggctgctggccgagcaggaggtgctggccccacaggtggtgctggcaccaggaggaggagcagcctacaatctgggagcacctccaaggacccagttttccggactgagaagggacctgccacctagcgtgcacctgctgacactggcatcctggggaccagagatggtgctgctgaggctggagcaccagttcgcagtgggagaggatagcggaaggaacctgtccgccccagtgaccctgaatctgagggacctgttttctaccttcacaatcacccggctgcaggagaccacactggtggccaaccagctgagagaggccgccagcaggctgaagtggaccacaaataccggccccacacctcaccagacaccttaccagctggaccccgccaacatcaccctggagcccatggagatcagaacattcctggccagcgtgcagtggaaggaggtggacggctgagtctgctgggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 33 - DNA, 9184 bp, synthetic construct
  source 1..9184: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatatcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatctgactcactatttgttttcgcgcccagttgcaaaaagtgtcgccgccaccatgccggggttcctggttcgcatcctccctctgttgctggttctgctgcttctgggccctacgcgcggcttgcgcaatgccacccagaggatgtttgaaattgactatagccgggactccttcctcaaggatggccagccatttcgctacatctcaggaagcattcactactcccgtgtgccccgcttctactggaaggaccggctgctgaagatgaagatggctgggctgaacgccatccagacgtatgtgccctggaactttcatgagccctggccaggacagtaccagttttctgaggaccatgatgtggaatattttcttcggctggctcatgagctgggactgctggttatcctgaggcccgggccctacatctgtgcagagtgggaaatgggaggattacctgcttggctgctagagaaagagtctattcttctccgctcctccgacccagattacctggcagctgtggacaagtggttgggagtccttctgcccaagatgaagcctctcctctatcagaatggagggccagttataacagtgcaggttgaaaatgaatatggcagctactttgcctgtgattttgactacctgcgcttcctgcagaagcgctttcgccaccatctgggggatgatgtggttctgtttaccactgatggagcacataaaacattcctgaaatgtggggccctgcagggcctctacaccacggtggactttggaacaggcagcaacatcacagatgctttcctaagccagaggaagtgtgagcccaaaggacccttgatcaattctgaattctatactggctggctagatcactggggccaacctcactccacaatcaagaccgaagcagtggcttcctccctctatgatatacttgcccgtggggcgagtgtgaacttgtacatgtttataggtgggaccaattttgcctattggaatggggccaactcaccctatgcagcacagcccaccagctacgactatgatgccccactgagtgaggctggggacctcactgagaagtattttgctctgcgaaacatcatccagaagtttgaaaaagtaccagaaggtcctatccctccatctacaccaaagtttgcatatggaaaggtcactttggaaaagttaaagacagtgggagcagctctggacattctgtgtccctctgggcccatcaaaagcctttatcccttgacatttatccaggtgaaacagcattatgggtttgtgctgtaccggacaacacttcctcaagattgcagcaacccagcacctctctcttcacccctcaatggagtccacgatcgagcatatgttgctgtggatgggatcccccagggagtccttgagcgaaacaatgtgatcactctgaacataacagggaaagctggagccactctggaccttctggtagagaacatgggacgtgtgaactatggtgcatatatcaacgattttaagggtttggtttctaacctgactctcagttccaatatcctcacggactggacgatctttccactggacactgaggatgcagtgcgcagccacctggggggctggggacaccgtgacagtggccaccatgatgaagcctgggcccacaactcatccaactacacgctcccggccttttatatggggaacttctccattcccagtgggatcccagacttgccccaggacacctttatccagtttcctggatggaccaagggccaggtctggattaatggctttaaccttggccgctattggccagcccggggccctcagttgaccttgtttgtgccccagcacatcctgatgacctcggccccaaacaccatcaccgtgctggaactggagtgggcaccctgcagcagtgatgatccagaactatgtgctgtgacgttcgtggacaggccagttattggctcatctgtgacctacgatcatccctccaaacctgttgaaaaaagactcatgcccccacccccgcaaaaaaacaaagattcatggctggaccatgtatgatgatgaaaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 34 - DNA, 9226 bp, synthetic construct
  source 1..9226: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatatcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatccagagcgcccactgcctaacggagagaccccatcgtggcgcgatcatgctccgggtccccctgtgtacgccgctcccgctcctggcactgctgcaactgctgggcgctgcgcacggcatctataatgtcacccagaggacatttaagctcgactacagccgggaccgcttcctcaaggatggacagccattccgatacatctcgggaagcattcattacttccggataccccgcttctactgggaggaccggctgctgaagatgaagatggctgggctgaatgctatccagatgtacgtgccctggaacttccatgaaccccaaccaggacaatatgagttttctggggaccgtgatgtggagcatttcatccagctggctcatgagctgggactcctggtgatcctgaggcctgggccctacatctgtgcagagtgggacatggggggcttacctgcttggctactagagaaacaatctatcgttctccggtcttctgacccagactaccttgtagctgtggataaatggctggcagtccttctgcccaagatgaagcccctgctctaccagaacggaggaccgatcataaccgtgcaggttgagaatgagtacgggtcctactttgcctgcgattacgactacctacgcttcctggtgcaccgcttccgctaccatctgggtaatgacgtcattctcttcaccaccgacggagcaagtgaaaaaatgctgaagtgtgggaccctgcaggacctgtacgccacagtggattttggaacaggcaacaatatcacacaagctttcctggtccagaggaagtttgaacctaaaggacctttgatcaattctgagttctatactggctggctagaccactggggtaaaccccattccacggtgaaaactaaaacactggctacctccctctataacctgcttgcccgtggggccaacgtgaacttgtacatgtttataggtgggaccaattttgcctattggaatggtgccaacacgccctatgagccacagcccaccagctatgactacgacgccccactgagcgaggctggggacctcactaagaagtattttgctcttcgagaagtcattcagatgtttaaagaagtcccagaaggccctatccctccgtctacacccaaatttgcatatggaaaagttgctctgagaaagttcaagacagtggctgaagctctgggtatcctgtgtcccaatgggccagtgaaaagcctctatcccctgacattcactcaggtaaaacagtattttgggtatgtgctgtaccgaacaacgcttcctcaagattgcagtaacccgaaacccattttctcttcacccttcaatggtgtccgtgatcgggcttacgtctctgtggacggggtcccccaaggaatccttgatcgaaacctcatgacagctctgaacatacgggggaaggctggagccacgctggacatcctggtggagaacatggggcgtgtgaactatggcagattcatcaatgacttcaagggtttgatttccaacatgactatcaactccactgtcctcaccaactggacggtcttcccactgaacactgaggccatggtacgcaaccatctctggggccgggaggccagtgatgagggtcaccttgacggacggtcgacctccaattcttcggacctcatactccccaccttttacgtgggcaacttctccatcccctcgggcatcccagacctgccacaggacaccttcatccagtttcctgggtggtccaagggtcaagtatggatcaatggctttaacctcggccgatactggcccacaatgggcccacaaaagaccttgttcgtgccaaggaacatcctgaccacttcagccccaaacaacatcacagtgttggagctagagtttgcaccctgcagcgaggggaccccagagctgtgtacagtagagtttgttgacactccggtcatttcctgacctgacttgaccatcggtggccattttccaagccagtctggtcaagactcatggctgaacctctgagactgagccttggggagcacagctctactctggttacacggatcacctttgttgtgctagaatggaagctacatctcgaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 35 - DNA, 1569 bp, Homo sapiens
  source 1..1569: mol_type = other DNA
atggcggcggttgtcgcggcgacgaggtggtggcagctgttgctggtgctcagcgccgcggggatgggggcctcgggcgccccgcagccccccaacatcctgctcctgctcatggacgacatgggatggggtgacctcggggtgtatggagagccctccagagagaccccgaatttggaccggatggctgcagaagggctgcttttcccaaacttctattctgccaaccctctgtgctcgccatcgagggcggcactgctcacaggacggctacccatccgcaatggcttctacaccaccaacgcccatgccagaaacgcctacacaccgcaggagattgtgggcggcatcccagactcggagcagctcctgccggagcttctgaagaaggccggctacgtcagcaagattgtcggcaagtggcatctgggtcacaggccccagttccaccccctgaagcacggatttgatgagtggtttggatcccccaactgccactttggaccttatgacaacaaggccaggcccaacatccctgtgtacagggactgggagatggttggcagatattatgaagaatttcctattaatctgaagacgggggaagccaacctcacccagatctacctgcaggaagccctggacttcattaagagacaggcacggcaccacccctttttcctctactgggctgtcgacgccacgcacgcacccgtctatgcctccaaacccttcttgggcaccagtcagcgagggcggtatggagacgccgtccgggagattgatgacagcattgggaagatactggagctcctccaagacctgcacgtcgcggacaacaccttcgtcttcttcacgtcggacaacggcgctgccctcatttccgcccccgaacaaggtggcagcaacggcccctttctgtgtgggaagcagaccacgtttgaaggagggatgagggagcctgccctcgcatggtggccagggcacgtcactgcaggccaggtgagccaccagctgggcagcatcatggacctcttcaccaccagcctggcccttgcgggcctgacgccgcccagcgacagggccattgatggcctcaacctcctccccaccctcctgcagggccggctgatggacaggcctatcttctattaccgtggcgacacgctgatggcggccaccctcgggcagcacaaggctcacttctggacctggaccaactcctgggagaacttcagacagggcattgatttctgccctgggcagaacgtttcaggggtcacaactcacaatctggaagaccacacgaagctgcccctgatcttccacctgggacgggacccaggggagaggttccccctcagctttgccagcgccgagtaccaggaggccctcagcaggatcacctcggtcgtccagcagcaccaggaggccttggtccccgcgcagccccagctcaacgtgtgcaactgggcggtcatgaactgggcacctccgggctgtgaaaagttagggaagtgtctgacacctccagaatccattcccaagaagtgcctctggtcccactag

SEQ ID NO: 36 - DNA, 1569 bp, synthetic construct
  source 1..1569: mol_type = other DNA
atggcggcggttgtggcggcgacccgttggtggcaactgctgctggttctgagcgcggcgggcatgggtgcgagcggtgcgccgcaaccgccaaacatcctgctgctgctgatggacgatatgggttggggcgacctgggtgtgtatggtgaaccgagccgtgaaaccccgaacctggatcgtatggcggcggagggtctgctgttcccgaacttttatagcgcgaacccgctgtgcagcccgagccgtgcggcgctgctgaccggtcgtctgccgatccgtaacggcttctacaccaccaacgcgcacgcgcgtaacgcgtataccccgcaggaaatcgttggtggcattccggacagcgagcaactgctgccggaactgctgaagaaagcgggttacgtgagcaagattgttggtaaatggcatctgggtcaccgtccgcagttccacccgctgaagcacggtttcgatgagtggtttggcagcccgaactgccactttggcccgtacgacaacaaagcgcgtccgaacatcccggtgtatcgtgattgggaaatggttggtcgttactatgaggaattcccgattaacctgaagaccggcgaggcgaacctgacccagatctacctgcaagaagcgctggactttattaaacgtcaggcgcgtcaccacccgttctttctgtattgggcggtggatgcgacccatgcgccggtttatgcgagcaagccgttcctgggtaccagccagcgtggtcgttatggcgacgcggtgcgtgagatcgacgatagcatcggcaaaattctggaactgctgcaagacctgcacgtggcggataacacctttgttttctttaccagcgataacggtgcggcgctgattagcgcgccggagcagggtggcagcaacggtccgttcctgtgcggcaagcaaaccacctttgagggtggcatgcgtgaaccggcgctggcgtggtggccgggtcacgtgaccgcgggccaggttagccaccaactgggtagcatcatggacctgttcaccaccagcctggcgctggcgggcctgaccccgccgagcgaccgtgcgattgatggtctgaacctgctgccgaccctgctgcagggccgtctgatggaccgtccgattttctactatcgtggtgataccctgatggcggcgaccctgggtcaacacaaggcgcacttttggacctggaccaacagctgggaaaacttccgtcagggtatcgatttttgtccgggtcaaaacgtgagcggcgttaccacccacaacctggaggaccacaccaaactgccgctgatttttcacctgggtcgtgatccgggcgaacgtttcccgctgagctttgcgagcgcggagtaccaggaagcgctgagccgtattaccagcgtggttcagcaacatcaagaggcgctggttccggcgcagccgcaactgaacgtgtgcaactgggcggttatgaactgggcgccgccgggttgcgaaaaactgggcaagtgcctgaccccgccggaaagcatcccgaaaaagtgcctgtggagccattaa

SEQ ID NO: 37 - DNA, 2134 bp, synthetic construct
  source 1..2134: mol_type = other DNA
atcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggggggccgccaccatggcggcggttgtcgcggcgacgaggtggtggcagctgttgctggtgctcagcgccgcggggatgggggcctcgggcgccccgcagccccccaacatcctgctcctgctcatggacgacatgggatggggtgacctcggggtgtatggagagccctccagagagaccccgaatttggaccggatggctgcagaagggctgcttttcccaaacttctattctgccaaccctctgtgctcgccatcgagggcggcactgctcacaggacggctacccatccgcaatggcttctacaccaccaacgcccatgccagaaacgcctacacaccgcaggagattgtgggcggcatcccagactcggagcagctcctgccggagcttctgaagaaggccggctacgtcagcaagattgtcggcaagtggcatctgggtcacaggccccagttccaccccctgaagcacggatttgatgagtggtttggatcccccaactgccactttggaccttatgacaacaaggccaggcccaacatccctgtgtacagggactgggagatggttggcagatattatgaagaatttcctattaatctgaagacgggggaagccaacctcacccagatctacctgcaggaagccctggacttcattaagagacaggcacggcaccacccctttttcctctactgggctgtcgacgccacgcacgcacccgtctatgcctccaaacccttcttgggcaccagtcagcgagggcggtatggagacgccgtccgggagattgatgacagcattgggaagatactggagctcctccaagacctgcacgtcgcggacaacaccttcgtcttcttcacgtcggacaacggcgctgccctcatttccgcccccgaacaaggtggcagcaacggcccctttctgtgtgggaagcagaccacgtttgaaggagggatgagggagcctgccctcgcatggtggccagggcacgtcactgcaggccaggtgagccaccagctgggcagcatcatggacctcttcaccaccagcctggcccttgcgggcctgacgccgcccagcgacagggccattgatggcctcaacctcctccccaccctcctgcagggccggctgatggacaggcctatcttctattaccgtggcgacacgctgatggcggccaccctcgggcagcacaaggctcacttctggacctggaccaactcctgggagaacttcagacagggcattgatttctgccctgggcagaacgtttcaggggtcacaactcacaatctggaagaccacacgaagctgcccctgatcttccacctgggacgggacccaggggagaggttccccctcagctttgccagcgccgagtaccaggaggccctcagcaggatcacctcggtcgtccagcagcaccaggaggccttggtccccgcgcagccccagctcaacgtgtgcaactgggcggtcatgaactgggcacctccgggctgtgaaaagttagggaagtgtctgacacctccagaatccattcccaagaagtgcctctggtcccactagcacctgcgcctcgag

SEQ ID NO: 38 - DNA, 2135 bp, synthetic construct
  source 1..2135: mol_type = other DNA
atcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatccgccgccaccatggcggcggttgtggcggcgacccgttggtggcaactgctgctggttctgagcgcggcgggcatgggtgcgagcggtgcgccgcaaccgccaaacatcctgctgctgctgatggacgatatgggttggggcgacctgggtgtgtatggtgaaccgagccgtgaaaccccgaacctggatcgtatggcggcggagggtctgctgttcccgaacttttatagcgcgaacccgctgtgcagcccgagccgtgcggcgctgctgaccggtcgtctgccgatccgtaacggcttctacaccaccaacgcgcacgcgcgtaacgcgtataccccgcaggaaatcgttggtggcattccggacagcgagcaactgctgccggaactgctgaagaaagcgggttacgtgagcaagattgttggtaaatggcatctgggtcaccgtccgcagttccacccgctgaagcacggtttcgatgagtggtttggcagcccgaactgccactttggcccgtacgacaacaaagcgcgtccgaacatcccggtgtatcgtgattgggaaatggttggtcgttactatgaggaattcccgattaacctgaagaccggcgaggcgaacctgacccagatctacctgcaagaagcgctggactttattaaacgtcaggcgcgtcaccacccgttctttctgtattgggcggtggatgcgacccatgcgccggtttatgcgagcaagccgttcctgggtaccagccagcgtggtcgttatggcgacgcggtgcgtgagatcgacgatagcatcggcaaaattctggaactgctgcaagacctgcacgtggcggataacacctttgttttctttaccagcgataacggtgcggcgctgattagcgcgccggagcagggtggcagcaacggtccgttcctgtgcggcaagcaaaccacctttgagggtggcatgcgtgaaccggcgctggcgtggtggccgggtcacgtgaccgcgggccaggttagccaccaactgggtagcatcatggacctgttcaccaccagcctggcgctggcgggcctgaccccgccgagcgaccgtgcgattgatggtctgaacctgctgccgaccctgctgcagggccgtctgatggaccgtccgattttctactatcgtggtgataccctgatggcggcgaccctgggtcaacacaaggcgcacttttggacctggaccaacagctgggaaaacttccgtcagggtatcgatttttgtccgggtcaaaacgtgagcggcgttaccacccacaacctggaggaccacaccaaactgccgctgatttttcacctgggtcgtgatccgggcgaacgtttcccgctgagctttgcgagcgcggagtaccaggaagcgctgagccgtattaccagcgtggttcagcaacatcaagaggcgctggttccggcgcagccgcaactgaacgtgtgcaactgggcggttatgaactgggcgccgccgggttgcgaaaaactgggcaagtgcctgaccccgccggaaagcatcccgaaaaagtgcctgtggagccattaacacctgcgcgtcgac

SEQ ID NO: 39 - DNA, 8684 bp, synthetic construct
  source 1..8684: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatatcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggggggccgccaccatggcggcggttgtcgcggcgacgaggtggtggcagctgttgctggtgctcagcgccgcggggatgggggcctcgggcgccccgcagccccccaacatcctgctcctgctcatggacgacatgggatggggtgacctcggggtgtatggagagccctccagagagaccccgaatttggaccggatggctgcagaagggctgcttttcccaaacttctattctgccaaccctctgtgctcgccatcgagggcggcactgctcacaggacggctacccatccgcaatggcttctacaccaccaacgcccatgccagaaacgcctacacaccgcaggagattgtgggcggcatcccagactcggagcagctcctgccggagcttctgaagaaggccggctacgtcagcaagattgtcggcaagtggcatctgggtcacaggccccagttccaccccctgaagcacggatttgatgagtggtttggatcccccaactgccactttggaccttatgacaacaaggccaggcccaacatccctgtgtacagggactgggagatggttggcagatattatgaagaatttcctattaatctgaagacgggggaagccaacctcacccagatctacctgcaggaagccctggacttcattaagagacaggcacggcaccacccctttttcctctactgggctgtcgacgccacgcacgcacccgtctatgcctccaaacccttcttgggcaccagtcagcgagggcggtatggagacgccgtccgggagattgatgacagcattgggaagatactggagctcctccaagacctgcacgtcgcggacaacaccttcgtcttcttcacgtcggacaacggcgctgccctcatttccgcccccgaacaaggtggcagcaacggcccctttctgtgtgggaagcagaccacgtttgaaggagggatgagggagcctgccctcgcatggtggccagggcacgtcactgcaggccaggtgagccaccagctgggcagcatcatggacctcttcaccaccagcctggcccttgcgggcctgacgccgcccagcgacagggccattgatggcctcaacctcctccccaccctcctgcagggccggctgatggacaggcctatcttctattaccgtggcgacacgctgatggcggccaccctcgggcagcacaaggctcacttctggacctggaccaactcctgggagaacttcagacagggcattgatttctgccctgggcagaacgtttcaggggtcacaactcacaatctggaagaccacacgaagctgcccctgatcttccacctgggacgggacccaggggagaggttccccctcagctttgccagcgccgagtaccaggaggccctcagcaggatcacctcggtcgtccagcagcaccaggaggccttggtccccgcgcagccccagctcaacgtgtgcaactgggcggtcatgaactgggcacctccgggctgtgaaaagttagggaagtgtctgacacctccagaatccattcccaagaagtgcctctggtcccactagcacctgcgcctcgaggtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 40 - DNA, 8679 bp, synthetic construct
  source 1..8679: mol_type = other DNA
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgatatcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatccgccgccaccatggcggcggttgtggcggcgacccgttggtggcaactgctgctggttctgagcgcggcgggcatgggtgcgagcggtgcgccgcaaccgccaaacatcctgctgctgctgatggacgatatgggttggggcgacctgggtgtgtatggtgaaccgagccgtgaaaccccgaacctggatcgtatggcggcggagggtctgctgttcccgaacttttatagcgcgaacccgctgtgcagcccgagccgtgcggcgctgctgaccggtcgtctgccgatccgtaacggcttctacaccaccaacgcgcacgcgcgtaacgcgtataccccgcaggaaatcgttggtggcattccggacagcgagcaactgctgccggaactgctgaagaaagcgggttacgtgagcaagattgttggtaaatggcatctgggtcaccgtccgcagttccacccgctgaagcacggtttcgatgagtggtttggcagcccgaactgccactttggcccgtacgacaacaaagcgcgtccgaacatcccggtgtatcgtgattgggaaatggttggtcgttactatgaggaattcccgattaacctgaagaccggcgaggcgaacctgacccagatctacctgcaagaagcgctggactttattaaacgtcaggcgcgtcaccacccgttctttctgtattgggcggtggatgcgacccatgcgccggtttatgcgagcaagccgttcctgggtaccagccagcgtggtcgttatggcgacgcggtgcgtgagatcgacgatagcatcggcaaaattctggaactgctgcaagacctgcacgtggcggataacacctttgttttctttaccagcgataacggtgcggcgctgattagcgcgccggagcagggtggcagcaacggtccgttcctgtgcggcaagcaaaccacctttgagggtggcatgcgtgaaccggcgctggcgtggtggccgggtcacgtgaccgcgggccaggttagccaccaactgggtagcatcatggacctgttcaccaccagcctggcgctggcgggcctgaccccgccgagcgaccgtgcgattgatggtctgaacctgctgccgaccctgctgcagggccgtctgatggaccgtccgattttctactatcgtggtgataccctgatggcggcgaccctgggtcaacacaaggcgcacttttggacctggaccaacagctgggaaaacttccgtcagggtatcgatttttgtccgggtcaaaacgtgagcggcgttaccacccacaacctggaggaccacaccaaactgccgctgatttttcacctgggtcgtgatccgggcgaacgtttcccgctgagctttgcgagcgcggagtaccaggaagcgctgagccgtattaccagcgtggttcagcaacatcaagaggcgctggttccggcgcagccgcaactgaacgtgtgcaactgggcggttatgaactgggcgccgccgggttgcgaaaaactgggcaagtgcctgaccccgccggaaagcatcccgaaaaagtgcctgtggagccattaacacctgcgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 41 - DNA, 2128 bp, Mus musculus
  source 1..2128: mol_type = other DNA
agagcgcccactgcctaacggagagaccccatcgtggcgcgatcatgctccgggtccccctgtgtacgccgctcccgctcctggcactgctgcaactgctgggcgctgcgcacggcatctataatgtcacccagaggacatttaagctcgactacagccgggaccgcttcctcaaggatggacagccattccgatacatctcgggaagcattcattacttccggataccccgcttctactgggaggaccggctgctgaagatgaagatggctgggctgaatgctatccagatgtacgtgccctggaacttccatgaaccccaaccaggacaatatgagttttctggggaccgtgatgtggagcatttcatccagctggctcatgagctgggactcctggtgatcctgaggcctgggccctacatctgtgcagagtgggacatggggggcttacctgcttggctactagagaaacaatctatcgttctccggtcttctgacccagactaccttgtagctgtggataaatggctggcagtccttctgcccaagatgaagcccctgctctaccagaacggaggaccgatcataaccgtgcaggttgagaatgagtacgggtcctactttgcctgcgattacgactacctacgcttcctggtgcaccgcttccgctaccatctgggtaatgacgtcattctcttcaccaccgacggagcaagtgaaaaaatgctgaagtgtgggaccctgcaggacctgtacgccacagtggattttggaacaggcaacaatatcacacaagctttcctggtccagaggaagtttgaacctaaaggacctttgatcaattctgagttctatactggctggctagaccactggggtaaaccccattccacggtgaaaactaaaacactggctacctccctctataacctgcttgcccgtggggccaacgtgaacttgtacatgtttataggtgggaccaattttgcctattggaatggtgccaacacgccctatgagccacagcccaccagctatgactacgacgccccactgagcgaggctggggacctcactaagaagtattttgctcttcgagaagtcattcagatgtttaaagaagtcccagaaggccctatccctccgtctacacccaaatttgcatatggaaaagttgctctgagaaagttcaagacagtggctgaagctctgggtatcctgtgtcccaatgggccagtgaaaagcctctatcccctgacattcactcaggtaaaacagtattttgggtatgtgctgtaccgaacaacgcttcctcaagattgcagtaacccgaaacccattttctcttcacccttcaatggtgtccgtgatcgggcttacgtctctgtggacggggtcccccaaggaatccttgatcgaaacctcatgacagctctgaacatacgggggaaggctggagccacgctggacatcctggtggagaacatggggcgtgtgaactatggcagattcatcaatgacttcaagggtttgatttccaacatgactatcaactccactgtcctcaccaactggacggtcttcccactgaacactgaggccatggtacgcaaccatctctggggccgggaggccagtgatgagggtcaccttgacggacggtcgacctccaattcttcggacctcatactccccaccttttacgtgggcaacttctccatcccctcgggcatcccagacctgccacaggacaccttcatccagtttcctgggtggtccaagggtcaagtatggatcaatggctttaacctcggccgatactggcccacaatgggcccacaaaagaccttgttcgtgccaaggaacatcctgaccacttcagccccaaacaacatcacagtgttggagctagagtttgcaccctgcagcgaggggaccccagagctgtgtacagtagagtttgttgacactccggtcatttcctgacctgacttgaccatcggtggccattttccaagccagtctggtcaagactcatggctgaacctctgagactgagccttggggagcacagctctactctggttacacggatcacctttgttgtgctagaatggaagctacat

SEQ ID NO: 42 - DNA, 2670 bp, synthetic construct
  source 1..2670: mol_type = other DNA
atcatcgatttccgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaacgcggatccagagcgcccactgcctaacggagagaccccatcgtggcgcgatcatgctccgggtccccctgtgtacgccgctcccgctcctggcactgctgcaactgctgggcgctgcgcacggcatctataatgtcacccagaggacatttaagctcgactacagccgggaccgcttcctcaaggatggacagccattccgatacatctcgggaagcattcattacttccggataccccgcttctactgggaggaccggctgctgaagatgaagatggctgggctgaatgctatccagatgtacgtgccctggaacttccatgaaccccaaccaggacaatatgagttttctggggaccgtgatgtggagcatttcatccagctggctcatgagctgggactcctggtgatcctgaggcctgggccctacatctgtgcagagtgggacatggggggcttacctgcttggctactagagaaacaatctatcgttctccggtcttctgacccagactaccttgtagctgtggataaatggctggcagtccttctgcccaagatgaagcccctgctctaccagaacggaggaccgatcataaccgtgcaggttgagaatgagtacgggtcctactttgcctgcgattacgactacctacgcttcctggtgcaccgcttccgctaccatctgggtaatgacgtcattctcttcaccaccgacggagcaagtgaaaaaatgctgaagtgtgggaccctgcaggacctgtacgccacagtggattttggaacaggcaacaatatcacacaagctttcctggtccagaggaagtttgaacctaaaggacctttgatcaattctgagttctatactggctggctagaccactggggtaaaccccattccacggtgaaaactaaaacactggctacctccctctataacctgcttgcccgtggggccaacgtgaacttgtacatgtttataggtgggaccaattttgcctattggaatggtgccaacacgccctatgagccacagcccaccagctatgactacgacgccccactgagcgaggctggggacctcactaagaagtattttgctcttcgagaagtcattcagatgtttaaagaagtcccagaaggccctatccctccgtctacacccaaatttgcatatggaaaagttgctctgagaaagttcaagacagtggctgaagctctgggtatcctgtgtcccaatgggccagtgaaaagcctctatcccctgacattcactcaggtaaaacagtattttgggtatgtgctgtaccgaacaacgcttcctcaagattgcagtaacccgaaacccattttctcttcacccttcaatggtgtccgtgatcgggcttacgtctctgtggacggggtcccccaaggaatccttgatcgaaacctcatgacagctctgaacatacgggggaaggctggagccacgctggacatcctggtggagaacatggggcgtgtgaactatggcagattcatcaatgacttcaagggtttgatttccaacatgactatcaactccactgtcctcaccaactggacggtcttcccactgaacactgaggccatggtacgcaaccatctctggggccgggaggccagtgatgagggtcaccttgacggacggtcgacctccaattcttcggacctcatactccccaccttttacgtgggcaacttctccatcccctcgggcatcccagacctgccacaggacaccttcatccagtttcctgggtggtccaagggtcaagtatggatcaatggctttaacctcggccgatactggcccacaatgggcccacaaaagaccttgttcgtgccaaggaacatcctgaccacttcagccccaaacaacatcacagtgttggagctagagtttgcaccctgcagcgaggggaccccagagctgtgtacagtagagtttgttgacactccggtcatttcctgacctgacttgaccatcggtggccattttccaagccagtctggtcaagactcatggctgaacctctgagactgagccttggggagcacagctctactctggttacacggatcacctttgttgtgctagaatggaagctacat

(19) United States
(12) Patent Application Publication
(10) Pub. No.: US 2026/0048148 A1
(43) Pub. Date: Feb. 19, 2026

(54) GENE THERAPY

(71) Applicants: FONDAZIONE TELETHON ETS (50%), Roma (IT); OSPEDALE SAN RAFFAELE S.R.L. (50%), Milan (IT)

(72) Inventors: Alessandro Aiuti, Milano (IT); Maria Ester Bernardo, Milano (IT); Bernhard Gentner, Milano (IT); Stefania Crippa, Milano (IT); Serena Scala, Milano (IT); Pamela Quaranta, Milano (IT)

(73) Assignees: FONDAZIONE TELETHON ETS (50%), Roma (IT); OSPEDALE SAN RAFFAELE S.R.L. (50%), Milan (IT)

(21) Appl. No.: 19/102,919

(22) PCT Filed: Aug. 8, 2023
(86) PCT No.: PCT/IB2023/057998

§ 371 Date: Feb. 11, 2025

(30) Foreign Application Priority Data
Aug. 11, 2022 (EP) 22189961.0
Sep. 22, 2022 (EP) 22197233.4

(51) Int. Cl. A61K 48/00; A61K 35/17; A61K 38/46; A61K 38/47; A61P 3/00; C12N 15/86

(52) U.S. Cl. CPC A61K 48/0066; A61K 35/17; A61K 38/465; A61K 38/47; A61P 3/00; C12N 15/86; C12Y 301/06004; C12Y 302/01023; C12Y 302/01024; C12N 2740/15043; C12N 2800/22; C12N 2830/15

(57) ABSTRACT

The invention relates to means and methods for gene therapy of lysosomal storage disorders (LSDs), preferably a LSD with skeletal involvement, based on an ex vivo gene therapy approach comprising transduction of autologous hematopoietic stem and progenitor cells (HSPCs) with viral vectors for expressing enzymes that are deficient in the disorders. The final formulation is a suspension of transduced cells in culture medium for the administration to patients affected by the LSDs, preferably preceded by a conditioning regimen.

### FIELD OF THE INVENTION

The present invention relates to means and methods for gene therapy of a lysosomal storage disorder (LSD), preferably of a LSD with skeletal involvement, based on an ex vivo gene therapy approach comprising transduction of autologous hematopoietic stem and progenitor cells (HSPCs) with viral vectors for expressing enzymes that are deficient in the disorder.

The final formulation is a suspension of transduced cells in culture medium for the administration to patients affected by the LSD, preferably preceded by a conditioning regimen.

### BACKGROUND

Lysosomal storage disorders (LSDs) are a group of inherited metabolic diseases that are caused for the most part by lysosomal enzyme deficiencies resulting in accumulation of undegraded substrate. This storage process leads to a broad spectrum of clinical manifestations depending on the specific substrate and site of accumulation.

In general, LSDs have been proven to simultaneously affect multiple cellular pathways and signalling cascades, each contributing to disease pathophysiology and to clinical manifestations.

In particular, mitochondrial dysfunction, oxidative stress, storage of secondary substrates unrelated to the defective enzyme, abnormal composition of membranes, aberrant fusion and intracellular trafficking of vesicles, impairment of autophagy, dysregulation of signalling pathways and activation of inflammation, abnormalities of calcium homeostasis and signalling are now considered important factors in the pathogenesis of several LSDs.

Many of the LSDs have skeletal abnormalities causing significant morbidity. Skeletal abnormalities are in particular an early and prominent feature of most mucopolysaccharide disorders (mucopolysaccharidoses, MPS): most patients affected by MPS exhibit a constellation of radiographic abnormalities, known as dysostosis multiplex, that are actually helpful in diagnosing the disorder.

MPSs are characterized by the deficiency of enzymes required for the stepwise breakdown of glycosaminoglycans (GAGs).

α-Mannosidosis (OMIM 248500) is a rare inherited lysosomal storage disorder with an autosomal recessive inheritance caused by mutations in the MAN2B1 gene encoding for the lysosomal α-D-mannosidase (MAN2B), which leads to a limited expression and consequently to a reduced activity of the corresponding enzymes. Lysosomal α-mannosidase activity with an acidic pH optimum is ubiquitous in human tissues where it occurs as two major forms, A and B, product of the single gene MAN2B1, that can be separated by ion-exchange chromatography on diethylaminoethyl-cellulose (DEAE-cellulose). In alpha-mannosidosis both A and B forms are lacking. Lysosomal alpha-D-mannosidase from humans, rats, cattle, and cats can catalyze the hydrolysis of alpha(1,2)-, alpha(1-3)-, and alpha(1-6)-mannosidic linkages present in the N-linked oligosaccharides. Historically, α-mannosidosis was classified as a mucopolysaccharidosis due to similar coarse facial features, but later was identified as a distinct entity.

Lack or deficiency of lysosomal alpha-mannosidase results in the multisystemic accumulation of undigested oligosaccharides in the lysosomes. Alpha-mannosidosis is characterized by immune deficiency, facial and skeletal abnormalities, hearing impairment, and intellectual disability. It occurs in approximately 1 of 500,000 live births.

Individuals with alpha-mannosidosis have typically coarse facial features, macrocephaly with a prominent forehead, highly arched eyebrows, depressed nasal bridge, widely spaced teeth, macroglossia, and prognathism. Bone disease ranges from asymptomatic osteopenia to focal lytic or sclerotic lesions and osteonecrosis. Clinical or radiographic evidence of mild-to-moderate dysostosis multiplex occurs in 90% of individuals diagnosed with alpha-mannosidosis; these changes are present at birth and may decrease with age. Genu valgus is common and may be treated with epiphyseal arthrodesis at a young age before the epiphyseal lineation of the knee is closed. Over time, from the second until the fourth decade of life the patients may develop destructive polyarthropathy, especially coxarthrosis, but also gonarthrosis. These are often so serious that orthopaedic corrections are needed. Ataxia is the most characteristic and specific motor disturbance. In addition to joint abnormalities and a metabolic myopathy, the disease particularly affects those areas of the brain responsible for fine motor function and muscular coordination. Muscular hypotonia is common. Almost all patients also show some degree of mental retardation with onset of symptoms that vary from 6 months to 3 years. Individuals are late in initiating speech (sometimes as late as the second decade) and have restricted vocabulary and difficult-to-understand pronunciation, possibly the results of congenital and/or later-onset hearing loss. Brain MRI including sagittal T1 and axial T2 sections reveals a partially empty sella turcica, cerebellar atrophy, and white matter signal modifications. Progressive cortico-subcortical atrophy, especially in the cerebellar vermis, has been described while communicating hydrocephalus can occur at any age. High signal abnormalities involving the parieto-occipital white matter are identified on axial T2-weighted scans in some individuals and are probably related to demyelination and associated gliosis.

The first decade of life is characterized by a high incidence of recurrent infections, including the common cold, pneumonia, gastroenteritis, and more rarely, infections of the urinary tract. Serous otitis media is common and is usually not bacterial. The immunodeficiency is due to decreased ability to produce specific antibodies in response to antigen presentation. In addition, leukocytes have a decreased capacity for intracellular killing, which may contribute to the often-serious outcome of bacterial infections. The infections diminish in the second and third decade, when ataxia and muscular weakness are more prominent. The long-term prognosis is poor. There is an insidiously slow progression of neuromuscular and skeletal deterioration over several decades, making most patients wheel-chair dependent. Early death of patients can occur from primary central nervous system involvement or myopathy.

Mucopolysaccharidosis type IVA (MPSIVA), also known as Morquio A syndrome, belongs to the family of LSDs. MPS IVA (OMIM 253000) is an autosomal recessive disease, caused by mutations in the gene encoding N-acetylgalactosamine-6-sulfatase (GALNS) enzyme, which is located on chromosome 16q24.3. To date, more than 300 different mutations in the GALNS gene have been identified, mainly missense point mutations and small deletions. This heterogeneity in GALNS gene mutations is consistent with the broad spectrum of clinical phenotypes of MPS IVA patients. MPSIVA is in fact a severe, multi-system pathology. Clinical presentation is heterogeneous and includes skeletal and joint abnormalities, short stature, coarsening of facial features, cardiorespiratory impairment, hearing and vision loss, fine corneal clouding, dental abnormalities and hepatomegaly. Patients with a severe phenotype have a shortened lifespan and often do not survive beyond their second or third decade of life, while patients with mild MPSIVA survive into their seventh decade of life.

Prevalence and incidence of MPSIVA has not been estimated accurately, but it is reported a worldwide prevalence at birth from 1 per 76,000 to 1 per 1,179,000 (Leadley et al., 2014), with estimated disease incidence ranging from 1 in 200,000 to 300,000 individuals (The National MPS Society, http://www.mpssociety.org/mps/mps-iv/). The incidence of MPS IVA in Italy is estimated at 1:300,000 live births (Caciotti et al., 2015). Nevertheless, the implementation of newborn screening (NBS) programs for MPS IVA in the future will reveal the real incidence and prevalence rates of Morquio A syndrome.

GALNS catalyses the degradation of glycosaminoglycans (GAGs), keratan sulfate (KS) and chondroitin-6-sulfate (C6S), by cleavage at the N-linked sulfate moiety of the GAGs keratan sulfate and chondroitin-6-sulfate.

GALNS enzyme deficiency thus leads to an abnormal intracellular accumulation of KS and C6S in lysosomes of a wide range of tissues and subsequently in progressive cellular damage and multiple organ failure.

Since these GAGs are mainly produced in the cartilage and the undegraded substrates are stored primarily in cartilage and extracellular matrix (ECM), their primary accumulation occurs in the lysosomes of chondrocytes, associated ligaments, and the neighbouring ECM, directly impacting on cartilage and bone development and subsequently leading to systemic skeletal dysplasia. Moreover, in MPSIVA patients, KS is mostly synthesized and accumulated in the cartilage and cornea. Since C6S is mainly located in the growth plates, aorta and cornea, sites of accumulation in patients with MPS IVA are also heart valves and aorta.

MPSIVA patients present one or more of: skeletal dysplasia includes short neck and trunk, cervical spinal cord compression, odontoid hypoplasia with subsequent cervical instability, pectus carinatum, kyphoscoliosis, hip dysplasia, coxa valga and genu valgum; respiratory compromission, adeno-tonsillar hypertrophy, tracheal distortion, tracheo-and broncho-malacia and obstructive sleep; ligamentous laxity of joints and joint hypermobility, short stature; cardiac complications, which mainly include ventricular hypertrophy and early-onset severe valvular involvement and sometimes coronary intimal sclerosis; hearing impairment, ophthalmologic disturbances, such as corneal clouding, astigmatism, cataracts, punctate lens opacities, open-angle glaucoma, optic disc swelling, optic atrophy, and/or retinopathy; dental abnormalities, hepatomegaly and coarse facial features.

Excessive C6S and KS are secreted in blood and excreted in the urine of patients with MPS IVA. The levels of blood and urine KS was proven to correlate with clinical severity during the early and progressive stage of the disease and therefore, it is a good prognostic biomarker.

In MPSIVA children, the most common initial signs of the severe form usually appear between one and three years of age including kyphoscoliosis, genu valgum, short stature, pectus carinatum and abnormal gait. The slowly progressive form of MPS IVA may become evident in late childhood or adolescence, often manifesting as hip problems (pain, stiffness, and Legg Perthes disease). In both the severe form and the slowly progressive form, the initial presentations vary and individuals may present with only a single finding or several findings.

Untreated patient affected with the severe form often become wheelchair bound during adolescence and die in their second or third decade of life due to cardiorespiratory problems or cervical spinal cord complications.

MPSIVA patients presented a reduction of antioxidant defence levels, assessed by a decrease in glutathione content and by an increase in superoxide dismutase activity in erythrocytes.

Concerning lipid and protein damage, it was shown that MPSIVA patients have increased urine isoprostanes and dityrosine levels and decreased plasma sulfhydryl groups compared to controls. Moreover, MPS IVA patients showed higher DNA damage than control group and this damage had an oxidative origin in both pyrimidine and purine bases. Interleukin 6 was increased in patients and presented an inverse correlation with reduced glutathione (GSH) levels, showing a possible link between inflammation and oxidative stress in MPS IVA disease. These data suggest that pro-inflammatory and pro-oxidant states occur in MPS IVA patients.

Mucopolysaccharidosis IVB (MPSIVB), also known as Morquio B disease (MBD), is another LSD, caused by mutations in the GLB1 gene (3p21.33), resulting in deficiency of the beta galactosidase lysosomal enzyme (β-GAL, hereinafter also indicated as GLB1). The GLB1 gene results in two alternatively spliced mRNAs: a transcript of 2.5 kb, encoding the lysosomal enzyme and a transcript of 2.0 kb encoding the Elastin Binding Protein (EBP), which is located in the endosomal compartment. It has been demonstrated that a depletion of EBP in arterial smooth muscle, fibroblasts and chondroblasts interferes with elastic-fibre assembly.

GM1 gangliosidosis also arise from mutations in the GLB1 gene. Depending on the location of the mutations in the GLB1 gene and on their combination in compound heterozygous individuals, the molecular pathophysiology of the resulting β-galactosidase protein can produce a spectrum of phenotypic presentations, ranging from primarily neurologic manifestations in GM1-gangliosidosis, to primarily skeletal involvement in MBD. Mutations associated with GM1-15 gangliosidosis, for the most part, are located in the core protein region, causing β-galactosidase instability, whereas mutations associated with types 2 and 3 GM1-gangliosidosis, tend to be on the protein surface.

W273L mutation is consistently associated with MBD-related skeletal dysostosis, in particular with MBD without neurological involvement (or pure MBD) and can also serve as a predictor of the Morquio B phenotype. W273L occurs in a highly conserved region of the GLB1 gene where the amino acid residue Trp-273 resides at the entrance of the ligand-binding pocket, which acts as a holder of substrates for catalytic reactions. W273L affects the degradation of keratan sulfate more severely than the turnover of GM1-ganglioside. In fact, in Morquio B patients' cells and tissues, the GLB1 enzyme is capable of degrading the terminal beta-linked galactose of GM1 gangliosides and oligosaccharides, but it is unable to degrade KS.

Keratan sulfate is then the main storage product in MBD. Quantitative measurements of KS using LC-MS/MS-based technologies have recently become available. Whereas in GM1 gangliosidosis the main accumulating substrates (GM1 and GA1 gangliosides) affect the central nervous system, in MBD, there is then a preponderance of the accumulation of keratan sulfate in bones and cartilage, explaining the predominance of skeletal manifestations. However, skeletal involvement is also reported in GMI gangliosidosis patients, especially in those affected by the most severe type 1 infantile form.

In vitro and animal studies have further shown that intracellular glycosaminoglycan storage can potentially trigger and maintain the inflammatory response via apoptosis of connective tissue cells, cartilage destruction, subsequent elevation of pro-inflammatory cytokines (predominantly TNF-alpha, IL-1β, and inflammatory proteases), or the activation of the TLR4 pathway leading to the release of TNF-alpha and IL-1β.

Concerning the clinical manifestations, MBD is characterized by short stature with a disproportionally short trunk, kyphoscoliosis, pigeon chest (pectus carinatum), short neck, large appearing head with midface hypoplasia and mandibular protrusion, large appearing joints (elbows, wrists, knees, ankles), coxa and genua valga and flat feet. Joint laxity, corneal clouding, and cardiac valve disease and tracheal stenosis are additional findings. Characteristic radiological findings include platyspondyly and vertebral breaking, odontoid hypoplasia, spinal canal narrowing, hip dysplasia, dysplasia of the carpal and tarsal bones, as well as shortening and epi-and metaphyseal dysplasia of long bones (e.g., shortening of the ulna and sloping of the distal ends of radius and ulna). The skeletal involvement is then a main characteristic of MPSIVB, with below 80% of the GLB1-related MBD cases presenting with a pure skeletal phenotype (pure MBD) and the remaining cases showing additional primary neuronopathic manifestations.

First signs and symptoms of the disease may be apparent at birth. Bone involvement is progressive, with more than 84% of adults requiring ambulation aids. Based on current data, life span is not limited by this condition, but it is important to note that all GLB1-related disorders may be associated with anaesthetic risks.

Individuals affected by alpha-mannosidosis (a-mannosidosis) suffer from similar clinical symptoms, such as skeletal changes, as patients with MPSIVB.

Mucopolysaccharidosis IVA (herein also MPSIVA, for brevity), Mucopolysaccharidosis IVB (herein also MPSIVB or MBD, for brevity) and α-Mannosidosis (herein also a-MANN, for brevity) thus represent rare Lysosomal Storage Disorders with skeletal involvement, characterized by severe and potentially life-threatening manifestations.

Currently, enzyme replacement therapy (ERT) and allogeneic haematopoietic stem cell transplantation (HSCT) from a healthy donor are both available for patients with Morquio A syndrome and α-Mannosidosis. The ERT is in particular available for the treatment of non-neurological manifestations in patients with mild to moderate alpha-mannosidosis. However, ERT has important limitations including immunogenicity, short half-life requiring weekly intravenous infusions, and the inability to target some organs and tissues, especially the bone and central nervous system. Moreover, so far, ERT has not yet been shown to improve bone pathology in MPSIVA and a-MANN patients.

For MPSIVB and GM1 gangliosidosis, supportive and symptomatic therapy is instead the only currently available treatment option, with orthopaedic surgeries being the mainstay of therapies. Non-surgical therapeutic approaches are therefore not available or have no significant impact on MPS-related skeletal lesions and the challenge of preventing and reversing bone and cartilage lesions of MPS IVA, MPSIVB, GM1 gangliosidosis and α-Mannosidosis patients remains unmet. Also, α-Mannosidosis patients that underwent HSCT made developmental progress, but normal development was not achieved and concerns remain about the effectiveness over neurologic and skeletal manifestations of the disease.

On the other hand, HSCT for Morquio A syndrome has been reported only in small case series, resulting in improvement in some clinical features but still being associated with significant morbidity and mortality, especially in the absence of fully matched donors. In particular, the therapeutic effect of allogeneic HSCT on the skeleton in MPSIVA patients remains limited. Effective therapies are thus highly desirable for treating said diseases.

Gene therapy in hematopoietic stem and progenitor cells, GT-HSPCs, is a developing treatment modality for lysosomal storage diseases. Autologous cells may be genetically modified to constitutively express the therapeutic enzyme and become an effective source of functional enzyme in multiple tissues. However, there is still a need for a treatment based on an ex vivo gene therapy which is capable of providing the otherwise deficient enzymes in affected tissues, and in particular to the skeletal resident cells, of patients affected by specific LSDs, in particular with skeletal involvement, such as MPSIVA, MPSIVB, GM1 gangliosidosis and α-Mannosidosis.

In particular, the delivery of an enzyme that is in sufficient amounts and/or sufficiently effective to cross-correct the resident cells of the affected tissues, such as skeletal resident cells (mesenchymal stromal cells, osteoblasts and chondroblasts) is still needed in order to treat specific LSDs with skeletal involvement, such as MPSIVA, MPSIVB, GM1 gangliosidosis and α-Mannosidosis.

### BRIEF DESCRIPTION OF THE INVENTION

The limitations of the prior art are overcome by the present invention, providing effective means and methods for the treatment of LSDs, especially of LSDs with skeletal involvement, in particular of Mucopolysaccharidoses of type IVA and IVB, of GM1 gangliosidosis, or of alpha-mannosidosis, based on ex vivo gene therapy (GT).

The present invention provides new viral vectors for expressing functional lysosomal enzymes that are deficient in said LSDs, as set forth by the present claims.

In a first aspect of the present invention, the viral vector comprises a polynucleotide encoding a lysosomal enzyme selected from: alpha-D-mannosidase (MAN2B) lysosomal enzyme, beta galactosidase (GLB1) lysosomal enzyme, and galactosamine (N-acetyl)-6-sulfatase (GALNS), preferably a polynucleotide encoding a human or murine lysosomal enzyme selected from the enzymes listed above, or variants thereof.

Preferably, the viral vector is a lentiviral (LV) vector.

The invention is also directed to an engineered cell comprising said viral vector, preferably a cell being transduced ex vivo with a viral vector according to the invention, preferably a HSPC, more preferably a CD34+ HSPC.

Optionally, the cell is a T cell, preferably a CD4+ T cell.

The present invention is also directed to a pharmaceutical formulation comprising a therapeutically effective amount of the vector or the engineered cell of the invention.

Furthermore, the invention is directed to methods of manufacturing the viral vectors, the engineered cells and the pharmaceutical formulations of the invention.

The viral vector comprising a polynucleotide encoding a lysosomal enzyme, the cell and the pharmaceutical formulation of the invention comprising said viral vector, are suitable for being used in the treatment of an LSD. In particular, the viral vector comprising a polynucleotide encoding beta galactosidase (GLB1) lysosomal enzyme, the cell and the pharmaceutical formulation of the invention comprising said viral vector, are suitable for being used in the treatment of mucopolysaccharidosis type IVB, or of GM1 gangliosidosis; the viral vector comprising a polynucleotide encoding galactosamine (N-acetyl)-6-sulfatase (GALNS), the cell and the pharmaceutical formulation of the invention comprising said viral vector, are suitable for being used in the treatment of mucopolysaccharidosis type IVA or Morquio A syndrome, and the viral vector comprising a polynucleotide encoding alpha-D-mannosidase lysosomal enzyme, the cell and the pharmaceutical formulation of the invention comprising said viral vector, are suitable for being used in the treatment of α-mannosidosis.

Therefore, the invention is also directed to methods of treatment of LSDs, preferably of LSDs with skeletal involvement, and in particular of Mucopolysaccharidosis type IVA,

Mucopolysaccharidosis type IVB, GM1 gangliosidosis, or alpha-mannosidosis, said methods comprising administration to a subject in need thereof of a therapeutically effective amount of a viral vector, cell or pharmaceutical composition of the invention, expressing the relative enzyme that is deficient in the LSD to be treated.

Ex vivo gene therapy in HSPCs (shortly, HSPC-GT) according to preferred aspects of the present invention combines three unique features in a single treatment:
1) engineering of hematopoietic cells correcting the genetic defect and producing therapeutic enzyme at amounts, and/or with enzymatic activity, sufficient to restore the physiological functions of the enzyme;
2) the ability of the progeny of HSPC to circulate and become resident in all tissues including the bone and the brain;
3) the ability of the progeny of HSPC to produce locally therapeutic enzyme that can be up taken by non-hematopoietic cells, including cells of bone tissue and central nervous system.

The invention is best understood from the following detailed description when read in conjunction with the accompanying drawings.

### BRIEF DESCRIPTION OF FIGURES

FIG. 1: A) Schematic representation of a vector transgene bearing the expression cassette to express GLB1 (LV-GLB1 cassette); the expression cassette shown consists of GLB1 cDNA under the control of the human promoter phosphoglycerate kinase gene (PGK). The following cis-acting polynucleotide regulatory sequence are indicated: major splice donor site (SD), 5′ portion of the gag gene (GA); encapsidation signal (Ψ); splice acceptor sites (SA); central polypurine tract/chain termination sequence (cPPT/CTS); and post-transcriptional regulatory element of woodchuck hepatitis virus (WPRE). The arrow indicates the direction of transgene transcription. B) Schematic representation of the transfer vector bearing the transgene of FIG. 1A. Further to the expression cassette, the following viral cis-acting polynucleotide sequences required for viral production are indicated: truncated 5′ LTR, encapsidation signal (Ψ); Rev-response element (HIV_RRE); central polypurine tract/chain termination sequence (cPPT/CTS); post-transcriptional regulatory element of woodchuck hepatitis virus (Wpre): 3′ LTR (AU3); and poly (A) signal. The expression cassette consists of GLB1 cDNA under the control of the human PGK promoter. Neomycin and Kanamycin resistance (NeoR/KanR) is also indicated.

FIG. 2: A) Analysis of the integrated vector copy number (VCN) in the genome of the myeloid progeny of mobilized peripheral blood (mPB) CD34⁺ cells transduced with LV GLB1 WT and LV GLB1 OPT at different MOI (100, 30, 10). Untransduced cells (UT) were used as controls. B) Growth curves of human mobilized peripheral blood (mPB) CD34+ cells transduced with LV GLB1 WT (left panel) and LV GLB1 OPT (right panel) at different MOI (100, 30, 10) expanded for 14 days as myeloid liquid culture. Untransduced cells (UT) were used as. Values are reported as Log 10-fold increase compared to cell count at day 0. C) Clonogenic assay of (mPB) CD34+ cells transduced with LV GLB1 WT and LV GLB1 OPT at different MOI (100, 30, 10). Untransduced cells (UT) were used as controls. Each error bars show means±s.e.m. (n=3).

FIG. 3: A) Representative image of western blot analysis of GLB1 expression in the cell pellet of the myeloid progeny of human mPB CD34+ cells transduced with LV GLB1 WT and LV GLB1 OPT at different MOI (100, 30, 10). Untransduced cells (UT) were used as controls. Actin-beta (ACTB) was used as a normalizer. B) GLB1 enzymatic activity measured as nmol/mg/h in the cell pellet of the myeloid progeny of human mPB CD34+ cells transduced with LV GLB1 WT and LV GLB1 OPT at different MOI (100, 30, 10). Data are reported as Fold increase on untransduced cells (UT). Each error bars show means±s.e.m. (n≥3).

FIG. 4: A) Representative image of tartrate-resistant acid phosphatase (TRAP) assay performed on the myeloid liquid culture (LC) untransduced (UT) and transduced with LV GLB1 WT (MOI30) after 10 days in osteoclast-differentiation medium to detect the presence of bone reabsorbing osteoclasts. B) Expression analysis of MMP9 and TRAP5b as markers of osteoclast differentiation. C) qPCR analysis of GIB1 expression in the LV GLB1 (MOI30) transduced-myeloid liquid culture (LC) and LC-derived osteoclasts. Untransduced cells were used as controls. In B) and C) results are expressed as 2\hat{ } (-DCT), with the DCT calculated as CT gene of interest-CT ACTB.

Each error bars show means±s.e.m. (n≥3). D) Representative image of western blot analysis of GLB1 expression in the cell pellet (left panel) and medium (right panel) of osteoclasts derived from the differentiation of the myeloid liquid culture (LC) of untransduced and LV GLB1transduced HSPCs. E) GLB1 enzymatic activity measured in the cell pellet (nmol/mg/h) and medium (nmol/ml/h) of osteoclasts derived from the myeloid progeny of human mPB CD34+ cells transduced with LV GLB1 WT at an MOI of 30. Each error bars show means±s.e.m. (n≥2).

FIG. 5: A) Analysis of the integrated vector copy number (VCN) in the genome of human CD4⁺ T cells expanded in vitro for 10 days in the presence of human IL2 and IL7 after transduction with LV human GLB1 (hGLB1), human eIF4A-GLB1 (eIF4A-hGLB1) and murine GLB1 (mGLB1) at an MOI of 30. Untransduced cells (UT) were used as controls. B) qPCR analysis of human GLB1 expression in human CD4⁺ T cells transduced with LV hGLB1 and LV eIF4A-hGLB1 at an MOI of 30 (left panel). qPCR analysis of murine GIB1 expression in human CD4⁺ T cells transduced with LV mGLB1 (right panel). GLB1 expression analysis was performed after 10-day expansion of transduced cells. Untransduced cells were used as controls. Results are expressed as 2\hat{ } (-DCT), with the DCT calculated as CT gene of interest-CT ACTB. C) GLB1 enzymatic activity measured in the cell pellet (left panel) and medium (right panel) of human CD4⁺ T cells transduced with LV human GLB1 (hGLB1), human eIF4A GLB1 (eIF4A-hGLB1) and murine GLB1 (mGLB1) (MOI30). Results are expressed as fold increase on untransduced human CD4⁺ T cells. In all the figures, each error bars show means±s.e.m. (n≥2).

FIG. 6: A) Experimental scheme of in vivo transplantation of healthy donor-derived human mPB CD34″ cells transduced with LV GLB1 WT at an MOI of 30 and injected in sublethally irradiated NSG mice (GT) (4.3×10⁵ cells/mouse). NSG mice transplanted with the same dose of cultured untransduced mPB CD34⁺ cells (MOCK) were used as controls. After transduction, 1×10⁵ mPB CD34⁺ cells were expanded in vitro as a myeloid liquid culture to test LV GLB1 toxicity and transduction efficacy for the transplantation experiment. B) Cell proliferation and (C) integrated vector copy number analysis in the myeloid progeny of mPB CD34⁺ cells untransduced (UT) and transduced with LV GLB1 WT at an MOI of 30 (+LV GLB1). D) Western blot analysis and (E) enzymatic activity of GLB1 in the cell pellet and medium of the myeloid progeny derived from untransduced and LV GLB1-transduced mPB CD34⁺ cells used for the in vivo experiments. F) Body weight monitoring of NSG mice transplanted with untransduced (MOCK) or LV GLB1transduced mPB CD34⁺ cells (GT) at different time points after transplantation. G) Percentage of human cells engraftment (% human CD45⁺ cells/total cells) in the peripheral blood (PB) at 7 weeks after transplantation and in the bone marrow (BM) at 12 weeks after transplantation. H) Integrated vector copy number and (I) GLB1 enzymatic activity analysis in the BM cells isolated from NSG mice transplanted with untransduced (mock) and LV GLB1 transduced mPB CD34⁺ cells (GT).

FIG. 7: A) Schematic representation of a vector transgene bearing the expression cassette to express MAN2B (LV-MAN2B cassette); the expression cassette shown consists of MAN2B cDNA under the control of the human promoter phosphoglycerate kinase gene (PGK). The cis-acting polynucleotide regulatory sequence are indicated as in FIG. 1A. B) Schematic representation of the transfer vector bearing the transgene of FIG. 1A. Further to the expression cassette, the following viral cis-acting polynucleotide sequences required for viral production are indicated: truncated 5′ LTR, encapsidation signal (Ψ); Rev-response element (HIV_RRE); central polypurine tract/chain termination sequence (cPPT/CTS); post-transcriptional regulatory element of woodchuck hepatitis virus (Wpre): 3′ LTR (ΔU3); and poly (A) signal. The expression cassette consists of GLB1 cDNA under the control of the human PGK promoter. Neomycin and Kanamycin resistance (NeoR/KanR) is also indicated.

FIG. 8: A) Growth curve of human mobilized peripheral blood (mPB) CD34+ cells transduced with LV-MAN2B WT (left panel) or LV-MAN2B OPT (right panel) at different MOI (100, 30, 10) expanded for 14 days as myeloid liquid culture. Untransduced cells (UT) were used as controls to evaluate potential toxic effects on cell proliferation of LV-MAN2B WT and OPT HSPC progeny. Values are reported as fold increase compared to cell count at day 0. Each error bars show means±s.e.m. (n=3). B) Colony forming assay of human mPB CD34+ cells transduced with LV-MAN2B WT or LV-MAN2B OPT at different MOI (100, 30, 10). (BFU-E: erythroid burst-forming units; GM-CFU: granulocytes-monocyte colony forming units, GEMM-CFU: granulocyte, erythrocyte, monocyte, megakaryocyte colony forming units) Untransduced cells (UT) were used as controls to determine potential toxic effects on the clonogenic capacity of LV-MAN2B WT and OPT HSPCs. Each error bars show means±s.e.m. (n=3).

FIG. 9: A) Analysis of the number of integrated copies of the vector (VCN) into the genome of the myeloid progeny of mPB CD34+ cells transduced with LV-MAN2B WT and OPT at different MOI (100, 30, 10). Untransduced cells (UT) were used as controls. B) Transduction efficiency calculated as the percentage of vector-positive colonies. (CFC: Colony forming cells). C) MAN2B enzymatic activity measured in the cell pellet (intracellular) and medium (extracellular) of the myeloid progeny of human mPB CD34+ cells transduced with LV-MAN2B WT and OPT at different MOI (100, 30, 10). Data are reported as fold on untransduced cells (UT). Each error bars show means±s.e.m. (n≥3).

FIG. 10: A) Growth curve analysis of mPB hCD34+ transduced with LV-MAN2B WT (left panel) and LV-CTRL (right panel) at an MOI of 30 and expanded as a myeloid liquid culture for 14 days. Untransduced cells (UT) were used as controls. Values are reported as fold increase compared to cell count at day 0. Each error bars show means±s.e.m. (n=3). Cells transduced with a control vector expressing GFP (LV-CTRL) were used for comparison. B) Colony forming assay of mPB hCD34+ transduced with LV-MAN2B WT (left panel) and LV-CTRL (right panel) at a MOI of 30. Untransduced cells (UT) were used as controls. (BFU-E: erythroid burst-forming units; GM-CFU: granulocytes-monocyte colony forming units, GEMM-CFU: granulocyte, erythrocyte, monocyte, megakaryocyte colony forming units). C) VCN measurement of the myeloid progeny of human mPB hCD34+ cells transduced with LV-MAN2B WT. Each error bars show means±s.e.m. (n=3). D) Dosage of MAN2B enzymatic activity in the cell pellet and medium of the myeloid progeny of human mPB hCD34+ cells transduced with LV-MAN2B WT. Data are reported as Fold on untransduced cells (UT). Each error bars show means±s.e.m. (n=3).

FIG. 11: A) Schematic representation of the cross-correction assay. Cell medium conditioned by the myeloid progeny of human mPB CD34+ cells transduced with LV-MAN2B WT at a MOI of 30 was collected after 12-hour-conditioning. The cell medium conditioned by untransduced cells was used as a control. Fibroblasts from alpha-mannosidosis (a-MAN) patients were exposed to the conditioned medium for 12-16 hours and collected for western blot analysis and enzymatic activity dosage. B) MAN2B enzymatic activity in α-MAN fibroblasts from 2 patients (Pt1 and 2) upon exposure to the conditioned medium from the myeloid progeny of transduced (LV-MAN2B) and untransduced (UT) cells. MAN2B enzymatic activity was also measured in untreated fibroblasts (NT) from the same two patients and from 1 healthy donor as a control (HD).

FIG. 12: Analysis of GLB1 expression and enzymatic activity in HSPCs transduced with LV-human GLB1 WT and OPT. A) Representative image of western blot analysis of GLB1 expression in the cell pellet (intracellular) and in the medium (extracellular) from the myeloid progeny of human HSPCs transduced with LV-human GLB1 WT and OPT at different MOI (100, 30, 10). Untransduced cells (UT) were used as controls. Actin-beta (ACTB) was used as a normalizer. B) GLB1 enzymatic activity measured as nmol/mg/h in the cell pellet (intracellular) and in the medium (extracellular) from the myeloid progeny of human mPB CD34+ cells transduced with LV-human GLB1 WT and OPT at different MOI (100, 30, 10). Data are reported as Fold increase on untransduced cells (UT). Each error bars show means±s.e.m. (n≥3). Each dot represents a biological replicate. C) GLB1 enzymatic activity measured in MPSIVB fibroblasts exposed for 24 hours to the conditioned medium from the myeloid progeny of untransduced (UT) HSPCs and HSPCs transduced with LV-human WT and OPT at an MOI of 30. Untreated MPSIVB fibroblasts and healthy-donor (HD) fibroblasts were used as controls.

FIG. 13: Analysis of GLB1 expression. A) qPCR expression analysis of GLB1 RNA in different cell types transduced with the LV-human GLB1 at an MOI of 30. Results are expressed as fold change on untransduced cells (UT). Each error bars show means±s.e.m. (n≥3). Each dot represents a biological replicate. B) Representative image of western blot analysis of GLB1 expression in the cell pellet (intracellular) and in the medium (extracellular) from the myeloid progeny of human HSPCs transduced with LV-human GLB1 WT at a MOI of 30 upon treatment with 100□M chloroquine (+CL) to inhibit GLB1 lysosomal processing. Protein extracts from transduced untreated cells (NT) and from untransduced cells (UT) were used as controls. C) GLB1 enzymatic activity measured in the cell medium from the myeloid progeny of HSPCs cells transduced with LV-human GLB1 WT and LV-human GLB1 eIF4a at a MOI of 30. Each error bars show means±s.e.m. (n =2).

FIG. 14: Representative image showing the protein alignment of the murine GLB1 WT protein and the C2C12-specific protein isoform. Three amino acids substitutions in the C2C12-specific protein are indicated by black boxes.

FIG. 15: A) Analysis of the integrated vector copy number (VCN) in the genome of the myeloid progeny (LC) of human HSPCs transduced with LV-human GLB1 WT (hGLB1), LV-murine GLB1 C2C12 (mGLB1) and LV-murine GLB1 WT (mGLB1). LV GLB1 OPT at an MOI of 30. Untransduced cells (UT) were used as controls. B) Cell proliferation analysis of human HSPCs expanded for 14 days as myeloid liquid culture (LC) upon transduction with LV-human GLB1 (hGLB1), LV-murine GLB1 (mGLB1) C2C12 and LV-murine GLB1 (mGLB1) WT at an MOI of 30. Untransduced cells (UT) were used as controls. Values are reported as Log 10-fold increase compared to cell count at day 0. C) Clonogenic assay evaluating the number and composition of colonies formed by human HSPCs transduced with LV-human GLB1 (hGLB1), LV-murine GLB1 (mGLB1) C2C12 and LV-murine GLB1 (mGLB1) WT at an MOI of 30. Untransduced cells (UT) were used as controls. Each error bars show means±s.e.m. (n =3). D) GLB1 enzymatic activity measured in the cell pellet and extracellular medium of the myeloid progeny of HSPCs cells transduced with LV-human GLB1 (hGLB1), LV-murine GLB1 (mGLB1) C2C12 and LV-murine GLB1 (mGLB1) WT at an MOI of 30. Each error bars show means±s.e.m. (n≥3). Each dot represents a biological replicate. p-values were determined by Mann-Whitney test (*p<0.05; ** p<0.001).

FIG. 16: A) Representative pictures of TRAP assay performed on the myeloid progeny of human HSPCs transduced with LV-human GLB1 and LV-murine GLB1 C2C12 and at an MOI of 30 after 10 days of osteoclast differentiation in the presence of human RANKL (50ng/ml) and M-CSF (25ng/ml). B) GLB1 enzymatic activity measured in the cell pellet and extracellular medium of osteoclasts (OCs) derived from the differentiation of the myeloid progeny of human HSPCs transduced with LV-human GLB1 and LV-murine GLB1 C2C12 and at an MOI of 30. C) Cross-correction assay using MPSIVB fibroblasts exposed for 24 hours to the conditioned medium from the myeloid progeny and osteoclasts of HSPCs transduced with LV-human GLB1 and LV-murine GLB1 C2C12 and at an MOI of 30. D) Cross-correction assay using MPSIVB fibroblasts exposed for 24 hours to the conditioned medium from the myeloid progeny of HSPCs transduced with LV-human GLB1, LV-murine GLB1 C2C12, and LV-murine GLB1 WT at an MOI of 30.

FIG. 17: A) GLB 1 enzymatic activity measured in the cell pellet of osteoblasts (OBs) derived from the differentiation of HS5 stromal cells transduced with a LV co-expressing the Cas9 cDNA and a GLB1-specific gRNA to knock-out the expression of GLB1 enzyme (HS5 OBs GLB1 KO). HS5 OBs transduced with the same LV bearing a control gRNA were used as controls (HS5 OBs ctrl). HS5 OBs were exposed for 24 hours to the conditioned medium from the myeloid progeny of human HSPCs transduced with LV-human GLB1, LV-murine GLB1 C2C12, and LV-murine GLB1 WT at an MOI of 30. Each error bars show means±s.e.m. (n=3). Each dot represents a biological replicate. The absolute values of GLB1 enzymatic activity are reported at the top of each bar. B) ELISA assay to determine the level of keratan sulfate accumulation in the same experimental samples. Each error bars show means±s.e.m. (n=3). Each dot represents a biological replicate.

FIG. 18: A) Experimental scheme of xenotransplantation. Healthy donor-derived HSPCs were transduced with LV-human GLB1 (hGLB1), LV-murine GLB1 C2C12 (mGLB1 C2C12), and LV-murine GLB1 WT (mGLB1 WT) at a MOI of 30 and transplanted (1.95×10⁵ cells/mouse) into NOD.Cg-Kit^{W-41J} Prkdescid 112rgtml Wjl/WaskJ (NSGW41) mice. NSGW41 mice transplanted with the same dose of cultured untransduced HSPCs (MOCK) and untreated mice were used as controls. B) Body weight monitoring of NSGW41 mice transplanted with untransduced (MOCK) or transduced HSPCs at different time points after transplantation. C) Evaluation of human cell engraftment as percentage of human CD45 positive cells on total cells in the peripheral blood (PB) at 8- and 16-weeks post transplantation, in the bone marrow (BM) (D) and in the spleen (E) at 16 weeks after transplantation. F) Evaluation of hematopoietic differentiation of HSPCs transduced with LV-human GLB1, LV-murine GLB1 C2C12, and LV-murine GLB1 WT at a MOI of 30 into myeloid, T, B and NK cells. G) Percentage of human HSPCs (CD34+ CD38−) engraftment in the BM of transplanted mice. H) Integrated vector copy number (VCN) in the mononuclear cells isolated from the BM of transplanted NSGW41. I) GLB1 enzymatic activity measured in the cell pellet of mononuclear cells isolated from the BM of transplanted NSGW41 (left panel). The enzymatic activity was normalized on the VCN (right panel). Each error bars show means±s.e.m. (n=3). Each dot represents a biological replicate. p-values were determined by Mann-Whitney test (*p<0.05; ** p<0.001).

FIG. 19: GLB1 enzymatic activity measured in the cell pellet of MPSIVB fibroblasts (MPSIVB fibro) exposed to the conditioned medium (+LC medium) from the myeloid progeny of untransduced HSPCs (UT) and HSPCs transduced with LV-human GLB1 and LV-murine GLB1 at a MOI of 30 in the presence (+M6P) or absence of 5 mM mannose 6 phosphate (M6P). The presence of M6P inhibits the uptake of both the human and murine GLB1 enzyme. Each error bars show means±s.e.m. (n=3). Each dot represents a biological replicate.

FIG. 20: A) Short-term response of peripheral blood mononucleated cells (PBMNCs) from healthy donors to the murine (left panel) and human (right panel) GLB1 enzyme. The level of immunogenicity was evaluated as T cell proliferation (upper panel) and INF-γ production (lower panel) after 5-day PBMNC exposure to the conditioned medium from HEK293T cells transduced with LV-murine WT, LV-murine C2C12 and LV-human GLB1. B) Long-term immunogenicity response by second challenge exposure to the conditioned medium from HEK293T cells transduced with LV-murine WT (left panel), LV-murine C2C12 (left panel) and LV-human GLB1 (right panel). We determined T cell proliferation (upper panel) and IFN-y production (lower). Each error bars show means±s.e.m. (n=3). Each dot represents a biological replicate.

FIG. 21: A) Growth curve analysis of human mobilized peripheral blood (mPB) transduced with LV-MAN2B WT (left panel) and LV-CTRL (right panel) at MOI of 30 with 1-hit CsH (C, n=6) or 1-hit CsH+PGE2 (C+P, n=3) protocols and expanded as a myeloid liquid culture for 14 days. Values are reported as fold increase compared to cell count at day 0. B) Colony forming assay of mPB hCD34+ transduced with LV-MAN2B WT and LV-CTRL at a MOI of 30 with 1-hit CsH or 1-hit CsH+PGE2 protocols. C) Analysis of the number of integrated copies of the vector (VCN) into the genome of the myeloid progeny of mPB CD34+ cells transduced with LV-MAN2B WT and LV-CTRL at MOI of 30 with 1-hit CsH or 1-hit CsH+PGE2 protocols. D) Transduction efficiency calculated as the percentage of vector-positive colony forming cells (CFC). E) Dosage of MAN2B enzymatic activity in the cell pellet (intracellular) and medium (extracellular) of the myeloid progeny of human mPB hCD34+ cells transduced with LV-MAN2B WT (MOI 30) with 1-hit CsH or 1-hit CsH+PGE2 protocols. Untransduced cells (UT) and cells transduced with a control vector expressing GFP (LV-CTRL) are used as controls in all the experiments. Each error bar shows means±s.e.m.

FIG. 22: A) Schematic representation of the cross-correction assay. Cell medium conditioned by the myeloid progeny of human mPB CD34+ cells transduced with LV-MAN2B WT at an MOI of 30 with 1-hit CsH (C, n=3) or 1-hit CsH+PGE2 (C+P, n=3) protocols was collected after 12-hour-conditioning. The cell medium conditioned by untransduced (UT) cells is used as a control. Fibroblasts from alpha-mannosidosis (a-MAN) patients were exposed to the conditioned medium for 12-16 hours and collected for enzymatic activity dosage. B) MAN2B enzymatic activity in a-MAN fibroblasts from 2 patients (Pt1 and Pt2) upon exposure to the conditioned medium from the myeloid progeny of transduced (LV-MAN2B) and untransduced (UT) cells. MAN2B enzymatic activity was also measured in untreated fibroblasts (NT) from the same two patients and from 1 healthy donor as a control (HD).

FIG. 23: A) Representative images of tartrate-resistant acid phosphatase (TRAP) assay performed on osteoclasts differentiating from untransduced (UT) and transduced CD34+ cells. Cells were transduced with LV-MAN2B WT (MOI 30) with 1-hit CsH or 1-hit CsH+PGE2 protocols and TRAP assay was performed after 10 days in osteoclast-differentiation medium to detect the presence of bone reabsorbing osteoclasts. B) qPCR expression analysis of MMP9 and TRAP5b genes involved in osteoclast (OC) differentiation in myeloid liquid culture (LC) cells and differentiated OCs. Values are reported as 2\hat{ } (-DCT), where DCT is calculated as gene CT-CT ACTB. C) MAN2B enzymatic activity measured in the cell pellet (intracellular) and medium (extracellular) of myeloid liquid culture and osteoclasts derived from myeloid LC of human mPB CD34+ cells transduced with LV-MAN2B WT in presence of CsH or CsH+PGE2. Untransduced cells (UT) are used as control. Each error bar shows means±s.e.m (n=3).

FIG. 24: A) Schematic representation of the cross-correction assay. Cell medium conditioned by the osteoclasts derived from myeloid progeny of human mPB CD34+ cells transduced with LV-MAN2B WT at an MOI of 30 with 1-hit CsH or 1-hit CsH+PGE2 protocols was collected after 12-hour-conditioning. The cell medium conditioned by untransduced (UT) cells is used as a control. Fibroblasts from alpha-mannosidosis (a-MAN) patients were exposed to the conditioned medium for 12-16 hours and collected for enzymatic activity dosage. B) MAN2B enzymatic activity in α-MAN fibroblasts from 2 patients (Pt1 and Pt2) upon exposure to the conditioned medium from transduced (LV-MAN2B) and untransduced (UT) osteoclasts. MAN2B enzymatic activity was also measured in untreated fibroblasts (NT) from the same two patients and from 1healthy donor as a control (HD).

FIG. 25: A) Experimental scheme of the in vivo transplantation experiment using human mPB CD34+ cells transduced with LV-MAN2B WT and untransduced cells. Healthy donor-derived human mPB CD34+ cells are transduced with 1-hit CsH with LV-MAN2B WT at an MOI of 30 and injected in NBSGW mice (3×10⁵ CD34+ cells/mouse). As controls, NBSGW mice are transplanted with cultured untransduced mPB CD34+ cells (MOCK). B) Vector copy number (VCN) analysis in total bone marrow (BM) cells of mice transplanted with untransduced (MOCK) and transduced (LV-MAN2B) mPB CD34+ cells. C) Relative frequencies of human CD45+ cells detected in murine peripheral blood at 7 and 12 weeks after cell infusion (left panel), as well as in murine bone marrow at 12 weeks after transplant (right panel). D) MAN2B enzymatic activity (EA) in total BM cells of mice transplanted with untransduced (MOCK) and transduced (LV-MAN2B) mPB CD34+ cells at 12 weeks after transplant. Each error bar shows means±s.e.m.

FIG. 26: Results of toxicity assessment of wild-type (WT) and codon optimized (OPT) LV GALNS of Example 26. A) Growth curve of human mobilized peripheral blood (mPB) CD34+ cells transduced with LV GALNS WT (left panel) and LV GALNS OPT (right panel) at different MOI (100, 30, 10). Untransduced cells (UT) are used as controls. Values are reported as fold increase compared to cell count at day 0. Each error bars show means±s.e.m. (n=3). B) Colony forming assay of human mPBCD34+ cells transduced with LV GALNS WT and LV GALNS OPT at different MOI (100, 30, 10). Untransduced cells (UT) are used as controls. Each error bars show means±s.e.m. (n=3).

FIG. 27: Analysis of Example 27 of GALNS expression and enzymatic activity in human mPB CD34+ transduced with LV GALNS WT and OPT. A) Analysis of the number of integrated copies of the vector (VCN) into the genome of the myeloid progeny of mPB CD34+ cells transduced with LV GALNS WT and OPT at different MOI (100, 30, 10). B) Transduction efficiency calculated as the percentage of vector-positive colonies. C) Western blot analysis of GALNS expression in the cell pellet (intracellular) and medium (extracellular) of the myeloid progeny of human mPB CD34+ cells transduced with LV GALNS WT and OPT at different MOI (100, 30, 10). Actin-beta (ACTB) is used as a normalizer. D) GALNS enzymatic activity measured in the cell pellet (intracellular) and medium (extracellular) of the myeloid progeny of human mPB CD34+ cells transduced with LV GALNS WT and OPT at different MOI (100, 30, 10). Untransduced cells (UT) are used as controls for all the experiments. Each error bars show means±s.e.m. (n=3).

FIG. 28: Evaluation of Example 28 of LV GALNS WT toxicity and transduction efficiency in human mPB CD34+ cells. A) Growth curve analysis of mPB hCD34+ transduced with LV GALNS WT (left panel) and LV-CTRL (right panel) at MOI of 30 and expanded as a myeloid liquid culture for 14 days. Values are reported as fold increase compared to cell count at day 0. Each error bars show means±s.e.m. (n=3). Cells transduced with a control vector expressing GFP (LV-CTRL) are used for comparison. B) Colony forming assay of mPB hCD34+ transduced with LV GALNS WT (left panel) and LV-CTRL (right panel) at a MOI of 30. C) VCN measurement of the myeloid progeny of human mPB hCD34+ cells transduced with LV GALNS WT. Each error bars show means±s.e.m. (n=3). D) Dosage of GALNS enzymatic activity in the cell pellet and medium of the myeloid progeny of human mPB hCD34+ cells transduced with LV GALNS WT. Each error bars show means±s.e.m. (n=3). Untransduced cells (UT) are used as control in all the experiments.

FIG. 29: restoration of GALNS enzymatic activity in fibroblasts derived from MPSIVA patients of Example 29. A) Schematic representation of the cross-correction assay. Cell medium conditioned by the myeloid progeny of human mPB CD34+ cells transduced with LV GALNS WT at an MOI of 30, collected after 12-hour-conditioning. The cell medium conditioned by untransduced cells is used as a control. B) On the left, Western blot analysis for GALNS expression in fibroblasts derived from three different MPSIVA patients (n=3) exposed to the conditioned medium from the myeloid progeny of transduced (LV GALNS) and untransduced (UT) mPB CD34+ cells (n=3: mPB1; mPB2; mPB3). Calnexin (CNX) is used as a normalizer (left panel). On the right, the level of GALNS enzymatic activity in MPSIVA fibroblasts upon exposure to the conditioned medium from the myeloid progeny of transduced (LV GALNS) and untransduced (UT) cells is reported. GALNS enzymatic activity is also measured in fibroblasts from 1 healthy donor as a control (HD).

FIG. 30: Restoration of GALNS activity in MPSIVA-derived mesenchymal stromal cells (MSCs) and MSC-derived osteoblasts (OBs) of Example 30. A) Western blot analysis for GALNS expression in MSCs and MSC-derived OBs isolated from one MPSIVA patient. GALNS expression is also evaluated in MSCs derived from healthy-donor (HD) as a control. Actin-beta (ACTB) was used as a sample normalizer. B) Schematic representation of the cross-correction assay. C) Western blot analysis of GALNS expression in MPSIVA patient-derived MSCs (left) and OBs (right) after exposure to the conditioned medium (CM) from the progeny of human mPB CD34+ transduced with LV-GALNS. Conditioned medium from the myeloid progeny of untransduced (UT) human mPB CD34+ is used as a control. D) GALNS enzymatic activity measured in MPSIVA MSCs after exposure to conditioned medium (CM) from untransduced (UT) and LV GALNS transduced mPB CD34+ cells. GALNS activity in healthy donor (HD) MSCs is measured as control.

FIG. 31: Analysis of the molecular mechanisms mediating GALNS uptake in MPSIVA MSCs and MSC-derived OBs of Example 31. A) Western blot analysis of GALNS expression in MPSIVA MSCs and MSC-derived OBs exposed for 12 hours to the conditioned medium from HEK293T cell transduced with LV GALNS WT at an MOI of 30 (WT) in the presence (+) or absence (−) of a saturating dose of mannose-6-phosphate (M6P). The conditioned medium from untransduced HEK293T cell (UT) is used as a control. B) Western blot analysis of GALNS expression in MPSIVA MSCs and OBs exposed to the conditioned medium from HEK293T cell transduced with LV GALNS WT at an MOI of 30 (WT) in the presence (+) or absence (−) of a saturating dose of M6P for a shorter time (3 hours). C) GALNS expression in MSC-derived OBs from an MPSIVA patient exposed to increasing volume (1 or 2 ml) of conditioned medium from HEK293T cell transduced with LV GALNS WT at an MOI of 30 (WT), in the presence (+) or absence (−) of M6P. Cells exposed to the conditioned medium from untransduced HEK293T cells (UT) were used as controls for all the experiments. Calnexin (CNX) was used as a sample normalizer.

FIG. 32: Analysis of Example 32 of osteoclasts (OCs) derived from the myeloid progeny of human mPB CD34+ cells transduced with LV GALNS WT. A) TRAP assay for the presence of OCs after 10-day of in vitro differentiation of the myeloid progeny of mPB CD34+ cells transduced with LV GALNS at an MOI of 30. OCs derived from untransduced (UT) cells are used as a control. B) qPCR expression analysis of MMP9 and TRAP5b genes involved in OC differentiation. Values are reported as 2\hat{ } (-DCT), where DCT is calculated as gene CT-CT ACTB. C) Western blot analysis of GALNS expression in the pellet and cell medium of OCs derived from the myeloid culture of untransduced and LV GALNS WT transduced mPB CD34+ cells.

FIG. 33: Results of in vivo transplantation experiments of Example 33. A) Experimental scheme of in vivo transplantation experiment using human mPB CD34+ cells transduced with LV GALNS WT and untransduced cells. Healthy donor-derived human mPB CD34+ cells are transduced with 1-hit CsH protocol with LV GALNS WT at an MOI of 30 and injected in sub-lethally irradiated NSG mice (GT). As controls, NSG mice are transplanted with cultured untransduced mPB CD34+ cells (MOCK). 1×10⁵ mPB CD34+ cells are expanded in vitro as a myeloid liquid culture to test LV GALNS toxicity and transduction efficacy for the in vivo experiment. Cell proliferation (B), clonogenic capacity (C), transduction efficiency (D), and vector copy number analysis (E), in the myeloid progeny of mPB CD34+ cells untransduced and transduced with LV GALNS WT at a MOI of 30. Western blot analysis (F) and dosage of GALNS activity (G) in the cell pellet and medium of the myeloid progeny of mPB CD34+ cells untransduced and transduced with LV GALNS WT at a MOI of 30.

FIG. 34: Results of the analysis of Example 34 of in vivo human reconstitution of human mPB CD34+ cells transduced with LV GALNS WT and untransduced cells after xenotransplantation. A) Weight of transplanted mice at different time points after transplantation of mPB CD34+ cells untransduced (MOCK) and transduced with LV GALNS WT at a MOI of 30 (GT). B) Absolute count (cells/ul) and percentage (%) of human CD45+ cells in peripheral blood (PB) of transplanted mice at 7 and 12 weeks after cell infusion, as well as, in bone marrow BM (C) and SPLEEN (D) at 12 weeks after transplant. E) GALNS enzymatic activity in total BM cells in GT and MOCK group of mice at 12 weeks after transplant.

FIG. 35: A) Schematic representation of the vector transgene bearing the expression cassette to express GALNS (LV-GALNS cassette); the expression cassette consists of GALNS cDNA (wild-type, WT, or Optimized, OPT) under the control of the human promoter phosphoglycerate kinase gene (hPGK). The cis-acting polynucleotide regulatory sequence are indicated as in FIG. 1A. B) Schematic representation of the transfer vector transgene. Further to the expression cassette, the following viral cis-acting polynucleotide sequences required for viral production are indicated: truncated 5′ LTR, encapsidation signal (4′); Rev-response element (RRE); central polypurine tract/chain termination sequence (cPPT/CTS); post-transcriptional regulatory element of woodchuck hepatitis virus (Wpre): 3′ LTR (AU3); and poly-A sequence. The expression cassette consists of GALNS cDNA (wild-type or optimized) under the control of the human PGK promoter. Neomycin and Kanamycin resistance (NeoR/KanR) is also indicated.

FIG. 36: A) Experimental scheme for mPB HSPCs transduction. Human mPB HSPCs CD34+ from healthy donor were pre-stimulated in culture for 22 hours in the proper cell culture medium and transduced with the clinical grade LV-GALNS for 14 hours at different MOI (25, 50, 100) without transduction enhancer (TE) or in the presence of TE alone (PGE2, CsH, LB) or in combination (PGE2+LB, PGE2+CsH, CsH+LB). Untransduced (UT) cells were used as controls. At the end of the transduction, cells were collected for clonogenic assay in MethoCult and expansion as myeloid liquid culture. During cell expansion, cells were counted at different passages to determine their proliferation capacity (toxicity evaluation). After 14 days of expansion, myeloid cells were collected for VCN analysis and enzymatic activity measurement (efficiency evaluation). B) Clonogenic assay to evaluate the clonogenic potential of transduced cells as number and composition of colonies. C) Proliferation assay to determine the proliferation capacity of transduced cells. Values represent the fold change of total number of cells counted at different passages on the number of cells plated for LV transduction (do).

FIG. 37: A) Vector copy number evaluated by ddPCR in cells transduced at different MOI. B) Enzymatic activity measured as nmol after 17 hours incubation of the protein extract from transduced and untransduced (UT) cells with the proper substrate normalized on the amount of protein content (nmol/17 h/mg).

### DETAILED DESCRIPTION OF THE INVENTION

Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a cell” includes a plurality of such cells.

The term “Lysosomal Storage Disorders with skeletal involvement”, as used herein, refers to those LSDs whose clinical manifestations involve abnormalities and/or lesions of the skeletal tissue, including bones and cartilage, in particular MPS IVA, MPSIVB, GM1 gangliosidosis and a-MANN.

The terms “treatment”, “treating”, “treat” and the like, as used herein, refers to the administration of a compound, composition or formulation of the invention to obtain a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease or control of disease progression. The terms “prevent,” “preventing,” and “prevention”, as used herein, refer to inhibiting the inception or decreasing the occurrence of a disease in a subject. Prevention may be complete (e.g., the total absence of pathological cells in a subject) or partial. Prevention also refers to a reduced susceptibility to a clinical condition. Control of disease progression is understood as the achievement of the beneficial or desired clinical results that include, but are not limited to, reduction of the symptoms, reduction of the duration of the disease, stabilization of pathological states (specifically to avoid additional deterioration), delay of the progression of the disease, improvement in the pathological state, and remission (both partial and total). The control of progression of the disease also involves an extension of survival, compared with the expected survival if treatment is not applied.

In particular, in accordance with the present invention, the terms “treatment”, “treating”, “treat” and the like, as used herein, preferably refer to the administration of a compound, composition or formulation of the invention to cure, prevent, delay and/or control the clinical manifestations, including skeletal manifestations, further to CNS and metabolic manifestations, of a pathology. The term “effective amount” refers to an amount of a substance sufficient to achieve the intended purpose.

For example, an effective amount of produced and released lysosomal enzyme by engineered cells to increase an enzyme activity is an amount sufficient to reduce accumulation of the enzyme's substrate(s).

A “therapeutically effective amount” of a produced and released lysosomal enzyme by engineered cells or to treat a disease or disorder is an amount of the produced and released lysosomal enzyme by engineered cells sufficient to reduce or eradicate the signals and symptoms of the disease or disorder. The effective amount of a given substance will vary with factors such as the nature of the substance, the route of administration, the size and species of the animal to receive the substance and the purpose of giving the substance. The effective amount in each individual case may be determined empirically by a skilled artisan according to established methods in the art. For example, by “therapeutically effective dose or amount” of a compound, composition or formulation according to the invention, is intended an amount that, when administered as described herein, brings about a positive therapeutic response, such as improved recovery from the disease or from side conditions of the disease.

The term “individual” or “subject” herein refers to a mammal, preferably human or non-human mammal, more preferably mouse, rat, other rodents, rabbit, dog, cat, pig, cow, horse or primate, further more preferably human.

Those in need of treatment include those already inflicted as well as those in which prevention is desired (e.g., those with no symptoms but diagnosed with the genetic disorder, etc.).

The term “pharmaceutically acceptable excipient” refers to a non-toxic solid, semisolid, or liquid filler, diluent, encapsulating material, or formulation auxiliary of any conventional type that may optionally be included in the compositions of the invention and that causes no significant adverse toxicological effects to the patient. A pharmaceutically acceptable excipient is essentially non-toxic to recipients at the employed dosages and concentrations and is compatible with other ingredients of the formulation. The number and the nature of the pharmaceutically acceptable excipients depend on the desired administration form. Pharmaceutically acceptable excipients are known and may be prepared by methods well known in the art.

A pharmaceutical formulation according to the invention can be formulated in accordance with routine procedures as a pharmaceutical formulation adapted for intravenous, subcutaneous, intramuscular, intra-cerebrospinal fluid (CSF) e.g., intracisternal or intra-cerebroventricular, administration to human beings. In a preferred embodiment, the pharmaceutical formulation is for intravenous or intra-cerebrospinal fluid (CSF) administration. More preferably, the pharmaceutical formulation is for intravenous administration.

The term “unit dosage form,” as used herein, refers to physically discrete units suitable as unitary dosages for human and animal subjects, each unit containing a predetermined quantity of the compound, composition or formulation to be administered, calculated in an amount sufficient to produce the desired effect in association with a pharmaceutically acceptable diluent, carrier or vehicle. The specifications for the unit dosage forms for use in the present invention depend on the particular compound employed and the effect to be achieved, the pharmacodynamics associated with each compound in the host, and the like.

The terms “nucleotide sequence” or “isolated nucleotide sequence” or “polynucleotide sequence” or “polynucleotide” or “isolated polynucleotide sequence” are interchangeably used herein and refer to a nucleic acid molecule, either DNA or RNA, containing deoxyribonucleotides or ribonucleotides respectively. The nucleic acid may be double stranded, single stranded, or contain portions of both double stranded or single stranded sequence.

The terms “variant” refers to biologically active derivatives of the reference molecule that retain desired activity. In general, the term “variant” refers to molecules having a native sequence and structure with one or more additions, substitutions (generally conservative in nature) and/or deletions, relative to the native molecule, so long as the modifications do not destroy biological activity, and which are “substantially homologous” to the reference molecule. In general, the sequences of such variants will have a high degree of sequence homology to the reference sequence, e.g., sequence homology of more than 50%, generally more than 60%-70%, even more particularly 80%-85% or more, such as at least 90%-95% or more, when the two sequences are aligned. In accordance with the present invention, a variant of any biomolecule is a biomolecule that has a nucleic acid or aminoacidic sequence having a % of identity of 50%, 60%, 70%, 80%, 90%, 95%, or 99% to the wild-type nucleic acid or aminoacidic sequence and that retains the biological activity of the wild-type biomolecule. In preferred aspects, the term “variant” of a polynucleotide sequence is used herein to indicate a sequence having a % of identity of at least 90%, 95% or 99% to said polynucleotide sequence. In preferred aspects, the term “variant” of a 25 polynucleotide sequence is used herein to indicate a sequence that is a codon-optimized sequence for expressing the biomolecule encoded by said sequence. The terms “% sequence identity”, “% identity” or “% sequence homology” refer to the percentage of nucleotides or amino acids of a candidate sequence that are identical to the nucleotides or amino acids in the sequence of reference, after aligning the sequences to achieve the maximum % sequence identity. In a preferred embodiment, sequence identity is calculated based on the full length of two given sequences or on part thereof. The % sequence identity can be determined by any methods or algorithms established in the art, such as the ALIGN, BLAST and BLAST 2.0 algorithms and followings. Herein, the “% sequence identity”, “% identity” “or “% sequence homology” is calculated dividing the number of nucleotides or amino acids that are identical after aligning the sequence of reference and the candidate sequence, by the total number of nucleotides or amino acids in the sequence of reference and multiplying the result by 100. In accordance with degeneration of genetic code, variants include sequences where at least one base of the base sequence of a gene is replaced with a different type of base, without changing the amino acid sequence of the polypeptide expressed from the gene. Variants also include codon-optimized sequences and sequences comprising mutated or added nucleotides, e.g., for cloning needs. In accordance with the present invention, variants also include sequences encoding fragments of any biomolecule, i.e., a shorter form of the biomolecule, such as a truncated form, that retains the biological activity of the wild-type biomolecule.

The terms “codify” or “coding” refer to the genetic code that determines how a nucleotide sequence is translated into a polypeptide or a protein. The order of the nucleotides in a sequence determines the order of amino acids along a polypeptide or a protein.

The term “transcriptional regulatory region” or “regulatory element, or region”, as used herein, refers to a nucleic acid fragment capable of regulating the expression of one or more genes. The regulatory regions of the polynucleotides of the invention may include a promoter, plus response elements, activator and enhancer sequences for binding of transcription factors to aid RNA polymerase binding and promote expression, and operator or silencer sequences to which repressor proteins bind to block RNA polymerase attachment and prevent expression.

The term “promoter” must be understood as a nucleic acid fragment that functions to control the transcription of one or more polynucleotides e.g. coding sequences, which is placed 5′ upstream of the polynucleotide sequence(s), and which is structurally identified by the presence of a binding site for DNA dependent RNA polymerase, transcription initiation sites and, but not limited to, binding sites for transcription factors, repressors, and any other nucleotide sequences known in the art to act directly or indirectly to regulate the amount of transcription from the promoter. A promoter is said to operatively linked to a nucleotide sequence or to drive the expression of it when it can initiate transcription of said nucleotide sequence in an expression system using a gene construct comprising said promoter operably linked to a nucleotide sequence of interest using a suitable assay such a RT-qPCR or Northern blotting (detection of the transcript). The activity of said promoter may also be assessed at the protein level using a suitable assay for the encoded protein such as Western blotting or an ELISA. A promoter is said to be capable to initiate transcription if a transcript can be detected or if an increase in a transcript or protein level is found of at least 5%, 10%, 15%, 20%, 30%, 40%, 50%, 60%, 70%, 80%, 90%, 100%, 200%, 300%, 500%, 1000%, 1500% or 2000% as compared to transcription using a construct which only differs in that it is free of said promoter.

The term “constitutive” promoter refers to a promoter that is active under most physiological and developmental conditions. An “inducible” promoter is a promoter that is preferably regulated depending on physiological or developmental conditions. A “tissue-specific” promoter is preferably active in specific types of cells/tissues. A ubiquitous promoter may be defined as a promoter that is active in many or in any different tissue(s).

The term “post-transcriptional regulatory region” refers to any polynucleotide that facilitates the expression, stabilization, or localization of the sequences contained in the cassette or the resulting gene product.

The term “vector” refers to a particle capable of delivering, and optionally expressing, one or more polynucleotides of interest into a host cell. Examples of vectors include, but are not limited to, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells. The vector can be a cloning vector, suitable for propagation and for obtaining polynucleotides, gene constructs or expression vectors incorporated to several heterologous organisms. The term “expression vector” refers to a vector designed for gene expression in cells, i.e., the vector is used to introduce a specific gene into a target cell to produce the protein encoded by the gene.

A “vector” is capable of transferring nucleic acid sequences to target cells, therefore also viral vectors, non-viral vectors, particulate carriers, and liposomes are included in the term vector. Typically, “vector construct,” “expression vector”, and “gene transfer vector,” mean any nucleic acid construct capable of directing the expression of a nucleic acid of interest and which can transfer nucleic acid sequences to target cells. Thus, the term includes cloning and expression vehicles, as well as viral vectors. A vector contains at least one expression cassette consisting of one or more genes and regulatory sequence controlling their expression, to be expressed by a transfected cell. The expression cassette directs the cell's machinery to make RNA and protein(s). An expression cassette typically comprises at least three components: a promoter sequence, an open reading frame, and a 3′ untranslated region that, in eukaryotes, usually contains a polyadenylation site. The vector further comprises regulatory sequences that act as enhancer and/or promoter regions and lead to efficient transcription of the gene carried on the expression vector. Vectors include prokaryotic expression vectors, phages and shuttle vectors, eukaryotic expression vectors based on viral vectors, as well as non-viral vectors.

The term “recombinant plasmid” or “plasmid” refers to a small, circular, double-stranded, self-replicating DNA molecule obtained through genetic engineering techniques capable of transferring genetic material of interest to a cell, which results in production of the product encoded by that said genetic material (e.g., a protein polypeptide, peptide or functional RNA) in the target cell.

Furthermore, the term “recombinant plasmid” or “plasmid” also refers to a small, circular, double-stranded, self-replicating DNA molecule obtained through genetic engineering techniques used during the manufacturing of viral vectors as carriers of the recombinant vector genome.

The terms “engineered” or “genetically modified” as referred to transduced cells are herein used interchangeably.

The term “recombinant viral vector” or “viral vector” refers to an agent obtained from a naturally-occurring virus through genetic engineering techniques capable of transferring genetic material (e.g., DNA or RNA) of interest to a cell, which results in production of the product encoded by that said genetic material (e.g., a protein polypeptide, peptide or functional RNA) in the target cell.

Herein, the terms “vector transgene” or “recombinant vector transgene” refer to a transgene that is transferred to the recipient cell upon transduction. The term “viral vector” or “recombinant viral vector”, as used herein, also refers to the recombinant viral particles being a packaged viral vector, capable of binding to and entering recipient cells, delivering the vector transgene. “Recombinant host cells”, “host cells,” “cells”, “cell lines,” “cell cultures”, “engineered cells” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. The term “derived from” is used herein to identify the original source of a molecule but is not meant to limit the method by which the molecule is made which can be, for example, by chemical synthesis or recombinant means.

The term “gene therapy” refers to the transfer of genetic material (e.g., DNA or RNA) of interest into a cell to treat or prevent a genetic or acquired disease or condition. The genetic material of interest encodes a product (e.g., a protein polypeptide, peptide or functional RNA) whose production in vivo is desired. For example, the genetic material of interest can encode an enzyme, hormone, receptor, or polypeptide of therapeutic value. The term “transducing” or “transduction”, as used herein, refers to the process whereby a foreign nucleotide sequence is introduced into a cell via a viral vector. Transduction, where an exogenous polynucleotide is integrated into the host genome, i.e., the host cell genome, is preferred for the methods of gene transfer described herein. The present invention is directed to a recombinant viral vector, preferably a lentiviral vector (LV), comprising an expression cassette for expressing, in a cell, a polynucleotide encoding the lysosomal enzyme that is deficient in a LSD, preferably in a LSD with skeletal involvement, in particular in mucopolysaccharidoses of type IVA or IVB, in GM1 gangliosidosis, or in alpha-mannosidosis (herein also called “enzyme(s) of interest”), said expression cassette comprising a promoter and the polynucleotide encoding the enzyme of interest operably linked to said promoter.

The enzyme of interest is preferably selected from: beta-galactosidase (β-GAL, hereinafter also indicated as GLB1), which is deficient in mucopolysaccharidosis type IVB and GM1 gangliosidosis alpha-D-mannosidase (MAN2B), which is deficient in alpha-mannosidosis; and galactosamine (N-acetyl)-6-sulfatase (GALNS) which is deficient in mucopolysaccharidosis type IVA.

The term “deficient” referred to an enzyme of interest in relation to a pathology, as disclosed herein, is meant to indicate that the enzyme is in insufficient amount or insufficiently functional to provide its physiological functions.

The recombinant viral vector therefor comprises: a) a polynucleotide encoding the enzyme of interest; b) a promoter driving the expression of the operably linked polynucleotide encoding the enzyme of interest.

The viral vector of the invention is preferably a lentiviral vector (LV), more preferably a replication-defective 3rd generation pseudotyped vector made by a core of HIV-1 structural proteins and enzymes, i.e., a HIV-1 derived vector, the envelope of the Vesicular Stomatitis Virus (VSV) and a genome containing HIV-1 cis-acting sequences, no viral genes and one expression cassette for the gene of interest.

The lentiviral vector (LV) particles can be produced by transient transfection of vector-producing cells, such as a HEK293T cell, with four constructs expressing the vector components (core, envelope and transgene of interest). Preferably, said constructs are two core packaging constructs, an envelope construct and a transfer vector construct, which includes the vector transgene comprising the expression cassette (promoter+gene of interest). Only said transgene integrates into the genome of target cells for stable expression of the gene of interest. In the recombinant viral vector of the invention, the transfer vector construct has been optimized for maximal transduction efficiency and stable constitutive transgene expression in target cells. The LV is preferably replication-defective by design; no viral genes are transferred to target cells.

Because the vector is pseudotyped by the envelope of an unrelated virus, wild type HIV cannot be generated by recombination among the constructs used to make vectors. Moreover, the lack of homology between the envelope and the core packaging sequences makes recombination highly unlikely between these constructs (Vigna and Naldini 2000).

The recombinant lentiviral vector according to the invention further comprises viral cis-regulatory elements, preferably human immunodeficiency virus (HIV)-derived elements; more preferably, the recombinant lentiviral vector of the invention further comprises:
c) a 5′ long terminal repeat (5′ LTR), preferably having sequence SEQ ID NO: 9, or variants thereof;
d) an encapsidation signal (Ψ), preferably having sequence SEQ ID NO: 10, or variants thereof;
e) a Rev-response element (RRE), preferably having sequence SEQ ID NO: 11, or variants thereof;
f) a central polypurine tract
g) a central termination sequence (cPPT/CTS), preferably having sequence SEQ ID NO: 12, or variants thereof;
h) a post-transcriptional regulatory element of woodchuck hepatitis virus (Wpre), preferably having sequence SEQ ID NO: 13, or variants thereof;
i) a 3′ long terminal repeat region (3′LTR), preferably self-inactivating (SIN) 3′LTR, preferably having sequence SEQ ID NO: 14, or variants thereof;
j) a polyadenylation (polyA) signal, preferably having sequence SEQ ID NO: 15, or variants thereof;
k) an SV40 origin of replication), preferably having sequence SEQ ID NO: 16, or variants thereof; and
l) a bacterial high copy origin of replication (flori), preferably having sequence SEQ ID NO:17, or variants thereof.

Preferably the viral vector further comprises a resistance gene, such as a kanamycin and/or neomycin resistance gene.

Preferably the backbone of the recombinant lentiviral vector is of sequence SEQ ID NO: 19, or variants thereof.

With “backbone” of the recombinant lentiviral vector it is meant the empty transfer vector construct of the lentiviral vector (i.e., excluding the gene of interest).

The expression cassette is cloned in the backbone of the recombinant lentiviral vector between the LTRs, so that the expression cassette in the viral vector is flanked by a 5′LTR and a 3′LTR, optionally wherein further regulatory elements are cloned between the LTRs and the expression cassette.

Preferably, the viral vector is a self-inactivating (SIN) LV vector.

Preferably, the viral vector comprises: f) a central polypurine tract/central termination sequence (cPPT/CTS), g) a post-transcriptional regulatory element of woodchuck hepatitis virus (Wpre), and i) a poly A signal, more preferably having sequences as indicated above.

The expression cassette of the viral vector of the invention preferably comprises a Kozak sequence polynucleotide before the ATG transcription initiation site of the polynucleotide encoding the enzyme of interest, preferably a Kozak sequence polynucleotide having sequence SEQ ID NO: 18, or variants thereof.

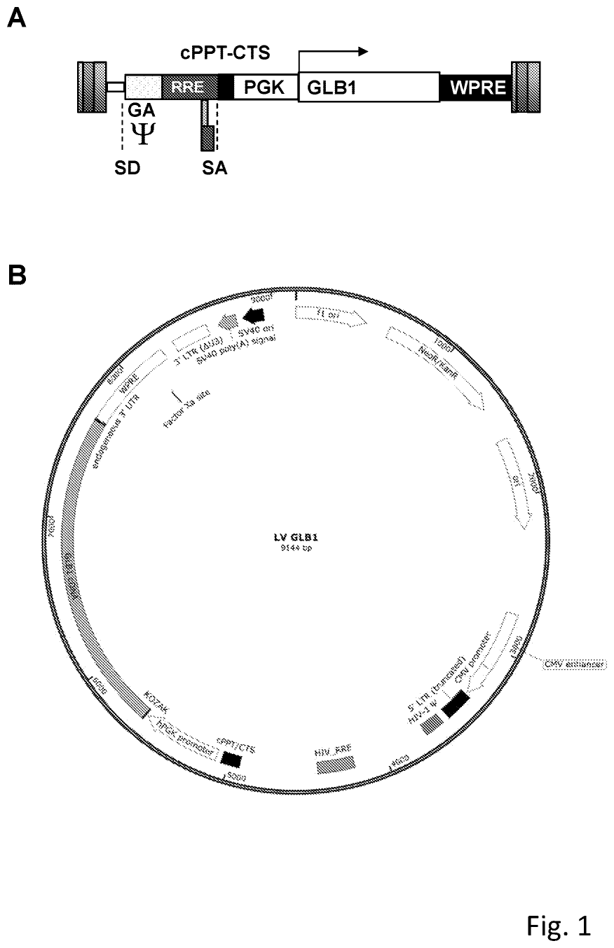

Preferably the expression cassette (vector transgene) and the lentiviral vector according to preferred embodiments of the invention comprise components arranged as depicted in the maps shown in FIGS. 1A, 7A, 35A and 1B, 7B, 35B respectively.

The viral vector of the invention can be produced in a cell according to techniques known in the art.

For example, the lentiviral vector can be produced by transient transfection of HEK293T cells with a packaging plasmid (such as pMDLg/pRRE), a Rev-expressing plasmid (such as pCMV-Rev), and a VSV-G envelop-encoding plasmid (such as pMD2. VSV-G plasmid), in combination with the proper transfer vector plasmid bearing the expression cassette of interest, as described in Dull et al., 1998, or Follenzi et al., 2000.

Preferably, the expression cassette of the lentiviral vector comprises a ubiquitous promoter.

Preferably, the promoter of the expression cassette of the lentiviral vector is selected from the group consisting of:
a) an isolated human PGK promoter, preferably of sequence SEQ ID NO: 3, or variants thereof;
b) an isolated eukaryotic translation elongation factor-1A (EIFIA) promoter, preferably of sequence SEQ ID NO: 6, or variants thereof;
c) an isolated CMV enhancer-containing promoter, preferably of sequence SEQ ID NO: 7, or variants thereof;
d) a CAG promoter, preferably of sequence SEQ ID NO: 8, or variants thereof;
e) the natural promoter of the gene of interest.

More preferably the promoter is an isolated human PGK promoter, preferably of sequence SEQ ID NO: 3, or variants thereof.

The viral vector of the invention is capable of safely transducing cells and of expressing the enzyme of interest at amounts and/or with enzyme activity that are suitable to correct the enzyme deficiency underlying the disease.

In a first aspect of the invention, the enzyme of interest is alpha-D-mannosidase (MAN2B) and the viral vector comprises a polynucleotide encoding MAN2B enzyme.

The term “polynucleotide encoding MAN2B (or alpha-D-mannosidase) enzyme”, as used herein, indicates a polynucleotide encoding an enzyme having alpha-mannosidase activity, capable of degrading the natural substrate(s) of the alpha-D-mannosidase enzyme that is deficient in alpha-mannosidosis.

Preferably said polynucleotide encoding MAN2B enzyme comprises a cDNA having sequence from nucleotide 42 to 3077 of the human MAN2B1 gene (GenBank reference #NM_000528.4) corresponding to the coding sequence (CDS) encoding the homo sapiens MAN2B enzyme. The MAN2B enzyme encoded by said sequence comprises 1011 amminoacids.

Preferably, the polynucleotide encoding the MAN2B enzyme has a codon-optimized (OPT) sequence for improved expression in a cell, preferably a HSPC.

Preferably, the expression cassette of the lentiviral vector comprises a polynucleotide encoding MAN2B enzyme having sequence SEQ ID NO: 21, 22, or variants thereof.

More preferably, the expression cassette has sequence SEQ ID NO: 24, 25, or variants thereof.

Said expression cassette is preferably cloned in a lentivirus transfer vector having sequence SEQ ID NO: 19, the resulting transfer vector having sequence SEQ ID NO: 31, 32, or variants thereof. Therefore, the transfer vector according to the present invention has preferably sequence SEQ ID NO: 31, 32, or variants thereof.

In a second aspect of the invention, the enzyme of interest is beta-galactosidase (GLB1) and the viral vector comprises a polynucleotide encoding GLB1 enzyme.

The term “polynucleotide encoding GLB1 (or beta-galactosidase) enzyme”, as used herein, indicates a polynucleotide encoding an enzyme having beta-galactosidase activity, capable of degrading the natural substrate(s) of the beta-galactosidase enzyme that is deficient in mucopolysaccharidosis type IVB and GM1 gangliosidosis.

Preferably said polynucleotide encoding GLB1 enzyme comprises a cDNA having sequence from nucleotide 62 to 2095 of the human GIB1 gene (GenBank reference #NM_000404.4) corresponding to the coding sequence (CDS) encoding the homo sapiens GLB1 enzyme. The GLB1 enzyme encoded by said sequence comprises 677 amminoacids.

Alternatively, the polynucleotide encoding GLB1 enzyme preferably comprises a cDNA having sequence from nucleotide 115 to 2058 of the or murine Glbl gene (GenBank reference #NM_009752.2) corresponding to the coding sequence (CDS) encoding the mus musculus GLB1enzyme. The GLB1 enzyme encoded by said sequence comprises 647 amminoacids.

Preferably, the polynucleotide encoding the human GLB1 enzyme has a codon-optimized (OPT) sequence that improves its expression in a cell, preferably a HSPC. Preferably, the polynucleotide in the viral vector of the invention encodes the murine GLB1 enzyme, more preferably the wild-type murine GLB1 enzyme or its C2C12 isoform (see FIG. 14).

Preferably, the expression cassette of the lentiviral vector comprises a polynucleotide encoding GLB1 enzyme having sequence SEQ ID NO: 1, 2, 41, 20, or variants thereof.

Optionally, the polynucleotide encoding the GLB1 enzyme has sequence further comprising the eukaryotic translation initiation factors 4A (eIF4A) sequence SEQ ID NO: 26, more preferably immediately upstream the Kozak sequence (SEQ ID NO: 18) to favor translation initiation, thus improving protein expression of GLB1 enzyme.

More preferably, the expression cassette has sequence comprising or consisting of SEQ ID NO: 4, 5, 23, 27, or variants thereof. Said expression cassette is preferably cloned in a lentivirus transfer vector having sequence SEQ ID NO: 19, the resulting transfer vector having sequence SEQ ID NO: 28, 29, 30, 33, or variants thereof. Therefore, the transfer vector according to the present invention has preferably sequence SEQ ID NO: 28, 29, 30, 33, or variants thereof.

In a third aspect of the invention, the enzyme of interest is preferably GALNS enzyme and the polynucleotide is a polynucleotide encoding GALNS enzyme, more preferably comprising a cDNA sequence from nucleotide 71 to 1639 of the GALNS gene (GenBank reference #NM_000512.5), corresponding to the coding sequence (CDS) encoding the GALNS enzyme. The GALNS enzyme encoded by said sequence comprises 522 amminoacids.

The term “polynucleotide encoding GALNS enzyme”, as used herein, indicates a polynucleotide encoding an enzyme having GLANS enzymatic activity, capable of degrading the natural substrate(s) of the GLANS enzyme that is deficient in MPS IVA.

Preferably, the polynucleotide encoding GALNS enzyme has a codon-optimized sequence for expression in a cell, preferably a HSPC.

Preferably, the expression cassette of the lentiviral vector comprises a polynucleotide encoding human GALNS enzyme having sequence SEQ ID NO: 35, or 36, or variants thereof.

More preferably, the expression cassette has sequence SEQ ID NO: 37 or 38, or variants thereof. Sai expression cassette is preferably cloned in a lentivirus transfer vector having sequence SEQ ID NO: 19; more preferably the resulting transfer vector has sequence SEQ ID NO: 39 or 40, or variants thereof. Therefore, the transfer vector according to the present invention has preferably sequence SEQ ID NO: 39 or 40, or variants thereof.

The present invention is further directed to a genetically modified cell comprising the LV vector of the invention, preferably a cell transduced with the LV vector of the invention, thus integrating in its genome the expression cassette for expressing the gene of interest.

Said cell is preferably a stem cell, more preferably a HSPC, most preferably a CD34+ HSPC. CD34 is a transmembrane phosphoglycoprotein transmembrane protein encoded by the CD34 gene in humans, mice, rats and other species. CD34+ is used clinically to indicate haemopoietic stem cells expressing CD34 protein.

In further preferred embodiments, said cell is a T cell, preferably a CD4⁺ T cell.

In preferred embodiments of the invention, said cell is an autologous cell isolated from a subject affected by a lysosomal storage disorder, therefore in need of receiving the ex vivo gene therapy of the invention.

The invention is further directed to the use of said viral vector or of said genetically modified cell in a method of treatment of a LSD, preferably of a LSD with skeletal involvement, more preferably for the treatment of a-MANN, of MPSIVA, of MPSIVB or GM1 gangliosidosis, said viral vector being respectively a viral vector for expressing MAN2B, GALNS, or GLB1 enzymes.

The invention is further directed to a formulation of a medical product comprising hematopoietic stem and progenitor cells (HSPCs), genetically modified with a viral vector according to the invention, to express the enzyme of interest, preferably resuspended in a freezing medium, for further application.

The invention is also directed to the use of said formulation in a method of treatment of a LSD with skeletal involvement, more preferably for the treatment of a-MANN, MPSIVA, MPSIVB, or of GM1 gangliosidosis, said method of treatment preferably comprising a chemotherapy-based conditioning regimen preceding administration of the formulation to a subject in need thereof.

The formulation of the invention is preferably obtained by a manufacturing method comprising the steps of: 1) providing isolated hematopoietic stem and progenitor cells (HSPCs), based on CD34+ expression; and 2) transducing the isolated cells with a viral vector according to the invention, obtaining the genetically modified cells according to the invention. The transduction method includes a stimulation of autologous CD34+ cells in the presence of a human cytokine mix (preferably IL-3, TPO, SCF, and FLT3-1), more preferably for about 22-hour, followed by the addition of the viral particles, preferably for about 14 hours. Upon transduction, the genetically modified CD34+ cells are preferably resuspended in a freezing medium and frozen.

The term “autologous cell” is relative to the recipient of the engineered cell, meaning that cells are obtained from the patient, genetically modified in vitro and reinfused in the patient, which is the recipient of the same cells, once it is genetically modified. The cells can be obtained and isolated by leukapheresis (after mobilization by mobilizing agents such as G-CSF and Plerixafor) or bone marrow harvest (for patients unsuitable for mobilization/leukapheresis) from the patient itself (autologous) The purification process typically involves the use techniques for separating a population of cells expressing a specific marker, such as CD34+ cells; said techniques of separation specific cell populations including: magnetic bead-based separation technologies (e.g. closed circuit magnetic bead-based separation, immunomagnetic beads), flow cytometry, fluorescence-activated cell sorting (FACS), affinity tag purification (e.g. using affinity columns or beads, such biotin columns to separate avidin-labelled agents) and microscopy-based techniques. Clinical grade separation may be performed, for example, using the CliniMACS® system (Miltenyi), which is a closed-circuit magnetic bead-based separation technology.

It is also be possible to perform the separation using a combination of different techniques, such as a magnetic bead-based separation step followed by sorting of the resulting population of cells for one or more additional (positive or negative) markers by flow cytometry.

Preferably, in a method of treatments according to the present invention, the formulation of the invention is administered to a patient in need thereof at a dose providing 4-35×10⁶ cells/kg of body weight, preferably at 14-30×10⁶ cells/kg of body weight, said cells being preferably CD34+ HSPCs, with a median vector copy number of 1-6 per genome, preferably of 1,5-5 per genome, more preferably 2-4 per genome.

The present invention is also directed to the process of manufacturing the genetically modified cells of the invention described above.

According to preferred embodiments, the cells are autologous CD34+ cells and the step of stimulating the isolated cells with cytokines comprises: on day 0, seeding CD34+ cells in cell culture bags (e.g., in RetroNectin®-coated bags) using a serum-free medium (e.g., CellGro (Cell Genix) medium) and stimulating the cells with cytokines for a suitable time, preferably for 22±2hours.

According to this preferred embodiment, on day 1 CD34+ cells are transduced by exposure to the LV supernatant, preferably overnight, more preferably for 14±1 hours, in the same culture medium (1-hit transduction protocol). Optionally, cells are transduced with a 2-hits transduction protocol. According to preferred embodiments of the process of manufacturing of the invention, cells, such as CD34+ cells, are transduced at a multiplicity of infection (MOI) of 1-100, more preferably of 10-100, most preferably of 10-40. Particularly preferred is a MOI of about 30. Immediately after the transduction process, without holding time, the genetically modified CD34+ cells are collected, washed, and resuspended, preferably at a concentration of 2.5-10×10⁶ cells/ml, in a minimum volume of freezing medium (e.g., 20 ml) and cryopreserved under vapor of liquid nitrogen in cryobags.

Preferably, a viral transduction enhancer, is added to the cell culture before transduction according to optimized protocols, for instance as described in WO2013049615, WO2018193118, WO2013127964 and in Delville et al.

Suitable transduction enhancers include prostaglandin E2 (PGE2), protamine sulfate (PS), Vectofusin-1, ViraDuctin, RetroNectin, staurosporine (Stauro), 7-hydroxy-stauro, human serum albumin, polyvinyl alcohol, cyclosporin H (CsH), cyclosporin A (CsA), poloxamines and poloxamers.

Preferably, the viral transduction enhancer added to the cell culture before transduction is PGE2, CsH, poloxamers, or mixtures thereof.

With transduction enhancers, the percentage of cells transduced is increased and/or the vector copy number per cell is increased (VCN).

Before infusion into a subject in need thereof, the suspension of frozen CD34+ HSPCs genetically modified is thawed under controlled conditions at the clinical site.

In some embodiments, the formulation comprising the engineered cells of the invention is substantially purified and free of other cells. In some embodiments, the formulation further comprises one or more of pharmaceutically acceptable excipients.

Surprisingly, HSPCs transduced with a lentivirus vector according to the present invention are capable of restoring the physiological function of the enzyme that is deficient in the disease in various tissues, and most surprisingly even in bone and CNS, without significant toxicity, and even when the enzyme is not expressed or released at super-physiological levels.

Still the capability of the viral vectors to produce high levels of expression of the enzyme of interest permits to reduce the viral load, thus being advantageous in terms of safety and costs.

Moreover, a conditioned supernatant from transduced cells, comprising the enzyme, is capable of cross-correcting non-hematopoietic patient-derived cells obtained from subjects affected by a lysosomal storage disorder, in particular of cross-correcting skeletal resident cells, such as mesenchymal stromal cells, osteoblasts and chondroblasts.

The present invention is then directed also to a method of ex vivo gene therapy for treating a lysosomal storage disorder, in particular an LSD with skeletal involvement, comprising the step of administering a therapeutically effective amount of the viral vector or engineered cell or formulation of the invention to a subject in need thereof.

The inventors have also developed an in vitro cross-correction model using both fibroblasts and cells of skeletal origin (MSCs and osteoblasts) derived from relevant patients exposed to the supernatant from mobilized peripheral blood (mPB) CD34+ transduced with a lentiviral vector according to the invention. Said model comprises fibroblasts, mesenchymal stromal cells (MSCs) and/or osteoblasts, derived from the differentiation of MSCs, isolated from patients affected by an LSD with skeletal involvement. Conditioned medium collected from genetically modified HSPCs was used to correct patients' cells. Conditioned medium from non-transduced cells was employed as control. Patients' cells were exposed to the said medium for a suitable time. Protein extract from conditioned cells was obtained and analyzed for the expression and activity of the enzyme of interest.

This model is particularly useful to predict the efficacy of ex vivo gene therapies with genetically modified cells expressing and releasing in the medium an enzyme of interest, such as the genetically modified cells of the present invention.

The invention is then directed also to a kit and a method for predicting the efficacy of ex vivo gene therapies for treating a disease, based on the in vitro cross-correction model described herein.

It should be understood that all the possible combinations of the preferred aspects of the present invention are also described, and therefore similarly preferred.

Examples of preferred embodiments of the present invention and analyses of their efficacy are provided below for illustrative and non-limiting purposes.

### EXAMPLES

### Materials and Methods

### LV production and Titration

Third generation lentiviral vector (LV) stocks were prepared, concentrated and titred as previously described (Dull et al., 1998, Follenzi et al., 2000). Briefly, self-inactivating (SIN) LV vectors were produced by transient transfection of HEK293T cells with the packaging plasmids, pMDLg/pRRE,

Rev-expressing pCMV-Rev and the VSV-G envelop-encoding pMD2. VSV-G plasmids, in combination with the proper transfer vector. Vector titer was determined by Droplet Digital PCR on the genomic DNA of HEK293T cells transduced with serially dilution of the virus preparations. The concentration of viral p24 was measured by ELISA.

### In-Vitro Transduction of Mobilized Peripheral Blood (mPB) CD34+ Cells

Granulocyte colony-stimulating factor (G-CSF)-mobilized peripheral blood CD34+ cells were placed in culture on retronectin-coated non-tissue culture-treated wells (T100A Takara) in CellGro medium (Cell Genix) supplemented with the following cytokines: 60 ng/ml interleukine-3 (IL-3), 100 ng/ml thrombopoietin (TPO), 300 ng/ml stem cell factor (SCF), and 300 ng/ml FLT3-L (all from Cell Peprotech). After 22 hours of pre-stimulation, cells were transduced at a specific multiplicity of infection (MOI) with a single hit of the proper lentiviral vectors for 16 hours in the same cytokine-containing medium in the presence of 8uM Cyclosporine H (CsH) (Merck), as transduction enhancer. After transduction, cells were collected, washed and plated for colony-forming cell (CFC) assay and myeloid liquid culture. Myeloid liquid culture. Human transduced and untransduced HSPCs were cultured in Iscove's modified Dulbecco's medium (IMDM) with 10% fetal bovine serum (Cambrex, East Rutherford, NJ, USA), 300 ng/ml SCF, 60 ng/ml interleukine-6 (IL-6) and 60 ng/ml IL-3 (all from Preprotech). After 15 days of culture cells were harvested to perform VCN analysis, western blot (WB), qPCR and enzymatic activity assays on supernatants and cell pellets.

Colony-forming cell assay. Colony-forming cell assays were performed by plating 1000 human transduced and untransduced HSPC in a methylcellulose-based medium (Methocult GF4434; Stem Cell Technologies). Fifteen days later colonies were scored by light microscopy for colony numbers and morphology as erythroid, myeloid, and erythroid/myeloid. Moreover, they were collected as a pool and as a single colony and lysed for molecular analysis to evaluate transduction efficiencies by VCN analysis performed by droplet digital PCR on individual colonies. Determination of vector copy number (VCN) by droplet digital PCR. Genomic DNA was extracted using the QIAamp DNA Blood Mini or Micro Kit (QIAGEN) according to the manufacturer's instructions. To evaluate the number of lentiviral vector copies integrated per genome, the droplet digital (ddPCR) technique was used. In brief, the ddPCR assay is based on a primer/probe set designed to detect DNA sequences on the common packaging signal region of lentivirus (human immunodeficiency virus, HIV system). To normalize for the exact amount of template used in each reaction, an endogenous control assay is set up using a DNA sequence specific to a region of the human GAPDH gene (GAPDH system). The target and reference molecule concentrations are calculated in an end-point measurement that enables the quantification of nucleic acids without the use of standard curves and independent of reaction efficiency. The VCN is determined by calculating the ratio of the target molecule concentration to the reference molecule concentration, times the number of copies of the reference species in the genome. All the reactions were performed according to the manufacturer's instructions and were analyzed with a QX200 ddPCR system (Bio-Rad).

### RNA Extraction, qPCR and Gene Expression Analysis

RNA extraction from cells was performed using the RNeasy micro Kit (QIAGEN) or PureLink RNA mini Kit (Thermofisher) according to manufacturer's instructions. 1 ug of RNA was reverse transcribed (RT) using the High-Capacity cDNA Reverse Transcription kit (ThermoFisher Scientific). SYBR Green based quantitative PCR was performed using QuantiFast SYBR Green PCR Kit (Qiagen, 1039712), starting from 10 ng of cDNA with a Viia7 real-time PCR system (Thermofisher) and using ACTB gene as housekeeping.

The following primers were used:

GALNS WT:
FOR:
GCTCATGGACGACATGGGAT;
REV:
AGTTTGGGAAAAGCAGCCCT;
GALNS OPT:
FOR:
ATTACCAGCGTGGTTCAGCAS;
REV:
CCAGTTCATAACCGCCCAGT;
MAN2B WT:
FOR:
GTAAATGCGCAGCAGGCAAA;
REV:
CTCCCAGAGGTAACAAGCGG;
MAN2B OPT:
FOR:
GCTGGAGATGGAGCAAGTGT;
REV:
TATAGCGTTAGGCAGCACG;
human GLB1 WT:
FOR:
GGCCAGGACAGTACCAGTTT;
REV:
TTCTCTAGCAGCCAAGCAGG;
human GLB1 OPT:
FOR:
TTTCGCGCTGCGTAACATC;
REV:
ACCTGAATGAAGGTCAGCGG;
murine GLB1 FOR:
GGTAAACCCCATTCCACGGT;
REV:
GTGGGGCGTCGTAGTCATAG.

The following primers were used to amplify ACTB housekeeping gene:

ACTB:
FOR:
ACAGAGCCTCGCCTTTGCC;
REV:
GATATCATCATCCATGGTGAGCTGG.

To determine gene expression, the difference (ΔCt) between the threshold cycle (Ct) of each gene and that of the reference gene was calculated by applying an equal threshold. Relative quantification values were calculated as the fold-change expression of the gene of interest over its expression in the reference sample (UT), by the formula 2\hat{ }-ΔΔCt.

Humanized mouse models for in vivo studies. Mouse studies were conducted according to protocols approved by the San Raffaele Scientific Institute and Institutional Animal Care and Use Committee, adhering to the Italian Ministry of Health guidelines for the use and the care of experimental animals. All efforts were made to minimize the mice's number and the pain or distress during and after experimental procedures. NOD.Cg-Prkdc^{scid}Il2rg^{tmlWjl/}SzJ (NSG, stock #005557), NOD.Cg-KitW^{W-41J}Tyr⁺Prkdc^{scid}Il2rg^{tmlWjl/}ThomJ (NBSGW, stock #026622) and NOD.Cg-Kit^{W-41J}Prkdc^{scid}Il2rg^{tmlWjl/}WaskJ (NSGW41) mice were purchased from the Jackson Laboratory. Mice were maintained in specific pathogen-free conditions.

### Transplantation of Human Mobilized Peripheral Blood CD34+ Cells in NBSGW Mice

Human mPB-CD34+ cells were pre-stimulated and transduced as described herein with LV-MAN2B WT at an MOI of 30 in presence of CsH. Untransduced cells were used as controls (MOCK). After transduction, 3×10⁵ cells MAN2B gene therapy were infused into the tail vein of 8-10-week-old NBSGW mice. Human cell engraftment and hematological reconstitution was followed by flow cytometry analysis of the peripheral blood (PB) at 7 and 12 weeks after transplantation. At 12 weeks, mice were euthanized and hematopoietic organs (bone marrow and spleen) were also analyzed for human cell engraftment, MAN2B enzymatic activity and viral integration (VCN).

### Transplantation of Human Mobilized Peripheral Blood CD34+ Cells in NSG Mice

Human mPB-CD34+ cells were pre-stimulated and transduced as described herein with LVs for gene therapy at an MOI of 30 in presence of CsH. Untransduced cells were used as controls. After transduction, 4.3×10⁵ cells for GLB1 gene therapy, or 5×10⁵ cells for GALNS gene therapy, were infused into the tail vein of sublethally irradiated (2 Gy) 8-10-week-old NSG mice. Human cell engraftment and hematological reconstitution was followed by flow cytometry analysis of the peripheral blood (PB) at 7 and 12 weeks after transplantation. At the end of the experiment (12 weeks), mice were euthanized and hematopoietic organs (bone marrow, spleen) were also analyzed for human cell engraftment, viral integration (VCN) and enzyme activity.

### Transplantation of Human HSPCs in NSGW41 Mice

Human HSPCs cells were placed in culture and transduced with LV-human GLB1 WT, LV-murine GLB1 WT and LV-murine GLB1 C2C12 at an MOI of 30 in presence CsH. (8 mM). After transduction, 1.95×10⁵ cells were infused into the tail vein of 6-8-week-old NSGW41 mice. Mice transplanted with untransduced cells and untreated mice were used as controls. Human cell engraftment was monitored by flow cytometry analysis of the peripheral blood (PB) at 8 and 16 weeks after transplantation, and in the bone marrow (BM) and spleen at the end of the experiment (16 weeks). BM cells were also analyzed for vector integration (VCN) and GLB1 enzymatic activity.

### Evaluation of Human Engraftment and Immune Reconstitution in Xenotransplant Mice

For GLB1 study in NSGW41: PB, BM and spleen samples were collected from transplanted mice and analyzed using a multi-parametric flow-cytometry assay. Briefly, after RBC lysis with ACK (STEMCELL Technologies #07850), BM cells were stained with fluorescent antibodies against human CD45 APC, CD19 PE-Cy7, CD56 BV510, CD90 PE-Cy5, CD38 APC-Cy7 (Biolegend); CD3 PE, CD34 FITC (BD Biosciences) and CD33 VioBlue (Miltenyi Biotec). PB cells underwent to the same preparation process and were stained with fluorescent antibodies against human CD45 APC, CD19 PE-Cy7, CD56 BV510, CD13 PerCP-Cy5.5 (Biolegend); CD3 PE, CD34 FITC (BD Biosciences) and CD33 VioBlue (Miltenyi Biotec). Absolute cell quantification was performed by adding precision count beads (Biolegend #424902) to the samples. All stained samples were acquired through BD FACSCanto II (BD Bioscience) cytofluorimeter after Rainbow beads (Spherotech #RCP-30-5A) calibration and raw data were collected through DIVA software (BD Biosciences). Data were subsequently analyzed with FlowJo software Version 10.9 (BD Biosciences) and the graphical output was automatically generated through Prism 10.0.0 (GraphPad software).

For MAN2B1 study in NBSGW and for GALNS study in irradiated NSG mice: peripheral blood and bone marrow samples collected from transplanted mice were analyzed using a newly developed multi-parametric flow-cytometry assay (Whole Blood Dissection) (Basso-Ricci et al, 2017). In brief, after red blood cell (RBC) lysis with ACK (STEMCELL Technologies #07850), cells were incubated with a mouse FcR blocking reagent (BD #6148596, dilution 1:100) before staining with fluorescent antibodies against human CD3, CD56, CD14, CD41/61, CD135, CD34, CD45RA (Biolegend) and CD33, CD66b, CD38, CD45, CD90, CD10, CD11c, CD19, CD7 and CD71 (BD Biosciences).

Titration assays were performed to assess the best antibody concentration. After surface marking, cells were incubated with PI (Biolegend #421301) to stain dead cells. Absolute cell quantification was performed by adding precision count beads (Biolegend #424902) to bone marrow (BM) or peripheral blood (PB) samples before WBD procedure. All stained samples were acquired through

BD Symphony A5 (BD Bioscience) cytofluorimeter after Rainbow beads (Spherotech #RCP-30-5A) calibration and raw data were collected through DIVA software (BD Biosciences). Data were subsequently analyzed with FlowJo software Version 10.5.3 (BD Biosciences) and the graphical output was automatically generated through Prism 9.0.0 (GraphPad software). Technically validated results were always included in the analyses, and we did not apply any exclusion criteria for outliers.

### GLB1 Enzymatic Activity

Cell pellets were resuspended in 50-150 ul of H₂O and sonicated for 25 seconds using the Sonoreaktor UTR200 (Hielscher) to obtain protein extract for GLB1 enzymatic activity. Protein concentration was determined using BCA Protein Assay kit (Biorad) using BSA standards

(Biorad). 0.1-5 ug of protein extract diluted in 10 ul of 0.2% BSA was incubated with 20 ul of (0.1M) 4-Methylumbelliferil-β-D-galactopyranoside (4MU-β-gal) for 1 h at 37C. For the enzymatic activity in the cell medium, we used 10 ul of conditioned medium from the myeloid progeny of human HSPCs plated at a concentration of 2×10^{6/}ml for 24 hours, from human osteoclasts plated at a concentration of 0.5×10^{6/}ml and from human CD4⁺ T cells plated at a concentration of 1×10^{6/}ml. At the end of the reaction, 200 ul of stopping buffer 2 (Carbonate 0.5M pH 10.7 Triton X-100) were added to each sample and enzymatic activity was measured as fluorescence emission (450/10). The level of enzymatic activity was calculated using the fluorescence emission based on known amount of 4-Methylumbelliferone (4-MU) standards (nmol) and protein amount (mg).

### GLB1 Enzymatic Activity on Bone Marrow Cells of Xenotransplant Mice

BM cell pellets (2×10⁶) were resuspended in 50 ul of H20 and sonicated for 25 seconds using the Sonoreaktor UTR200 (Hielscher) to obtain protein extract for GLB1 enzymatic activity. Protein concentration of BM samples was determined using Bradford protein assay kit (Biorad) using BSA standards (Thermo Scientific). 0.3 ug of protein extract diluted in 10 ul of 0.2% BSA was incubated with 20 ul of (0.1M) 4-Methylumbelliferil-β-D-galactopyranoside (4MU-β-gal) for 1 h at 37° C. Plasma samples (10 ul), were directly incubated with 20 ul of (0.1M) 4-Methylumbelliferil-β-D-galactopyranoside (4MU-β-gal) for 1 h at 37° C. At the end of the reaction, 200 ul of stopping buffer (Carbonate 0.5M pH 10.7 Triton X-100) were added to each sample and enzymatic activity was measured as fluorescence emission (450/10) (FLUOstar Omega). The level of enzymatic activity was calculated using the fluorescence emission based on known amount of 4-Methylumbelliferone (4-MU) standards (nmol) and protein amount (mg).

### α-mannosidase or GALNS Enzymatic Activity

Cell pellets were resuspended in 50-150 ul of H20 and sonicated for 25 seconds using the Sonoreaktor UTR200 (Hielscher) to obtain protein extract for α-mannosidase or GALNS enzymatic activity. Protein concentration was determined using BCA Protein Assay kit (Biorad) using BSA standards (Biorad). 0.1-lug of protein extract diluted in 10 ul of 0.2% BSA was incubated with 20 ul of 4 mM 4-methylumbelliferyl-α-D-mannopyranoside (4MU-α-mann) for 1 hour or 0.1-5 ug of protein extract diluted in 10 ul of 0.2% BSA was incubated with 20 ul of (10 mM) 4-methylumbelliferyl-β-D-galattoside-6-solfato (4MU-Gal-6S) for 17 hours at 37C. For the α-mannosidase enzymatic activity in the cell medium, 10 ul of conditioned medium from the myeloid progeny of human HSPCs plated at a concentration of 2×10\hat{ }6/ml for 24 hours were used. For GALNS enzymatic activity, after the addition of 5 ul of stopping buffer 1 (Na-Phosphate 0.9M pH4.3), 10 ul of beta-galactosidase (10U/ml) were added to each sample and incubated for 2 hours at 37C. At the end of the reaction, 200 ul of stopping buffer (Carbonate 0.5M pH 10.7 Triton X-100) were added to each sample and enzymatic activity was measured as fluorescence emission (450/10). The level of enzymatic activity was calculated using the fluorescence emission based on known amount of 4-Methylumbelliferone (4-MU) standards (nmol) and protein amount (mg). To measure the GALNS enzymatic activity in the cell medium, we incubated for 17 hours at 37C 20 ul of 4-Methylumbelliferyl B-D-Galactopyranoside-6-sulphate (4MU-Gal-6S) with 10 ul of conditioned medium from the myeloid progeny of human HSPCs plated at a concentration of 2×10⁶/ml for 24 hours, or from human osteoclasts plated at a concentration of 0.5×10⁶/ml for 17 hours at 37C. After 17 hour-incubation, the reaction was stopped by adding the stopping buffer 1 (Na-Phosphate 0.9 M pH 4.3). 10 uL β-Gal working solution (10U/mL) was added and samples were incubated for 2 hours at 37C. At the end of the second reaction, 200 ul of stopping buffer 2 (Carbonate 0.5M pH 10.7 Triton X-100) were added to each sample and enzymatic activity was measured as fluorescence emission (450/10). The level of enzymatic activity was calculated using the fluorescence emission based on known amount of 4-Methylumbelliferone (4-MU) standards (nmol) and protein amount (mg).

### ELISA for Keratan Sulfate Measurement

Accumulated keratan sulfate in HS5 cells were measured using the Mouse Keratan Sulphate (KS) ELISA kit provided by Assay Genie (MOEB2495). Cells were collected upon exposure to conditioned medium from untransduced and transduced cells (LV-human GLB1, LV-murine GLB1 WT, LV-murine GLB1 C2C12) and sonicated for 25 seconds using the ADV-00654PTEP Sonoreaktor UTR200 (Hielscher). Protein extract was quantified using Bradford reagent and a BSA-standard curves. 10ml of protein extract was diluted into 40ml sample diluent. 50 uL of diluted samples, blank (sample diluent alone) and standard were loaded in duplicates on a pre-coated 96-well micro-ELISA plate with a flat bottom. 50 uL of Detection reagent A working solution are added to each well immediately and incubated at 37° C. for 1 hour. After incubation, the wells were washed three times with 200 uL 1X Wash Buffer and 100 uL of Detection Buffer B working solution were added to each well. After incubation at 37° C. for 45 minutes, the plate was washed 5 times with 200 uL 1X Wash buffer. 90 uL of Substrate Solution were directly added to Stop Solution to each well. The plate is then loaded to a Multiskan and light emission is measured at 450nm. An optical density (OD) value is obtained for each well. OD results are then averaged, adjusted for the blank sample OD value and KS concentration is obtained from the standard curve equation. Results are normalized by the protein extract concentration.

### Generation of HS5 GLB1 KO and Control Osteoblasts

HS5 stromal cells were purchased from ATCC (ATCC CRL11882) and expanded in culture using IMDM 10% FBS. The Plasmid lentiCRISPR v2 was purchased from Addgene (52961). In this plasmid we cloned a GLB1 specific guide RNA to knock-out GLB1 expression. We also cloned a scramble guide RNA sequence (Ctrl) and generated LVs (LV GLB1 CRISPR and LV-CTRL CRISPR). HS5 cells were transduced with LV GLB1 CRISPR and LV-CTRL CRISPR at an MOI of 30 and in vitro selected by puromycin (2 mg/ml). Selected cells were induced to differentiate into osteoblasts for 10 days using the StemMACS OsteoDiff medium (130-091-678), Bone differentiation was assessed by Alizarin Red (Sigma-Aldrich) staining of calcium deposits, according to the manufacturer's instruction.

Western Blot analysis. Western blots were performed on protein extract from cells lysed in commercial RIPA buffer (ThermoFisher Scientific) supplemented with, protease inhibitor cocktail (ThermoFisher Scientific) at 4° C. for 20 minutes. Samples were centrifuged 15 min at 10.000 rpm at 4C. Protein lysates were collected and protein concentration was determined by BCA Protein Assay kit (Biorad) using BSA standards (Biorad). 20ug of protein lysates were dissolved in 4× Loading Buffer (CAT) supplemented with beta-mercaptoethanol (Sigma) diluted 1:10. For Western blot analysis of conditioned media, cells were plated at a density of 1×10⁶/ml for 24 hours in the absence of FBS. Conditioned medium was collected and cellular debris were removed by 5 min centrifuge at 2000 rpm at room temperature. 4 volumes of ice-cold acetone were added to the conditioned medium for protein precipitation. Proteins were precipitated by centrifuge at 4000rpm for 10 min at 4° C. Protein pellets were dissolved into an appropriate volume (1 ul/25000 conditioning cells) of 1x Loading Buffer (Biorad) supplemented with 1:10 diluted beta-mercaptoethanol.

Proteins were resolved on precast SDS-PAGE gel (Mini-PROTEAN® TGXTM Gels, Bio-Rad) in commercial Tris/glycine/SDS electrophoresis buffer (Biorad). Proteins were transferred to 0.2 uM PVDF membrane using the Trans-Blot Turbo Transfer system (Biorad). After 1 hour blocking in 5% milk dissolved in TBS-0.1% Tween (Biorad), membranes were incubated overnight with the appropriate primary antibody. The following antibodies were used for GALNS western blot: mouse monoclonal anti human GALNS (1:1000; Santa Cruz Biotechnology, sc-390713); polyclonal rabbit anti human Calnexin (1:1000; Sigma, C4731); mouse monoclonal anti human β-actin (1:50000; Sigma, A3864). After 5 washing of 5 min in TBS-0.1% Tween, membranes were incubated with the proper HRP-conjugated secondary antibody (anti-mouse 1:1500; anti-rabbit: 1:1500; Dako). The following antibodies were used for GLB1 western blot: mouse monoclonal anti human GLB1 (1:500; R&D Systems, MAB6464); mouse monoclonal anti human β-actin (1:50000; Sigma, A3864). After 5 washing of 5 min in TBS-0.1% Tween, membranes were incubated with the proper HRP-conjugated secondary antibody (anti-mouse 1:1500; anti-rabbit: 1:1500; Dako).

For MAN2B western blot, the following antibodies were used: rabbit polyclonal anti human MAN2B (1:500; AbCam, ab 104521); mouse monoclonal anti human beta-actin (1:50000; Sigma, A3864). After washing in TBS-0.1% Tween, membranes were incubated with the proper HRP-conjugated secondary antibody (anti-rabbit: 1:2000; Dako).

Blots were developed using Immobilon Western (Millipore) and images were acquired using UVITEC (Eppendorf).

### Cross-Correction Assay

For cross-correction of cells of non-hematopoietic origin used human healthy donor and LSD patient-derived fibroblasts, mesenchymal stromal cells (MSCs) and/or MSC-derived osteoblasts (OBs) were used. Fibroblasts were plated at a concentration of 25000/cm² in IMDM supplemented with 15% FBS and 1% PS. MSCs were plated at a concentration of 25000/cm2 in DMEM supplemented with 10% FBS and 1% PS. MSCs were differentiated into OBs by plating 40000cells into a well of a 6-well plate in osteogenic medium (MiltenyiBiotec) for 10 days. Differentiation medium was replenished every 2-3 days. Fibroblasts were exposed for 24 hours to the conditioned medium from untransduced and LV transduced cells. The conditioned medium from the myeloid progeny of human HSPCs plated at a concentration of 2×10⁶/ml was collected after 24 hour-conditioning.

After the exposure to the conditioned medium, cell pellet of fibroblasts, MSCs and MSC-derived OBs was collected for protein extraction and enzymatic activity dosage.

### Osteoclast Differentiation

Osteoclasts were differentiated from the myeloid progeny of untransduced and LV transduced human mobilized peripheral blood CD34+ cells. 5×10⁵ cells were plated in 200 ul of alpha-Minimum Essential Medium (aMEM, supplemented with 10% FBS, 1% PS, 1% Glut, and the following cytokines: 25 ng/ml human recombinant macrophage colony-stimulating factors (M-CSF); 50-100 ng/ml human recombinant receptor activator of nuclear factor kappa-B ligand (RankL). Half of the medium was changed twice a for 10 days. Osteoclasts differentiation was evaluated by TRAP assay using the Tartrate Resistant Acid Phosphatase (TRAP) Kit (Sigma-Aldrich), following the manufacturer's instruction, and by RT-qPCR expression of MMP9 and TRAP5b genes using the following primers:

MMP9:
FOR:
CTTTGAGTCCGGTGGACGAT;
REV:
TCGCCAGTACTTCCCATCCT;
TRAP5b:
FOR:
CCCATAGTGGAAGCGCAGAT;
REV:
CTGAGTGGGGCTGGGAATTT.

### Human CD4⁺ T Cell Isolation and Transduction

Peripheral blood mononucleated cells (PBMCs) were isolated from buffy coat of healthy donors by density gradient centrifugation using Ficoll-Paque. CD4⁺ T cells were isolated from PBMCs by negative selection using the CD4+ isolation kit, LS column and MidiMACS separator (Miltenyi Biotec). Human CD4⁺ T cells were activated using Dynabeads™ Human T-Activator CD3/CD28 (Gibco) and cultured in the X-VIVOTM 15 Serum-free Hematopoietic Cell Medium (Lonza) supplemented with 1% penicillin/streptomycin, 5% human serum, human IL-2 (40U/ml) and human IL-7 (10 ng/ml) at a density of 1×10⁶ cells/ml. After 24 hours, human CD4⁺ T cells transduction were transduced with the proper lentiviral vectors at an MOI of 30 (LV hGLB1 WT, LV mGLB1 WT, and LV eIF4A-hGLB1). Transduced cells were splitted twice a week for ten days before sample collection for further analysis.

### Example 1—Production of Lentiviral Vectors for Expressing GLB1 Genes

Four different GLB1 lentiviral transfer vectors were generated to overexpress four different versions of GLB1 cDNA in human mobilized peripheral blood (mPB) CD34+ cells: 1) a lentiviral transfer vector for expressing human GLB1 with wild-type sequence (expression cassette LV-hGLB1 WT, SEQ ID NO: 4); 2) a lentiviral transfer vector for expressing human GLB1 with optimized sequence (expression cassette LV-hGLB1 OPT, SEQ ID NO: 5); 3) a lentiviral transfer vector for expressing human GLB1 with wild-type sequence including eIF4alpha (expression cassette LV-eIF4A-hGLB1 WT, SEQ ID NO: 27); and 4) a lentiviral transfer vector for expressing murine GLB1 sequence (expression cassette LV-mGLB1, SEQ ID NO: 23).

Figure (FIG. 1A provides a schematic view of a vector transgene and FIG. 1B provides a schematic view of a transfer vector construct, bearing the expression cassette for expressing a GLB1 enzyme in transduced cells.

### Example 2—Production of Lentiviral Vectors for Expressing MAN2B Genes

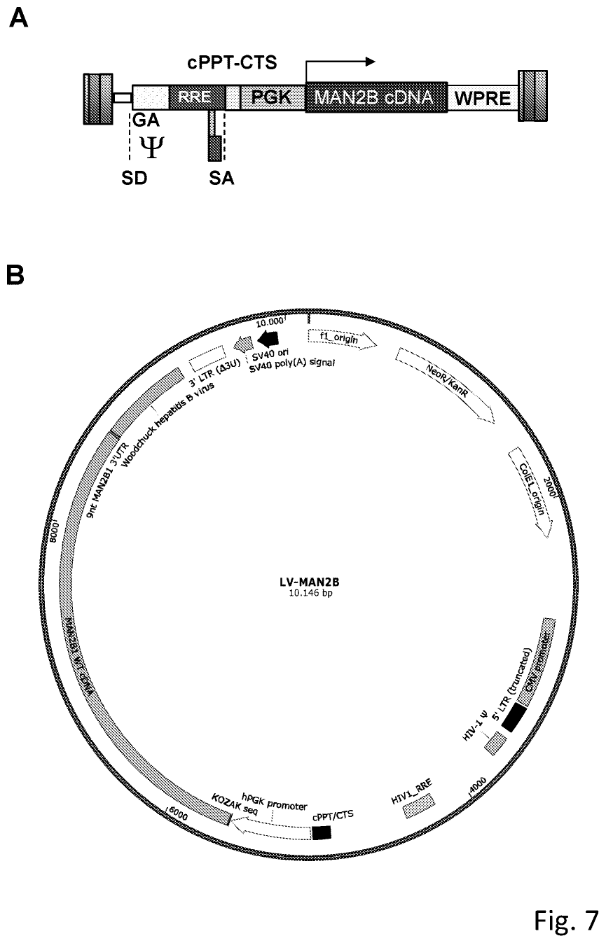

Two different LVs were produced to overexpress MAN2B cDNA: one with a transfer vector construct (pCCLsin.cPPT.hPGK.MAN2Bwt.Wpre) whose expression cassette comprises a wild-type polynucleotide encoding MAN2B (expression cassette LV-MAN2B WT, SEQ ID NO: 24), one with a transfer vector construct (pCCLsin.cPPT.hPGK.MAN2Bopt.Wpre) whose expression cassette comprises a codon-optimized polynucleotide encoding MAN2B (expression cassette “LV-MAN2B OPT, SEQ ID NO: 25). Also, it was produced a LV with a transfer vector construct (pCCLsin.cPPT.hPGK.eGFP.Wpre) comprising an expression cassette for expressing eGFP reporter gene under hPGK promoter. FIG. 7A provides a schematic view of a vector transgene and FIG. 7B provides a schematic view of a transfer vector construct, bearing the expression cassette for expressing a MAN2B in transduced cells.

### Example 3—Evaluation of LV GLB1 Wild-Type (WT) and Codon Optimized (OPT) Toxicity

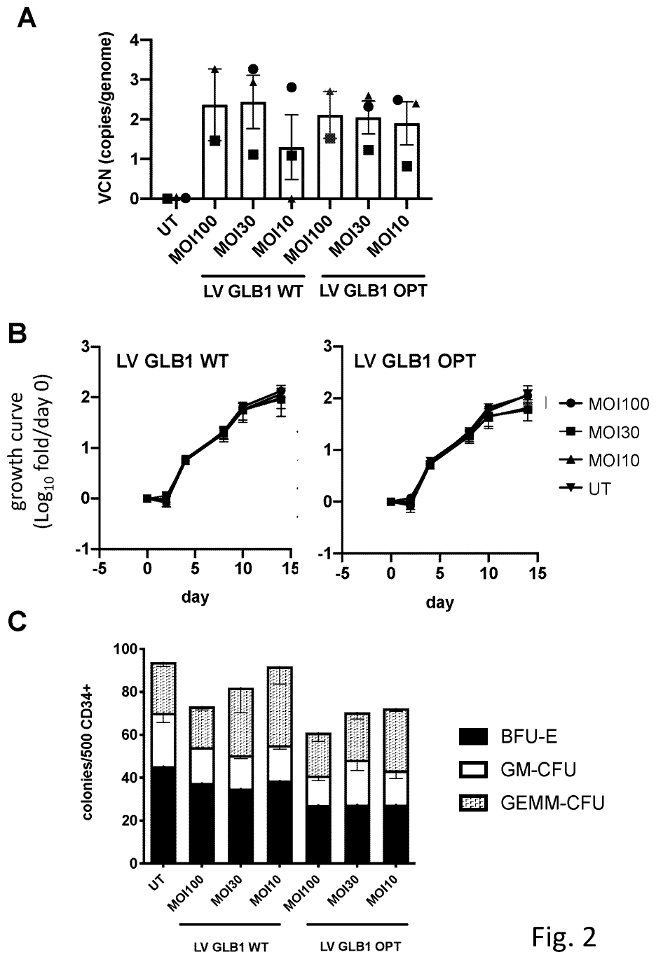

mPB CD34⁺ cells derived from healthy donors (n=3) were placed in culture on retronectin-coated non-tissue culture-treated wells (T100A Takara) in CellGro medium (Cell Genix) supplemented with the following human cytokines: 60 ng/ml interleukine-3 (IL-3), 100 ng/ml thrombopoietin (TPO), 300 ng/ml stem cell factor (SCF), and 300 ng/ml FLT3-L (all from Cell Peprotech). After 22 hours of pre-stimulation, cells were transduced at a specific multiplicity of infection (MOI 100, 30, or 10) with a single hit of lentiviral vector (LV) to overexpress human wild-type and codon-optimized GLB1 in the same cytokine-containing medium in the presence of 8uM Cyclosporine H (CsH) for 14 hours (Merck), as transduction enhancer. After transduction, cells were collected, washed, and plated for colony-forming cell (CFC) assay and in vitro expansion as myeloid liquid culture. Cells from myeloid liquid culture were counted twice a week to determine the proliferation capacity of transduced cells compared to untransduced control cells and passaged for 14 days at a concentration of 0.5×10⁵/ml. Cells resulted efficiently transduced (FIG. 2A) without signs of toxicity in terms of proliferation (FIG. 2B) and clonogenic capacity (FIG. 2C).

### Example 4—Analysis of GLB1 Expression and Enzymatic Activity in Human mPB CD34+ Transduced with LV GLB1 WT and OPT

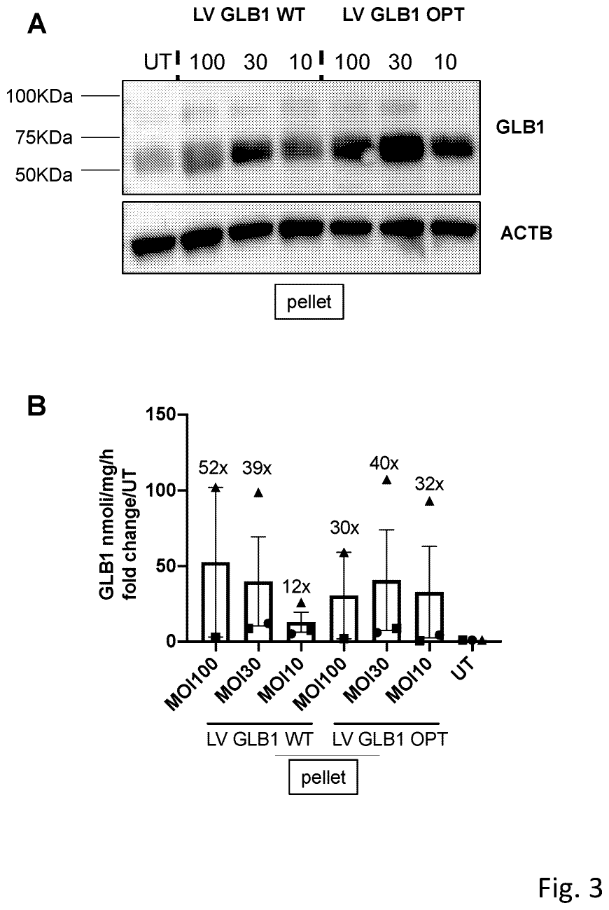

GLB1 expression was evaluated by Western Blot in the protein extract from the myeloid liquid culture of mPB CD34+ cells transduced with LV GLB1 WT and LV GLB1 OPT at different MOI (FIG. 3A). The expression of GLB1 protein increased in the transduced cells compared to untransduced control cells. The GLB 1 lysosomal protein (lower molecular weight) increased more robustly than the precursor protein (higher molecular weight) in transduced cells (FIG. 3A). Similarly, GLB1 enzymatic activity was higher in the transduced cells than controls (FIG. 3B).

### Example 5-Osteoclasts (OCs) Derived from the Myeloid Progeny of Human mPB CD34+ Cells Transduced with LV GLB1 WT

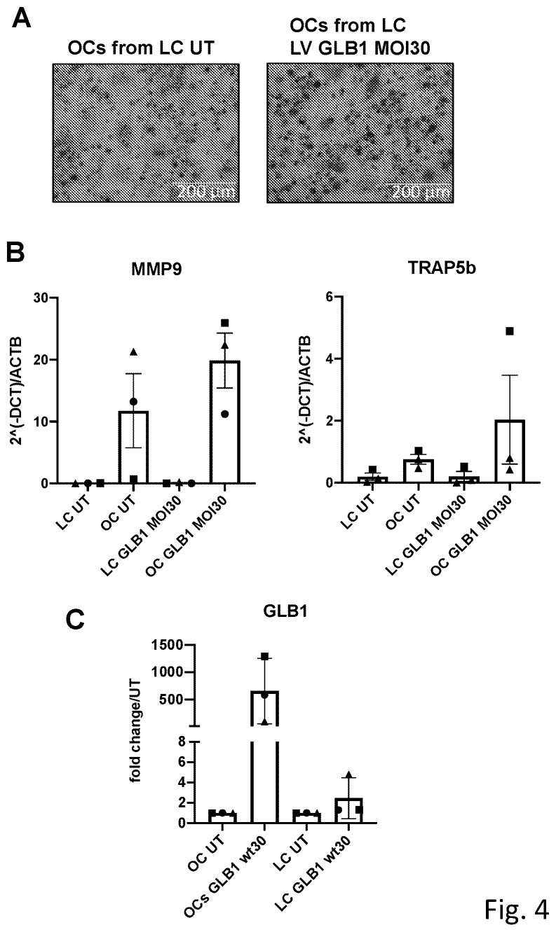

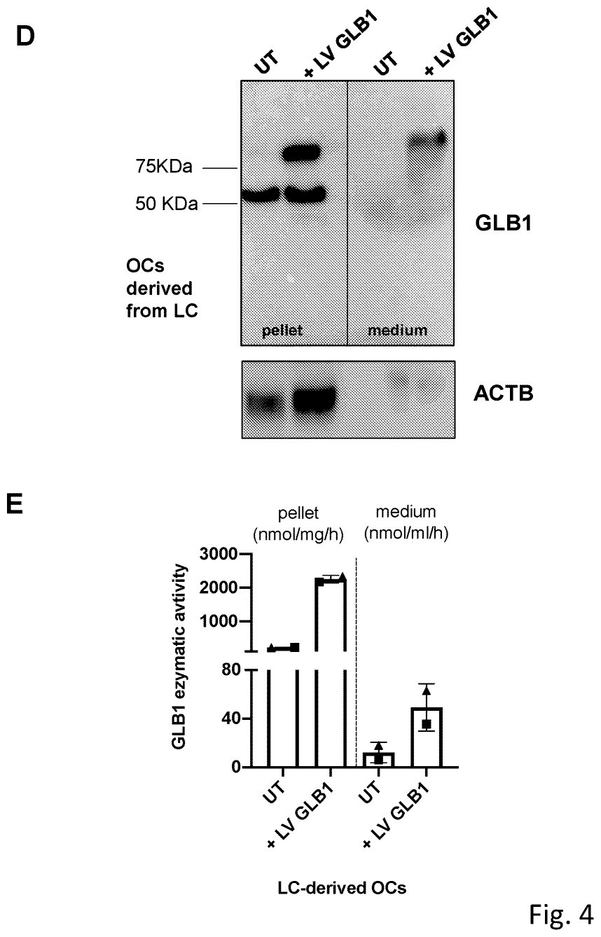

Myeloid cells were induced to differentiate into osteoclasts for ten days in a proper differentiation medium supplemented with human RANKL (50 ng/ml) and M-CSF (25 ng/ml). The capability of osteoclasts (OCs) to express and release GLB1 was investigated as in vitro system reproducing a resident source of GLB1 for the cross-correction of skeletal and cartilage cells. The presence of osteoclasts was evaluated upon differentiation by TRAP assay and qPCR analysis for the expression of osteoclast markers (MMP9, TRAP5b). Both untransduced (UT) and transduced myeloid liquid culture (LC) cells efficiently differentiate into osteoclasts (FIG. 4A) and overexpressed osteoclast genes (FIG. 4B) compared to their undifferentiated counterparts. Importantly, it was found that GLB1 expression increased during the differentiation process (FIG. 4C). Osteoclasts derived from LV GLB1 transduced mPB CD34+ cells express a higher level of human GLB1 than the myeloid liquid culture (LC) counterparts (FIG. 4C). The higher level of GLB1 gene correlated with increased expression of both lysosomal and precursor protein in the protein extracts from osteoclasts compared to controls (FIG. 4D). GLB1 protein was also robustly detected in the cell medium from derived osteoclasts, indicating that osteoclasts release GLB1 in the extracellular space. The dosage of GLB1 enzymatic activity confirmed the protein expression data. A significantly higher enzymatic activity was measured in the cell pellet and medium from mPB CD34+-derived osteoclasts than controls (FIG. 4E).

### Example 6-Analysis of GLB1 Expression and Enzymatic Activity in Human CD4⁺ T Cells Transduced with LV GLB1 Viral Vectors

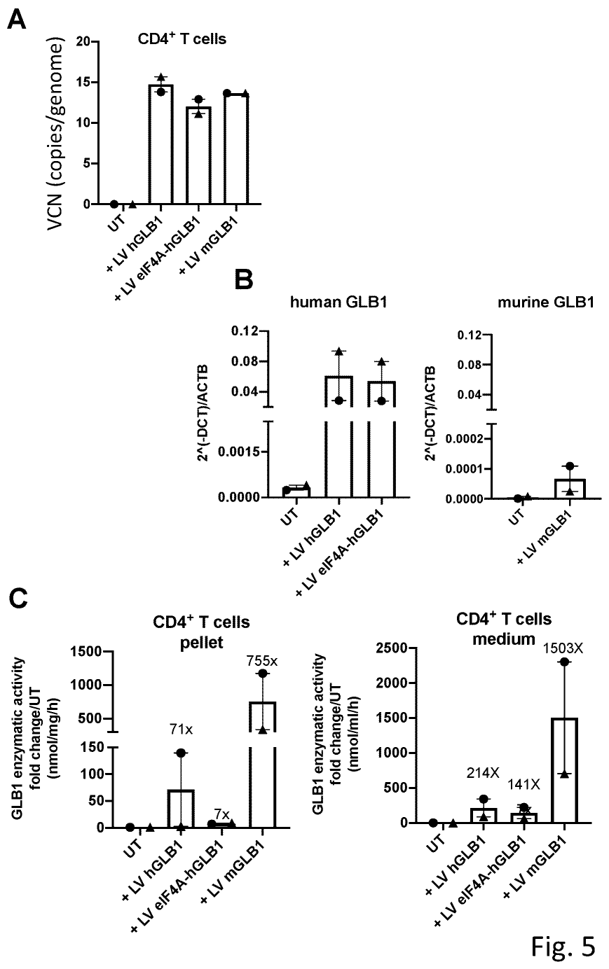

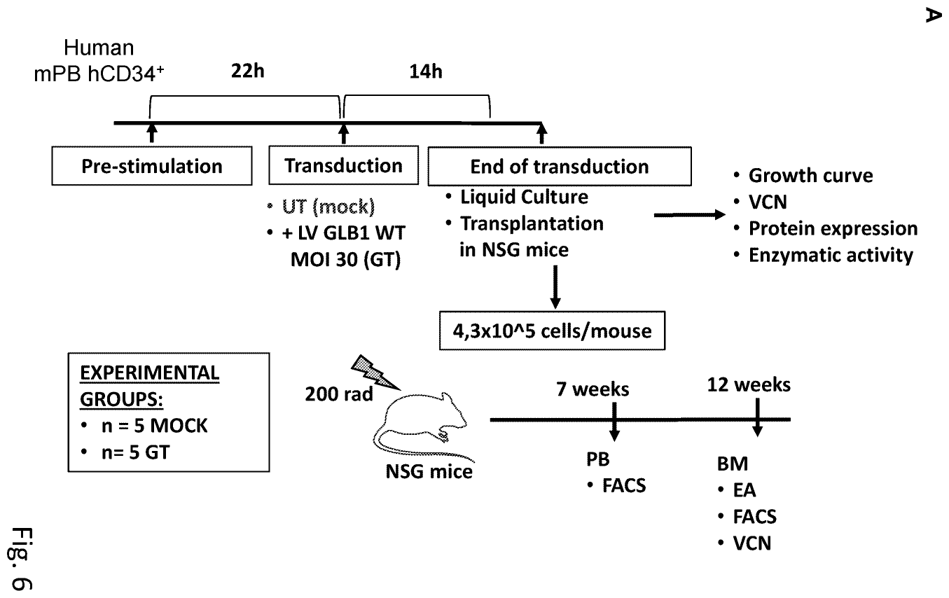

CD4⁺ T cells were transduced as a further cell system reproducing a systemic source of GLB1enzyme that could mediate the release of a super-physiological level of enzyme in the circulation. After isolation of CD4⁺ T cells from human healthy-donor blood samples, cells were transduced with different versions of LV GLB1 (human GLB1 WT, human eIF4A-GLB1 WT, murine GLB1). Untransduced cells were used as controls. CD4⁺ T cells were efficiently transduced (FIG. 5A). VCN were similar among all the conditions. Human and murine GLB1 expression was evaluated by qPCR. GLB1 gene was specifically induced in transduced T cells compared to control untransduced cells (FIG. 5B). GLB1 enzymatic activity was measured on both CD4⁺ T cell pellet and medium. For all the conditions, a higher enzymatic activity was observed in transduced CD4+ T than in untransduced cells. When analyzing the differences in GLB1 enzymatic activity upon transduction, it was observed that surprisingly GLB1 enzymatic activity was significantly higher in CD4⁺ T cells transduced with the murine version of LV GLB1 both in the cell pellet (755 fold compared to untransduced) and in the medium (1520 fold compared to untransduced) (FIG. 5C).

### Example 7-in Vivo Transplantation Experiment Using Human mPB CD34+ Cells Transduced With LV hGLB1 WT and Untransduced Cells

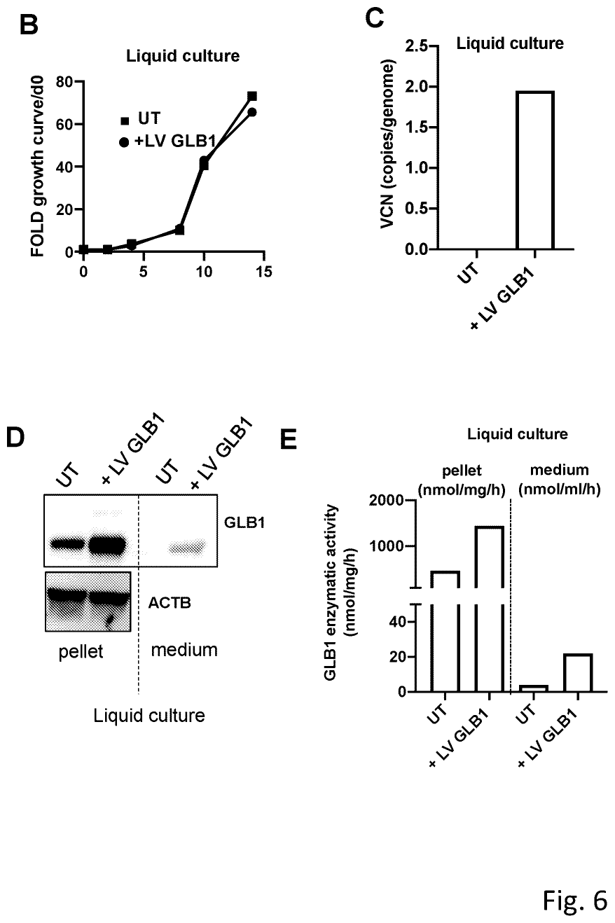

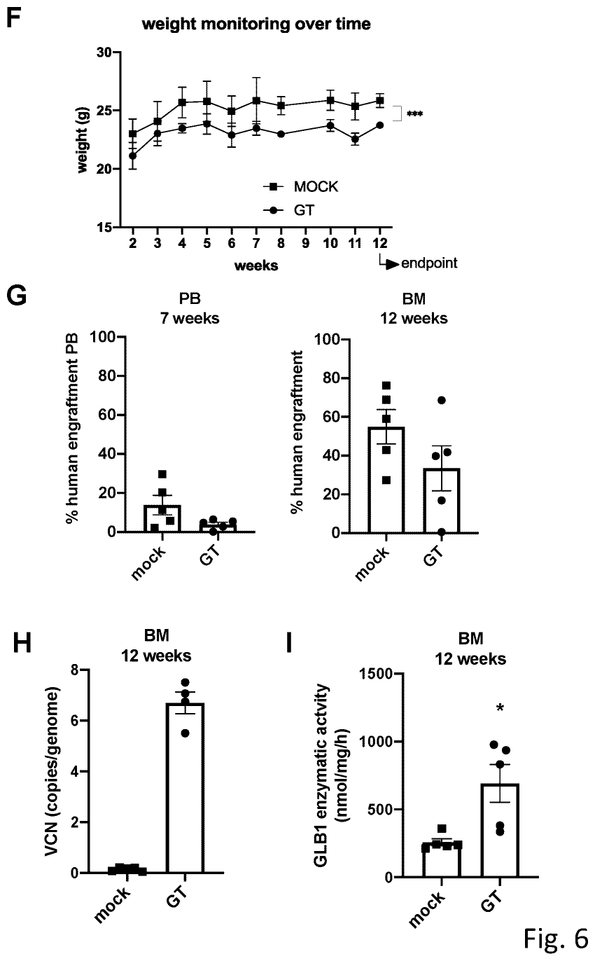

Healthy-donor mPB CD34+ cells transduced with the LV hGLB1 at an MOI of 30 were transplanted in the tail vein of sub-lethally irradiated (200 rad) 7-week-old NSG mice, as model of xenotransplantation to evaluate the hematological reconstitution of transduced cells and the level of GLB1 enzymatic activity upon gene-therapy (GT group). Cultured untransduced cells were similarly transplanted in NSG mice, referred as MOCK group, as for the hematopoietic stem cell transplantation, which is considered a standard therapy for other forms of LSDs. In detail, 4.3×105 cells/mouse were injected and the hematopoietic reconstitution was followed by analyzing the human cell engraftment in the peripheral blood (PB) at 7 and in the (BM) at 12 weeks after transplantation (FIG. 6A). The body weight of treated mice was also monitored over time. Moreover, 1×10⁵ cells of both conditions were expanded in vitro as myeloid liquid culture to determine potential toxic effect on cell proliferation, the efficiency of transduction (VCN) and the level of GLB1 expression and enzymatic activity. In vitro, no sign of toxicity was observed, considering that transduced cells grow as efficiently as untransduced cells (FIG. 6B); a VCN of 1.95 was measured in the myeloid cells derived from LV GLB1 transduced mPB CD34+ cells (FIG. 6C). The expression of GLB1 in the cell pellet and medium was evaluated by western blot, observing an increased expression of GLB1 in the cell pellet of LV GLB1 mPB CD34+ derived myeloid cells. In the cell medium, the presence of GLB1 protein only was observed in the conditioned medium from LV GLB1 myeloid cells (FIG. 6D). The dosage of GLB1 enzymatic activity confirmed an enrichment of GLB1 enzyme in the cell pellet and medium of myeloid cells derived from LV GLB1 transduced mPB CD34+ cells (FIG. 6E). In vivo, despite a reduced body weight overtime in the GT group compared to MOCK mice (FIG. 6F), all transplanted animals survived until the end of the experiments (12 weeks). Human engraftment in the PB of treated mice increased in the BM, from 7 weeks after transplantation to 12 weeks (FIG. 6G). Despite a reduced percentage of human CD45+cells in the BM of GT mice compared to MOCK group, a significantly higher enzymatic activity was measure in the total BM cells of GT mice (FIG. 6I).

This indicates that transduced cells expressed super-physiological level of enzyme, in accordance with the VCN determined in the genome of BM cells at 12 weeks after treatment (FIG. 6H). Altogether these data show that transduced mPB CD34+ cells efficiently engraft in the BM of recipient mice and overexpress GLB1 when transplanted in vivo. The expression level of GLB1 is considered sufficient to cross-correct cells of non-hematopoietic origins.

### Example 8—Evaluation of LV-MAN2B Wild-Type (WT) and Codon Optimized (OPT) Toxicity

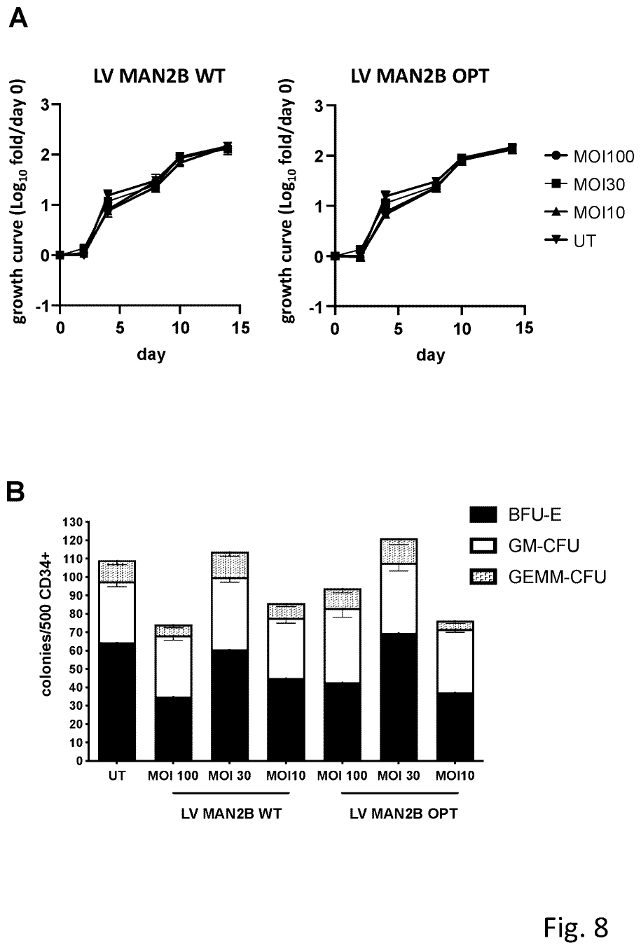

Human mobilized peripheral blood (mPB) CD34+ cells were transduced with LV-MAN2B WT and LV-MAN2B OPT (respectively, left and right panel of FIG. 8A) at different MOI (100, 30, 10) and expanded for 14 days as myeloid liquid culture and their growth curve was evaluated. Untransduced cells (UT) were used as controls to evaluate potential toxic effects on cell proliferation of transduced cells progeny; colony forming assay was also carried out (Fig. 8B), on human mPBCD34+ cells transduced with LV-MAN2B WT and OPT at different MOI (100, 30, 10). Untransduced cells (UT) were used as controls to determine potential toxic effects on the clonogenic capacity of LV-MAN2B WT and OPT HSPCs. LV-MAN2B WT or OPT did not show any significant toxicity in transduced cells.

### Example 9—Analysis of MAN2B Expression and Enzymatic Activity in Human mPB CD34+ Transduced with LV-MAN2B WT and OPT

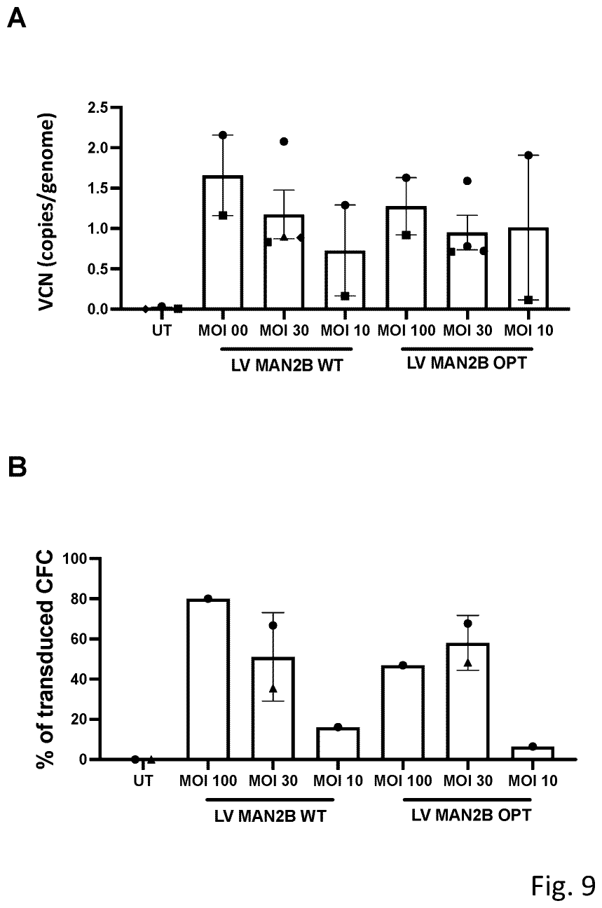

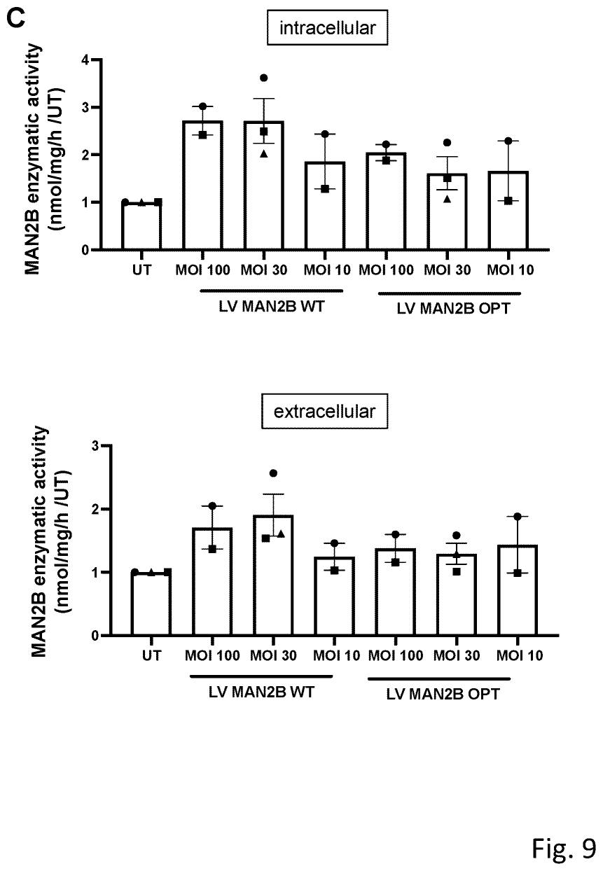

MAN2B enzymatic activity was tested in the progeny of mPBCD34+ transduced with LV-MAN2B WT and OPT at different MOI. In front of a similar vector copy number (VCN) integrated per cell (see FIG. 9A), LV-MAN2B OPT and WT showed comparable MAN2B enzymatic activity; also, it is noted that the transduction with a MOI of 30 (see FIG. 9B) is sufficient to obtain significant expression level and enzymatic activity (FIG. 9C), so that low amounts of the vector can be used.

### Example 10—Evaluation of LV-MAN2B WT Toxicity and Transduction Efficiency in Human mPB CD34+ Cells

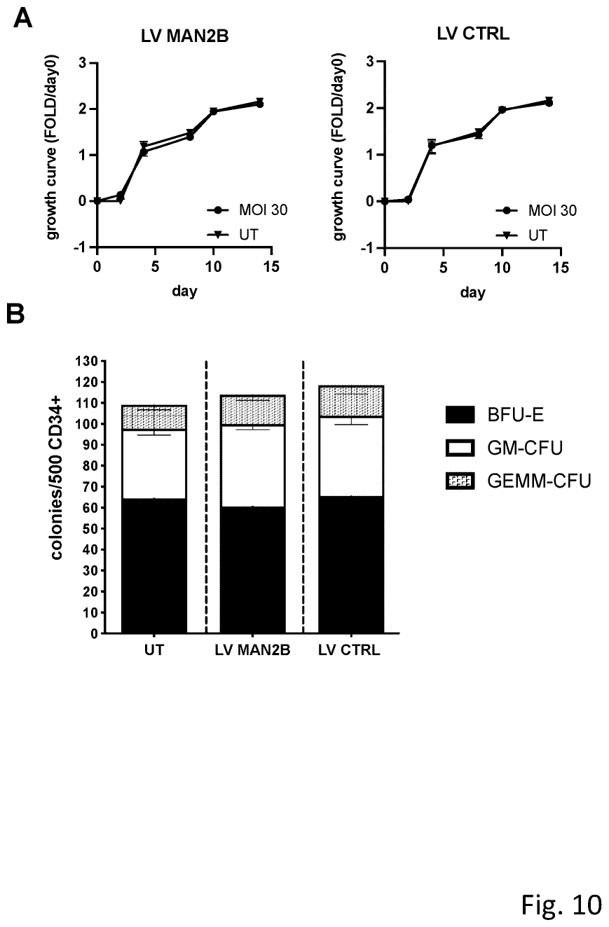

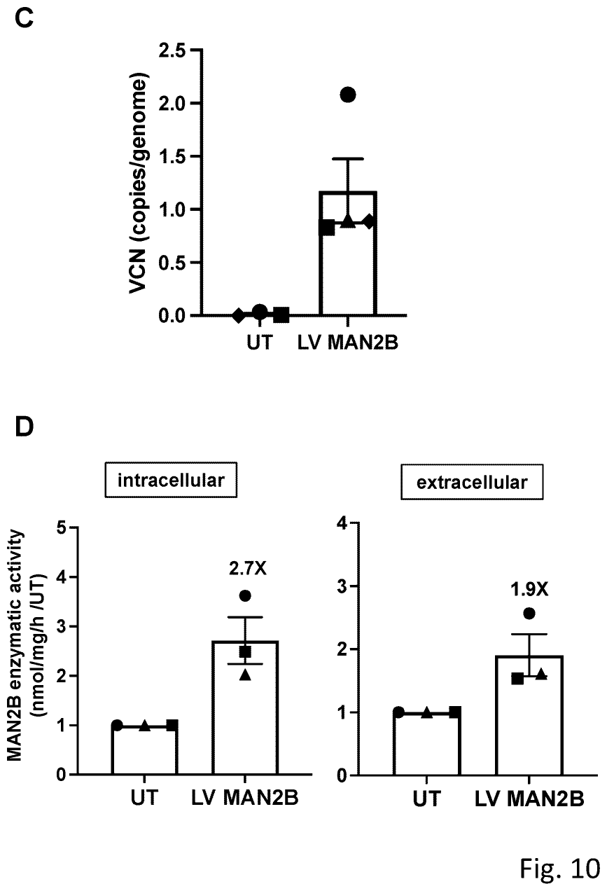

Growth curve analysis (FIG. 10A) and Colony forming assay (FIG. 10B) have been carried out on mPB hCD34+ transduced with LV-MAN2B WT (left panel of FIG. 10A) and LV-CTRL (right panel of FIG. 10A), at a MOI of 30, expanded as a myeloid liquid culture for 14 days. VCN measurement of the myeloid progeny of human mPB hCD34+ cells transduced with LV-MAN2B WT and dosage of MAN2B enzymatic activity in the cell pellet and medium of the myeloid progeny of human mPB hCD34+ cells transduced with LV-MAN2B WT have been also carried out (FIGS. 10C and D, respectively). mPB CD34+ cells untransduced (UT) were used as controls. Cells resulted efficiently transduced without signs of toxicity and expressed and released MAN2B enzyme at supraphysiological levels.

### Example 11—Restoration of MAN2B Enzymatic Activity in Fibroblasts Derived from Alpha-Mannosidosis Patients

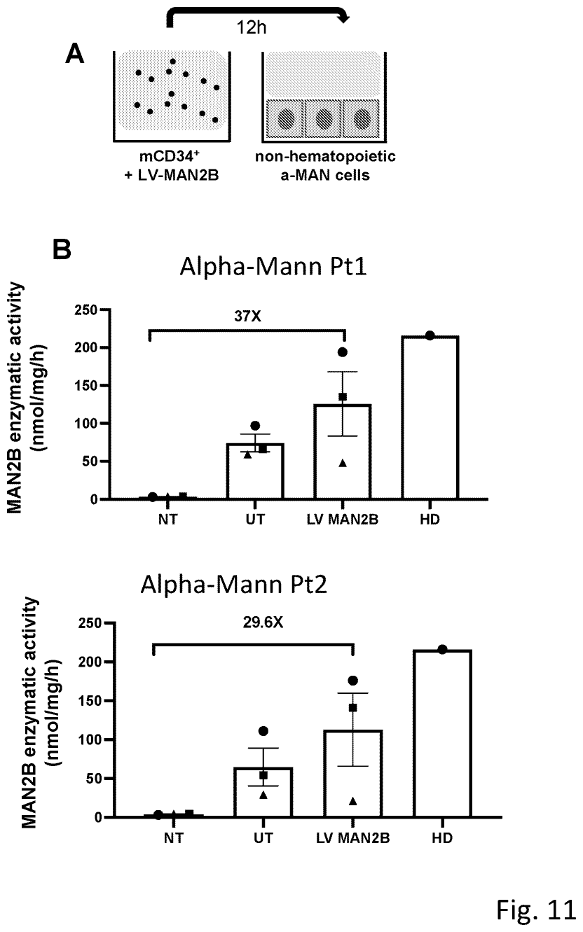

In an in vitro cross-correction model, fibroblasts derived from a-MANN patients (Pt1 and Pt2) have been exposed to the supernatant from mobilized peripheral blood (mPB) CD34+ cells transduced with a MOI of 30 with LV-MAN2B. A schematic representation of the cross-correction assay is sown in FIG. 11A. The cell medium conditioned by the myeloid progeny of human mPB CD34+ cells transduced with LV-MAN2B WT at an MOI of 30 was collected after 12-hour-conditioning. The cell medium conditioned by untransduced cells was used as a control. Fibroblasts from MAN2B patients were exposed to the conditioned medium for 12-16 hours and collected for western blot analysis and enzymatic activity dosage. Untransduced (UT) cells were used as control; also, MAN2B enzymatic activity was measured in both untreated patients' fibroblasts and fibroblasts from 1 healthy donor as a control (HD). The enzymatic activity was efficiently restored, underlying the capacity of mPB CD34+ transduced with LV-MAN2B to cross-correct patient cells of non-hematopoietic origin (see FIG. 11B).

### Example 12-Analysis of GLB1 Expression and Enzymatic Activity in HSPCs Transduced with LV GLB1 WT and OPT

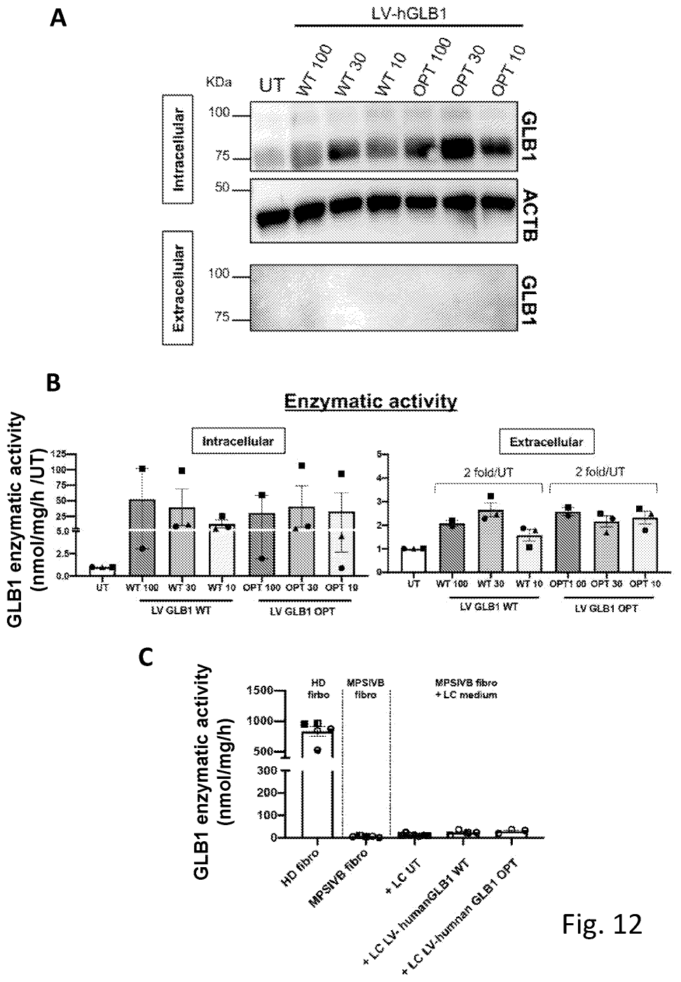

The level of GLB1 expression was analyzed also in the conditioned medium from transduced cells after 14 day-expansion in myeloid liquid culture. While a significantly higher intracellular overexpression of the GLB1 enzyme was observed compared to untransduced (UT) cells, the myeloid progeny of transduced HSPCs released a low amount of the enzyme in the cell medium. A 2-fold increase of GLB1 enzymatic activity was measured in the conditioned medium from the myeloid progeny of transduced cells (FIGS. 12A, B).

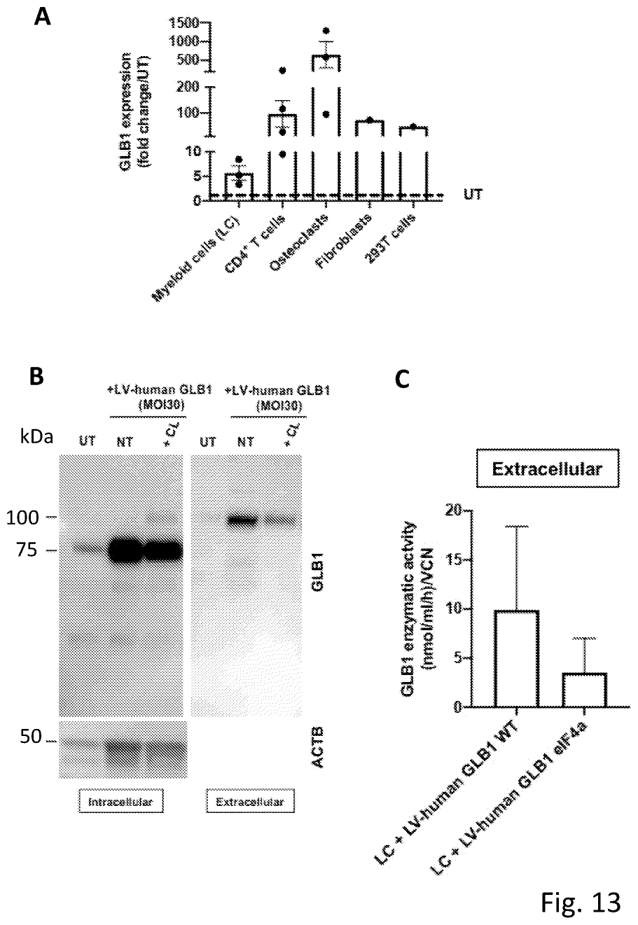

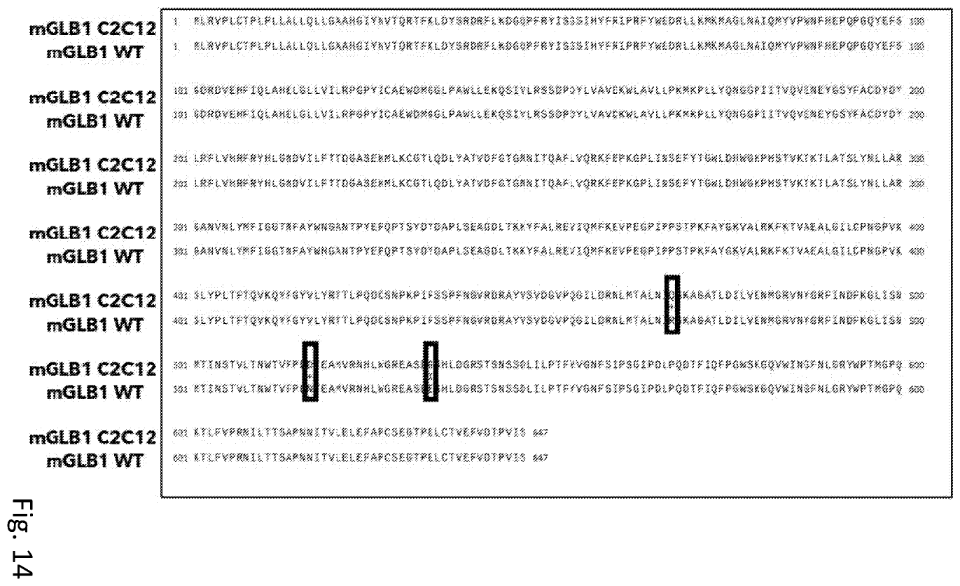

The level of GLB1 RNA expression was cell type-dependent (FIG. 13A). The GLB1 enzyme was processed along the ER-Golgi pathway into the lysosomal form (64 kDa) (FIG. 12A) and, thus, retained in the lysosomal compartment of myeloid cells from LV-human GLB1 transduced HSPCs. Indeed, the addition of Chloroquine (+CL) (100 mM) caused GLB1 precursor accumulation improving the GLB1 release (FIG. 13B). A similar level of protein release was found using the LV-human GLB1 eIF4a compared to LV-human GLB1 (FIG. 13C).

### Example 13-Production of Further Lentiviral Vectors for Expressing GLB1 genes

Third-generation lentiviral vectors were generated bearing polynucleotides encoding human GLB1 wild-type (LV-human GLB1 WT), murine GLB1 wild-type (LV-murine GLB1 WT) or the C2C12 isoform (LV-murine GLB1 C2C12), characterized by three amino acid substitutions (R468Q, N517D, and E534G) compared to the WT protein (FIG. 14A). The safety, efficiency, and efficacy of the LV vectors, and were determined, compared to the LV-human GLB1 WT in vitro 30) and in vivo in the Examples 14-18 that follow.

### Example 14—Analysis of GLB1 Expression and Enzymatic Activity in HSPCs Transduced with Lentiviral Vectors for Expressing GLB1 Genes

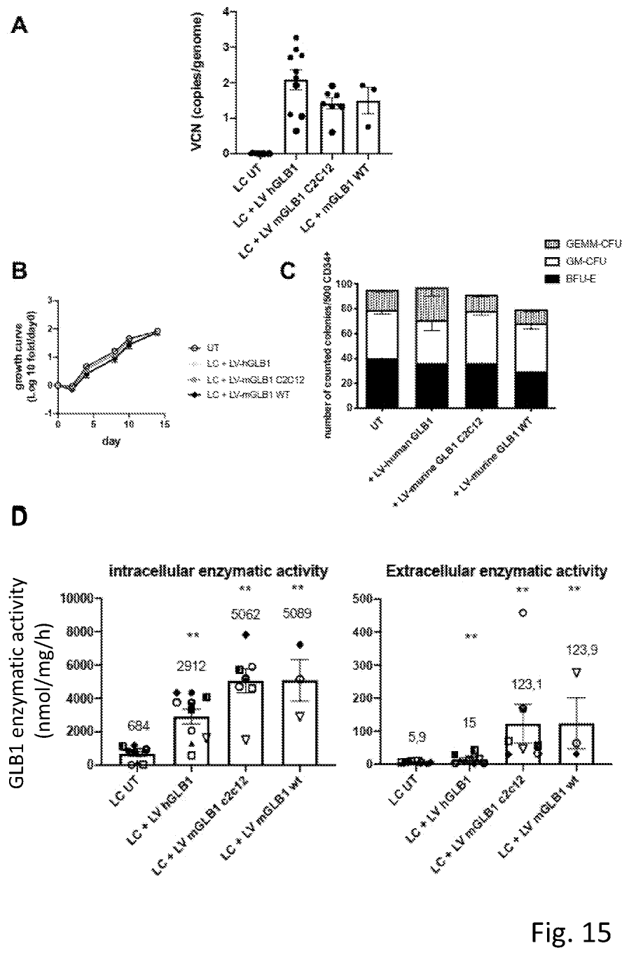

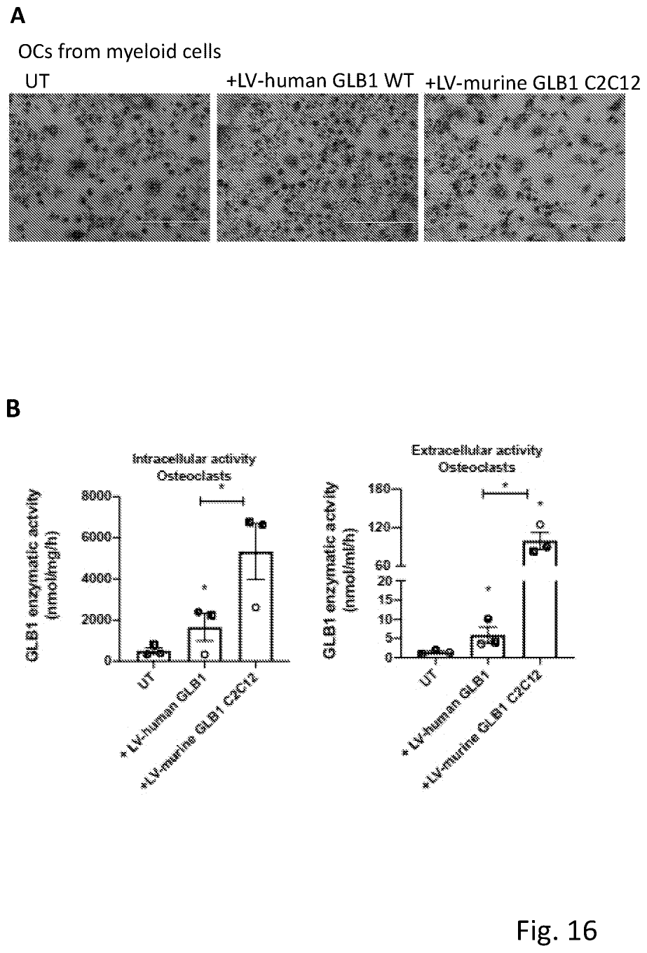

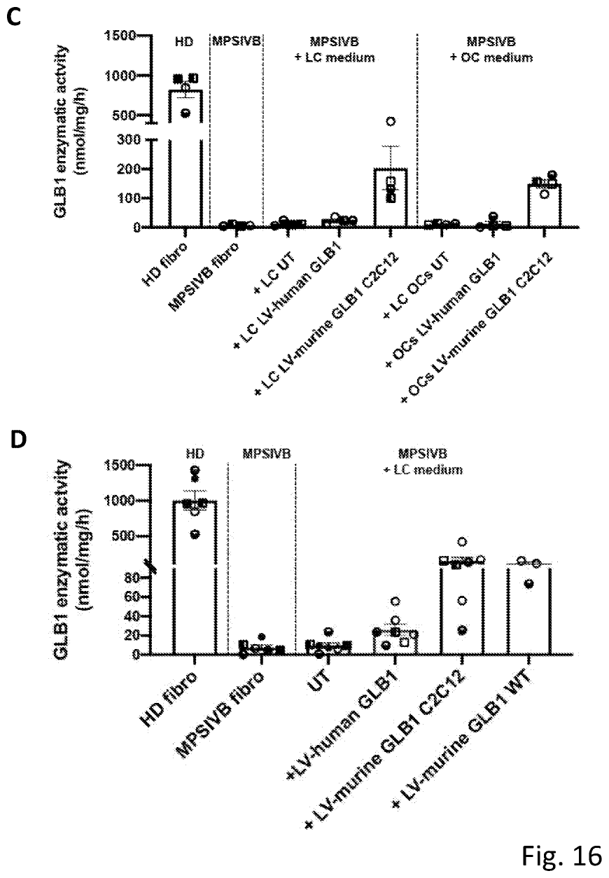

In vitro, human HSPCs from healthy donors were transduced with LV-murine GLB1 WT, LV-murine GLB1 C2C12, and LV-human GLB1 WT at an MOI of 30 according to the single hit transduction protocol in the presence of cyclosporin H (CsH) (8 mM) as transduction enhancer. After transduction, cells were collected, washed, and plated for colony-forming cell (CFC) assay and in vitro expansion as myeloid liquid culture. Human HSPCs were efficiently transduced as shown by VCN in the liquid culture (FIG. 15A), without signs of toxicity in terms of clonogenic capacity (FIG. 15B) and proliferation (FIG. 15C). The GLB1 expression was evaluated in the protein extract and conditioned medium from the myeloid liquid culture cells by enzymatic activity. A significantly higher GLB1 enzymatic activity was observed in the protein extract of myeloid cells from human HSPCs transduced with the LV-murine GLB1 (5062 nmol/mg/h for LV-murine GLB1 C2C12; 5089 nmol/mg/h for LV-murine GLB1 WT) than in cells transduced with the LV-human GLB1 (2912 nmol/mg/h for LV-human GLB1), leading to 7.4-fold and 4.3-fold increase in enzymatic activity compared to untransduced (UT) cells using the LV-murine GLB1 (both WT and C2C12) and the LV-human GLB1, respectively (FIG. 15D, left panel). A significant overexpression of GLB1 enzyme was also measured in the conditioned medium from the myeloid cells derived from the differentiation of human HSPCs transduced with LV-murine GLB1 (123.1 nmol/mg/h for LV-murine GLB1 c2c12; 123.9 nmol/mg/h for LV-murine GLB1 C2C12) corresponding to a 20-fold increase of enzymatic activity compared to untransduced (UT) cells. A 2.5-fold increase of enzymatic activity was reached in the myeloid cell-conditioned medium using the LV-human GLB1 (FIG. 15D, right panel). Further myeloid cells derived from human HSPCs+LV-murine GLB1 C2C12 were differentiated into osteoclasts to exclude any alterations associated with the expression of the murine enzyme. Myeloid cells transduced with LV-murine GLB1 C2C12 differentiated into TRAP-positive osteoclasts (OCs) similarly to untransduced (UT) and LV-human GLB1 myeloid cells (FIG. 16A), and overexpressed GLB1enzyme in the cell medium, reaching 60-fold higher GLB1 enzymatic activity compared to untransduced OCs. OCs from the myeloid cells transduced with LV-human GLB1 only showed a 4-fold higher GLB 1 activity (FIG. 16B). The conditioned medium from myeloid cells+LV-murine GLB1 C2C12 and myeloid cells +LV-murine GLB1 C2C12-derived osteoclasts was used to prove the cross-correction of MPSIVB fibroblasts (FIG. 16C). The GLB1 enzymatic activity was restored after 24 hour-exposure to the conditioned medium of LV murine GLB1 myeloid cells (fold change on untreated MPSIVB fibroblasts: 31,37) and osteoclasts (fold change on untreated MPSIVB fibroblasts: 23.11) (FIG. 16C). The level of enzymatic activity in fibroblasts exposed to the conditioned medium from LV-human GLB1 and untransduced myeloid cells was similar to the GLB1 enzymatic activity measured in untreated MPSIVB fibroblasts (fold change on untreated: 3.7 and 1.93, respectively). The conditioned medium from LV-human GLB1 and untransduced osteoclasts also showed a reduced cross-correction efficacy compared to the conditioned medium from LV-murine GLB1 transduced osteoclasts failed to restore the GLB1 enzymatic activity in MPSIVB fibroblasts (FIG. 16C). The efficacy of cross-correction was similar for the LV-murine WT and LV-murine C2C12. Indeed, no significant enzymatic activity differences (114.8 nmol/mg/h for LV-murine GLB1 WT; 151.8 nmol/mg/h for LV-murine GLB1 c2c12) was observed in MPSIVB fibroblasts exposed to the conditioned medium from LV-murine GLB1 WT and LV-murine GLB1 C2C12 myeloid cells (FIG. 16D).

### Example 15—Analysis of GLB1 Expression and Enzymatic Activity in Skeletal Cells Transduced with Lentiviral Vectors for Expressing GLB1 Genes

The functionality of the murine compared to the human GLB1 enzyme was further tested in skeletal cells.

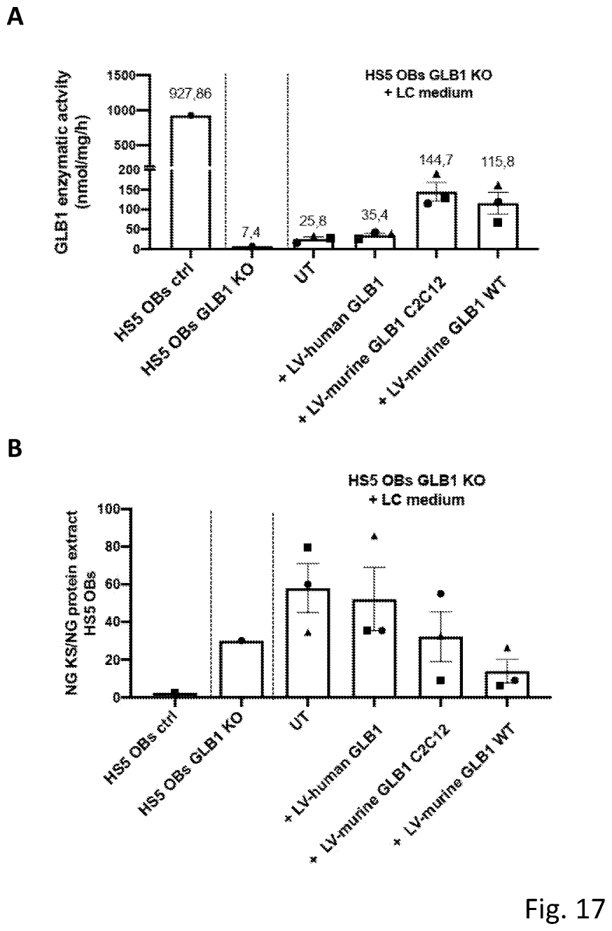

To this aim, GLB1 KO HS5 stromal cells were generated by using an LV expressing the Cas9 cDNA and a GLB1-specific gRNA (5′ CAGATACTATATGAACGGGCACAAA 3′). A control LV CRISPR, bearing a scrambled gRNA was also produced to generate CRISPR control HS5 cells. First, a significant reduction of the GLB1 enzymatic activity in GLB1 KO HS5 cells was proved (99% reduction compared to control cells). Further to inducing GLB1 KO and control HS5 cells to differentiate into osteoblasts, the cross-correction assay was performed on differentiated cells, incubating the GLB1 KO and CTRL osteoblasts with the conditioned medium from myeloid cells for 24 hours. The enzymatic activity was efficiently restored upon incubation with the conditioned medium from LV-murine GLB1 WT and LV-murine C2C12 transduced cells (FIG. 17A). The level of keratan sulfate was also measured by ELISA, showing a trend of keratan sulfate reduction in GLB1 KO HS5 cells exposed to the conditioned medium from LV-murine GLB1 WT and C2C12 myeloid cells (FIG. 17B). In vitro results demonstrated that human HSPCs transduced with the LV-murine GLB1 (WT and C2C12) generated myeloid cells and osteoclasts capable of releasing a high level of a stable GLB1 enzyme, which was uptake by target GLB1 deficient cells (MPSIVB fibroblast and GLB1 KO HS5-derived osteoblasts), restoring their enzymatic activity. The results sustain the use of LV-murine GLB1 for HSPC-GT.

### Example 16—In Vivo Transplantation Experiment using HSPCs Transduced with LV-GLB1 Vectors

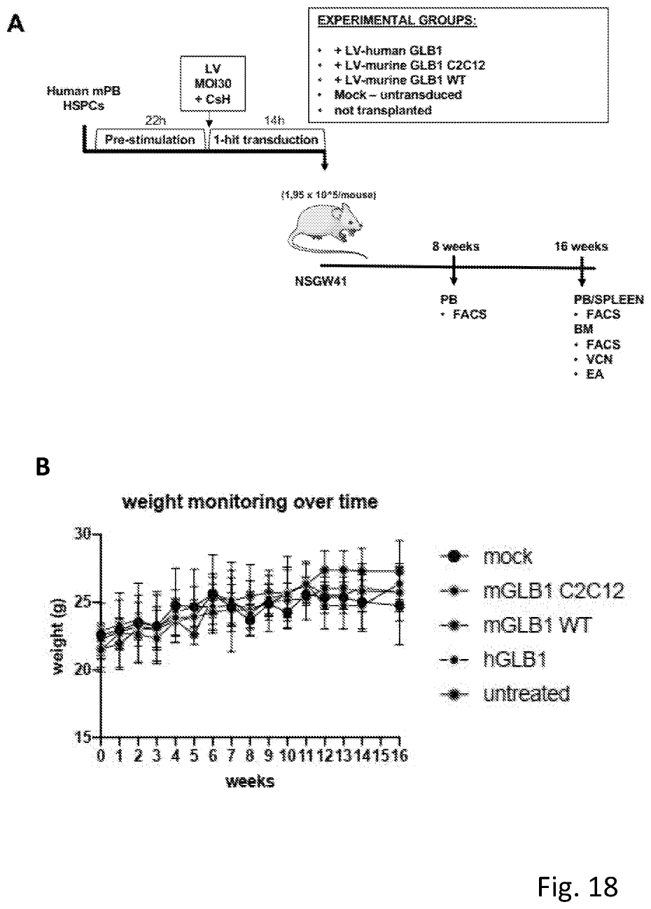

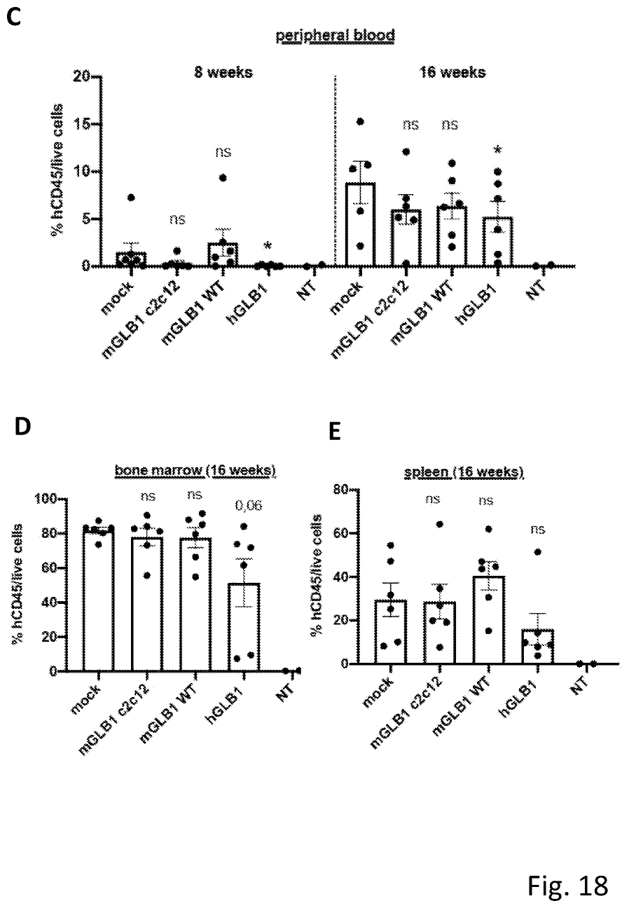

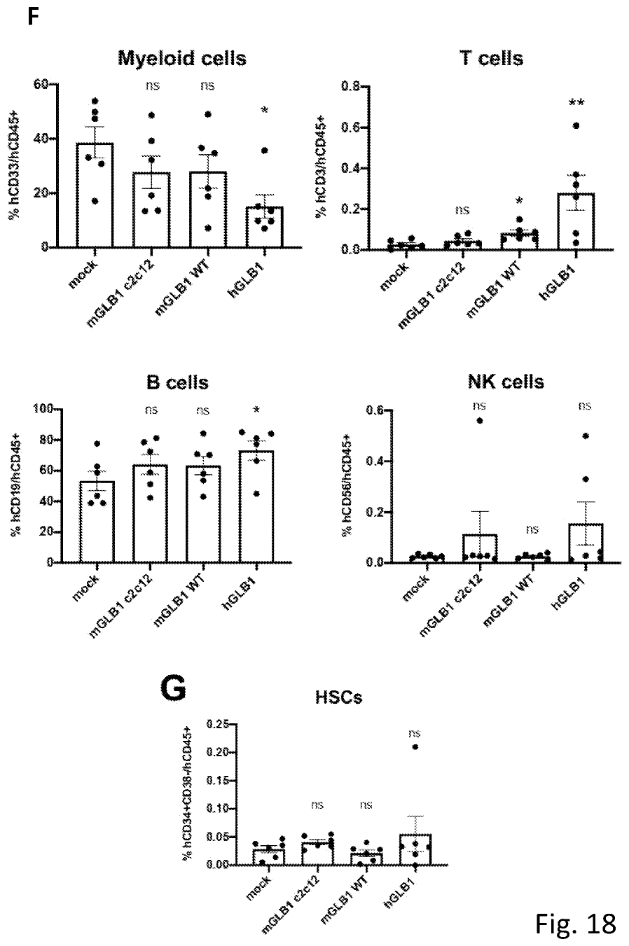

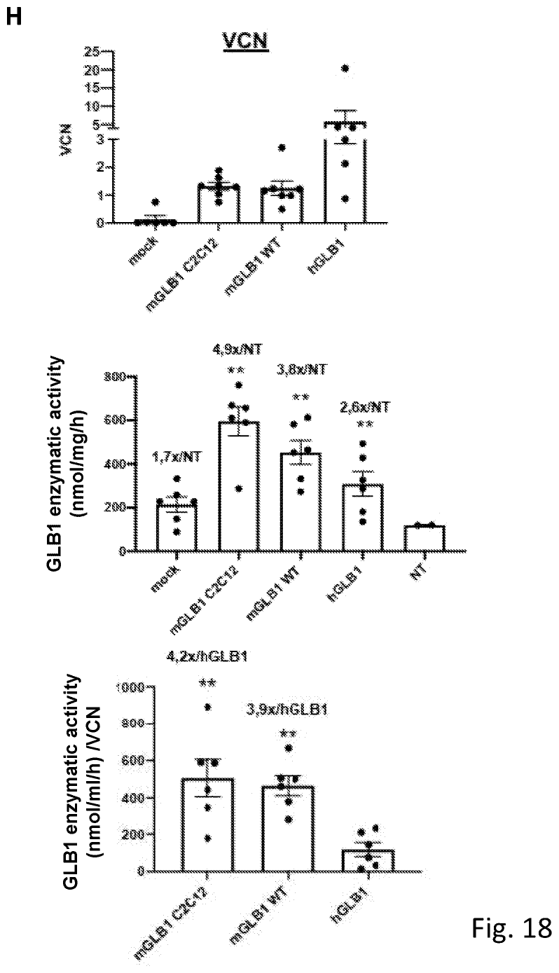

To further support the in vitro data, human HSPCs transduced in vitro at an MOI of 30 with LV-human GLB1, LV-murine GLB1 WT, or LV-murine GLB1 C2C12 vectors, were transplanted to exclude any alterations in the engraftment and reconstitution capacity of human cells overexpressing the murine enzyme and to evaluate the level of enzymatic activity in the bone-marrow achieved with the different L Vs. Transduced cells (1.95×10⁵/mouse for all the conditions) were xenotransplanted into 7-week-old immunodeficient NOD.Cg-Kit^{W-41J} Prkdc^{scid} Il2rg^{tmlWjl/}WaskJ (NSGW41). Untreated mice and mice transplanted with cultured untransduced cells were used as controls (FIG. 18A). The body weight of transplanted mice was monitored over time (FIG. 18B) and the level of human engraftment evaluated as the percentage of human CD45+ cells (% hCD45) in the peripheral blood at 8 and 16 weeks after transplantation. A slight but significant reduction of human engraftment was found in mice transplanted with HSPCs-LV-human GLB1 (mean hCD45+%=5.3) compared to mock transplanted mice (mean hCD45+%=8,9). On the contrary, similar percentages of human CD45+ cells were measured in mice transplanted with human HSPCs+LV-murine GLB1 WT and C2C12 compared to mock controls (FIG. 18C). At 16 weeks, a similar level of human engraftment was detected in the bone marrow and spleen of transplanted mice (FIGS. 18D, E), demonstrating that the expression of the murine GLB1 enzyme did not impact the biodistribution capacity of transduced cells (FIGS. 18 C-E). It was also demonstrated that in the bone marrow, engrafted human HSPCs transduced with the different LVs properly differentiated into myeloid, T-, B-, and NK cells similar to mock control cells (FIG. 18F). A similar percentage of human HSCs (CD34+, CD38−) was measured in the bone marrow of transplanted mice, proving that the in vitro gene correction procedure did not alter the primitive HSC compartment (FIG. 18G). In addition, the VCN in the bone marrow of transplanted mice was measured to assess the efficiency of human HSPC transduction, showing a higher VCN in mice transplanted with human HSPCs+LV-human GLB1 (FIG. 18H). However, a significantly higher level of GLB1 enzymatic activity was detected in the BM of mice transplanted with HSPCs transduced with the LV-murine GLB1 C2C12 and WT (4.9 and 3.8-fold compared to not treated mice, respectively) and compared to mice injected with HSPCs+LV-human GLB1 (2,6-fold compared to not treated mice) (FIG. 18I, upper panel). These differences were more evident when the enzymatic activity was normalized to the VCN (FIG. 18I, lower panel). Altogether, the in vivo data confirmed the capacity of transduced cells to engraft and reconstitute the hematopoietic system similarly to untransduced cells. They also confirmed a mild induction of GLB1 enzymatic activity achieved in the bone marrow of transplanted mice when using the LV-human GLB1. Moreover, the transplantation of human HSPCs transduced with the LV-murine GLB1 allowed to reach a significantly higher expression of the GLB1 enzyme in vivo compared to untreated HSPCs and HSPCs+LV-human GLB1.

### Example 17—Evaluation of Cross-Correction with HSPCs Transduced with LV-GLB1 Vectors

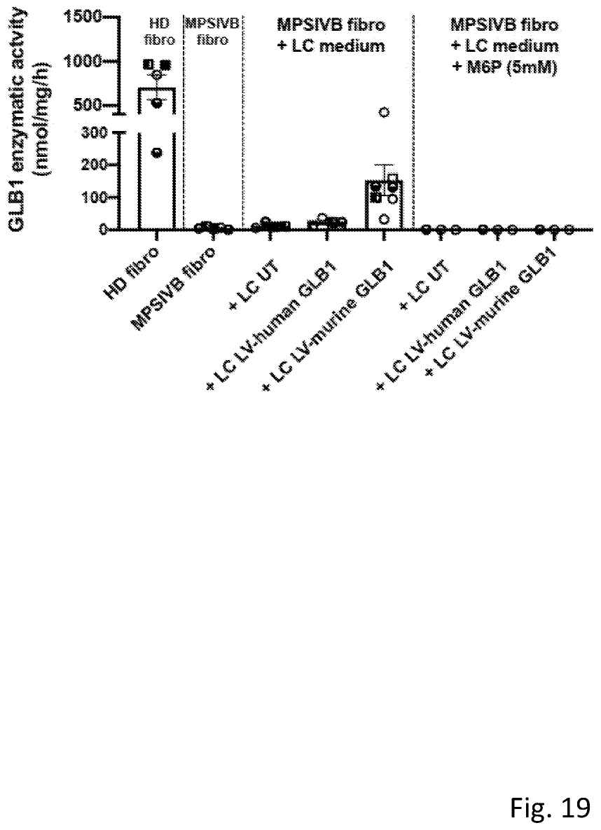

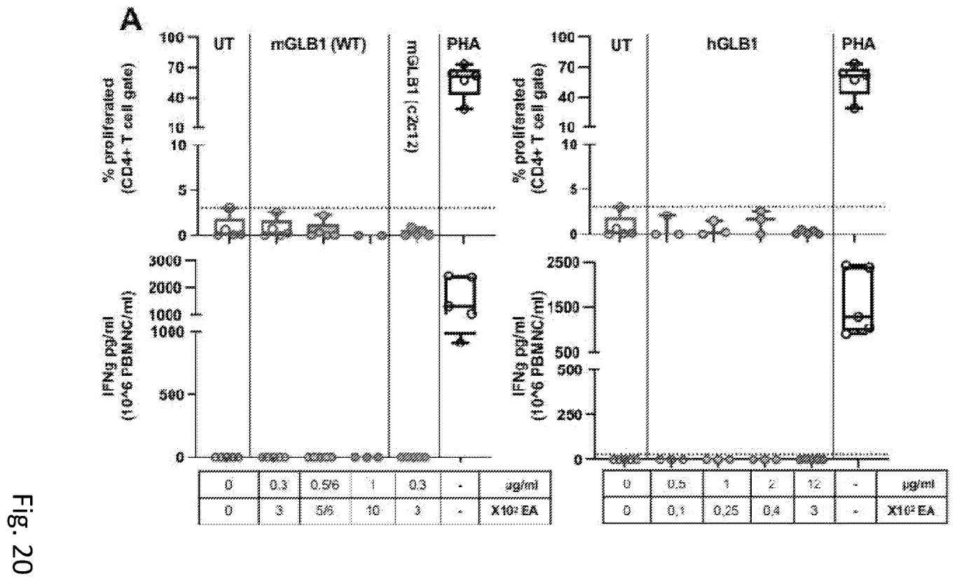

An additional study was performed to compare the mechanism of cross-correction. The murine enzyme was internalized by MPSIVB fibroblasts via the mannose 6P receptor (M6PR). The presence of M6P in the conditioned medium from myeloid cell+LV-murine GLB1 inhibited the enzyme uptake, preventing the restoration of the GLB1 enzymatic activity, similar to LV-human GLB1 (FIG. 19A). Also, the murine and human GLB1 enzyme were detected in fibroblasts exposed to the conditioned medium from myeloid cell+LV-murine GLB1 or +LV-human GLB1 by immunofluorescence, in combination with Vimentin (cytoplasm marker) and LAMP-1 (lysosomal marker) (FIGS. 20A-C). Both the murine and human enzymes co-localized with LAMP-1, demonstrating that the internalized enzymes were correctly targeted into the lysosomal compartment.

### Example 18—Immunogenicity Assays with LV-GLB1 Vectors

The protein structure of the murine and human GLB1 enzySme show a 70% homology in the amino acidic sequence, with a low percentage of highly biochemical different amino acids in the corresponding positions. Considering these differences, a short and long-term immunogenicity assay was performed using peripheral blood mononuclear cells (PBMNCs) from healthy donors. In the short-term response, PBMCs were exposed to the conditioned media from HEK-293T cells transduced with LV-human GLB1, LV-murine GLB1 WT, and LV-murine GLB1 C2C12 for 5 days. T cell proliferation and INFγ production were evaluated to compare the immunogenicity of the human and murine GLB1 enzymes (FIG. 20A). In the secondary challenge experiment, PBMCs were exposed to conditioned media for 13 days. At day 0 autologous CD14+ cells were isolated and differentiated into autologous dendritic cells (DCs) in the presence of rh-IL-4 and rh-GM-CSF for 7 days. PBMCs, previously exposed for 13 days to the conditioned medium, were pulsed with the conditioned media from HEK-293T cells transduced with LV human or murine GLB1 (tetatuns toxoid was used as positive control) for 3 hours and cultured with autologous DCs for 3 days, prior to T cell response evaluation (proliferation and INFγ production) (FIG. 20B). Results of both short and long-term immunogenicity assays show an undetectable T-cell response to vector-derived human or murine GLB 1 in both short and long-term immunogenicity assay. These analyses further sustained the use of LV-murine GLB1 for the development of HSPC-GT for MPSIVB patients.

### Example 19—Evaluation of Toxicity and MAN2B Activity in Human mPB CD34+ Transduced with LV-MAN2B.

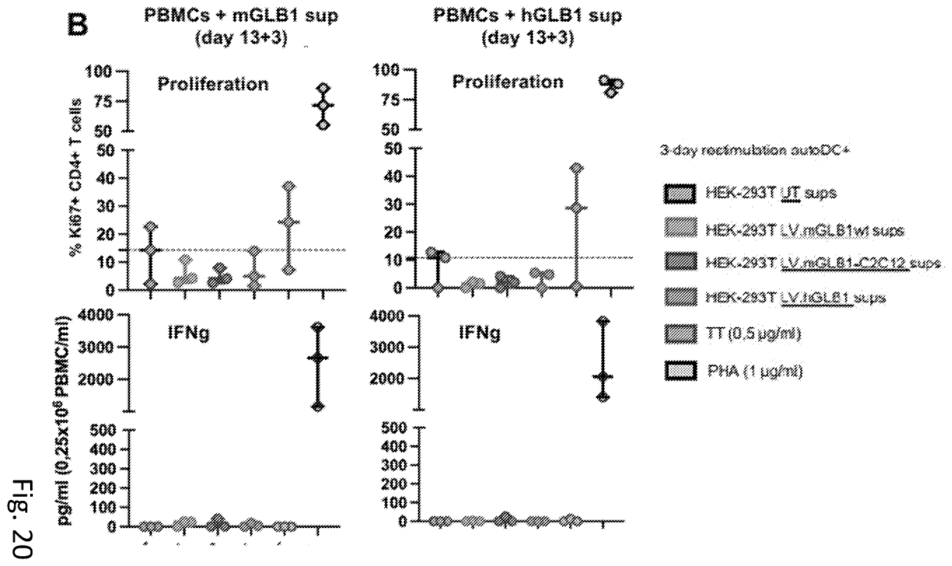

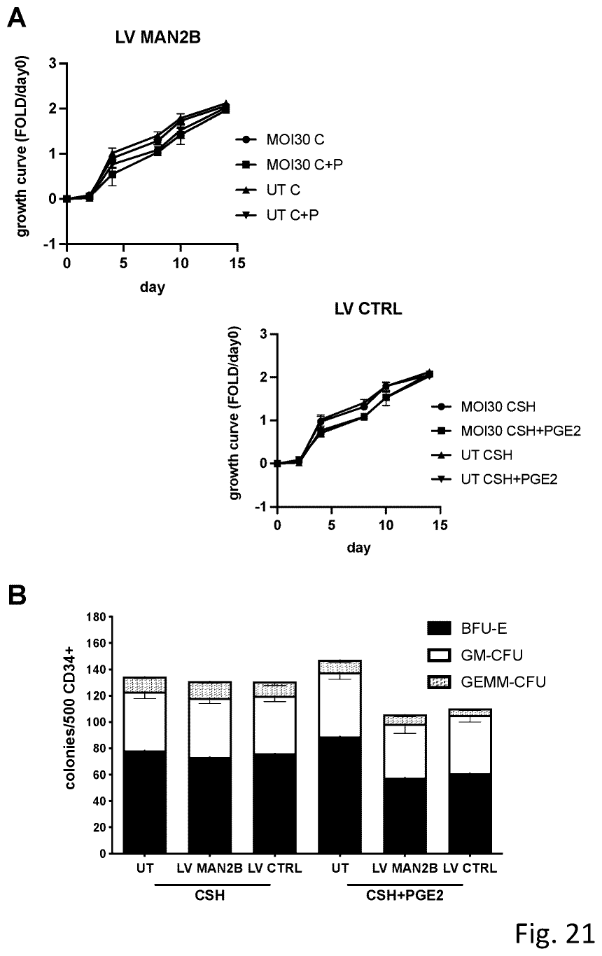

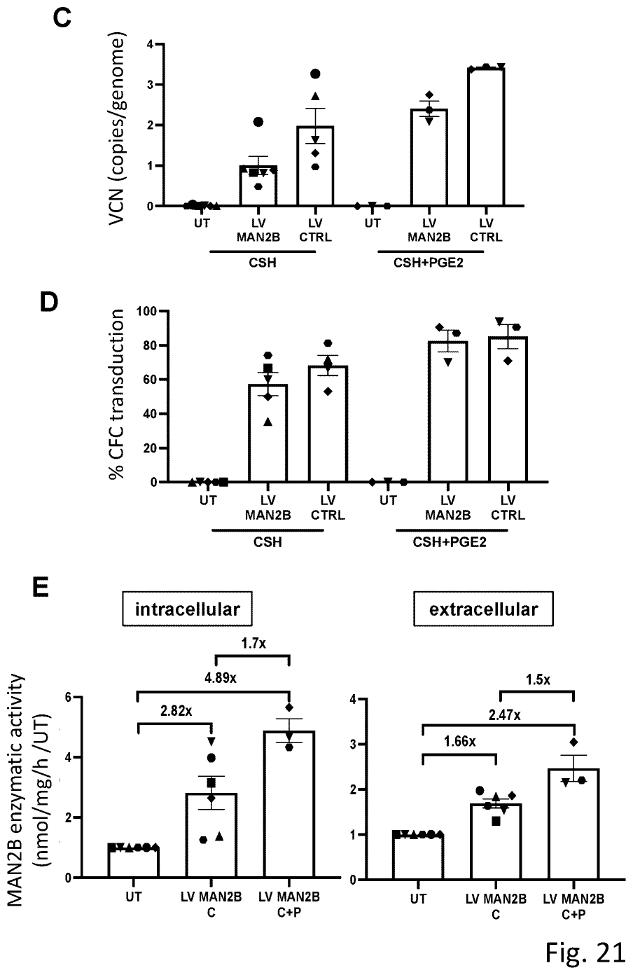

Human mobilized peripheral blood (mPB) CD34+ cells were transduced with LV-MAN2B WT and a control vector (LV-CTRL) at MOI 30 in the presence of Cyclosporine H (8 uM, CsH condition) alone or in combination with Prostaglandin E2 (10 uM, CsH+PGE2 condition) as transduction enhancers. The 1-hit CsH+PGE2 protocol was tested with the aim of increasing the vector copy number (VCN) per cell and the target enzyme activity in comparison with 1-hit CsH protocol. After transduction, cells were expanded for 14 days as myeloid liquid culture and plated for colony-forming cell (CFC) assay to evaluate potential toxic effects on cell proliferation and clonogenic potential. Untransduced cells (UT) were used as controls. Transduced and untransduced cells proliferate at same rate along liquid culture and preserved clonogenic potential, with slight decrease of the cell growth and number of colonies in the CsH+PGE2 condition (FIGS. 21A-B). Cells resulted efficiently transduced and the combination of CsH and PGE2 resulted in 2-fold higher VCN per cell and increased percentage of transduction with respect to CsH alone (FIGS. 21C-D). Moreover, myeloid progeny deriving from LV-MAN2B transduced mPB-CD34+ cells expressed and released MAN2B enzyme at supraphysiological levels, according to their VCN (2.8-fold with CsH and 4.9-fold with CsH+PGE2, intracellular; 1.7-fold with CsH and 2.5-fold with CsH+PGE2, extracellular) (FIG. 21E).

### Example 20—Restoration of MAN2B Enzymatic Activity in Fibroblasts Derived from Alpha-Mannosidosis Patients Using Conditioned Media from LV-MAN2B Liquid Cultures

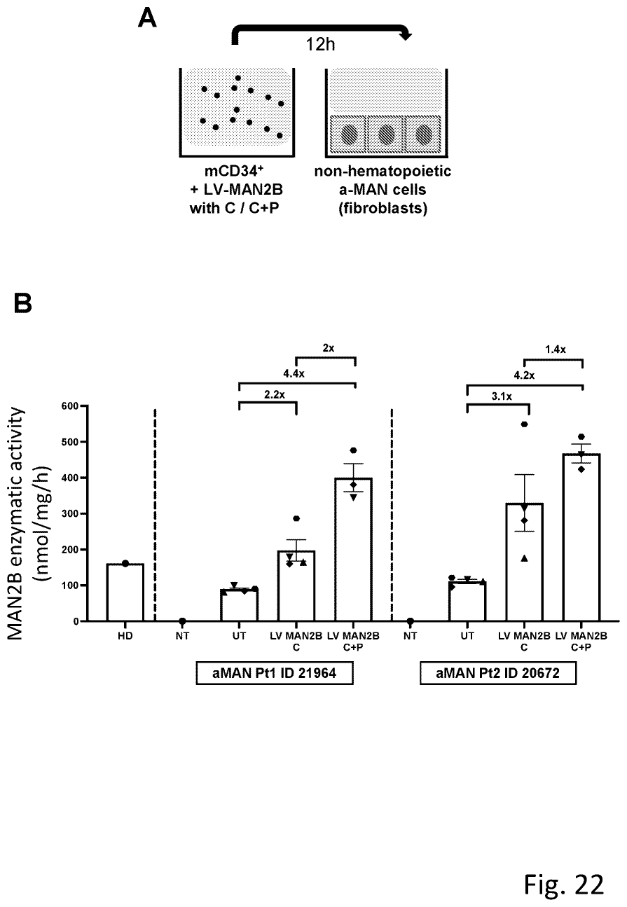

In an in vitro cross-correction model, fibroblasts derived from alpha-mannosidosis patients have been exposed to the supernatant from mobilized mPB CD34+ cells transduced with LV-MAN2B at MOI 30 with either CsH or CsH+PGE2 transduction protocols. A schematic representation of the cross-correction assay is shown in FIG. 22A. The cell medium conditioned by the myeloid progeny of transduced human mPB CD34+ cells was collected after 12-hour-conditioning. The cell medium conditioned by untransduced cells was used as control. Fibroblasts from MAN2B patients were exposed to the conditioned medium for 12-16 hours and collected for enzymatic activity dosage. Untransduced (UT) cells were used as control; also, MAN2B enzymatic activity was measured in both untreated patients' fibroblasts and fibroblasts from 1 healthy donor as control (HD). The results show a full restoration of MAN2B enzymatic activity in patients' fibroblasts reaching comparable or even superior levels with respect to HD control. A cross correction>1.4-fold higher was observed after exposure to CsH+PGE2-derived medium with respect to CsH-derived one (FIG. 22B). These results underly the capacity of mPB CD34+ transduced with LV-MAN2B to cross-correct patient cells of non-hematopoietic origin.

### Example 21—Osteoclasts (OCs) Derived from the Myeloid Progeny of human mpb CD34+ Cells Transduced with LV-MAN2B Released MAN2B Enzyme

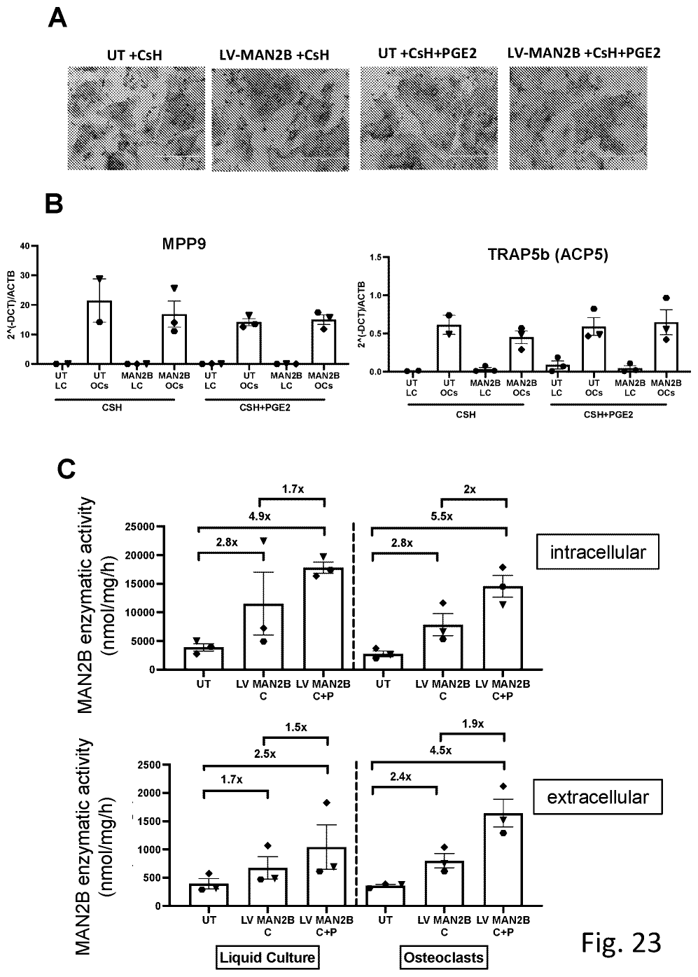

TRAP assay was performed to evaluate the presence of OCs after 10-day of in vitro differentiation of the myeloid progeny of mPB CD34+ cells transduced with LV-MAN2B at MOI 30 with either CsH or CsH+PGE2 protocols (FIG. 23A). OCs derived from untransduced (UT) cells were used as 30) a control. qPCR expression analysis of MMP9 and TRAP5b genes involved in OC differentiation was performed (FIG. 23B), together with measurement of MAN2B enzymatic activity in the cell pellet and medium of OCs (FIG. 23C). Osteoclasts deriving from transduced human mPB CD34+ cells showed higher MAN2B enzymatic activity compared to UT control. The highest VCN was reached with CsH+PGE2 transduction protocol resulted in a>1.9-fold increased enzymatic activity both intracellularly and extracellularly with respect to CsH alone.

### Example 22—Restoration of MAN2B Enzymatic Activity in Fibroblasts Derived from Alpha-Mannosidosis Patients Using Conditioned Media of Osteoclasts Transduced with LV-MAN2B

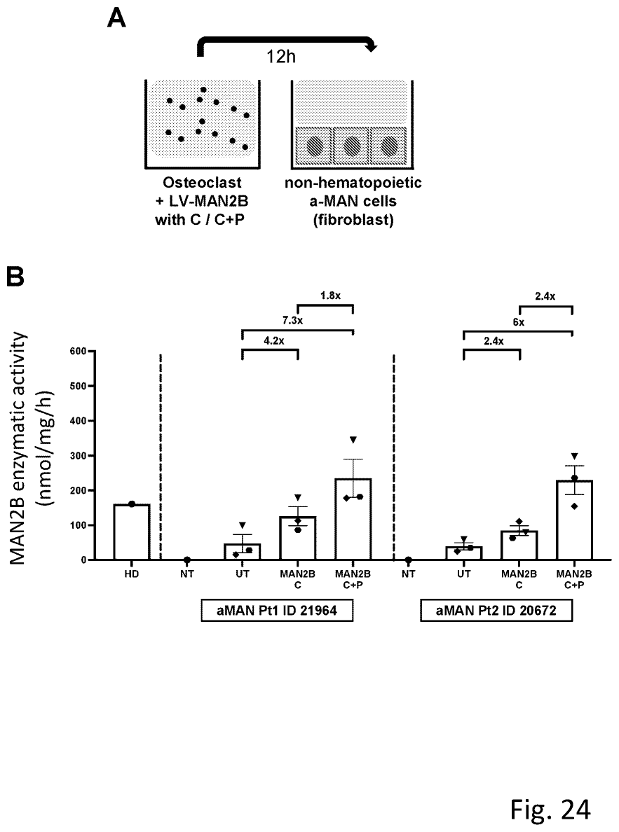

Following an in vitro cross-correction model, fibroblasts derived from alpha-mannosidosis patients have been exposed to the cell media conditioned by the osteoclasts derived from myeloid progeny of human mPB CD34+ cells transduced with LV-MAN2B WT at MOI 30 with 1-hit CsH or 1-hit CsH+PGE2 protocols. A schematic representation of the cross-correction assay is shown in FIG. 24A. The cell medium conditioned by osteoclasts was collected after 12-hour-conditioning. The cell medium conditioned by untransduced cells was used as control. Fibroblasts from MAN2B patients were exposed to the conditioned medium for 12-16 hours and collected for enzymatic activity dosage. Untransduced (UT) cells were used as control; also, MAN2B enzymatic activity was measured in both untreated patients' fibroblasts and fibroblasts from 1 healthy donor as control (HD). The results show a restoration of MAN2B enzymatic activity in patients' fibroblasts at comparable or even superior levels with respect to HD control. Also, a>1.8-fold higher cross-correction was observed after exposure to CsH+PGE2-derived medium with respect to CsH-derived one (FIG. 24B). These results imply that osteoclasts deriving from LV-MAN2B transduced human mPB CD34+ cells can function as a local source of enzyme within bones upon treatment.

### Example 23 In Vivo Transplantation Experiment Using Human mPB CD34+ Cells Transduced with LV-MAN2B and Untransduced Cells

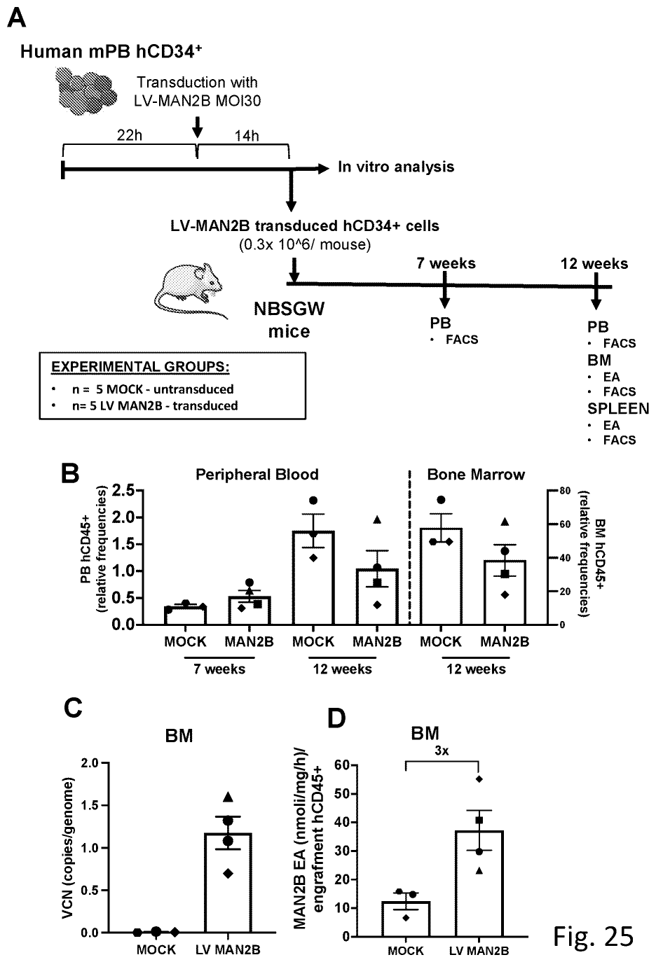

FIG. 25A shows the experimental scheme of the in vivo transplantation of engineered CD34+ cells derived from healthy donors and transduced by 1-hit CsH protocol with LV-MAN2B WT at MOI 30 in NBSGW mice, to assess the capability of transduced cells to overexpress MAN2B in vivo. In detail, injection of 0.3*10⁶ cells/mouse was followed by the analysis of the human cell engraftment in the PB at 7 and 12 weeks after transplantation. At 12 weeks mice were euthanized and bone marrow (BM) samples were analyzed for human cell content and MAN2B enzymatic activity. Mice transplanted with untransduced cultured mPB CD34+ cells (MOCK group) were used as controls. MAN2B and MOCK groups displayed not statistically-significant different human hematopoietic content in PB over time as well as in BM at termination, providing initial evidence that MAN2B overexpression does not 30) alter human HSPC engraftment and differentiation (FIG. 25B). At termination, we detected transduced cells in the BM of mice transplanted with LV-MAN2B-engineered human HSPC measuring a mean VCN of 1 (FIG. 25C). In addition, a significantly higher MAN2B enzymatic activity (3-fold) was measured in the total BM cells from the MAN2B group compared to the MOCK group. Altogether these data show that transduced mPB CD34+ cells efficiently engraft in the BM of immunocompromised mice and overexpress MAN2B when transplanted in vivo.

### Example 24—Production of Lentiviral Vectors for Expressing GALNS Genes

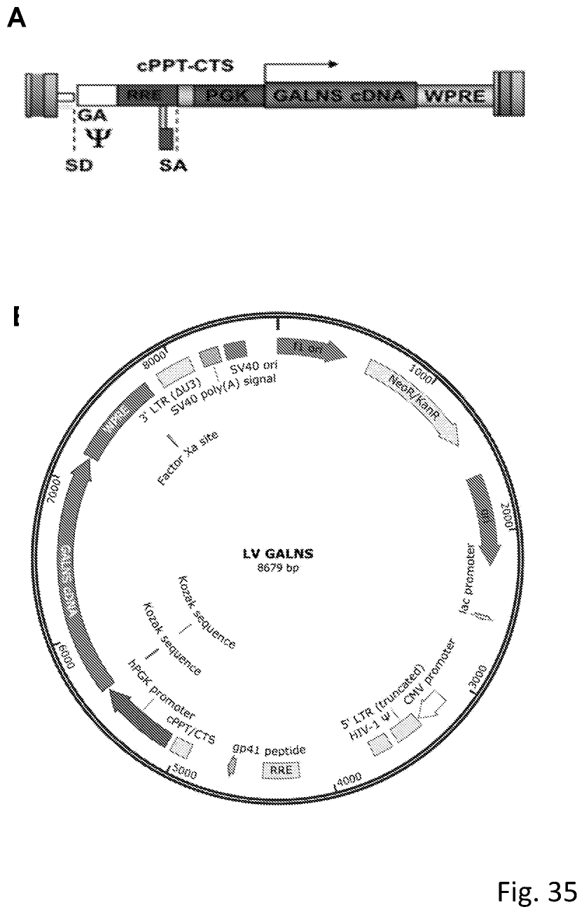

Three different LVs were produced: one with a transfer vector construct pCCLsin.cPPT.hPGK.GALNSwt.Wpre whose expression cassette comprises a wild-type polynucleotide encoding GALNS (SEQ ID NO: 35), herein indicated as “LV GALNS WT cassette”, one with a transfer vector construct pCCLsin.cPPT.hPGK.GALNSopt. Wpre whose expression cassette comprises a codon-optimized polynucleotide encoding GALNS (SEQ ID NO: 36), herein indicated as “LV GALNS OPT cassette”, and one with a transfer vector construct pCCLsin.cPPT.hPGK.eGFP.Wpre comprising an expression cassette for expressing eGFP reporter gene. FIG. 35B provides a schematic view of a transfer vector construct; FIG. 35A provides a schematic view of an expression cassette in the transfer vector construct.

### Example 25—In-Vitro Transduction of LVs for Expressing GALNS Genes in Mobilized Peripheral Blood (mPB) CD34+ Cells

Granulocyte colony-stimulating factor (G-CSF)-mobilized peripheral blood CD34+ cells were placed in culture on retronectin-coated non-tissue culture-treated wells (T100A Takara) in CellGro medium (Cell Genix) supplemented with the following cytokines: 60 ng/ml interleukine-3 (IL-3), 100 ng/ml thrombopoietin (TPO), 300 ng/ml stem cell factor (SCF), and 300 ng/ml FLT3-L (all from Cell Peprotech). After 22 hours of pre-stimulation, cells were transduced at a specific multiplicity of infection (MOI) with a single hit of the lentiviral vectors (LV) of Example 24 for 16 hours in the same cytokine-containing medium in the presence of 8 uM Cyclosporine H (CsH) (Merck), as transduction enhancer. After transduction, cells were collected, washed and plated for colony-forming cell (CFC) assay and myeloid liquid culture.

### Example 26—Evaluation of LV GALNS Wild-Type (WT) and Codon Optimized (OPT) Toxicity

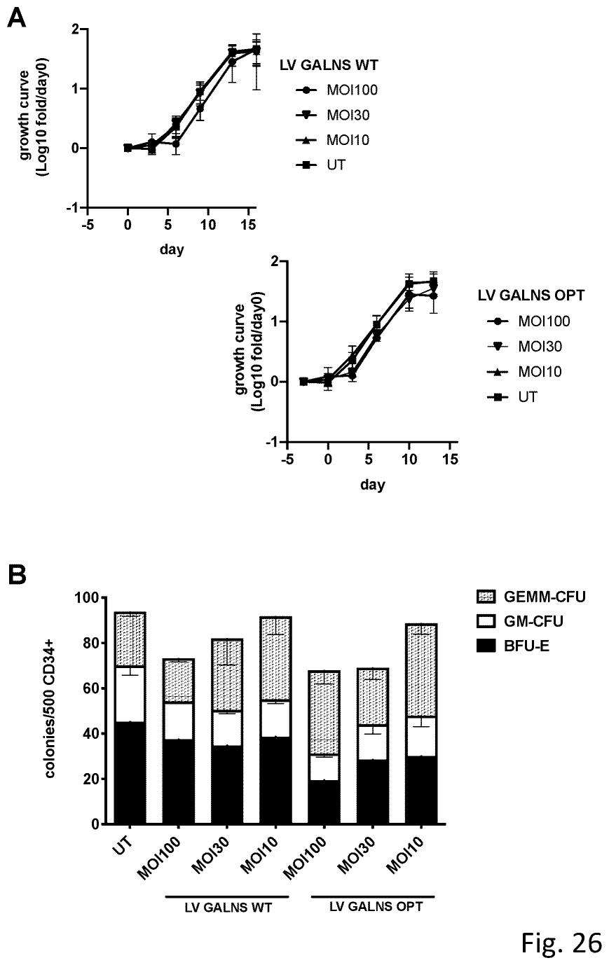

Human mobilized peripheral blood (mPB) CD34+ cells were transduced with LV GALNS WT (up-left panel of FIG. 26A) and LV GALNS OPT (low-right panel of FIG. 26A) at different MOI (100, 30, 10) and expanded for 14 days as myeloid liquid culture and their growth curve was evaluated. Untransduced cells (UT) were used as controls to evaluate potential toxic effects on cell proliferation of transduced cells progeny; colony forming assay was also carried out, on human mPBCD34+ cells transduced with LV GALNS WT and LV GALNS OPT at different MOI (100, 30, 10). Untransduced cells (UT) were used as controls to determine potential toxic effects on the clonogenic capacity of LV GALNS WT and OPT HSPCs. As shown in FIG. 26, LV GALNS WT or OPT did not show any significant toxicity in transduced cells.

### Example 27FIG. 25Analysis of GALNS Expression and Enzymatic Activity in Human mPB CD34+ Transduced with LV GALNS WT and OPT

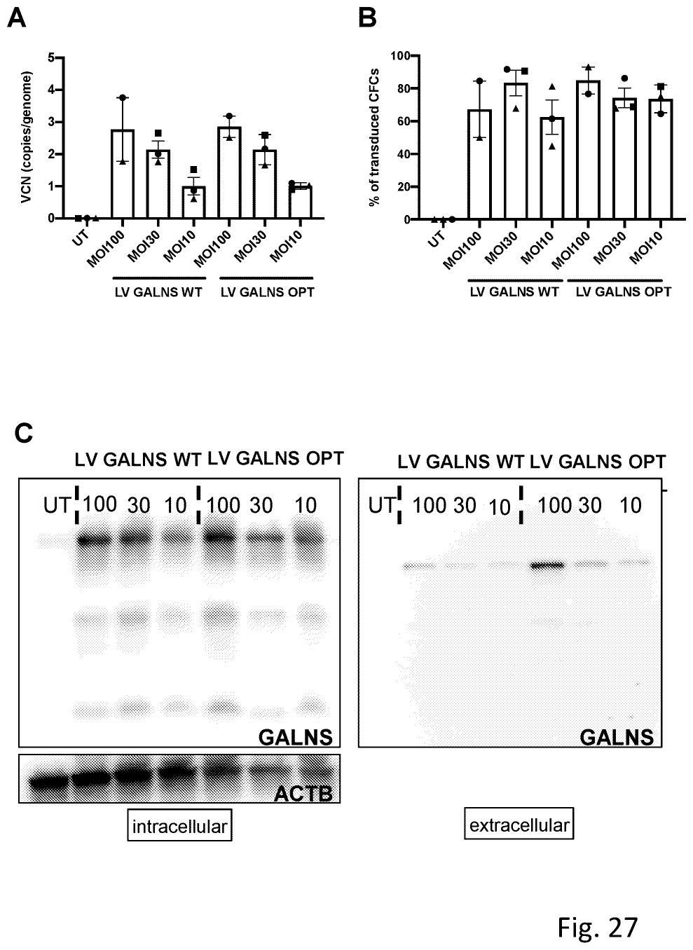

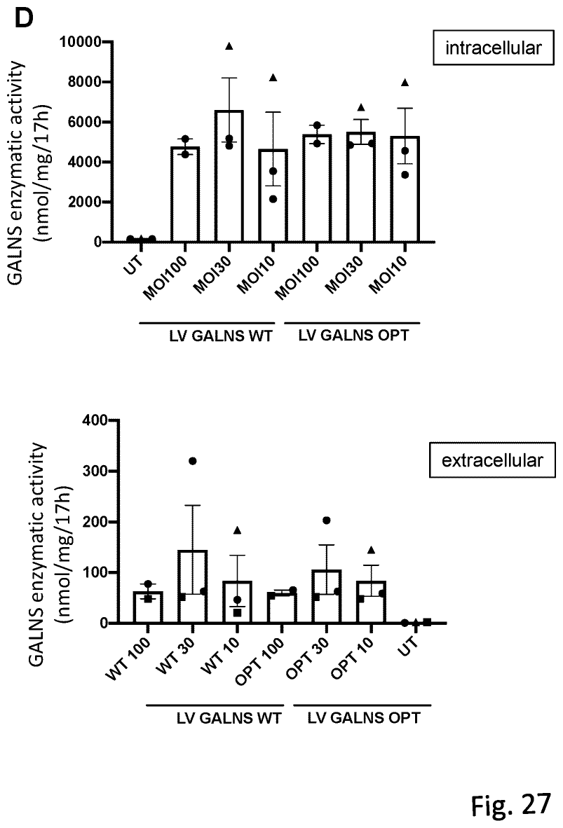

GALNS expression and enzymatic activity was tested in the progeny of mPBCD34+ transduced with LV GALNS WT and OPT at different MOI. In front of a similar vector copy number (VCN) integrated per cell (see FIG. 27A), LV GALNS OPT and WT showed comparable GALNS expression and enzymatic activity; also, it is noted that the transduction with a MOI of 30 (see FIG. 27B) is sufficient to obtain significant expression level and enzymatic activity (FIGS. 27 C and D), so that low amounts of the vector can be used.

### Example 28FIG. 25Evaluation of LV GALNS WT Toxicity and Transduction Efficiency in Human mPB CD34+ Cells

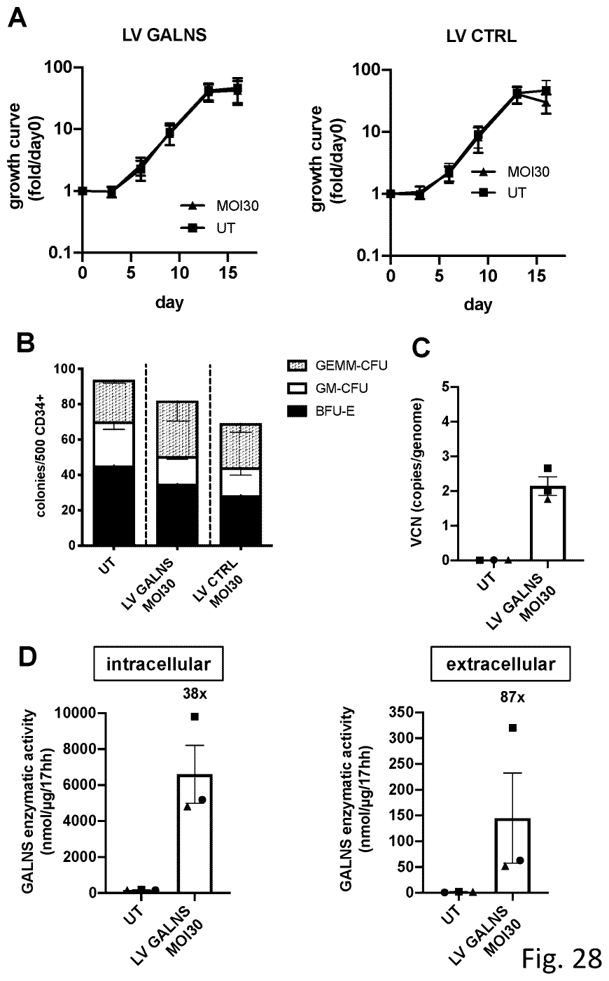

Growth curve analysis (FIG. 28A) and Colony forming assay (FIG. 28B) have been carried out on mPB hCD34+ transduced with LV GALNS WT (left panel of FIG. 28A) and LV-CTRL, at a MOI of 30, expanded as a myeloid liquid culture for 14 days. VCN measurement of the myeloid progeny of human mPB hCD34+ cells transduced with LV GALNS WT and dosage of GALNS enzymatic activity in the cell pellet and medium of the myeloid progeny of human mPB hCD34+ cells transduced with LV GALNS WT have been also carried out (FIG. 28C and D, respectively). mPB CD34+ cells untransduced (UT) and transduced with a control vector (LV GFP) were used as controls.

Cells resulted efficiently transduced without signs of toxicity and expressed and released GALNS enzyme at supraphysiological levels.

### Example 29—Restoration of GALNS Enzymatic Activity in Fibroblasts Derived from MPSIVA Patients

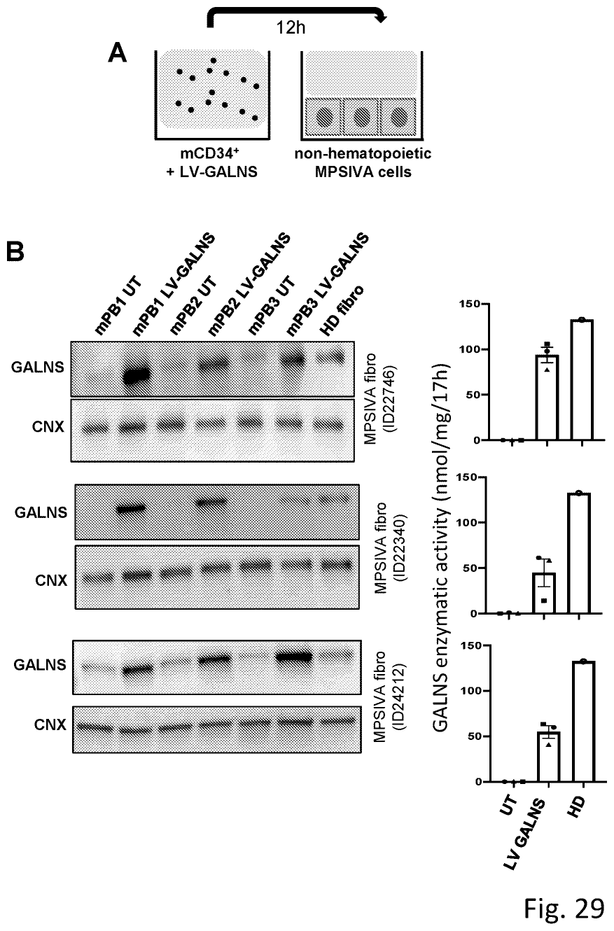

In an in vitro cross-correction model, fibroblasts, MSCs and osteoblasts derived from relevant patients have been exposed to the supernatant from mobilized peripheral blood (mPB) CD34+ cells transduced with a MOI of 30 with LV-GALNS. A schematic representation of the cross-correction assay is sown in FIG. 29A. The cell medium conditioned by the myeloid progeny of human mPB CD34+ cells transduced with LV GALNS WT at an MOI of 30 was collected after 12-hour-conditioning. The cell medium conditioned by untransduced cells was used as a control. Fibroblasts from MPSIVA patients were exposed to the conditioned medium for 12-16 hours and collected for western blot analysis and enzymatic activity dosage. Untransduced (UT) cells were used as control; also, GALNS enzymatic activity was measured in fibroblasts from 1 healthy donor as a control (HD). The enzymatic activity was efficiently restored, underlying the capacity of mPB CD34+ transduced with LV-GALNS to cross-correct patient cells of non-hematopoietic origin (see FIG. 29B).

### Example 30—Restoration of GALNS Activity in MPSIVA-Derived Mesenchymal Stromal Cells (MSCs) and MSC-Derived Osteoblasts (OBs)

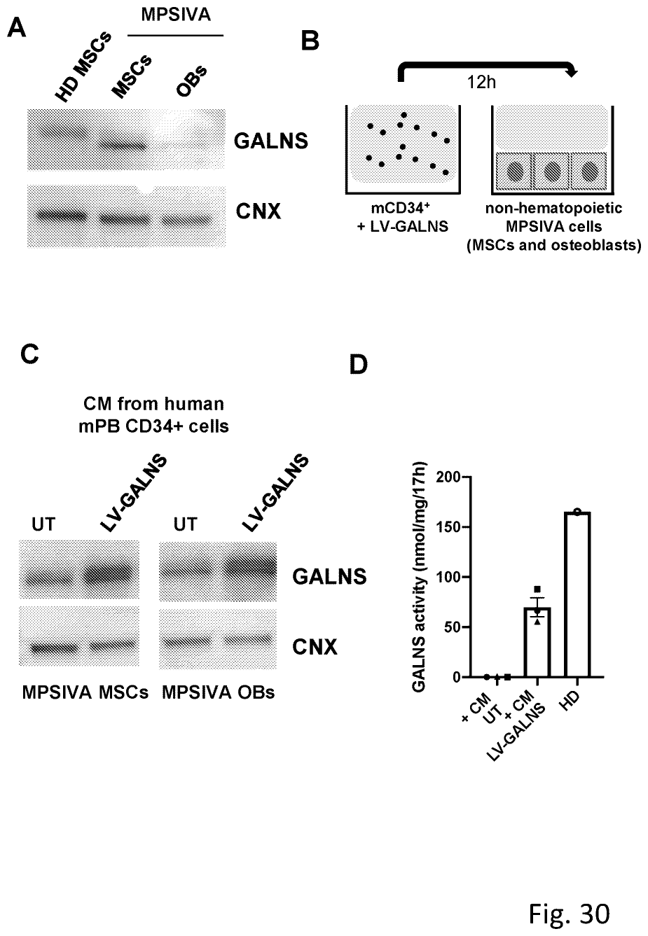

Restoration of GALNS Activity by LV GALNS WT was also assessed in mesenchymal stromal cells (MSCs) and MSC-derived osteoblasts (OBs) isolated from a MPSIVA patient, after obtaining informed consent. GALNS expression was also evaluated in MSCs derived from healthy-donor as a control. Actin-beta (ACTB) was used as a sample normalizer. FIG. 30B provides a schematic representation of the cross-correction assay.

mPB CD34+ cells were transduced with LV GALNS WT at an MOI of 30 and expanded for 14 days as myeloid liquid culture. After this, cells were plated at a concentration of 1×10⁶/ml for medium conditioning. The cell medium conditioned by transduced mPB CD34+ and untransduced cells was collected after 24-hour-conditioning. MSCs and MSC-derived OBs from MPSIVA patient were exposed to the conditioned medium for 12-16 hours and collected for western blot analysis (FIG. 30C) and enzymatic activity dosage (FIG. 30D). The cell medium conditioned by untransduced cells was used as a control.

Patient cells do uptake GALNS extracellular enzyme. Enzymatic activity was in fact efficiently restored, underlying the capacity of mPB CD34+ transduced with LV-GALNS to cross-correct patient cells of non-hematopoietic origin.

### Example 31—Analysis of the Molecular Mechanisms Mediating GALNS Uptake in MPSIVA MSCs and MSC-derived OBs

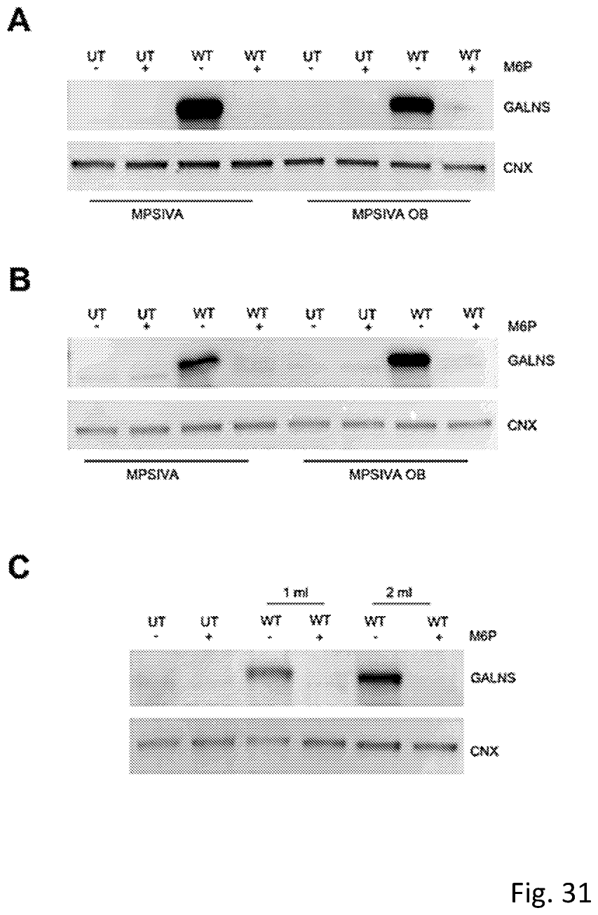

MPSIVA patient-derived MSCs and osteoblasts were exposed to the conditioned medium from HEK293T cells transduced with LV GALNS at a MOI of 30, reproducing the mPB CD34+ transduction conditions of Example 30. Different protocols of exposure were tested: 1) in the presence or absence of mannose 6 phosphates (M6P), the ligand of mannose 6 phosphate receptor (M6PR), which controls lysosomal enzyme trafficking; 2) exposure to different volumes of the conditioned medium; 3) different time of exposure. The results indicated that the uptake of GALNS from the conditioned medium increased after prolonged exposure and in the presence of a higher volume of conditioned medium. The presence of M6P inhibited GALNS uptake, indicating that GALNS uptake is M6P-dependent (FIG. 31).

### Example 32—Osteoclasts (OCs) Derived from the Myeloid Progeny of Human mPB CD34+ Cells Transduced with LV GALNS WT Expressed and Released GALNS Enzyme

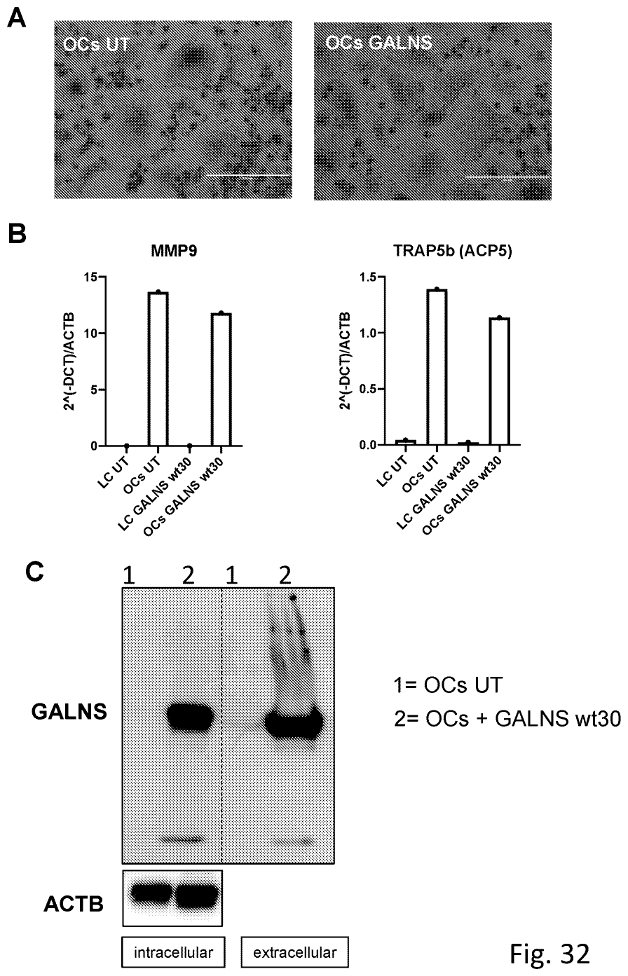

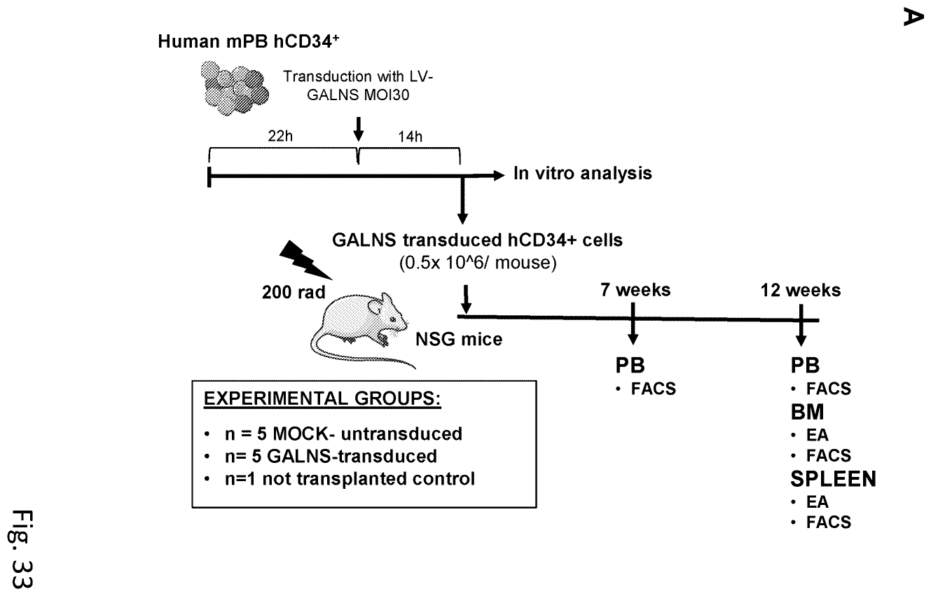

TRAP assay was carried out to evaluate the presence of OCs after 10-day of in vitro differentiation of the myeloid progeny of mPB CD34+ cells transduced with LV GALNS at an MOI of 30. OCs derived from untransduced (UT) cells were used as a control. qPCR expression analysis of MMP9 and TRAP5b genes involved in OC differentiation was performed, together with Western blot analysis of GALNS expression in the pellet and cell medium of OCs derived from the myeloid culture of untransduced and LV GALNS WT transduced mPB CD34+ cells (FIGS. 32 B, C). The osteoclast deriving from transduced human mPB CD34+ cells expressed and released GALNS enzyme, potentially functioning as a local source of enzyme in the bones upon the treatment.

### Example 33—In Vivo Transplantation Experiment Using Human mPB CD34+ Cells Transduced with LV GALNS WT and Untransduced Cells.

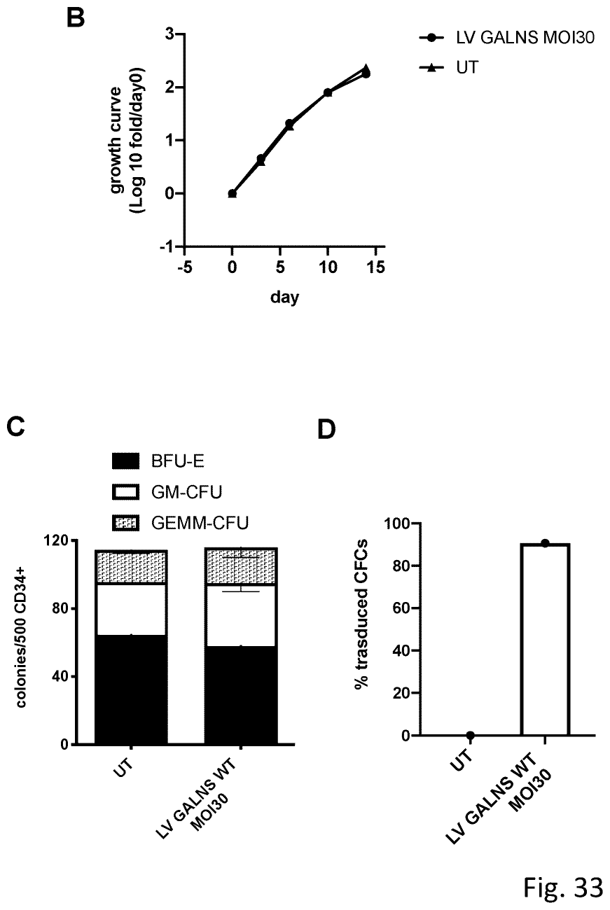

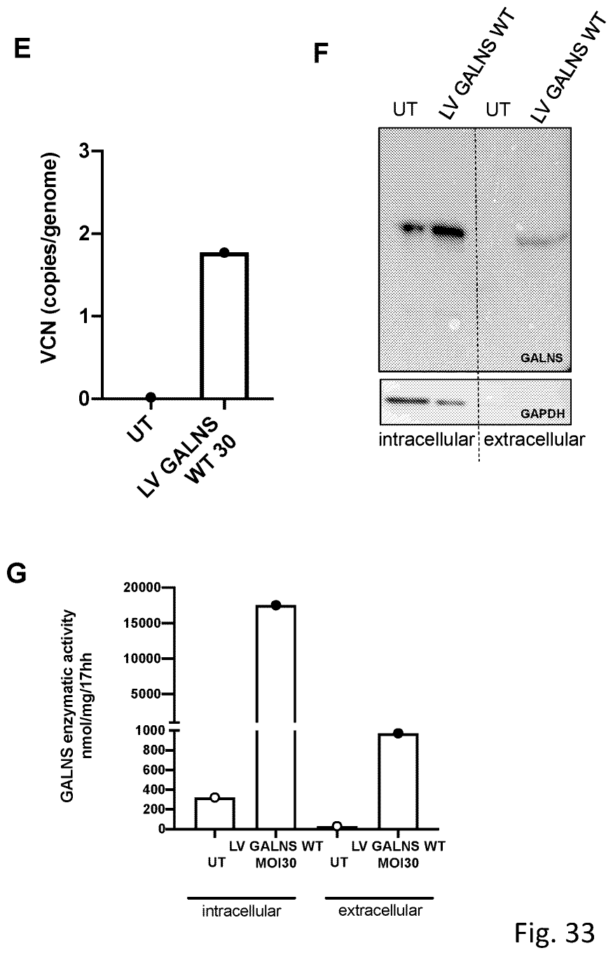

FIG. 33A shows the experimental scheme of in vivo transplantation of engineered CD34+ cells derived from healthy donors and transduced by 1-hit CsH protocol with LV GALNS WT at an MOI of 30 in in sublethally irradiated NSG mice (GT), to assess the capability of transduced cells to overexpress GALNS in vivo. In detail, injection of 0.5*10⁶ cells/mouse in sub-lethally irradiated NSG mice (200 rad) was followed with the hematopoietic reconstitution by analyzing the human cell engraftment in the PB at 7 and 12 weeks after transplantation. At 12 weeks mice were euthanized and hematopoietic organs (BM and spleen) were analyzed for human cell content and GALNS enzymatic activity. Mice transplanted with untransduced cultured mPB CD34+ cells (MOCK group) were used as controls.

In vivo data obtained in NSG mice transplanted with LV-GALNS-engineered human HSPCs show an increased expression of GALNS enzyme in their BM, compared to mice transplanted with untransduced control HSC (HSCT mice group) despite a similar level of human engraftment (FIGS. 33B-G).

### Example 34—In Vivo Human Reconstitution of Human mPB CD34+ Cells Transduced with LV GALNS WT and Untransduced Cells after Xenotransplantation

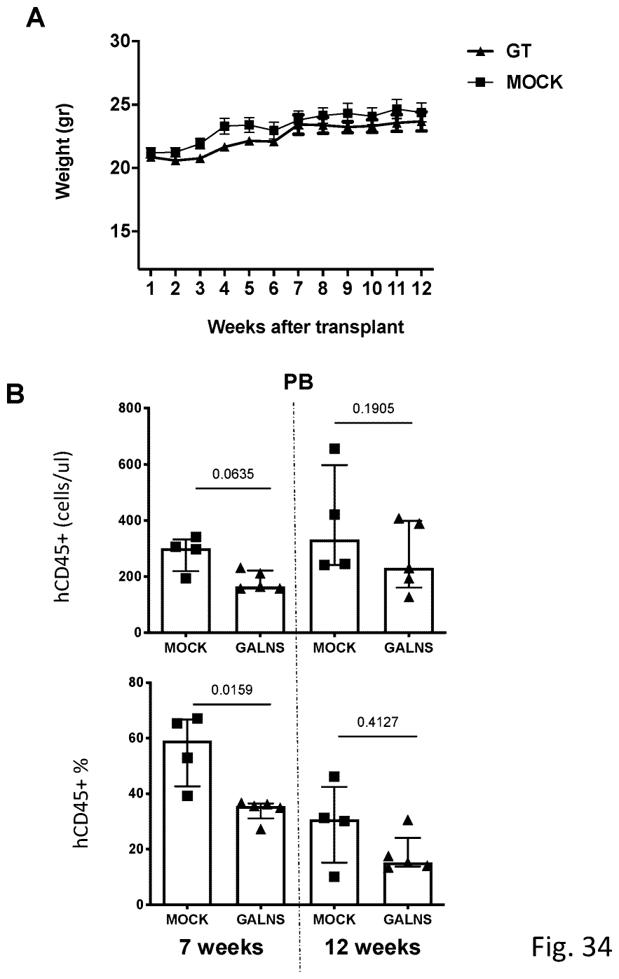

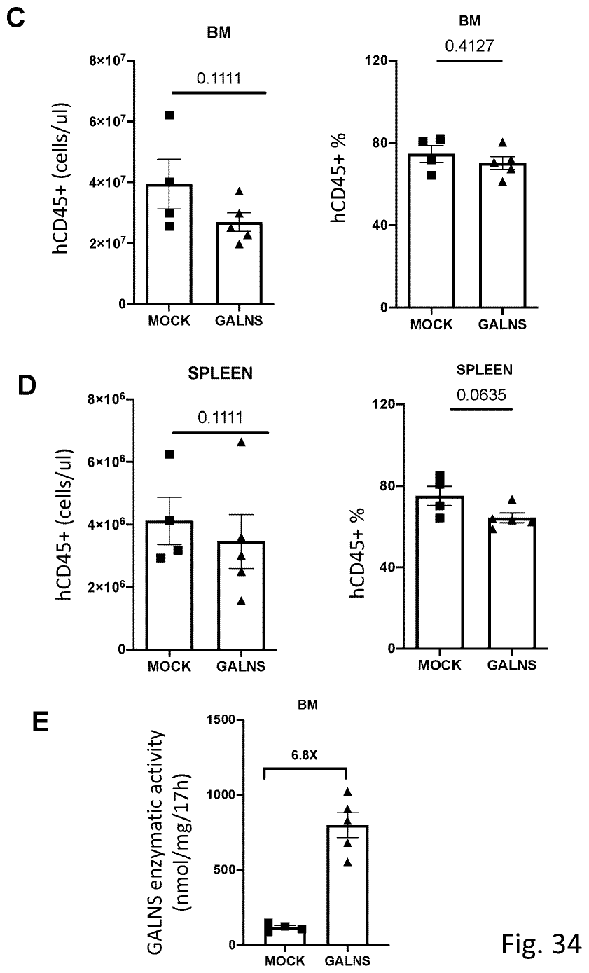

Count (FIG. 34B, upper panel) and percentage (FIG. 34B, lower panel) of human CD45+ cells in peripheral blood (PB) of transplanted mice of example 33, at 7 and 12 weeks after cell infusion, as well as, in bone marrow BM and SPLEEN at 12 weeks after transplant were measured; GALNS enzymatic activity in total BM cells in GT and MOCK group of mice at 12 weeks after transplant was also measured. These data show that both GALNS and MOCK groups display comparable human hematopoietic content in PB over time and hematopoietic organs at termination, providing initial evidence that GALNS overexpression does not alter human HSC engraftment and differentiation (FIGS. 34 B-D). In addition, a significantly higher GALNS enzymatic activity (6.8-Fold) was measured in the total BM cells from the GALNS compared to MOCK group of mice (FIG. 34E). Altogether these data show that transduced mPB CD34+ cells efficiently engraft in the BM of recipient mice and overexpress GALNS when transplanted in vivo. Based on these data, it can be concluded also that the expression level of GALNS is to cross-correct cells of non-hematopoietic origins.

### Example 35—In Vivo Assay in Knock-Out Mice Models

HSPCs will be isolated from GALNS KO mice and ex vivo transduced with LV GALNS designed to overexpress the human enzyme in human CD34+ cells, according to the transduction protocol for mouse cells according to Biffi et al. 2013 and Visigalli et al. 2010. Transduced cells will be transplanted into the tail vein of KO recipient mice upon conditioning. The restoration of enzyme activity in PBMNCs, and the reduction of GAG levels in the urine at different time points after transplantation (4, 6, 8, 12, 16, 20, 24 weeks) will be determined. The level of transplanted cells engraftment and hematological reconstitution in peripheral blood samples will be also determined by flow cytometry. Further, macroscopic correction of bone defects by XR will be assessed. Histopathological and immunofluorescence analysis of skeletal tissues will be performed to evaluate the cellular organization and interactions, upon euthanasia, 8, 16, and 24 weeks after treatment. The level of GAG accumulation will be measured by immunohistochemistry as well as the presence of vacuolated lysosomes in skeletal tissues. Restoration of bone characteristics will be finally be assessed the by microCT. All the analyses will be also carried out in GALNS KO mice transplanted with wild-type HSCs (HSCT); GALNS KO mice transplanted with GALNS KO HSCs transduced with a control vector (LV-GFP) and in untreated mice.

### Example 36—Optimization of HSPC-GT Protocol for Clinical Translation

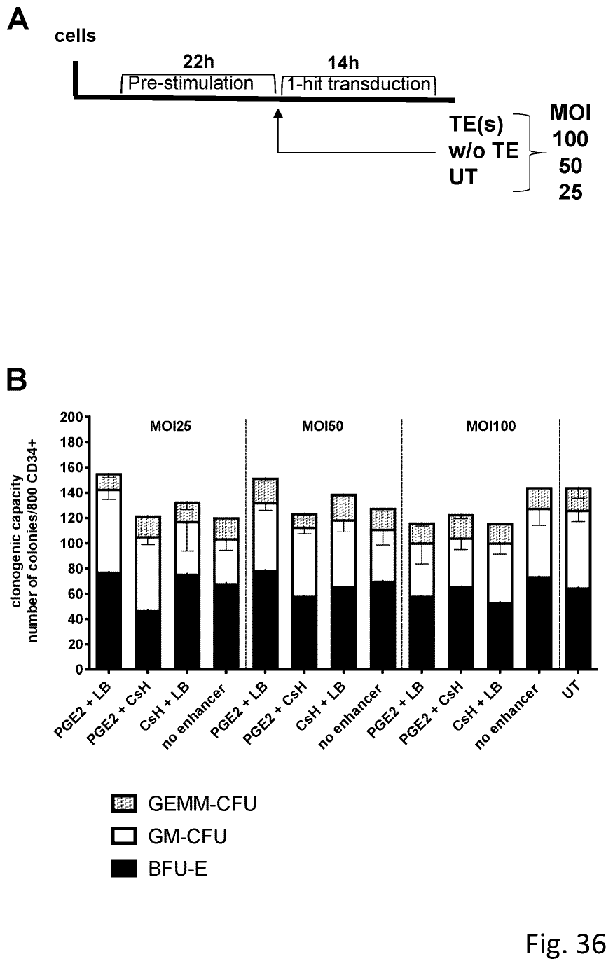

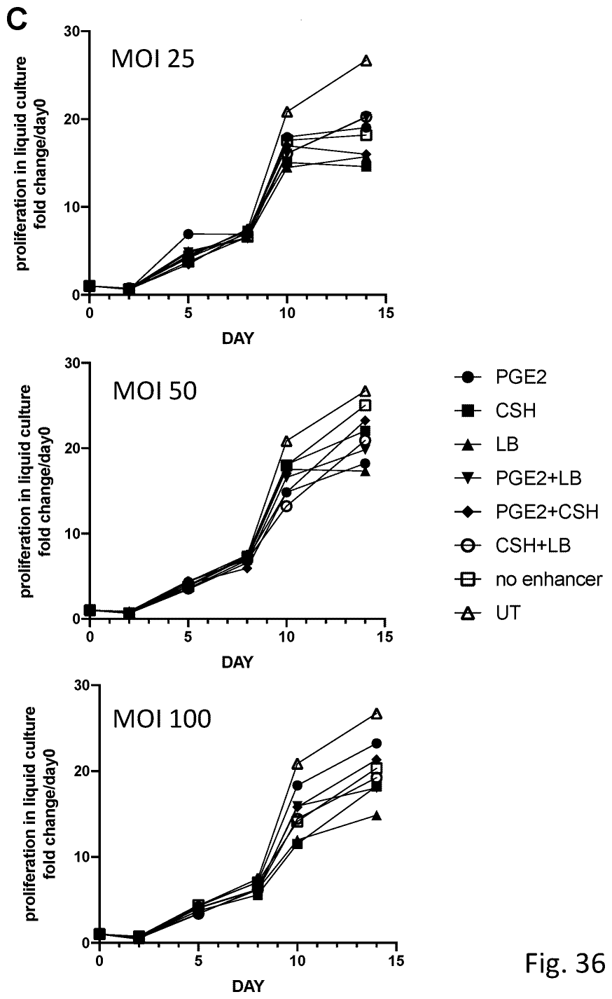

For the clinical translation of HSPC-GT to treat LSDs patients in accordance with the present invention, a clinical grade LV-GALNS was used as test. Different protocols of single hit transduction using transduction enhancers (TEs), alone and in combination, were tested and the best performing in terms of VCN, percentage of positive colonies, and level of enzymatic activity was identified. A VCN>2 and 80% of positive colonies in the peripheral blood mononuclear cells were shown to provide a better outcome of disease correction. Peripheral blood mobilized (mPB) human HSPCs from healthy-donor were placed in culture on retronectin-coated non-tissue culture-treated wells (T100A Takara) in CellGro medium (Cell Genix) supplemented with the following human cytokines: 60 ng/ml interleukine-3 (IL-3), 100 ng/ml thrombopoietin (TPO)), 300 ng/ml stem cell factor (SCF), and 300 ng/ml FLT3-L (all from Cell Genix). After 22 hours of pre-stimulation, cells were transduced for 14 hours at a specific multiplicity of infection (MOI 25, 50, or 100) with a single hit of LV-GALNS using the following TEs alone and in combination: 10 mM Prostaglandin2 (PGE2) (Cayman Chemical), 8 mM cyclosporin-H (CsH) (Sigma-Aldrich) and 1× LentiBoost (LB) (Sirion-Biotech) (FIG. 36A). At the end of the transduction, cells were collected and plated for clonogenic assay in MethoCult (Stemcell Tech) and differentiated into myeloid cells in liquid culture, using IMDM+10% FBS with the addition of 300 ng/ml stem cell factor (SCF), 60 ng/ml interleukine-3 (IL-3) and 60 ng/ml interleukine-6 (IL-6) (all from Cell Genix). We used cells transduced without TE and untransduced (UT) cells as control.

The toxicity of the different transduction protocols was evaluated as clonogenic capacity and myeloid cell proliferation of transduced cells. A trend of reduced colony number was observed in human HSPCs transduced at an MOI of 100 compared to controls (UT cells and cells transduced in the absence of TE), while no significant difference in the colony number was observed for all the other conditions. Importantly, all the transduction protocol did not affect the colony composition (GEMM, GM, BFU) was not affected (FIG. 36B). Only a slight decrease in the proliferation efficiency in transduced cells compared to UT cells was observed, especially at late passages. The level of proliferation was similar in the cells transduced with the single TE and with the TE combination (FIG. 36C).

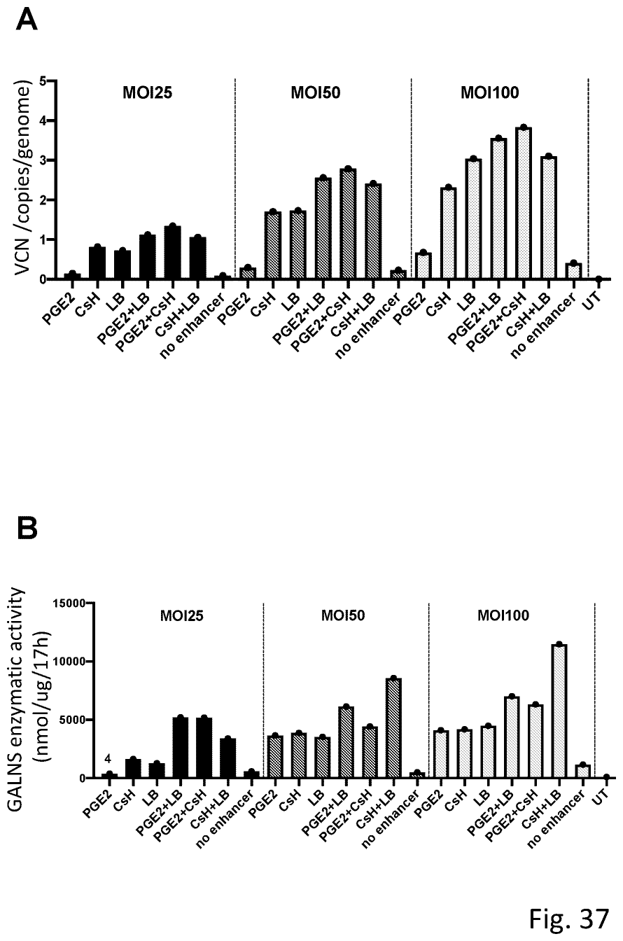

The VCN was determined in transduced cells by digital droplet PCR. A VCN<2 was found in mPB HSPCs transduced at an MOI of 25 for all the conditions. At an MOI of 50, the use of TE combination allows reaching a VCN>2 (PGE2+ LB: 2,56; PGE2+ CsH: 2,79; CsH+LB: 2,415), which further increased in cells transduced with a MOI of 100 (PGE2+ LB: 3.56; PGE2+ CsH: 3.83; CsH+LB: 3.105). In this setting (MOI100), the use of CsH and LB alone permitted to achieve a VCN>2 in transduced cells (CsH: 2.32; LB: 3.04) (FIG. 37A). Finally, the GALNS enzymatic activity was measured in transduced cells, showing the highest level of enzymatic activity in cells transduced at an MOI of 100 with the TE combination (FIG. 37B). It was concluded that the TE combination improved the cell transduction efficiency without causing toxicity.

### SEQUENCES

Sequences disclosed in conjunction with the present invention are enclosed and also displayed hereafter, in accordance with the following legend:

PROMOTER, restriction sites post ligation, KOZAK SEQUENCE, CODING SEQUENCE,
3′UTR
(hGLB1 WT)
Seq ID NO: 1
ATGCCGGGGTTCCTGGTTCGCATCCTCCCTCTGTTGCTGGTTCTGCTGCTTCTGGGCCCTACGCGCGGCTTGCGCAA
TGCCACCCAGAGGATGTTTGAAATTGACTATAGCCGGGACTCCTTCCTCAAGGATGGCCAGCCATTTCGCTACATCT
CAGGAAGCATTCACTACTCCCGTGTGCCCCGCTTCTACTGGAAGGACCGGCTGCTGAAGATGAAGATGGCTGGGCTG
AACGCCATCCAGACGTATGTGCCCTGGAACTTTCATGAGCCCTGGCCAGGACAGTACCAGTTTTCTGAGGACCATGA
TGTGGAATATTTTCTTCGGCTGGCTCATGAGCTGGGACTGCTGGTTATCCTGAGGCCCGGGCCCTACATCTGTGCAG
AGTGGGAAATGGGAGGATTACCTGCTTGGCTGCTAGAGAAAGAGTCTATTCTTCTCCGCTCCTCCGACCCAGATTAC
CTGGCAGCTGTGGACAAGTGGTTGGGAGTCCTTCTGCCCAAGATGAAGCCTCTCCTCTATCAGAATGGAGGGCCAGT
TATAACAGTGCAGGTTGAAAATGAATATGGCAGCTACTTTGCCTGTGATTTTGACTACCTGCGCTTCCTGCAGAAGC
GCTTTCGCCACCATCTGGGGGATGATGTGGTTCTGTTTACCACTGATGGAGCACATAAAACATTCCTGAAATGTGGG
GCCCTGCAGGGCCTCTACACCACGGTGGACTTTGGAACAGGCAGCAACATCACAGATGCTTTCCTAAGCCAGAGGAA
GTGTGAGCCCAAAGGACCCTTGATCAATTCTGAATTCTATACTGGCTGGCTAGATCACTGGGGCCAACCTCACTCCA
CAATCAAGACCGAAGCAGTGGCTTCCTCCCTCTATGATATACTTGCCCGTGGGGCGAGTGTGAACTTGTACATGTTT
ATAGGTGGGACCAATTTTGCCTATTGGAATGGGGCCAACTCACCCTATGCAGCACAGCCCACCAGCTACGACTATGA
TGCCCCACTGAGTGAGGCTGGGGACCTCACTGAGAAGTATTTTGCTCTGCGAAACATCATCCAGAAGTTTGAAAAAG
TACCAGAAGGTCCTATCCCTCCATCTACACCAAAGTTTGCATATGGAAAGGTCACTTTGGAAAAGTTAAAGACAGTG
GGAGCAGCTCTGGACATTCTGTGTCCCTCTGGGCCCATCAAAAGCCTTTATCCCTTGACATTTATCCAGGTGAAACA
GCATTATGGGTTTGTGCTGTACCGGACAACACTTCCTCAAGATTGCAGCAACCCAGCACCTCTCTCTTCACCCCTCA
ATGGAGTCCACGATCGAGCATATGTTGCTGTGGATGGGATCCCCCAGGGAGTCCTTGAGCGAAACAATGTGATCACT
CTGAACATAACAGGGAAAGCTGGAGCCACTCTGGACCTTCTGGTAGAGAACATGGGACGTGTGAACTATGGTGCATA
TATCAACGATTTTAAGGGTTTGGTTTCTAACCTGACTCTCAGTTCCAATATCCTCACGGACTGGACGATCTTTCCAC
TGGACACTGAGGATGCAGTGCGCAGCCACCTGGGGGGCTGGGGACACCGTGACAGTGGCCACCATGATGAAGCCTGG
GCCCACAACTCATCCAACTACACGCTCCCGGCCTTTTATATGGGGAACTTCTCCATTCCCAGTGGGATCCCAGACTT
GCCCCAGGACACCTTTATCCAGTTTCCTGGATGGACCAAGGGCCAGGTCTGGATTAATGGCTTTAACCTTGGCCGCT
ATTGGCCAGCCCGGGGCCCTCAGTTGACCTTGTTTGTGCCCCAGCACATCCTGATGACCTCGGCCCCAAACACCATC
ACCGTGCTGGAACTGGAGTGGGCACCCTGCAGCAGTGATGATCCAGAACTATGTGCTGTGACGTTCGTGGACAGGCC
AGTTATTGGCTCATCTGTGACCTACGATCATCCCTCCAAACCTGTTGAAAAAAGACTCATGCCCCCACCCCCGCAAA
AAAACAAAGATTCATGGCTGGACCATGTATGATGATGAAAG
(hGLB1 OPT)
Seq ID NO: 2
ATGCCGGGTTTCCTGGTGCGTATCCTGCCGCTGCTGCTGGTTCTGCTGCTGCTGGGTCCGACCCGTGGCCTGCGTAA
CGCGACCCAGCGTATGTTTGAGATTGACTACAGCCGTGATAGCTTCCTGAAGGATGGTCAACCGTTTCGTTACATCA
GCGGCAGCATTCACTATAGCCGTGTGCCGCGTTTCTACTGGAAAGACCGTCTGCTGAAGATGAAAATGGCGGGTCTG
AACGCGATCCAGACCTATGTTCCGTGGAACTTTCACGAGCCGTGGCCGGGTCAATATCAATTCAGCGAGGACCACGA
TGTGGAATACTTTCTGCGTCTGGCGCACGAACTGGGTCTGCTGGTTATCCTGCGTCCGGGCCCGTACATTTGCGCGG
AGTGGGAAATGGGTGGCCTGCCGGCGTGGCTGCTGGAGAAGGAAAGCATCCTGCTGCGTAGCAGCGACCCGGATTAT
CTGGCGGCGGTGGATAAATGGCTGGGTGTTCTGCTGCCGAAGATGAAACCGCTGCTGTACCAGAACGGTGGCCCGGT
GATTACCGTGCAAGTTGAGAACGAATACGGCAGCTATTTCGCGTGCGACTTTGATTACCTGCGTTTCCTGCAGAAGC
GTTTTCGTCACCACCTGGGTGACGATGTGGTTCTGTTCACCACCGACGGCGCGCACAAGACCTTTCTGAAATGCGGT
GCGCTGCAGGGCCTGTATACCACCGTTGACTTCGGTACCGGCAGCAACATCACCGATGCGTTTCTGAGCCAGCGTAA
GTGCGAGCCGAAAGGTCCGCTGATTAACAGCGAATTCTACACCGGTTGGCTGGATCACTGGGGCCAACCGCACAGCA
CCATCAAGACCGAGGCGGTGGCGAGCAGCCTGTATGACATTCTGGCGCGTGGTGCGAGCGTTAACCTGTACATGTTC
ATCGGTGGCACCAACTTTGCGTATTGGAACGGTGCGAACAGCCCGTATGCGGCGCAGCCGACCAGCTACGACTATGA
TGCGCCGCTGAGCGAGGCGGGTGACCTGACCGAAAAATATTTCGCGCTGCGTAACATCATTCAAAAGTTTGAGAAAG
TTCCGGAAGGTCCGATCCCGCCGAGCACCCCGAAGTTTGCGTACGGCAAAGTGACCCTGGAAAAGCTGAAAACCGTT
GGTGCGGCGCTGGATATCCTGTGCCCGAGCGGTCCGATTAAGAGCCTGTATCCGCTGACCTTCATTCAGGTGAAACA
ACACTACGGTTTTGTTCTGTATCGTACCACCCTGCCGCAGGACTGCAGCAACCCGGCGCCGCTGAGCAGCCCGCTGA
ACGGCGTTCACGACCGTGCGTACGTGGCGGTTGATGGTATCCCGCAAGGCGTGCTGGAGCGTAACAACGTTATCACC
CTGAACATTACCGGTAAAGCGGGTGCGACCCTGGACCTGCTGGTGGAAAACATGGGTCGTGTTAACTACGGCGCGTA
TATTAACGATTTCAAAGGTCTGGTGAGCAACCTGACCCTGAGCAGCAACATCCTGACCGACTGGACCATTTTTCCGC
TGGACACCGAGGATGCGGTTCGTAGCCACCTGGGTGGCTGGGGTCACCGTGACAGCGGTCACCACGATGAAGCGTGG
GCGCACAACAGCAGCAACTACACCCTGCCGGCGTTCTATATGGGTAACTTTAGCATTCCGAGCGGCATTCCGGACCT
GCCGCAGGATACCTTCATCCAATTTCCGGGTTGGACCAAAGGCCAAGTGTGGATTAACGGTTTCAACCTGGGTCGTT
ATTGGCCGGCGCGTGGTCCGCAGCTGACCCTGTTTGTGCCGCAACACATCCTGATGACCAGCGCGCCGAACACCATT
ACCGTTCTGGAGCTGGAATGGGCGCCGTGCAGCAGCGATGATCCGGAGCTGTGCGCGGTGACCTTCGTTGACCGTCC
GGTGATCGGCAGCAGCGTTACCTACGATCACCCGAGCAAGCCGGTGGAAAAACGTCTGATGCCGCCGCCGCCGCAGA
AGAACAAAGACAGCTGGCTGGATCACGTTTAATGATGAAAG
(hPGK promoter)
SEQ ID NO: 3
ATCATCGATTTCCGAATTCCACGGGGTTGGGGTTGCGCCTTTTCCAAGGCAGCCCTGGGTTTGCGCAGGGACGCGGC
TGCTCTGGGCGTGGTTCCGGGAAACGCAGCGGCGCCGACCCTGGGTCTCGCACATTCTTCACGTCCGTTCGCAGCGT
CACCCGGATCTTCGCCGCTACCCTTGTGGGCCCCCCGGCGACGCTTCCTGCTCCGCCCCTAAGTCGGGAAGGTTCCT
TGCGGTTCGCGGCGTGCCGGACGTGACAAACGGAAGCCGCACGTCTCACTAGTACCCTCGCAGACGGACAGCGCCAG
GGAGCAATGGCAGCGCGCCGACCGCGATGGGCTGTGGCCAATAGCGGCTGCTCAGCAGGGCGCGCCGAGAGCAGCGG
CCGGGAAGGGGCGGTGCGGGAGGCGGGGTGTGGGGCGGTAGTGTGGGCCCTGTTCCTGCCCGCGCGGTGTTCCGCAT
TCTGCAAGCCTCCGGAGCGCACGTCGGCAGTCGGCTCCCTCGTTGACCGAATCACCGACCTCTCTCCCCAACGC
(LV-hGLB1 WT cassette)
Seq ID NO: 4
ATCATCGATTTCCGAATTCCACGGGGTTGGGGTTGCGCCTTTTCCAAGGCAGCCCTGGGTTTGCGCAGGGACGCGGC
TGCTCTGGGCGTGGTTCCGGGAAACGCAGCGGCGCCGACCCTGGGTCTCGCACATTCTTCACGTCCGTTCGCAGCGT
CACCCGGATCTTCGCCGCTACCCTTGTGGGCCCCCCGGCGACGCTTCCTGCTCCGCCCCTAAGTCGGGAAGGTTCCT
TGCGGTTCGCGGCGTGCCGGACGTGACAAACGGAAGCCGCACGTCTCACTAGTACCCTCGCAGACGGACAGCGCCAG
GGAGCAATGGCAGCGCGCCGACCGCGATGGGCTGTGGCCAATAGCGGCTGCTCAGCAGGGCGCGCCGAGAGCAGCGG
CCGGGAAGGGGCGGTGCGGGAGGCGGGGTGTGGGGCGGTAGTGTGGGCCCTGTTCCTGCCCGCGCGGTGTTCCGCAT
TCTGCAAGCCTCCGGAGCGCACGTCGGCAGTCGGCTCCCTCGTTGACCGAATCACCGACCTCTCTCCCCAACGCgga
tctGCCGCCACCATGCCGGGGTTCCTGGTTCGCATCCTCCCTCTGTTGCTGGTTCTGCTGCTTCTGGGCCCTACGCG
CGGCTTGCGCAATGCCACCCAGAGGATGTTTGAAATTGACTATAGCCGGGACTCCTTCCTCAAGGATGGCCAGCCAT
TTCGCTACATCTCAGGAAGCATTCACTACTCCCGTGTGCCCCGCTTCTACTGGAAGGACCGGCTGCTGAAGATGAAG
ATGGCTGGGCTGAACGCCATCCAGACGTATGTGCCCTGGAACTTTCATGAGCCCTGGCCAGGACAGTACCAGTTTTC
TGAGGACCATGATGTGGAATATTTTCTTCGGCTGGCTCATGAGCTGGGACTGCTGGTTATCCTGAGGCCCGGGCCCT
ACATCTGTGCAGAGTGGGAAATGGGAGGATTACCTGCTTGGCTGCTAGAGAAAGAGTCTATTCTTCTCCGCTCCTCC
GACCCAGATTACCTGGCAGCTGTGGACAAGTGGTTGGGAGTCCTTCTGCCCAAGATGAAGCCTCTCCTCTATCAGAA
TGGAGGGCCAGTTATAACAGTGCAGGTTGAAAATGAATATGGCAGCTACTTTGCCTGTGATTTTGACTACCTGCGCT
TCCTGCAGAAGCGCTTTCGCCACCATCTGGGGGATGATGTGGTTCTGTTTACCACTGATGGAGCACATAAAACATTC
CTGAAATGTGGGGCCCTGCAGGGCCTCTACACCACGGTGGACTTTGGAACAGGCAGCAACATCACAGATGCTTTCCT
AAGCCAGAGGAAGTGTGAGCCCAAAGGACCCTTGATCAATTCTGAATTCTATACTGGCTGGCTAGATCACTGGGGCC
AACCTCACTCCACAATCAAGACCGAAGCAGTGGCTTCCTCCCTCTATGATATACTTGCCCGTGGGGCGAGTGTGAAC
TTGTACATGTTTATAGGTGGGACCAATTTTGCCTATTGGAATGGGGCCAACTCACCCTATGCAGCACAGCCCACCAG
CTACGACTATGATGCCCCACTGAGTGAGGCTGGGGACCTCACTGAGAAGTATTTTGCTCTGCGAAACATCATCCAGA
AGTTTGAAAAAGTACCAGAAGGTCCTATCCCTCCATCTACACCAAAGTTTGCATATGGAAAGGTCACTTTGGAAAAG
TTAAAGACAGTGGGAGCAGCTCTGGACATTCTGTGTCCCTCTGGGCCCATCAAAAGCCTTTATCCCTTGACATTTAT
CCAGGTGAAACAGCATTATGGGTTTGTGCTGTACCGGACAACACTTCCTCAAGATTGCAGCAACCCAGCACCTCTCT
CTTCACCCCTCAATGGAGTCCACGATCGAGCATATGTTGCTGTGGATGGGATCCCCCAGGGAGTCCTTGAGCGAAAC
AATGTGATCACTCTGAACATAACAGGGAAAGCTGGAGCCACTCTGGACCTTCTGGTAGAGAACATGGGACGTGTGAA
CTATGGTGCATATATCAACGATTTTAAGGGTTTGGTTTCTAACCTGACTCTCAGTTCCAATATCCTCACGGACTGGA
CGATCTTTCCACTGGACACTGAGGATGCAGTGCGCAGCCACCTGGGGGGCTGGGGACACCGTGACAGTGGCCACCAT
GATGAAGCCTGGGCCCACAACTCATCCAACTACACGCTCCCGGCCTTTTATATGGGGAACTTCTCCATTCCCAGTGG
GATCCCAGACTTGCCCCAGGACACCTTTATCCAGTTTCCTGGATGGACCAAGGGCCAGGTCTGGATTAATGGCTTTA
ACCTTGGCCGCTATTGGCCAGCCCGGGGCCCTCAGTTGACCTTGTTTGTGCCCCAGCACATCCTGATGACCTCGGCC
CCAAACACCATCACCGTGCTGGAACTGGAGTGGGCACCCTGCAGCAGTGATGATCCAGAACTATGTGCTGTGACGTT
CGTGGACAGGCCAGTTATTGGCTCATCTGTGACCTACGATCATCCCTCCAAACCTGTTGAAAAAAGACTCATGCCCC
CACCCCCGCAAAAAAACAAAGATTCATGGCTGGACCATGTATGATGATGAAAGgtcgac
(LV-hGLB1 OPT cassette)
Seq ID NO: 5
ATCATCGATTTCCGAATTCCACGGGGTTGGGGTTGCGCCTTTTCCAAGGCAGCCCTGGGTTTGCGCAGGGACGCGGC
TGCTCTGGGCGTGGTTCCGGGAAACGCAGCGGCGCCGACCCTGGGTCTCGCACATTCTTCACGTCCGTTCGCAGCGT
CACCCGGATCTTCGCCGCTACCCTTGTGGGCCCCCCGGCGACGCTTCCTGCTCCGCCCCTAAGTCGGGAAGGTTCCT
TGCGGTTCGCGGCGTGCCGGACGTGACAAACGGAAGCCGCACGTCTCACTAGTACCCTCGCAGACGGACAGCGCCAG
GGAGCAATGGCAGCGCGCCGACCGCGATGGGCTGTGGCCAATAGCGGCTGCTCAGCAGGGCGCGCCGAGAGCAGCGG
CCGGGAAGGGGCGGTGCGGGAGGCGGGGTGTGGGGCGGTAGTGTGGGCCCTGTTCCTGCCCGCGCGGTGTTCCGCAT
TCTGCAAGCCTCCGGAGCGCACGTCGGCAGTCGGCTCCCTCGTTGACCGAATCACCGACCTCTCTCCCCAACGCgga
tctGCCGCCACCATGCCGGGTTTCCTGGTGCGTATCCTGCCGCTGCTGCTGGTTCTGCTGCTGCTGGGTCCGACCCG
TGGCCTGCGTAACGCGACCCAGCGTATGTTTGAGATTGACTACAGCCGTGATAGCTTCCTGAAGGATGGTCAACCGT
TTCGTTACATCAGCGGCAGCATTCACTATAGCCGTGTGCCGCGTTTCTACTGGAAAGACCGTCTGCTGAAGATGAAA
ATGGCGGGTCTGAACGCGATCCAGACCTATGTTCCGTGGAACTTTCACGAGCCGTGGCCGGGTCAATATCAATTCAG
CGAGGACCACGATGTGGAATACTTTCTGCGTCTGGCGCACGAACTGGGTCTGCTGGTTATCCTGCGTCCGGGCCCGT
ACATTTGCGCGGAGTGGGAAATGGGTGGCCTGCCGGCGTGGCTGCTGGAGAAGGAAAGCATCCTGCTGCGTAGCAGC
GACCCGGATTATCTGGCGGCGGTGGATAAATGGCTGGGTGTTCTGCTGCCGAAGATGAAACCGCTGCTGTACCAGAA
CGGTGGCCCGGTGATTACCGTGCAAGTTGAGAACGAATACGGCAGCTATTTCGCGTGCGACTTTGATTACCTGCGTT
TCCTGCAGAAGCGTTTTCGTCACCACCTGGGTGACGATGTGGTTCTGTTCACCACCGACGGCGCGCACAAGACCTTT
CTGAAATGCGGTGCGCTGCAGGGCCTGTATACCACCGTTGACTTCGGTACCGGCAGCAACATCACCGATGCGTTTCT
GAGCCAGCGTAAGTGCGAGCCGAAAGGTCCGCTGATTAACAGCGAATTCTACACCGGTTGGCTGGATCACTGGGGCC
AACCGCACAGCACCATCAAGACCGAGGCGGTGGCGAGCAGCCTGTATGACATTCTGGCGCGTGGTGCGAGCGTTAAC
CTGTACATGTTCATCGGTGGCACCAACTTTGCGTATTGGAACGGTGCGAACAGCCCGTATGCGGCGCAGCCGACCAG
CTACGACTATGATGCGCCGCTGAGCGAGGCGGGTGACCTGACCGAAAAATATTTCGCGCTGCGTAACATCATTCAAA
AGTTTGAGAAAGTTCCGGAAGGTCCGATCCCGCCGAGCACCCCGAAGTTTGCGTACGGCAAAGTGACCCTGGAAAAG
CTGAAAACCGTTGGTGCGGCGCTGGATATCCTGTGCCCGAGCGGTCCGATTAAGAGCCTGTATCCGCTGACCTTCAT
TCAGGTGAAACAACACTACGGTTTTGTTCTGTATCGTACCACCCTGCCGCAGGACTGCAGCAACCCGGCGCCGCTGA
GCAGCCCGCTGAACGGCGTTCACGACCGTGCGTACGTGGCGGTTGATGGTATCCCGCAAGGCGTGCTGGAGCGTAAC
AACGTTATCACCCTGAACATTACCGGTAAAGCGGGTGCGACCCTGGACCTGCTGGTGGAAAACATGGGTCGTGTTAA
CTACGGCGCGTATATTAACGATTTCAAAGGTCTGGTGAGCAACCTGACCCTGAGCAGCAACATCCTGACCGACTGGA
CCATTTTTCCGCTGGACACCGAGGATGCGGTTCGTAGCCACCTGGGTGGCTGGGGTCACCGTGACAGCGGTCACCAC
GATGAAGCGTGGGCGCACAACAGCAGCAACTACACCCTGCCGGCGTTCTATATGGGTAACTTTAGCATTCCGAGCGG
CATTCCGGACCTGCCGCAGGATACCTTCATCCAATTTCCGGGTTGGACCAAAGGCCAAGTGTGGATTAACGGTTTCA
ACCTGGGTCGTTATTGGCCGGCGCGTGGTCCGCAGCTGACCCTGTTTGTGCCGCAACACATCCTGATGACCAGCGCG
CCGAACACCATTACCGTTCTGGAGCTGGAATGGGCGCCGTGCAGCAGCGATGATCCGGAGCTGTGCGCGGTGACCTT
CGTTGACCGTCCGGTGATCGGCAGCAGCGTTACCTACGATCACCCGAGCAAGCCGGTGGAAAAACGTCTGATGCCGC
CGCCGCCGCAGAAGAACAAAGACAGCTGGCTGGATCACGTTTAATGATGAAAGgtcgac
(Eukaryotic Translation Elongation Factor 1 alpha 1, EIF1a, promoter)
SEQ ID NO: 6
GGCTCCGGTGCCCGTCAGTGGGCAGAGCGCACATCGCCCACAGTCCCCGAGAAGTTGGGGGGAGGGGTCGGCAATTG
AACCGGTGCCTAGAGAAGGTGGCGCGGGGTAAACTGGGAAAGTGATGTCGTGTACTGGCTCCGCCTTTTTCCCGAGG
GTGGGGGAGAACCGTATATAAGTGCAGTAGTCGCCGTGAACGTTCTTTTTCGCAACGGGTTTGCCGCCAGAACACAG
GTAAGTGCCGTGTGTGGTTCCCGCGGGCCTGGCCTCTTTACGGGTTATGGCCCTTGCGTGCCTTGAATTACTTCCAC
CTGGCTGCAGTACGTGATTCTTGATCCCGAGCTTCGGGTTGGAAGTGGGTGGGAGAGTTCGAGGCCTTGCGCTTAAG
GAGCCCCTTCGCCTCGTGCTTGAGTTGAGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTGATGACCTGCTGCGACGCTTTTTTTCTG
GCAAGATAGTCTTGTAAATGCGGGCCAAGATCTGCACACTGGTATTTCGGTTTTTGGGGCCGCGGGCGGCGACGGGG
CCCGTGCGTCCCAGCGCACATGTTCGGCGAGGCGGGGCCTGCGAGCGCGGCCACCGAGAATCGGACGGGGGTAGTCT
CAAGCTGGCCGGCCTGCTCTGGTGCCTGGCCTCGCGCCGCCGTGTATCGCCCCGCCCTGGGCGGCAAGGCTGGCCCG
GTCGGCACCAGTTGCGTGAGCGGAAAGATGGCCGCTTCCCGGCCCTGCTGCAGGGAGCTCAAAATGGAGGACGCGGC
GCTCGGGAGAGCGGGCGGGTGAGTCACCCACACAAAGGAAAAGGGCCTTTCCGTCCTCAGCCGTCGCTTCATGTGAC
TCCACGGAGTACCGGGCGCCGTCCAGGCACCTCGATTAGTTCTCGAGCTTTTGGAGTACGTCGTCTTTAGGTTGGGG
GGAGGGGTTTTATGCGATGGAGTTTCCCCACACTGAGTGGGTGGAGACTGAAGTTAGGCCAGCTTGGCACTTGATGT
AATTCTCCTTGGAATTTGCCCTTTTTGAGTTTGGATCTTGGTTCATTCTCAAGCCTCAGACAGTGGTTCAAAGTTTT
TTTCTTCCATTTCAGGTGTCGTGA
(Cytomegalovirus, CMV, enhancer + promoter)
SEQ ID NO: 7
GACATTGATTATTGACTAGTTATTAATAGTAATCAATTACGGGGTCATTAGTTCATAGCCCATATATGGAGTTCCGC
GTTACATAACTTACGGTAAATGGCCCGCCTGGCTGACCGCCCAACGACCCCCGCCCATTGACGTCAATAATGACGTA
TGTTCCCATAGTAACGCCAATAGGGACTTTCCATTGACGTCAATGGGTGGAGTATTTACGGTAAACTGCCCACTTGG
CAGTACATCAAGTGTATCATATGCCAAGTACGCCCCCTATTGACGTCAATGACGGTAAATGGCCCGCCTGGCATTAT
GCCCAGTACATGACCTTATGGGACTTTCCTACTTGGCAGTACATCTACGTATTAGTCATCGCTATTACCATGGTGAT
GCGGTTTTGGCAGTACATCAATGGGCGTGGATAGCGGTTTGACTCACGGGGATTTCCAAGTCTCCACCCCATTGACG
TCAATGGGAGTTTGTTTTGGCACCAAAATCAACGGGACTTTCCAAAATGTCGTAACAACTCCGCCCCATTGACGCAA
ATGGGCGGTAGGCGTGTACGGTGGGAGGTCTATATAAGCAGAGCT
(CAG synthetic promoter, combining CMV enhancer with chicken beta-actin
promoter)
SEQ ID NO: 8
GCGTTACATAACTTACGGTAAATGGCCCGCCTGGCTGACCGCCCAACGACCCCCGCCCATTGACGTCAATAATGACG
TATGTTCCCATAGTAACGCCAATAGGGACTTTCCATTGACGTCAATGGGTGGAGTATTTACGGTAAACTGCCCACTT
GGCAGTACATCAAGTGTATCATATGCCAAGTACGCCCCCTATTGACGTCAATGACGGTAAATGGCCCGCCTGGCATT
ATGCCCAGTACATGACCTTATGGGACTTTCCTACTTGGCAGTACATCTACGTATTAGTCATCGCTATTACCATGGTC
GAGGTGAGCCCCACGTTCTGCTTCACTCTCCCCATCTCCCCCCCCTCCCCACCCCCAATTTTGTATTTATTTATTTT
TTAATTATTTTGTGCAGCGATGGGGGCGGGGGGGGGGGGGGGGCGCGCGCCAGGCGGGGCGGGGCGGGGCGAGGGGC
GGGGCGGGGCGAGGCGGAGAGGTGCGGCGGCAGCCAATCAGAGCGGCGCGCTCCGAAAGTTTCCTTTTATGGCGAGG
CGGCGGCGGCGGCGGCCCTATAAAAAGCGAAGCGCGCGGCGGGCG
(5′LTR)
SEQ ID NO: 9
ggtctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagaccctt
ttagtcagtgtggaaaatctctagcag
(encapsidation signal Ψ)
SEQ ID NO: 10
tcgacgcaggactcggcttgctgaagcgcgcacggcaagaggcgaggggcggcgactggtgagtacgccaaaaattt
tgactagcggaggctagaaggagagagatgggtgcgagagcgtcagtattaagcgggggag
(RRE)
SEQ ID NO: 11
tccttgggttcttgggagcagcaggaagcactatgggcgcagcgtcaatgacgctgacggtacaggccagacaatta
ttgtctggtatagtgcagcagcagaacaatttgctgagggctattgaggcgcaacagcatctgttgcaactcacagt
ctggggcatcaagcagctccaggcaagaatcctggctgtggaaagatacct
(cPPT/CTS)
SEQ ID NO: 12
ttttaaaagaaaaggggggattggggggtacagtgcaggggaaagaatagtagacataatagcaacagacatacaaa
ctaaagaattacaaaaacaaattacaaaattcaaaatttt
(WPRE)
SEQ ID NO: 13
Aatcaacctctggattacaaaatttgtgaaagattgactggtattcttaactatgttgctccttttacgctatgtgg
atacgctgctttaatgcctttgtatcatgctattgcttcccgtatggctttcattttctcctccttgtataaatcct
ggttgctgtctctttatgaggagttgtggcccgttgtcaggcaacgtggcgtggtgtgcactgtgtttgctgacgca
acccccactggttggggcattgccaccacctgtcagctcctttccgggactttcgctttccccctccctattgccac
ggcggaactcatcgccgcctgccttgcccgctgctggacaggggctcggctgttgggcactgacaattccgtggtgt
tgtcggggaaatcatcgtcctttccttggctgctcgcctgtgttgccacctggattctgcgcgggacgtccttctgc
tacgtcccttcggccctcaatccagcggaccttccttcccgcggcctgctgccggctctgcggcctcttccgcgtct
tcgccttcgccctcagacgagtcggatctccctttgggccgcctccccgcctg
(3′ LTR)
SEQ ID NO: 14
tggaagggctaattcactcccaacgaagacaagatctgctttttgcttgtactgggtctctctggttagaccagatc
tgagcctgggagctctctggctaactagggaacccactgcttaagcctcaataaagcttgccttgagtgcttcaagt
agtgtgtgcccgtctgttgtgtgactctggtaactagagatccctcagacccttttagtcagtgtggaaaatctcta
gcag
(SV40 polyA)
SEQ ID NO: 15
aacttgtttattgcagcttataatggttacaaataaagcaatagcatcacaaatttcacaaataaagcatttttttc
actgcattctagttgtggtttgtccaaactcatcaatgtatcttatcatgtctg
(SV40 origin of replication)
SEQ ID NO: 16
taattttttttatttatgcagaggccgaggccgcctcggcctctgagctattccagaagtagtgaggaggctttttt
ggaggcc
(F1 ori)
SEQ ID NO: 17
acgcgccctgtagcggcgcattaagcgcggcgggtgtggtggttacgcgcagcgtgaccgctacacttgccagcgcc
ctagcgcccgctcctttcgctttcttcccttcctttctcgccacgttcgccggctttccccgtcaagctctaaatcg
ggggctccctttagggttccgatttagtgctttacggcacctcgaccccaaaaaacttgattagggtgatggttcac
gtagtgggccatcgccctgatagacggtttttcgccctttgacgttggagtccacgttctttaatagtggactcttg
ttccaaactggaacaacactcaaccctatctcggtctattcttttgatttataagggattttgccgatttcggccta
ttggttaaaaaatgagctgatttaacaaaaatttaacgcgaattttaacaaaatattaacgcttacaattt
(Kozak sequence)
SEQ ID NO: 18
GCCGCCACC
(LV empty)
SEQ ID NO: 19
ACGCGCCCTGTAGCGGCGCATTAAGCGCGGCGGGTGTGGTGGTTACGCGCAGCGTGACCGCTACACTTGCCAGCGCC
CTAGCGCCCGCTCCTTTCGCTTTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAACTTGATTAGGGTGATGGTTCAC
GTAGTGGGCCATCGCCCTGATAGACGGTTTTTCGCCCTTTGACGTTGGAGTCCACGTTCTTTAATAGTGGACTCTTG
TTCCAAACTGGAACAACACTCAACCCTATCTCGGTCTATTCTTTTGATTTATAAGGGATTTTGCCGATTTCGGCCTA
TTGGTTAAAAAATGAGCTGATTTAACAAAAATTTAACGCGAATTTTAACAAAATATTAACGCTTACAATTTAGGTGG
CACTTTTCGGGGAAATGTGCGCGGAACCCCTATTTGTTTATTTTTCTAAATACATTCAAATATGTATCCGCTCATGA
GACAATAACCCTGATAAATGCTTCAATAATAGCACCTAGATCAAGAGACAGGATGAGGATCGTTTCGCATGATTGAA
CAAGATGGATTGCACGCAGGTTCTCCGGCCGCTTGGGTGGAGAGGCTATTCGGCTATGACTGGGCACAACAGACAAT
CGGCTGCTCTGATGCCGCCGTGTTCCGGCTGTCAGCGCAGGGGCGCCCGGTTCTTTTTGTCAAGACCGACCTGTCCG
GTGCCCTGAATGAACTGCAAGACGAGGCAGCGCGGCTATCGTGGCTGGCCACGACGGGCGTTCCTTGCGCAGCTGTG
CTCGACGTTGTCACTGAAGCGGGAAGGGACTGGCTGCTATTGGGCGAAGTGCCGGGGCAGGATCTCCTGTCATCTCA
CCTTGCTCCTGCCGAGAAAGTATCCATCATGGCTGATGCAATGCGGCGGCTGCATACGCTTGATCCGGCTACCTGCC
CATTCGACCACCAAGCGAAACATCGCATCGAGCGAGCACGTACTCGGATGGAAGCCGGTCTTGTCGATCAGGATGAT
CTGGACGAAGAGCATCAGGGGCTCGCGCCAGCCGAACTGTTCGCCAGGCTCAAGGCGAGCATGCCCGACGGCGAGGA
TCTCGTCGTGACCCATGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCTGAAGAGCTTGGCGGC
GAATGGGCTGACCGCTTCCTCGTGCTTTACGGTATCGCCGCTCCCGATTCGCAGCGCATCGCCTTCTATCGCCTTCT
TGACGAGTTCTTCTGAATTATTAACGCTTACAATTTCCTGATGCGGTATTTTCTCCTTACGCATCTGTGCGGTATTT
CACACCGCATCAGGTGGCACTTTTCGGGGAAATGTGCGCGGAACCCCTATTTGTTTATTTTTCTAAATACATTCAAA
TATGTATCCGCTCATGACCAAAATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAATAC
TGTTCTTCTAGTGTAGCCGTAGTTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGACTCAAGACGATAGTTACCGGAT
AAGGCGCAGCGGTCGGGCTGAACGGGGGGGTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGAGCGCAGCGAGTCAGTGAGCGAG
GAAGCGGAAGAGCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAATTTCACACAGGAAACAGCTATGAC
CATGATTACGCCAAGCGCGCAATTAACCCTCACTAAAGGGAACAAAAGCTGGAGCTGCAAGCTTGGCCATTGCATAC
GTTGTATCCATATCATAATATGTACATTTATATTGGCTCATGTCCAACATTACCGCCATGTTGACATTGATTATTGA
CTAGTTATTAATAGTAATCAATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAATGACGTATGTTCCCATAGTAAC
GCCAATAGGGACTTTCCATTGACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGATGCGGTTTTGGCAGTA
CATCAATGGGCGTGGATAGCGGTTTGACTCACGGGGATTTCCAAGTCTCCACCCCATTGACGTCAATGGGAGTTTGT
TTTGGCACCAAAATCAACGGGACTTTCCAAAATGTCGTAACAACTCCGCCCCATTGACGCAAATGGGCGGTAGGCGT
GTACGGTGGGAGGTCTATATAAGCAGAGCTCGTTTAGTGAACCGGGGTCTCTCTGGTTAGACCAGATCTGAGCCTGG
GAGCTCTCTGGCTAACTAGGGAACCCACTGCTTAAGCCTCAATAAAGCTTGCCTTGAGTGCTTCAAGTAGTGTGTGC
CCGTCTGTTGTGTGACTCTGGTAACTAGAGATCCCTCAGACCCTTTTAGTCAGTGTGGAAAATCTCTAGCAGTGGCG
CCCGAACAGGGACTTGAAAGCGAAAGGGAAACCAGAGGAGCTCTCTCGACGCAGGACTCGGCTTGCTGAAGCGCGCA
CGGCAAGAGGCGAGGGGCGGCGACTGGTGAGTACGCCAAAAATTTTGACTAGCGGAGGCTAGAAGGAGAGAGATGGG
TGCGAGAGCGTCAGTATTAAGCGGGGGAGAATTAGATCGCGATGGGAAAAAATTCGGTTAAGGCCAGGGGGAAAGAA
AAAATATAAATTAAAACATATAGTATGGGCAAGCAGGGAGCTAGAACGATTCGCAGTTAATCCTGGCCTGTTAGAAA
CATCAGAAGGCTGTAGACAAATACTGGGACAGCTACAACCATCCCTTCAGACAGGATCAGAAGAACTTAGATCATTA
TATAATACAGTAGCAACCCTCTATTGTGTGCATCAAAGGATAGAGATAAAAGACACCAAGGAAGCTTTAGACAAGAT
AGAGGAAGAGCAAAACAAAAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGGAGTAGCACCCACCAAGGC
AAAGAGAAGAGTGGTGCAGAGAGAAAAAAGAGCAGTGGGAATAGGAGCTTTGTTCCTTGGGTTCTTGGGAGCAGCAG
GAAGCACTATGGGCGCAGCGTCAATGACGCTGACGGTACAGGCCAGACAATTATTGTCTGGTATAGTGCAGCAGCAG
AACAATTTGCTGAGGGCTATTGAGGCGCAACAGCATCTGTTGCAACTCACAGTCTGGGGCATCAAGCAGCTCCAGGC
AAGAATCCTGGCTGTGGAAAGATACCTAAAGGATCAACAGCTCCTGGGGATTTGGGGTTGCTCTGGAAAACTCATTT
GCACCACTGCTGTGCCTTGGAATGCTAGTTGGAGTAATAAATCTCTGGAACAGATTTGGAATCACACGACCTGGATG
GAGTGGGACAGAGAAATTAACAATTACACAAGCTTAATACACTCCTTAATTGAAGAATCGCAAAACCAGCAAGAAAA
GAATGAACAAGAATTATTGGAATTAGATAAATGGGCAAGTTTGTGGAATTGGTTTAACATAACAAATTGGCTGTGGT
ATATAAAATTATTCATAATGATAGTAGGAGGCTTGGTAGGTTTAAGAATAGTTTTTGCTGTACTTTCTATAGTGAAT
AGAGTTAGGCAGGGATATTCACCATTATCGTTTCAGACCCACCTCCCAACCCCGAGGGGACCCGACAGGCCCGAAGG
AATAGAAGAAGAAGGTGGAGAGAGAGACAGAGACAGATCCATTCGATTAGTGAACGGATCTCGACGGTATCGGTTAA
CTTTTAAAAGAAAAGGGGGGATTGGGGGGTACAGTGCAGGGGAAAGAATAGTAGACATAATAGCAACAGACATACAA
ACTAAAGAATTACAAAAACAAATTACAAAATTCAAAATTTTATCGATAAGCTTGATATCATCGATTTCCGAATTCCA
CGGGGTTGGGGTTGCGCCTTTTCCAAGGCAGCCCTGGGTTTGCGCAGGGACGCGGCTGCTCTGGGCGTGGTTCCGGG
AAACGCAGCGGCGCCGACCCTGGGTCTCGCACATTCTTCACGTCCGTTCGCAGCGTCACCCGGATCTTCGCCGCTAC
CCTTGTGGGCCCCCCGGCGACGCTTCCTGCTCCGCCCCTAAGTCGGGAAGGTTCCTTGCGGTTCGCGGCGTGCCGGA
CGTGACAAACGGAAGCCGCACGTCTCACTAGTACCCTCGCAGACGGACAGCGCCAGGGAGCAATGGCAGCGCGCCGA
CCGCGATGGGCTGTGGCCAATAGCGGCTGCTCAGCAGGGCGCGCCGAGAGCAGCGGCCGGGAAGGGGCGGTGCGGGA
GGCGGGGTGTGGGGCGGTAGTGTGGGCCCTGTTCCTGCCCGCGCGGTGTTCCGCATTCTGCAAGCCTCCGGAGCGCA
CGTCGGCAGTCGGCTCCCTCGTTGACCGAATCACCGACCTCTCTCCCCAACGCggatccGCCGCCACCCACCTGCGC
GTCGACAATCAACCTCTGGATTACAAAATTTGTGAAAGATTGACTGGTATTCTTAACTATGTTGCTCCTTTTACGCT
ATGTGGATACGCTGCTTTAATGCCTTTGTATCATGCTATTGCTTCCCGTATGGCTTTCATTTTCTCCTCCTTGTATA
AATCCTGGTTGCTGTCTCTTTATGAGGAGTTGTGGCCCGTTGTCAGGCAACGTGGCGTGGTGTGCACTGTGTTTGCT
GACGCAACCCCCACTGGTTGGGGCATTGCCACCACCTGTCAGCTCCTTTCCGGGACTTTCGCTTTCCCCCTCCCTAT
TGCCACGGCGGAACTCATCGCCGCCTGCCTTGCCCGCTGCTGGACAGGGGCTCGGCTGTTGGGCACTGACAATTCCG
TGGTGTTGTCGGGGAAATCATCGTCCTTTCCTTGGCTGCTCGCCTGTGTTGCCACCTGGATTCTGCGCGGGACGTCC
TTCTGCTACGTCCCTTCGGCCCTCAATCCAGCGGACCTTCCTTCCCGCGGCCTGCTGCCGGCTCTGCGGCCTCTTCC
GCGTCTTCGCCTTCGCCCTCAGACGAGTCGGATCTCCCTTTGGGCCGCCTCCCCGCCTGGAATTCGAGCTCGGTACC
TTTAAGACCAATGACTTACAAGGCAGCTGTAGATCTTAGCCACTTTTTAAAAGAAAAGGGGGGACTGGAAGGGCTAA
TTCACTCCCAACGAAGACAAGATCTGCTTTTTGCTTGTACTGGGTCTCTCTGGTTAGACCAGATCTGAGCCTGGGAG
CTCTCTGGCTAACTAGGGAACCCACTGCTTAAGCCTCAATAAAGCTTGCCTTGAGTGCTTCAAGTAGTGTGTGCCCG
TCTGTTGTGTGACTCTGGTAACTAGAGATCCCTCAGACCCTTTTAGTCAGTGTGGAAAATCTCTAGCAGTAGTAGTT
CATGTCATCTTATTATTCAGTATTTATAACTTGCAAAGAAATGAATATCAGAGAGTGAGAGGAACTTGTTTATTGCA
GCTTATAATGGTTACAAATAAAGCAATAGCATCACAAATTTCACAAATAAAGCATTTTTTTCACTGCATTCTAGTTG
TGGTTTGTCCAAACTCATCAATGTATCTTATCATGTCTGGCTCTAGCTATCCCGCCCCTAACTCCGCCCATCCCGCC
CCTAACTCCGCCCAGTTCCGCCCATTCTCCGCCCCATGGCTGACTAATTTTTTTTATTTATGCAGAGGCCGAGGCCG
CCTCGGCCTCTGAGCTATTCCAGAAGTAGTGAGGAGGCTTTTTTGGAGGCCTAGGGACGTACCCAATTCGCCCTATA
GTGAGTCGTATTACGCGCGCTCACTGGCCGTCGTTTTACAACGTCGTGACTGGGAAAACCCTGGCGTTACCCAACTT
AATCGCCTTGCAGCACATCCCCCTTTCGCCAGCTGGCGTAATAGCGAAGAGGCCCGCACCGATCGCCCTTCCCAACA
GTTGCGCAGCCTGAATGGCGAATGGG
(mGLB1 C2C12)
SEQ ID NO: 20
ATGCTCCGGGTCCCCCTGTGTACGCCGCTCCCGCTCCTGGCACTGCTGCAACTGCTGGGCGCTGCGCACGGCATCTA
TAATGTCACCCAGAGGACATTTAAGCTCGACTACAGCCGGGACCGCTTCCTCAAGGATGGACAGCCATTCCGATACA
TCTCGGGAAGCATTCATTACTTCCGGATACCCCGCTTCTACTGGGAGGACCGGCTGCTGAAGATGAAGATGGCTGGG
CTGAATGCTATCCAGATGTACGTGCCCTGGAACTTCCATGAACCCCAACCAGGACAATATGAGTTTTCTGGGGACCG
TGATGTGGAGCATTTCATCCAGCTGGCTCATGAGCTGGGACTCCTGGTGATCCTGAGGCCTGGGCCCTACATCTGTG
CAGAGTGGGACATGGGGGGCTTACCTGCTTGGCTACTAGAGAAACAATCTATCGTTCTCCGGTCTTCTGACCCAGAC
TACCTTGTAGCTGTGGATAAATGGCTGGCAGTCCTTCTGCCCAAGATGAAGCCCCTGCTCTACCAGAACGGAGGACC
GATCATAACCGTGCAGGTTGAGAATGAGTACGGGTCCTACTTTGCCTGCGATTACGACTACCTACGCTTCCTGGTGC
ACCGCTTCCGCTACCATCTGGGTAATGACGTCATTCTCTTCACCACCGACGGAGCAAGTGAAAAAATGCTGAAGTGT
GGGACCCTGCAGGACCTGTACGCCACAGTGGATTTTGGAACAGGCAACAATATCACACAAGCTTTCCTGGTCCAGAG
GAAGTTTGAACCTAAAGGACCTTTGATCAATTCTGAGTTCTATACTGGCTGGCTAGACCACTGGGGTAAACCCCATT
CCACGGTGAAAACTAAAACACTGGCTACCTCCCTCTATAACCTGCTTGCCCGTGGGGCCAACGTGAACTTGTACATG
TTTATAGGTGGGACCAATTTTGCCTATTGGAATGGTGCCAACACGCCCTATGAGCCACAGCCCACCAGCTATGACTA
CGACGCCCCACTGAGCGAGGCTGGGGACCTCACTAAGAAGTATTTTGCTCTTCGAGAAGTCATTCAGATGTTTAAAG
AAGTCCCAGAAGGCCCTATCCCTCCGTCTACACCCAAATTTGCATATGGAAAAGTTGCTCTGAGAAAGTTCAAGACA
GTGGCTGAAGCTCTGGGTATCCTGTGTCCCAATGGGCCAGTGAAAAGCCTCTATCCCCTGACATTCACTCAGGTAAA
ACAGTATTTTGGGTATGTGCTGTACCGAACAACGCTTCCTCAAGATTGCAGTAACCCGAAACCCATTTTCTCTTCAC
CCTTCAATGGTGTCCGTGATCGGGCTTACGTCTCTGTGGACGGGGTCCCCCAAGGAATCCTTGATCGAAACCTCATG
ACAGCTCTGAACATACAGGGGAAGGCTGGAGCCACGCTGGACATCCTGGTGGAGAACATGGGGCGTGTGAACTATGG
CAGATTCATCAATGACTTCAAGGGTTTGATTTCCAACATGACTATCAACTCCACTGTCCTCACCAACTGGACGGTCT
TCCCACTGGACACTGAGGCCATGGTACGCAACCATCTCTGGGGCCGGGAGGCCAGTGATGGGGGTCACCTTGACGGA
CGGTCGACCTCCAATTCTTCGGACCTCATACTCCCCACCTTTTACGTGGGCAACTTCTCCATCCCCTCGGGCATCCC
AGACCTGCCACAGGACACCTTCATCCAGTTTCCTGGGTGGTCCAAGGGTCAAGTATGGATCAATGGCTTTAACCTCG
GCCGATACTGGCCCACAATGGGCCCACAAAAGACCTTGTTCGTGCCAAGGAACATCCTGACCACTTCAGCCCCAAAC
AACATCACAGTGTTGGAGCTAGAGTTTGCACCCTGCAGCGAGGGGACCCCAGAGCTGTGTACAGTAGAGTTTGTTGA
CACTCCGGTCATTTCCTGACCTGACTTGACCATCGGTGGCCATTTTCCAAGCCAGTCTGGTCAAGACTCATGGCTGA
ACCTCTGAGACTGAGCCTTGGGGAGCACAGCTCTACTCTGGTTACACGGATCACCTTTGTTGTGCTAGAATGGAAGC
TACAT
(MAN2B WT)
SEQ ID NO: 21
ATGGGCGCCTACGCGCGGGCTTCGGGGGTCTGCGCTCGCGGCTGCCTGGACTCAGCAGGCCCCTGGACCATGTCCCG
CGCCCTGCGGCCACCGCTCCCGCCTCTCTGCTTTTTCCTTTTGTTGCTGGCGGCTGCCGGTGCTCGGGCCGGGGGAT
ACGAGACATGCCCCACAGTGCAGCCGAACATGCTGAACGTGCACCTGCTGCCTCACACACATGATGACGTGGGCTGG
CTCAAAACCGTGGACCAGTACTTTTATGGAATCAAGAATGACATCCAGCACGCCGGTGTGCAGTACATCCTGGACTC
GGTCATCTCTGCCTTGCTGGCAGATCCCACCCGTCGCTTCATTTACGTGGAGATTGCCTTCTTCTCCCGTTGGTGGC
ACCAGCAGACAAATGCCACACAGGAAGTCGTGCGAGACCTTGTGCGCCAGGGGCGCCTGGAGTTCGCCAATGGTGGC
TGGGTGATGAACGATGAGGCAGCCACCCACTACGGTGCCATCGTGGACCAGATGACACTTGGGCTGCGCTTTCTGGA
GGACACATTTGGCAATGATGGGCGACCCCGTGTGGCCTGGCACATTGACCCCTTCGGCCACTCTCGGGAGCAGGCCT
CGCTGTTTGCGCAGATGGGCTTCGACGGCTTCTTCTTTGGGCGCCTTGATTATCAAGATAAGTGGGTACGGATGCAG
AAGCTGGAGATGGAGCAGGTGTGGCGGGCCAGCACCAGCCTGAAGCCCCCGACCGCGGACCTCTTCACTGGTGTGCT
TCCCAATGGTTACAACCCGCCAAGGAATCTGTGCTGGGATGTGCTGTGTGTCGATCAGCCGCTGGTGGAGGACCCTC
GCAGCCCCGAGTACAACGCCAAGGAGCTGGTCGATTACTTCCTAAATGTGGCCACTGCCCAGGGCCGGTATTACCGC
ACCAACCACACTGTGATGACCATGGGCTCGGACTTCCAATATGAGAATGCCAACATGTGGTTCAAGAACCTTGACAA
GCTCATCCGGCTGGTAAATGCGCAGCAGGCAAAAGGAAGCAGTGTCCATGTTCTCTACTCCACCCCCGCTTGTTACC
TCTGGGAGCTGAACAAGGCCAACCTCACCTGGTCAGTGAAACATGACGACTTCTTCCCTTACGCGGATGGCCCCCAC
CAGTTCTGGACCGGTTACTTTTCCAGTCGGCCGGCCCTCAAACGCTACGAGCGCCTCAGCTACAACTTCCTGCAGGT
GTGCAACCAGCTGGAGGCGCTGGTGGGCCTGGCGGCCAACGTGGGACCCTATGGCTCCGGAGACAGTGCACCCCTCA
ATGAGGCGATGGCTGTGCTCCAGCATCACGACGCCGTCAGCGGCACCTCCCGCCAGCACGTGGCCAACGACTACGCG
CGCCAGCTTGCGGCAGGCTGGGGGCCTTGCGAGGTTCTTCTGAGCAACGCGCTGGCGCGGCTCAGAGGCTTCAAAGA
TCACTTCACCTTTTGCCAACAGCTAAACATCAGCATCTGCCCGCTCAGCCAGACGGCGGCGCGCTTCCAGGTCATCG
TTTATAATCCCCTGGGGCGGAAGGTGAATTGGATGGTACGGCTGCCGGTCAGCGAAGGCGTTTTCGTTGTGAAGGAC
CCCAATGGCAGGACAGTGCCCAGCGATGTGGTAATATTTCCCAGCTCAGACAGCCAGGCGCACCCTCCGGAGCTGCT
GTTCTCAGCCTCACTGCCCGCCCTGGGCTTCAGCACCTATTCAGTAGCCCAGGTGCCTCGCTGGAAGCCCCAGGCCC
GCGCACCACAGCCCATCCCCAGAAGATCCTGGTCCCCTGCTTTAACCATCGAAAATGAGCACATCCGGGCAACGTTT
GATCCTGACACAGGGCTGTTGATGGAGATTATGAACATGAATCAGCAACTCCTGCTGCCTGTTCGCCAGACCTTCTT
CTGGTACAACGCCAGTATAGGTGACAACGAAAGTGACCAGGCCTCAGGTGCCTACATCTTCAGACCCAACCAACAGA
AACCGCTGCCTGTGAGCCGCTGGGCTCAGATCCACCTGGTGAAGACACCCTTGGTGCAGGAGGTGCACCAGAACTTC
TCAGCTTGGTGTTCCCAGGTGGTTCGCCTGTACCCAGGACAGCGGCACCTGGAGCTAGAGTGGTCGGTGGGGCCGAT
ACCTGTGGGCGACACCTGGGGGAAGGAGGTCATCAGCCGTTTTGACACACCGCTGGAGACAAAGGGACGCTTCTACA
CAGACAGCAATGGCCGGGAGATCCTGGAGAGGAGGCGGGATTATCGACCCACCTGGAAACTGAACCAGACGGAGCCC
GTGGCAGGAAACTACTATCCAGTCAACACCCGGATTTACATCACGGATGGAAACATGCAGCTGACTGTGCTGACTGA
CCGCTCCCAGGGGGGCAGCAGCCTGAGAGATGGCTCGCTGGAGCTCATGGTGCACCGAAGGCTGCTGAAGGACGATG
GACGCGGAGTATCGGAGCCACTAATGGAGAACGGGTCGGGGGCGTGGGTGCGAGGGCGCCACCTGGTGCTGCTGGAC
ACAGCCCAGGCTGCAGCCGCCGGACACCGGCTCCTGGCGGAGCAGGAGGTCCTGGCCCCTCAGGTGGTGCTGGCCCC
GGGTGGCGGCGCCGCCTACAATCTCGGGGCTCCTCCGCGCACGCAGTTCTCAGGGCTGCGCAGGGACCTGCCGCCCT
CGGTGCACCTGCTCACGCTGGCCAGCTGGGGCCCCGAAATGGTGCTGCTGCGCTTGGAGCACCAGTTTGCCGTAGGA
GAGGATTCCGGACGTAACCTGAGCGCCCCCGTTACCTTGAACTTGAGGGACCTGTTCTCCACCTTCACCATCACCCG
CCTGCAGGAGACCACGCTGGTGGCCAACCAGCTCCGCGAGGCAGCCTCCAGGCTCAAGTGGACAACAAACACAGGCC
CCACACCCCACCAAACTCCGTACCAGCTGGACCCGGCCAACATCACGCTGGAACCCATGGAAATCCGCACTTTCCTG
GCCTCAGTTCAATGGAAGGAGGTGGATGGTTAGGTCTGCTGG
(MAN2B OPT)
SEQ ID NO: 22
ATGGGCGCCTACGCAAGGGCATCCGGCGTGTGCGCAAGGGGCTGTCTGGACTCTGCCGGACCATGGACCATGAGCAG
AGCCCTGAGGCCACCTCTGCCACCACTGTGCTTCTTTCTGCTGCTGCTGGCAGCAGCAGGAGCAAGGGCAGGAGGCT
ATGAGACATGTCCTACCGTGCAGCCAAACATGCTGAATGTGCACCTGCTGCCTCACACCCACGACGATGTGGGCTGG
CTGAAGACAGTGGACCAGTACTTTTATGGCATCAAGAATGATATCCAGCACGCCGGCGTGCAGTACATCCTGGACTC
TGTGATCAGCGCCCTGCTGGCCGATCCAACCCGGAGATTCATCTATGTGGAGATCGCCTTCTTTAGCAGATGGTGGC
ACCAGCAGACAAACGCCACTCAGGAGGTGGTGAGGGATCTGGTGCGCCAGGGCCGGCTGGAGTTTGCAAACGGAGGA
TGGGTCATGAATGATGAGGCCGCCACACACTACGGCGCCATCGTGGACCAGATGACCCTGGGCCTGCGCTTTCTGGA
GGACACATTCGGCAACGATGGCAGACCCAGGGTGGCATGGCACATCGATCCTTTCGGCCACTCTCGGGAGCAGGCCA
GCCTGTTTGCCCAGATGGGCTTCGACGGCTTCTTTTTCGGCCGCCTGGACTACCAGGATAAGTGGGTGCGGATGCAG
AAGCTGGAGATGGAGCAAGTGTGGAGGGCCTCTACCAGCCTGAAGCCTCCAACCGCCGACCTGTTTACAGGCGTGCT
GCCTAACGGCTATAATCCCCCTCGCAATCTGTGCTGGGACGTGCTGTGCGTGGACCAGCCACTGGTGGAGGACCCCC
GGAGCCCAGAGTACAACGCCAAGGAGCTGGTGGACTATTTCCTGAATGTGGCCACCGCCCAGGGCAGATACTATAGG
ACAAACCACACCGTGATGACAATGGGCTCCGATTTTCAGTACGAGAACGCCAATATGTGGTTCAAGAACCTGGACAA
GCTGATCCGCCTGGTGAATGCACAGCAGGCAAAGGGCAGCTCCGTGCACGTGCTGTACAGCACCCCAGCCTGCTATC
TGTGGGAGCTGAACAAGGCCAATCTGACATGGTCCGTGAAGCACGACGACTTCTTCCCATACGCCGATGGCCCCCAC
CAGTTTTGGACCGGCTATTTCTCTAGCCGGCCCGCCCTGAAGCGGTACGAGCGGCTGTCCTATAACTTCCTGCAGGT
GTGCAATCAGCTGGAGGCCCTGGTGGGACTGGCAGCAAACGTGGGACCATACGGCTCCGGCGACTCTGCCCCCCTGA
ATGAGGCCATGGCCGTGCTGCAGCACCACGATGCCGTGAGCGGCACCTCCAGACAGCACGTGGCCAACGACTATGCA
AGGCAGCTGGCAGCAGGATGGGGACCATGCGAGGTGCTGCTGTCTAATGCCCTGGCCAGACTGAGGGGCTTTAAGGA
TCACTTTACCTTCTGCCAGCAGCTGAACATCTCCATCTGTCCTCTGTCTCAGACAGCCGCCAGATTCCAGGTCATCG
TGTACAACCCTCTGGGCAGAAAAGTGAATTGGATGGTGAGGCTGCCAGTGAGCGAGGGCGTGTTTGTGGTGAAGGAC
CCCAATGGCAGGACCGTGCCTTCTGACGTGGTCATCTTCCCATCCTCTGATAGCCAGGCCCACCCACCCGAGCTGCT
GTTTTCCGCCTCTCTGCCCGCCCTGGGCTTCTCTACCTATAGCGTGGCCCAGGTGCCAAGGTGGAAGCCTCAGGCAA
GGGCACCACAGCCAATCCCTAGGCGCAGCTGGTCCCCCGCCCTGACAATCGAGAACGAGCACATCAGAGCCACCTTC
GACCCTGATACAGGCCTGCTGATGGAGATCATGAACATGAATCAGCAGCTGCTGCTGCCCGTGAGGCAGACCTTTTT
CTGGTACAACGCCTCCATCGGCGACAATGAGTCCGATCAGGCCTCTGGCGCCTATATCTTTAGACCCAATCAGCAGA
AGCCACTGCCCGTGTCTAGATGGGCCCAGATCCACCTGGTGAAGACACCTCTGGTGCAGGAGGTGCACCAGAACTTC
AGCGCCTGGTGTTCCCAGGTGGTGAGACTGTACCCTGGCCAGAGGCACCTGGAGCTGGAGTGGAGCGTGGGACCTAT
CCCAGTGGGCGATACCTGGGGCAAGGAAGTGATCTCCAGATTTGACACCCCTCTGGAGACAAAGGGCCGCTTCTACA
CAGATTCTAACGGCCGGGAGATCCTGGAGCGGAGAAGGGACTATAGACCAACCTGGAAGCTGAATCAGACAGAGCCT
GTGGCCGGCAACTACTATCCAGTGAATACCAGGATCTATATCACAGACGGCAATATGCAGCTGACCGTGCTGACAGA
TAGATCCCAGGGAGGCAGCTCCCTGAGGGACGGCTCTCTGGAGCTGATGGTGCACCGCCGGCTGCTGAAGGACGATG
GAAGGGGCGTGTCCGAGCCACTGATGGAGAACGGCTCTGGAGCATGGGTGCGCGGCCGGCACCTGGTGCTGCTGGAT
ACCGCACAGGCAGCAGCAGCAGGACACAGGCTGCTGGCCGAGCAGGAGGTGCTGGCCCCACAGGTGGTGCTGGCACC
AGGAGGAGGAGCAGCCTACAATCTGGGAGCACCTCCAAGGACCCAGTTTTCCGGACTGAGAAGGGACCTGCCACCTA
GCGTGCACCTGCTGACACTGGCATCCTGGGGACCAGAGATGGTGCTGCTGAGGCTGGAGCACCAGTTCGCAGTGGGA
GAGGATAGCGGAAGGAACCTGTCCGCCCCAGTGACCCTGAATCTGAGGGACCTGTTTTCTACCTTCACAATCACCCG
GCTGCAGGAGACCACACTGGTGGCCAACCAGCTGAGAGAGGCCGCCAGCAGGCTGAAGTGGACCACAAATACCGGCC
CCACACCTCACCAGACACCTTACCAGCTGGACCCCGCCAACATCACCCTGGAGCCCATGGAGATCAGAACATTCCTG
GCCAGCGTGCAGTGGAAGGAGGTGGACGGCTGAGTCTGCTGG
(LV-mGLB1 C2C12 cassette)
SEQ ID NO: 23
ATCATCGATTTCCGAATTCCACGGGGTTGGGGTTGCGCCTTTTCCAAGGCAGCCCTGGGTTTGCGCAGGGACGCGGC
TGCTCTGGGCGTGGTTCCGGGAAACGCAGCGGCGCCGACCCTGGGTCTCGCACATTCTTCACGTCCGTTCGCAGCGT
CACCCGGATCTTCGCCGCTACCCTTGTGGGCCCCCCGGCGACGCTTCCTGCTCCGCCCCTAAGTCGGGAAGGTTCCT
TGCGGTTCGCGGCGTGCCGGACGTGACAAACGGAAGCCGCACGTCTCACTAGTACCCTCGCAGACGGACAGCGCCAG
GGAGCAATGGCAGCGCGCCGACCGCGATGGGCTGTGGCCAATAGCGGCTGCTCAGCAGGGCGCGCCGAGAGCAGCGG
CCGGGAAGGGGCGGTGCGGGAGGCGGGGTGTGGGGCGGTAGTGTGGGCCCTGTTCCTGCCCGCGCGGTGTTCCGCAT
TCTGCAAGCCTCCGGAGCGCACGTCGGCAGTCGGCTCCCTCGTTGACCGAATCACCGACCTCTCTCCCCAACGCgga
tccAGAGCGCCCACTGCCTAACGGAGAGACCCCATCGTGGCGCGATCATGCTCCGGGTCCCCCTGTGTACGCCGCTC
CCGCTCCTGGCACTGCTGCAACTGCTGGGCGCTGCGCACGGCATCTATAATGTCACCCAGAGGACATTTAAGCTCGA
CTACAGCCGGGACCGCTTCCTCAAGGATGGACAGCCATTCCGATACATCTCGGGAAGCATTCATTACTTCCGGATAC
CCCGCTTCTACTGGGAGGACCGGCTGCTGAAGATGAAGATGGCTGGGCTGAATGCTATCCAGATGTACGTGCCCTGG
AACTTCCATGAACCCCAACCAGGACAATATGAGTTTTCTGGGGACCGTGATGTGGAGCATTTCATCCAGCTGGCTCA
TGAGCTGGGACTCCTGGTGATCCTGAGGCCTGGGCCCTACATCTGTGCAGAGTGGGACATGGGGGGCTTACCTGCTT
GGCTACTAGAGAAACAATCTATCGTTCTCCGGTCTTCTGACCCAGACTACCTTGTAGCTGTGGATAAATGGCTGGCA
GTCCTTCTGCCCAAGATGAAGCCCCTGCTCTACCAGAACGGAGGACCGATCATAACCGTGCAGGTTGAGAATGAGTA
CGGGTCCTACTTTGCCTGCGATTACGACTACCTACGCTTCCTGGTGCACCGCTTCCGCTACCATCTGGGTAATGACG
TCATTCTCTTCACCACCGACGGAGCAAGTGAAAAAATGCTGAAGTGTGGGACCCTGCAGGACCTGTACGCCACAGTG
GATTTTGGAACAGGCAACAATATCACACAAGCTTTCCTGGTCCAGAGGAAGTTTGAACCTAAAGGACCTTTGATCAA
TTCTGAGTTCTATACTGGCTGGCTAGACCACTGGGGTAAACCCCATTCCACGGTGAAAACTAAAACACTGGCTACCT
CCCTCTATAACCTGCTTGCCCGTGGGGCCAACGTGAACTTGTACATGTTTATAGGTGGGACCAATTTTGCCTATTGG
AATGGTGCCAACACGCCCTATGAGCCACAGCCCACCAGCTATGACTACGACGCCCCACTGAGCGAGGCTGGGGACCT
CACTAAGAAGTATTTTGCTCTTCGAGAAGTCATTCAGATGTTTAAAGAAGTCCCAGAAGGCCCTATCCCTCCGTCTA
CACCCAAATTTGCATATGGAAAAGTTGCTCTGAGAAAGTTCAAGACAGTGGCTGAAGCTCTGGGTATCCTGTGTCCC
AATGGGCCAGTGAAAAGCCTCTATCCCCTGACATTCACTCAGGTAAAACAGTATTTTGGGTATGTGCTGTACCGAAC
AACGCTTCCTCAAGATTGCAGTAACCCGAAACCCATTTTCTCTTCACCCTTCAATGGTGTCCGTGATCGGGCTTACG
TCTCTGTGGACGGGGTCCCCCAAGGAATCCTTGATCGAAACCTCATGACAGCTCTGAACATACAGGGGAAGGCTGGA
GCCACGCTGGACATCCTGGTGGAGAACATGGGGCGTGTGAACTATGGCAGATTCATCAATGACTTCAAGGGTTTGAT
TTCCAACATGACTATCAACTCCACTGTCCTCACCAACTGGACGGTCTTCCCACTGGACACTGAGGCCATGGTACGCA
ACCATCTCTGGGGCCGGGAGGCCAGTGATGGGGGTCACCTTGACGGACGGTCGACCTCCAATTCTTCGGACCTCATA
CTCCCCACCTTTTACGTGGGCAACTTCTCCATCCCCTCGGGCATCCCAGACCTGCCACAGGACACCTTCATCCAGTT
TCCTGGGTGGTCCAAGGGTCAAGTATGGATCAATGGCTTTAACCTCGGCCGATACTGGCCCACAATGGGCCCACAAA
AGACCTTGTTCGTGCCAAGGAACATCCTGACCACTTCAGCCCCAAACAACATCACAGTGTTGGAGCTAGAGTTTGCA
CCCTGCAGCGAGGGGACCCCAGAGCTGTGTACAGTAGAGTTTGTTGACACTCCGGTCATTTCCTGACCTGACTTGAC
CATCGGTGGCCATTTTCCAAGCCAGTCTGGTCAAGACTCATGGCTGAACCTCTGAGACTGAGCCTTGGGGAGCACAG
CTCTACTCTGGTTACACGGATCACCTTTGTTGTGCTAGAATGGAAGCTACAT
(LV-MAN2B WT cassette)
SEQ ID NO: 24
ATCATCGATTTCCGAATTCCACGGGGTTGGGGTTGCGCCTTTTCCAAGGCAGCCCTGGGTTTGCGCAGGGACGCGGC
TGCTCTGGGCGTGGTTCCGGGAAACGCAGCGGCGCCGACCCTGGGTCTCGCACATTCTTCACGTCCGTTCGCAGCGT
CACCCGGATCTTCGCCGCTACCCTTGTGGGCCCCCCGGCGACGCTTCCTGCTCCGCCCCTAAGTCGGGAAGGTTCCT
TGCGGTTCGCGGCGTGCCGGACGTGACAAACGGAAGCCGCACGTCTCACTAGTACCCTCGCAGACGGACAGCGCCAG
GGAGCAATGGCAGCGCGCCGACCGCGATGGGCTGTGGCCAATAGCGGCTGCTCAGCAGGGCGCGCCGAGAGCAGCGG
CCGGGAAGGGGCGGTGCGGGAGGCGGGGTGTGGGGCGGTAGTGTGGGCCCTGTTCCTGCCCGCGCGGTGTTCCGCAT
TCTGCAAGCCTCCGGAGCGCACGTCGGCAGTCGGCTCCCTCGTTGACCGAATCACCGACCTCTCTCCCCAACGCgga
tccGCCGCCACCATGGGCGCCTACGCGCGGGCTTCGGGGGTCTGCGCTCGCGGCTGCCTGGACTCAGCAGGCCCCTG
GACCATGTCCCGCGCCCTGCGGCCACCGCTCCCGCCTCTCTGCTTTTTCCTTTTGTTGCTGGCGGCTGCCGGTGCTC
GGGCCGGGGGATACGAGACATGCCCCACAGTGCAGCCGAACATGCTGAACGTGCACCTGCTGCCTCACACACATGAT
GACGTGGGCTGGCTCAAAACCGTGGACCAGTACTTTTATGGAATCAAGAATGACATCCAGCACGCCGGTGTGCAGTA
CATCCTGGACTCGGTCATCTCTGCCTTGCTGGCAGATCCCACCCGTCGCTTCATTTACGTGGAGATTGCCTTCTTCT
CCCGTTGGTGGCACCAGCAGACAAATGCCACACAGGAAGTCGTGCGAGACCTTGTGCGCCAGGGGCGCCTGGAGTTC
GCCAATGGTGGCTGGGTGATGAACGATGAGGCAGCCACCCACTACGGTGCCATCGTGGACCAGATGACACTTGGGCT
GCGCTTTCTGGAGGACACATTTGGCAATGATGGGCGACCCCGTGTGGCCTGGCACATTGACCCCTTCGGCCACTCTC
GGGAGCAGGCCTCGCTGTTTGCGCAGATGGGCTTCGACGGCTTCTTCTTTGGGCGCCTTGATTATCAAGATAAGTGG
GTACGGATGCAGAAGCTGGAGATGGAGCAGGTGTGGCGGGCCAGCACCAGCCTGAAGCCCCCGACCGCGGACCTCTT
CACTGGTGTGCTTCCCAATGGTTACAACCCGCCAAGGAATCTGTGCTGGGATGTGCTGTGTGTCGATCAGCCGCTGG
TGGAGGACCCTCGCAGCCCCGAGTACAACGCCAAGGAGCTGGTCGATTACTTCCTAAATGTGGCCACTGCCCAGGGC
CGGTATTACCGCACCAACCACACTGTGATGACCATGGGCTCGGACTTCCAATATGAGAATGCCAACATGTGGTTCAA
GAACCTTGACAAGCTCATCCGGCTGGTAAATGCGCAGCAGGCAAAAGGAAGCAGTGTCCATGTTCTCTACTCCACCC
CCGCTTGTTACCTCTGGGAGCTGAACAAGGCCAACCTCACCTGGTCAGTGAAACATGACGACTTCTTCCCTTACGCG
GATGGCCCCCACCAGTTCTGGACCGGTTACTTTTCCAGTCGGCCGGCCCTCAAACGCTACGAGCGCCTCAGCTACAA
CTTCCTGCAGGTGTGCAACCAGCTGGAGGCGCTGGTGGGCCTGGCGGCCAACGTGGGACCCTATGGCTCCGGAGACA
GTGCACCCCTCAATGAGGCGATGGCTGTGCTCCAGCATCACGACGCCGTCAGCGGCACCTCCCGCCAGCACGTGGCC
AACGACTACGCGCGCCAGCTTGCGGCAGGCTGGGGGCCTTGCGAGGTTCTTCTGAGCAACGCGCTGGCGCGGCTCAG
AGGCTTCAAAGATCACTTCACCTTTTGCCAACAGCTAAACATCAGCATCTGCCCGCTCAGCCAGACGGCGGCGCGCT
TCCAGGTCATCGTTTATAATCCCCTGGGGCGGAAGGTGAATTGGATGGTACGGCTGCCGGTCAGCGAAGGCGTTTTC
GTTGTGAAGGACCCCAATGGCAGGACAGTGCCCAGCGATGTGGTAATATTTCCCAGCTCAGACAGCCAGGCGCACCC
TCCGGAGCTGCTGTTCTCAGCCTCACTGCCCGCCCTGGGCTTCAGCACCTATTCAGTAGCCCAGGTGCCTCGCTGGA
AGCCCCAGGCCCGCGCACCACAGCCCATCCCCAGAAGATCCTGGTCCCCTGCTTTAACCATCGAAAATGAGCACATC
CGGGCAACGTTTGATCCTGACACAGGGCTGTTGATGGAGATTATGAACATGAATCAGCAACTCCTGCTGCCTGTTCG
CCAGACCTTCTTCTGGTACAACGCCAGTATAGGTGACAACGAAAGTGACCAGGCCTCAGGTGCCTACATCTTCAGAC
CCAACCAACAGAAACCGCTGCCTGTGAGCCGCTGGGCTCAGATCCACCTGGTGAAGACACCCTTGGTGCAGGAGGTG
CACCAGAACTTCTCAGCTTGGTGTTCCCAGGTGGTTCGCCTGTACCCAGGACAGCGGCACCTGGAGCTAGAGTGGTC
GGTGGGGCCGATACCTGTGGGCGACACCTGGGGGAAGGAGGTCATCAGCCGTTTTGACACACCGCTGGAGACAAAGG
GACGCTTCTACACAGACAGCAATGGCCGGGAGATCCTGGAGAGGAGGCGGGATTATCGACCCACCTGGAAACTGAAC
CAGACGGAGCCCGTGGCAGGAAACTACTATCCAGTCAACACCCGGATTTACATCACGGATGGAAACATGCAGCTGAC
TGTGCTGACTGACCGCTCCCAGGGGGGCAGCAGCCTGAGAGATGGCTCGCTGGAGCTCATGGTGCACCGAAGGCTGC
TGAAGGACGATGGACGCGGAGTATCGGAGCCACTAATGGAGAACGGGTCGGGGGCGTGGGTGCGAGGGCGCCACCTG
GTGCTGCTGGACACAGCCCAGGCTGCAGCCGCCGGACACCGGCTCCTGGCGGAGCAGGAGGTCCTGGCCCCTCAGGT
GGTGCTGGCCCCGGGTGGCGGCGCCGCCTACAATCTCGGGGCTCCTCCGCGCACGCAGTTCTCAGGGCTGCGCAGGG
ACCTGCCGCCCTCGGTGCACCTGCTCACGCTGGCCAGCTGGGGCCCCGAAATGGTGCTGCTGCGCTTGGAGCACCAG
TTTGCCGTAGGAGAGGATTCCGGACGTAACCTGAGCGCCCCCGTTACCTTGAACTTGAGGGACCTGTTCTCCACCTT
CACCATCACCCGCCTGCAGGAGACCACGCTGGTGGCCAACCAGCTCCGCGAGGCAGCCTCCAGGCTCAAGTGGACAA
CAAACACAGGCCCCACACCCCACCAAACTCCGTACCAGCTGGACCCGGCCAACATCACGCTGGAACCCATGGAAATC
CGCACTTTCCTGGCCTCAGTTCAATGGAAGGAGGTGGATGGTTAGGTCTGCTGGgtcgac
(LV- MAN2B OPT cassette)
SEQ ID NO: 25
ATCATCGATTTCCGAATTCCACGGGGTTGGGGTTGCGCCTTTTCCAAGGCAGCCCTGGGTTTGCGCAGGGACGCGGC
TGCTCTGGGCGTGGTTCCGGGAAACGCAGCGGCGCCGACCCTGGGTCTCGCACATTCTTCACGTCCGTTCGCAGCGT
CACCCGGATCTTCGCCGCTACCCTTGTGGGCCCCCCGGCGACGCTTCCTGCTCCGCCCCTAAGTCGGGAAGGTTCCT
TGCGGTTCGCGGCGTGCCGGACGTGACAAACGGAAGCCGCACGTCTCACTAGTACCCTCGCAGACGGACAGCGCCAG
GGAGCAATGGCAGCGCGCCGACCGCGATGGGCTGTGGCCAATAGCGGCTGCTCAGCAGGGCGCGCCGAGAGCAGCGG
CCGGGAAGGGGCGGTGCGGGAGGCGGGGTGTGGGGCGGTAGTGTGGGCCCTGTTCCTGCCCGCGCGGTGTTCCGCAT
TCTGCAAGCCTCCGGAGCGCACGTCGGCAGTCGGCTCCCTCGTTGACCGAATCACCGACCTCTCTCCCCAACGCgga
tccGCCGCCACCATGGGCGCCTACGCAAGGGCATCCGGCGTGTGCGCAAGGGGCTGTCTGGACTCTGCCGGACCATG
GACCATGAGCAGAGCCCTGAGGCCACCTCTGCCACCACTGTGCTTCTTTCTGCTGCTGCTGGCAGCAGCAGGAGCAA
GGGCAGGAGGCTATGAGACATGTCCTACCGTGCAGCCAAACATGCTGAATGTGCACCTGCTGCCTCACACCCACGAC
GATGTGGGCTGGCTGAAGACAGTGGACCAGTACTTTTATGGCATCAAGAATGATATCCAGCACGCCGGCGTGCAGTA
CATCCTGGACTCTGTGATCAGCGCCCTGCTGGCCGATCCAACCCGGAGATTCATCTATGTGGAGATCGCCTTCTTTA
GCAGATGGTGGCACCAGCAGACAAACGCCACTCAGGAGGTGGTGAGGGATCTGGTGCGCCAGGGCCGGCTGGAGTTT
GCAAACGGAGGATGGGTCATGAATGATGAGGCCGCCACACACTACGGCGCCATCGTGGACCAGATGACCCTGGGCCT
GCGCTTTCTGGAGGACACATTCGGCAACGATGGCAGACCCAGGGTGGCATGGCACATCGATCCTTTCGGCCACTCTC
GGGAGCAGGCCAGCCTGTTTGCCCAGATGGGCTTCGACGGCTTCTTTTTCGGCCGCCTGGACTACCAGGATAAGTGG
GTGCGGATGCAGAAGCTGGAGATGGAGCAAGTGTGGAGGGCCTCTACCAGCCTGAAGCCTCCAACCGCCGACCTGTT
TACAGGCGTGCTGCCTAACGGCTATAATCCCCCTCGCAATCTGTGCTGGGACGTGCTGTGCGTGGACCAGCCACTGG
TGGAGGACCCCCGGAGCCCAGAGTACAACGCCAAGGAGCTGGTGGACTATTTCCTGAATGTGGCCACCGCCCAGGGC
AGATACTATAGGACAAACCACACCGTGATGACAATGGGCTCCGATTTTCAGTACGAGAACGCCAATATGTGGTTCAA
GAACCTGGACAAGCTGATCCGCCTGGTGAATGCACAGCAGGCAAAGGGCAGCTCCGTGCACGTGCTGTACAGCACCC
CAGCCTGCTATCTGTGGGAGCTGAACAAGGCCAATCTGACATGGTCCGTGAAGCACGACGACTTCTTCCCATACGCC
GATGGCCCCCACCAGTTTTGGACCGGCTATTTCTCTAGCCGGCCCGCCCTGAAGCGGTACGAGCGGCTGTCCTATAA
CTTCCTGCAGGTGTGCAATCAGCTGGAGGCCCTGGTGGGACTGGCAGCAAACGTGGGACCATACGGCTCCGGCGACT
CTGCCCCCCTGAATGAGGCCATGGCCGTGCTGCAGCACCACGATGCCGTGAGCGGCACCTCCAGACAGCACGTGGCC
AACGACTATGCAAGGCAGCTGGCAGCAGGATGGGGACCATGCGAGGTGCTGCTGTCTAATGCCCTGGCCAGACTGAG
GGGCTTTAAGGATCACTTTACCTTCTGCCAGCAGCTGAACATCTCCATCTGTCCTCTGTCTCAGACAGCCGCCAGAT
TCCAGGTCATCGTGTACAACCCTCTGGGCAGAAAAGTGAATTGGATGGTGAGGCTGCCAGTGAGCGAGGGCGTGTTT
GTGGTGAAGGACCCCAATGGCAGGACCGTGCCTTCTGACGTGGTCATCTTCCCATCCTCTGATAGCCAGGCCCACCC
ACCCGAGCTGCTGTTTTCCGCCTCTCTGCCCGCCCTGGGCTTCTCTACCTATAGCGTGGCCCAGGTGCCAAGGTGGA
AGCCTCAGGCAAGGGCACCACAGCCAATCCCTAGGCGCAGCTGGTCCCCCGCCCTGACAATCGAGAACGAGCACATC
AGAGCCACCTTCGACCCTGATACAGGCCTGCTGATGGAGATCATGAACATGAATCAGCAGCTGCTGCTGCCCGTGAG
GCAGACCTTTTTCTGGTACAACGCCTCCATCGGCGACAATGAGTCCGATCAGGCCTCTGGCGCCTATATCTTTAGAC
CCAATCAGCAGAAGCCACTGCCCGTGTCTAGATGGGCCCAGATCCACCTGGTGAAGACACCTCTGGTGCAGGAGGTG
CACCAGAACTTCAGCGCCTGGTGTTCCCAGGTGGTGAGACTGTACCCTGGCCAGAGGCACCTGGAGCTGGAGTGGAG
CGTGGGACCTATCCCAGTGGGCGATACCTGGGGCAAGGAAGTGATCTCCAGATTTGACACCCCTCTGGAGACAAAGG
GCCGCTTCTACACAGATTCTAACGGCCGGGAGATCCTGGAGCGGAGAAGGGACTATAGACCAACCTGGAAGCTGAAT
CAGACAGAGCCTGTGGCCGGCAACTACTATCCAGTGAATACCAGGATCTATATCACAGACGGCAATATGCAGCTGAC
CGTGCTGACAGATAGATCCCAGGGAGGCAGCTCCCTGAGGGACGGCTCTCTGGAGCTGATGGTGCACCGCCGGCTGC
TGAAGGACGATGGAAGGGGCGTGTCCGAGCCACTGATGGAGAACGGCTCTGGAGCATGGGTGCGCGGCCGGCACCTG
GTGCTGCTGGATACCGCACAGGCAGCAGCAGCAGGACACAGGCTGCTGGCCGAGCAGGAGGTGCTGGCCCCACAGGT
GGTGCTGGCACCAGGAGGAGGAGCAGCCTACAATCTGGGAGCACCTCCAAGGACCCAGTTTTCCGGACTGAGAAGGG
ACCTGCCACCTAGCGTGCACCTGCTGACACTGGCATCCTGGGGACCAGAGATGGTGCTGCTGAGGCTGGAGCACCAG
TTCGCAGTGGGAGAGGATAGCGGAAGGAACCTGTCCGCCCCAGTGACCCTGAATCTGAGGGACCTGTTTTCTACCTT
CACAATCACCCGGCTGCAGGAGACCACACTGGTGGCCAACCAGCTGAGAGAGGCCGCCAGCAGGCTGAAGTGGACCA
CAAATACCGGCCCCACACCTCACCAGACACCTTACCAGCTGGACCCCGCCAACATCACCCTGGAGCCCATGGAGATC
AGAACATTCCTGGCCAGCGTGCAGTGGAAGGAGGTGGACGGCTGAGTCTGCTGGgtcgac
(EIF4A)
SEQ ID NO: 26
GACTCACTATTTGTTTTCGCGCCCAGTTGCAAAAAGTGTC
(eIF4A-GLB1 cassette)
SEQ ID NO: 27
ATCATCGATTTCCGAATTCCACGGGGTTGGGGTTGCGCCTTTTCCAAGGCAGCCCTGGGTTTGCGCAGGGACGCGGC
TGCTCTGGGCGTGGTTCCGGGAAACGCAGCGGCGCCGACCCTGGGTCTCGCACATTCTTCACGTCCGTTCGCAGCGT
CACCCGGATCTTCGCCGCTACCCTTGTGGGCCCCCCGGCGACGCTTCCTGCTCCGCCCCTAAGTCGGGAAGGTTCCT
TGCGGTTCGCGGCGTGCCGGACGTGACAAACGGAAGCCGCACGTCTCACTAGTACCCTCGCAGACGGACAGCGCCAG
GGAGCAATGGCAGCGCGCCGACCGCGATGGGCTGTGGCCAATAGCGGCTGCTCAGCAGGGCGCGCCGAGAGCAGCGG
CCGGGAAGGGGCGGTGCGGGAGGCGGGGTGTGGGGCGGTAGTGTGGGCCCTGTTCCTGCCCGCGCGGTGTTCCGCAT
TCTGCAAGCCTCCGGAGCGCACGTCGGCAGTCGGCTCCCTCGTTGACCGAATCACCGACCTCTCTCCCCAACGCgga
tctGACTCACTATTTGTTTTCGCGCCCAGTTGCAAAAAGTGTCGCCGCCACCATGCCGGGGTTCCTGGTTCGCATCC
TCCCTCTGTTGCTGGTTCTGCTGCTTCTGGGCCCTACGCGCGGCTTGCGCAATGCCACCCAGAGGATGTTTGAAATT
GACTATAGCCGGGACTCCTTCCTCAAGGATGGCCAGCCATTTCGCTACATCTCAGGAAGCATTCACTACTCCCGTGT
GCCCCGCTTCTACTGGAAGGACCGGCTGCTGAAGATGAAGATGGCTGGGCTGAACGCCATCCAGACGTATGTGCCCT
GGAACTTTCATGAGCCCTGGCCAGGACAGTACCAGTTTTCTGAGGACCATGATGTGGAATATTTTCTTCGGCTGGCT
CATGAGCTGGGACTGCTGGTTATCCTGAGGCCCGGGCCCTACATCTGTGCAGAGTGGGAAATGGGAGGATTACCTGC
TTGGCTGCTAGAGAAAGAGTCTATTCTTCTCCGCTCCTCCGACCCAGATTACCTGGCAGCTGTGGACAAGTGGTTGG
GAGTCCTTCTGCCCAAGATGAAGCCTCTCCTCTATCAGAATGGAGGGCCAGTTATAACAGTGCAGGTTGAAAATGAA
TATGGCAGCTACTTTGCCTGTGATTTTGACTACCTGCGCTTCCTGCAGAAGCGCTTTCGCCACCATCTGGGGGATGA
TGTGGTTCTGTTTACCACTGATGGAGCACATAAAACATTCCTGAAATGTGGGGCCCTGCAGGGCCTCTACACCACGG
TGGACTTTGGAACAGGCAGCAACATCACAGATGCTTTCCTAAGCCAGAGGAAGTGTGAGCCCAAAGGACCCTTGATC
AATTCTGAATTCTATACTGGCTGGCTAGATCACTGGGGCCAACCTCACTCCACAATCAAGACCGAAGCAGTGGCTTC
CTCCCTCTATGATATACTTGCCCGTGGGGCGAGTGTGAACTTGTACATGTTTATAGGTGGGACCAATTTTGCCTATT
GGAATGGGGCCAACTCACCCTATGCAGCACAGCCCACCAGCTACGACTATGATGCCCCACTGAGTGAGGCTGGGGAC
CTCACTGAGAAGTATTTTGCTCTGCGAAACATCATCCAGAAGTTTGAAAAAGTACCAGAAGGTCCTATCCCTCCATC
TACACCAAAGTTTGCATATGGAAAGGTCACTTTGGAAAAGTTAAAGACAGTGGGAGCAGCTCTGGACATTCTGTGTC
CCTCTGGGCCCATCAAAAGCCTTTATCCCTTGACATTTATCCAGGTGAAACAGCATTATGGGTTTGTGCTGTACCGG
ACAACACTTCCTCAAGATTGCAGCAACCCAGCACCTCTCTCTTCACCCCTCAATGGAGTCCACGATCGAGCATATGT
TGCTGTGGATGGGATCCCCCAGGGAGTCCTTGAGCGAAACAATGTGATCACTCTGAACATAACAGGGAAAGCTGGAG
CCACTCTGGACCTTCTGGTAGAGAACATGGGACGTGTGAACTATGGTGCATATATCAACGATTTTAAGGGTTTGGTT
TCTAACCTGACTCTCAGTTCCAATATCCTCACGGACTGGACGATCTTTCCACTGGACACTGAGGATGCAGTGCGCAG
CCACCTGGGGGGCTGGGGACACCGTGACAGTGGCCACCATGATGAAGCCTGGGCCCACAACTCATCCAACTACACGC
TCCCGGCCTTTTATATGGGGAACTTCTCCATTCCCAGTGGGATCCCAGACTTGCCCCAGGACACCTTTATCCAGTTT
CCTGGATGGACCAAGGGCCAGGTCTGGATTAATGGCTTTAACCTTGGCCGCTATTGGCCAGCCCGGGGCCCTCAGTT
GACCTTGTTTGTGCCCCAGCACATCCTGATGACCTCGGCCCCAAACACCATCACCGTGCTGGAACTGGAGTGGGCAC
CCTGCAGCAGTGATGATCCAGAACTATGTGCTGTGACGTTCGTGGACAGGCCAGTTATTGGCTCATCTGTGACCTAC
GATCATCCCTCCAAACCTGTTGAAAAAAGACTCATGCCCCCACCCCCGCAAAAAAACAAAGATTCATGGCTGGACCA
TGTATGATGATGAAAGgtcgac
(LV hGLB1 WT transfer vector)
SEQ ID NO: 28
ACGCGCCCTGTAGCGGCGCATTAAGCGCGGCGGGTGTGGTGGTTACGCGCAGCGTGACCGCTACACTTGCCAGCGCC
CTAGCGCCCGCTCCTTTCGCTTTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAACTTGATTAGGGTGATGGTTCAC
GTAGTGGGCCATCGCCCTGATAGACGGTTTTTCGCCCTTTGACGTTGGAGTCCACGTTCTTTAATAGTGGACTCTTG
TTCCAAACTGGAACAACACTCAACCCTATCTCGGTCTATTCTTTTGATTTATAAGGGATTTTGCCGATTTCGGCCTA
TTGGTTAAAAAATGAGCTGATTTAACAAAAATTTAACGCGAATTTTAACAAAATATTAACGCTTACAATTTAGGTGG
CACTTTTCGGGGAAATGTGCGCGGAACCCCTATTTGTTTATTTTTCTAAATACATTCAAATATGTATCCGCTCATGA
GACAATAACCCTGATAAATGCTTCAATAATAGCACCTAGATCAAGAGACAGGATGAGGATCGTTTCGCATGATTGAA
CAAGATGGATTGCACGCAGGTTCTCCGGCCGCTTGGGTGGAGAGGCTATTCGGCTATGACTGGGCACAACAGACAAT
CGGCTGCTCTGATGCCGCCGTGTTCCGGCTGTCAGCGCAGGGGCGCCCGGTTCTTTTTGTCAAGACCGACCTGTCCG
GTGCCCTGAATGAACTGCAAGACGAGGCAGCGCGGCTATCGTGGCTGGCCACGACGGGCGTTCCTTGCGCAGCTGTG
CTCGACGTTGTCACTGAAGCGGGAAGGGACTGGCTGCTATTGGGCGAAGTGCCGGGGCAGGATCTCCTGTCATCTCA
CCTTGCTCCTGCCGAGAAAGTATCCATCATGGCTGATGCAATGCGGCGGCTGCATACGCTTGATCCGGCTACCTGCC
CATTCGACCACCAAGCGAAACATCGCATCGAGCGAGCACGTACTCGGATGGAAGCCGGTCTTGTCGATCAGGATGAT
CTGGACGAAGAGCATCAGGGGCTCGCGCCAGCCGAACTGTTCGCCAGGCTCAAGGCGAGCATGCCCGACGGCGAGGA
TCTCGTCGTGACCCATGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCTGAAGAGCTTGGCGGC
GAATGGGCTGACCGCTTCCTCGTGCTTTACGGTATCGCCGCTCCCGATTCGCAGCGCATCGCCTTCTATCGCCTTCT
TGACGAGTTCTTCTGAATTATTAACGCTTACAATTTCCTGATGCGGTATTTTCTCCTTACGCATCTGTGCGGTATTT
CACACCGCATCAGGTGGCACTTTTCGGGGAAATGTGCGCGGAACCCCTATTTGTTTATTTTTCTAAATACATTCAAA
TATGTATCCGCTCATGACCAAAATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAATAC
TGTTCTTCTAGTGTAGCCGTAGTTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGACTCAAGACGATAGTTACCGGAT
AAGGCGCAGCGGTCGGGCTGAACGGGGGGGTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGAGCGCAGCGAGTCAGTGAGCGAG
GAAGCGGAAGAGCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAATTTCACACAGGAAACAGCTATGAC
CATGATTACGCCAAGCGCGCAATTAACCCTCACTAAAGGGAACAAAAGCTGGAGCTGCAAGCTTGGCCATTGCATAC
GTTGTATCCATATCATAATATGTACATTTATATTGGCTCATGTCCAACATTACCGCCATGTTGACATTGATTATTGA
CTAGTTATTAATAGTAATCAATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAATGACGTATGTTCCCATAGTAAC
GCCAATAGGGACTTTCCATTGACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGATGCGGTTTTGGCAGTA
CATCAATGGGCGTGGATAGCGGTTTGACTCACGGGGATTTCCAAGTCTCCACCCCATTGACGTCAATGGGAGTTTGT
TTTGGCACCAAAATCAACGGGACTTTCCAAAATGTCGTAACAACTCCGCCCCATTGACGCAAATGGGCGGTAGGCGT
GTACGGTGGGAGGTCTATATAAGCAGAGCTCGTTTAGTGAACCGGGGTCTCTCTGGTTAGACCAGATCTGAGCCTGG
GAGCTCTCTGGCTAACTAGGGAACCCACTGCTTAAGCCTCAATAAAGCTTGCCTTGAGTGCTTCAAGTAGTGTGTGC
CCGTCTGTTGTGTGACTCTGGTAACTAGAGATCCCTCAGACCCTTTTAGTCAGTGTGGAAAATCTCTAGCAGTGGCG
CCCGAACAGGGACTTGAAAGCGAAAGGGAAACCAGAGGAGCTCTCTCGACGCAGGACTCGGCTTGCTGAAGCGCGCA
CGGCAAGAGGCGAGGGGCGGCGACTGGTGAGTACGCCAAAAATTTTGACTAGCGGAGGCTAGAAGGAGAGAGATGGG
TGCGAGAGCGTCAGTATTAAGCGGGGGAGAATTAGATCGCGATGGGAAAAAATTCGGTTAAGGCCAGGGGGAAAGAA
AAAATATAAATTAAAACATATAGTATGGGCAAGCAGGGAGCTAGAACGATTCGCAGTTAATCCTGGCCTGTTAGAAA
CATCAGAAGGCTGTAGACAAATACTGGGACAGCTACAACCATCCCTTCAGACAGGATCAGAAGAACTTAGATCATTA
TATAATACAGTAGCAACCCTCTATTGTGTGCATCAAAGGATAGAGATAAAAGACACCAAGGAAGCTTTAGACAAGAT
AGAGGAAGAGCAAAACAAAAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGGAGTAGCACCCACCAAGGC
AAAGAGAAGAGTGGTGCAGAGAGAAAAAAGAGCAGTGGGAATAGGAGCTTTGTTCCTTGGGTTCTTGGGAGCAGCAG
GAAGCACTATGGGCGCAGCGTCAATGACGCTGACGGTACAGGCCAGACAATTATTGTCTGGTATAGTGCAGCAGCAG
AACAATTTGCTGAGGGCTATTGAGGCGCAACAGCATCTGTTGCAACTCACAGTCTGGGGCATCAAGCAGCTCCAGGC
AAGAATCCTGGCTGTGGAAAGATACCTAAAGGATCAACAGCTCCTGGGGATTTGGGGTTGCTCTGGAAAACTCATTT
GCACCACTGCTGTGCCTTGGAATGCTAGTTGGAGTAATAAATCTCTGGAACAGATTTGGAATCACACGACCTGGATG
GAGTGGGACAGAGAAATTAACAATTACACAAGCTTAATACACTCCTTAATTGAAGAATCGCAAAACCAGCAAGAAAA
GAATGAACAAGAATTATTGGAATTAGATAAATGGGCAAGTTTGTGGAATTGGTTTAACATAACAAATTGGCTGTGGT
ATATAAAATTATTCATAATGATAGTAGGAGGCTTGGTAGGTTTAAGAATAGTTTTTGCTGTACTTTCTATAGTGAAT
AGAGTTAGGCAGGGATATTCACCATTATCGTTTCAGACCCACCTCCCAACCCCGAGGGGACCCGACAGGCCCGAAGG
AATAGAAGAAGAAGGTGGAGAGAGAGACAGAGACAGATCCATTCGATTAGTGAACGGATCTCGACGGTATCGGTTAA
CTTTTAAAAGAAAAGGGGGGATTGGGGGGTACAGTGCAGGGGAAAGAATAGTAGACATAATAGCAACAGACATACAA
ACTAAAGAATTACAAAAACAAATTACAAAATTCAAAATTTTATCGATAAGCTTGATATCATCGATTTCCGAATTCCA
CGGGGTTGGGGTTGCGCCTTTTCCAAGGCAGCCCTGGGTTTGCGCAGGGACGCGGCTGCTCTGGGCGTGGTTCCGGG
AAACGCAGCGGCGCCGACCCTGGGTCTCGCACATTCTTCACGTCCGTTCGCAGCGTCACCCGGATCTTCGCCGCTAC
CCTTGTGGGCCCCCCGGCGACGCTTCCTGCTCCGCCCCTAAGTCGGGAAGGTTCCTTGCGGTTCGCGGCGTGCCGGA
CGTGACAAACGGAAGCCGCACGTCTCACTAGTACCCTCGCAGACGGACAGCGCCAGGGAGCAATGGCAGCGCGCCGA
CCGCGATGGGCTGTGGCCAATAGCGGCTGCTCAGCAGGGCGCGCCGAGAGCAGCGGCCGGGAAGGGGCGGTGCGGGA
GGCGGGGTGTGGGGCGGTAGTGTGGGCCCTGTTCCTGCCCGCGCGGTGTTCCGCATTCTGCAAGCCTCCGGAGCGCA
CGTCGGCAGTCGGCTCCCTCGTTGACCGAATCACCGACCTCTCTCCCCAACGCggatctGCCGCCACCATGCCGGGG
TTCCTGGTTCGCATCCTCCCTCTGTTGCTGGTTCTGCTGCTTCTGGGCCCTACGCGCGGCTTGCGCAATGCCACCCA
GAGGATGTTTGAAATTGACTATAGCCGGGACTCCTTCCTCAAGGATGGCCAGCCATTTCGCTACATCTCAGGAAGCA
TTCACTACTCCCGTGTGCCCCGCTTCTACTGGAAGGACCGGCTGCTGAAGATGAAGATGGCTGGGCTGAACGCCATC
CAGACGTATGTGCCCTGGAACTTTCATGAGCCCTGGCCAGGACAGTACCAGTTTTCTGAGGACCATGATGTGGAATA
TTTTCTTCGGCTGGCTCATGAGCTGGGACTGCTGGTTATCCTGAGGCCCGGGCCCTACATCTGTGCAGAGTGGGAAA
TGGGAGGATTACCTGCTTGGCTGCTAGAGAAAGAGTCTATTCTTCTCCGCTCCTCCGACCCAGATTACCTGGCAGCT
GTGGACAAGTGGTTGGGAGTCCTTCTGCCCAAGATGAAGCCTCTCCTCTATCAGAATGGAGGGCCAGTTATAACAGT
GCAGGTTGAAAATGAATATGGCAGCTACTTTGCCTGTGATTTTGACTACCTGCGCTTCCTGCAGAAGCGCTTTCGCC
ACCATCTGGGGGATGATGTGGTTCTGTTTACCACTGATGGAGCACATAAAACATTCCTGAAATGTGGGGCCCTGCAG
GGCCTCTACACCACGGTGGACTTTGGAACAGGCAGCAACATCACAGATGCTTTCCTAAGCCAGAGGAAGTGTGAGCC
CAAAGGACCCTTGATCAATTCTGAATTCTATACTGGCTGGCTAGATCACTGGGGCCAACCTCACTCCACAATCAAGA
CCGAAGCAGTGGCTTCCTCCCTCTATGATATACTTGCCCGTGGGGCGAGTGTGAACTTGTACATGTTTATAGGTGGG
ACCAATTTTGCCTATTGGAATGGGGCCAACTCACCCTATGCAGCACAGCCCACCAGCTACGACTATGATGCCCCACT
GAGTGAGGCTGGGGACCTCACTGAGAAGTATTTTGCTCTGCGAAACATCATCCAGAAGTTTGAAAAAGTACCAGAAG
GTCCTATCCCTCCATCTACACCAAAGTTTGCATATGGAAAGGTCACTTTGGAAAAGTTAAAGACAGTGGGAGCAGCT
CTGGACATTCTGTGTCCCTCTGGGCCCATCAAAAGCCTTTATCCCTTGACATTTATCCAGGTGAAACAGCATTATGG
GTTTGTGCTGTACCGGACAACACTTCCTCAAGATTGCAGCAACCCAGCACCTCTCTCTTCACCCCTCAATGGAGTCC
ACGATCGAGCATATGTTGCTGTGGATGGGATCCCCCAGGGAGTCCTTGAGCGAAACAATGTGATCACTCTGAACATA
ACAGGGAAAGCTGGAGCCACTCTGGACCTTCTGGTAGAGAACATGGGACGTGTGAACTATGGTGCATATATCAACGA
TTTTAAGGGTTTGGTTTCTAACCTGACTCTCAGTTCCAATATCCTCACGGACTGGACGATCTTTCCACTGGACACTG
AGGATGCAGTGCGCAGCCACCTGGGGGGCTGGGGACACCGTGACAGTGGCCACCATGATGAAGCCTGGGCCCACAAC
TCATCCAACTACACGCTCCCGGCCTTTTATATGGGGAACTTCTCCATTCCCAGTGGGATCCCAGACTTGCCCCAGGA
CACCTTTATCCAGTTTCCTGGATGGACCAAGGGCCAGGTCTGGATTAATGGCTTTAACCTTGGCCGCTATTGGCCAG
CCCGGGGCCCTCAGTTGACCTTGTTTGTGCCCCAGCACATCCTGATGACCTCGGCCCCAAACACCATCACCGTGCTG
GAACTGGAGTGGGCACCCTGCAGCAGTGATGATCCAGAACTATGTGCTGTGACGTTCGTGGACAGGCCAGTTATTGG
CTCATCTGTGACCTACGATCATCCCTCCAAACCTGTTGAAAAAAGACTCATGCCCCCACCCCCGCAAAAAAACAAAG
ATTCATGGCTGGACCATGTATGATGATGAAAGgtcgacAATCAACCTCTGGATTACAAAATTTGTGAAAGATTGACT
GGTATTCTTAACTATGTTGCTCCTTTTACGCTATGTGGATACGCTGCTTTAATGCCTTTGTATCATGCTATTGCTTC
CCGTATGGCTTTCATTTTCTCCTCCTTGTATAAATCCTGGTTGCTGTCTCTTTATGAGGAGTTGTGGCCCGTTGTCA
GGCAACGTGGCGTGGTGTGCACTGTGTTTGCTGACGCAACCCCCACTGGTTGGGGCATTGCCACCACCTGTCAGCTC
CTTTCCGGGACTTTCGCTTTCCCCCTCCCTATTGCCACGGCGGAACTCATCGCCGCCTGCCTTGCCCGCTGCTGGAC
AGGGGCTCGGCTGTTGGGCACTGACAATTCCGTGGTGTTGTCGGGGAAATCATCGTCCTTTCCTTGGCTGCTCGCCT
GTGTTGCCACCTGGATTCTGCGCGGGACGTCCTTCTGCTACGTCCCTTCGGCCCTCAATCCAGCGGACCTTCCTTCC
CGCGGCCTGCTGCCGGCTCTGCGGCCTCTTCCGCGTCTTCGCCTTCGCCCTCAGACGAGTCGGATCTCCCTTTGGGC
CGCCTCCCCGCCTGGAATTCGAGCTCGGTACCTTTAAGACCAATGACTTACAAGGCAGCTGTAGATCTTAGCCACTT
TTTAAAAGAAAAGGGGGGACTGGAAGGGCTAATTCACTCCCAACGAAGACAAGATCTGCTTTTTGCTTGTACTGGGT
CTCTCTGGTTAGACCAGATCTGAGCCTGGGAGCTCTCTGGCTAACTAGGGAACCCACTGCTTAAGCCTCAATAAAGC
TTGCCTTGAGTGCTTCAAGTAGTGTGTGCCCGTCTGTTGTGTGACTCTGGTAACTAGAGATCCCTCAGACCCTTTTA
GTCAGTGTGGAAAATCTCTAGCAGTAGTAGTTCATGTCATCTTATTATTCAGTATTTATAACTTGCAAAGAAATGAA
TATCAGAGAGTGAGAGGAACTTGTTTATTGCAGCTTATAATGGTTACAAATAAAGCAATAGCATCACAAATTTCACA
AATAAAGCATTTTTTTCACTGCATTCTAGTTGTGGTTTGTCCAAACTCATCAATGTATCTTATCATGTCTGGCTCTA
GCTATCCCGCCCCTAACTCCGCCCATCCCGCCCCTAACTCCGCCCAGTTCCGCCCATTCTCCGCCCCATGGCTGACT
AATTTTTTTTATTTATGCAGAGGCCGAGGCCGCCTCGGCCTCTGAGCTATTCCAGAAGTAGTGAGGAGGCTTTTTTG
GAGGCCTAGGGACGTACCCAATTCGCCCTATAGTGAGTCGTATTACGCGCGCTCACTGGCCGTCGTTTTACAACGTC
GTGACTGGGAAAACCCTGGCGTTACCCAACTTAATCGCCTTGCAGCACATCCCCCTTTCGCCAGCTGGCGTAATAGC
GAAGAGGCCCGCACCGATCGCCCTTCCCAACAGTTGCGCAGCCTGAATGGCGAATGGG
(LV hGLB1 OPT transfer vector)
SEQ ID NO: 29
ACGCGCCCTGTAGCGGCGCATTAAGCGCGGCGGGTGTGGTGGTTACGCGCAGCGTGACCGCTACACTTGCCAGCGCC
CTAGCGCCCGCTCCTTTCGCTTTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAACTTGATTAGGGTGATGGTTCAC
GTAGTGGGCCATCGCCCTGATAGACGGTTTTTCGCCCTTTGACGTTGGAGTCCACGTTCTTTAATAGTGGACTCTTG
TTCCAAACTGGAACAACACTCAACCCTATCTCGGTCTATTCTTTTGATTTATAAGGGATTTTGCCGATTTCGGCCTA
TTGGTTAAAAAATGAGCTGATTTAACAAAAATTTAACGCGAATTTTAACAAAATATTAACGCTTACAATTTAGGTGG
CACTTTTCGGGGAAATGTGCGCGGAACCCCTATTTGTTTATTTTTCTAAATACATTCAAATATGTATCCGCTCATGA
GACAATAACCCTGATAAATGCTTCAATAATAGCACCTAGATCAAGAGACAGGATGAGGATCGTTTCGCATGATTGAA
CAAGATGGATTGCACGCAGGTTCTCCGGCCGCTTGGGTGGAGAGGCTATTCGGCTATGACTGGGCACAACAGACAAT
CGGCTGCTCTGATGCCGCCGTGTTCCGGCTGTCAGCGCAGGGGCGCCCGGTTCTTTTTGTCAAGACCGACCTGTCCG
GTGCCCTGAATGAACTGCAAGACGAGGCAGCGCGGCTATCGTGGCTGGCCACGACGGGCGTTCCTTGCGCAGCTGTG
CTCGACGTTGTCACTGAAGCGGGAAGGGACTGGCTGCTATTGGGCGAAGTGCCGGGGCAGGATCTCCTGTCATCTCA
CCTTGCTCCTGCCGAGAAAGTATCCATCATGGCTGATGCAATGCGGCGGCTGCATACGCTTGATCCGGCTACCTGCC
CATTCGACCACCAAGCGAAACATCGCATCGAGCGAGCACGTACTCGGATGGAAGCCGGTCTTGTCGATCAGGATGAT
CTGGACGAAGAGCATCAGGGGCTCGCGCCAGCCGAACTGTTCGCCAGGCTCAAGGCGAGCATGCCCGACGGCGAGGA
TCTCGTCGTGACCCATGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCTGAAGAGCTTGGCGGC
GAATGGGCTGACCGCTTCCTCGTGCTTTACGGTATCGCCGCTCCCGATTCGCAGCGCATCGCCTTCTATCGCCTTCT
TGACGAGTTCTTCTGAATTATTAACGCTTACAATTTCCTGATGCGGTATTTTCTCCTTACGCATCTGTGCGGTATTT
CACACCGCATCAGGTGGCACTTTTCGGGGAAATGTGCGCGGAACCCCTATTTGTTTATTTTTCTAAATACATTCAAA
TATGTATCCGCTCATGACCAAAATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAATAC
TGTTCTTCTAGTGTAGCCGTAGTTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGACTCAAGACGATAGTTACCGGAT
AAGGCGCAGCGGTCGGGCTGAACGGGGGGGTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGAGCGCAGCGAGTCAGTGAGCGAG
GAAGCGGAAGAGCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAATTTCACACAGGAAACAGCTATGAC
CATGATTACGCCAAGCGCGCAATTAACCCTCACTAAAGGGAACAAAAGCTGGAGCTGCAAGCTTGGCCATTGCATAC
GTTGTATCCATATCATAATATGTACATTTATATTGGCTCATGTCCAACATTACCGCCATGTTGACATTGATTATTGA
CTAGTTATTAATAGTAATCAATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAATGACGTATGTTCCCATAGTAAC
GCCAATAGGGACTTTCCATTGACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGATGCGGTTTTGGCAGTA
CATCAATGGGCGTGGATAGCGGTTTGACTCACGGGGATTTCCAAGTCTCCACCCCATTGACGTCAATGGGAGTTTGT
TTTGGCACCAAAATCAACGGGACTTTCCAAAATGTCGTAACAACTCCGCCCCATTGACGCAAATGGGCGGTAGGCGT
GTACGGTGGGAGGTCTATATAAGCAGAGCTCGTTTAGTGAACCGGGGTCTCTCTGGTTAGACCAGATCTGAGCCTGG
GAGCTCTCTGGCTAACTAGGGAACCCACTGCTTAAGCCTCAATAAAGCTTGCCTTGAGTGCTTCAAGTAGTGTGTGC
CCGTCTGTTGTGTGACTCTGGTAACTAGAGATCCCTCAGACCCTTTTAGTCAGTGTGGAAAATCTCTAGCAGTGGCG
CCCGAACAGGGACTTGAAAGCGAAAGGGAAACCAGAGGAGCTCTCTCGACGCAGGACTCGGCTTGCTGAAGCGCGCA
CGGCAAGAGGCGAGGGGCGGCGACTGGTGAGTACGCCAAAAATTTTGACTAGCGGAGGCTAGAAGGAGAGAGATGGG
TGCGAGAGCGTCAGTATTAAGCGGGGGAGAATTAGATCGCGATGGGAAAAAATTCGGTTAAGGCCAGGGGGAAAGAA
AAAATATAAATTAAAACATATAGTATGGGCAAGCAGGGAGCTAGAACGATTCGCAGTTAATCCTGGCCTGTTAGAAA
CATCAGAAGGCTGTAGACAAATACTGGGACAGCTACAACCATCCCTTCAGACAGGATCAGAAGAACTTAGATCATTA
TATAATACAGTAGCAACCCTCTATTGTGTGCATCAAAGGATAGAGATAAAAGACACCAAGGAAGCTTTAGACAAGAT
AGAGGAAGAGCAAAACAAAAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGGAGTAGCACCCACCAAGGC
AAAGAGAAGAGTGGTGCAGAGAGAAAAAAGAGCAGTGGGAATAGGAGCTTTGTTCCTTGGGTTCTTGGGAGCAGCAG
GAAGCACTATGGGCGCAGCGTCAATGACGCTGACGGTACAGGCCAGACAATTATTGTCTGGTATAGTGCAGCAGCAG
AACAATTTGCTGAGGGCTATTGAGGCGCAACAGCATCTGTTGCAACTCACAGTCTGGGGCATCAAGCAGCTCCAGGC
AAGAATCCTGGCTGTGGAAAGATACCTAAAGGATCAACAGCTCCTGGGGATTTGGGGTTGCTCTGGAAAACTCATTT
GCACCACTGCTGTGCCTTGGAATGCTAGTTGGAGTAATAAATCTCTGGAACAGATTTGGAATCACACGACCTGGATG
GAGTGGGACAGAGAAATTAACAATTACACAAGCTTAATACACTCCTTAATTGAAGAATCGCAAAACCAGCAAGAAAA
GAATGAACAAGAATTATTGGAATTAGATAAATGGGCAAGTTTGTGGAATTGGTTTAACATAACAAATTGGCTGTGGT
ATATAAAATTATTCATAATGATAGTAGGAGGCTTGGTAGGTTTAAGAATAGTTTTTGCTGTACTTTCTATAGTGAAT
AGAGTTAGGCAGGGATATTCACCATTATCGTTTCAGACCCACCTCCCAACCCCGAGGGGACCCGACAGGCCCGAAGG
AATAGAAGAAGAAGGTGGAGAGAGAGACAGAGACAGATCCATTCGATTAGTGAACGGATCTCGACGGTATCGGTTAA
CTTTTAAAAGAAAAGGGGGGATTGGGGGGTACAGTGCAGGGGAAAGAATAGTAGACATAATAGCAACAGACATACAA
ACTAAAGAATTACAAAAACAAATTACAAAATTCAAAATTTTATCGATAAGCTTGATATCATCGATTTCCGAATTCCA
CGGGGTTGGGGTTGCGCCTTTTCCAAGGCAGCCCTGGGTTTGCGCAGGGACGCGGCTGCTCTGGGCGTGGTTCCGGG
AAACGCAGCGGCGCCGACCCTGGGTCTCGCACATTCTTCACGTCCGTTCGCAGCGTCACCCGGATCTTCGCCGCTAC
CCTTGTGGGCCCCCCGGCGACGCTTCCTGCTCCGCCCCTAAGTCGGGAAGGTTCCTTGCGGTTCGCGGCGTGCCGGA
CGTGACAAACGGAAGCCGCACGTCTCACTAGTACCCTCGCAGACGGACAGCGCCAGGGAGCAATGGCAGCGCGCCGA
CCGCGATGGGCTGTGGCCAATAGCGGCTGCTCAGCAGGGCGCGCCGAGAGCAGCGGCCGGGAAGGGGCGGTGCGGGA
GGCGGGGTGTGGGGCGGTAGTGTGGGCCCTGTTCCTGCCCGCGCGGTGTTCCGCATTCTGCAAGCCTCCGGAGCGCA
CGTCGGCAGTCGGCTCCCTCGTTGACCGAATCACCGACCTCTCTCCCCAACGCggatccGCCGCCACCATGCCGGGT
TTCCTGGTGCGTATCCTGCCGCTGCTGCTGGTTCTGCTGCTGCTGGGTCCGACCCGTGGCCTGCGTAACGCGACCCA
GCGTATGTTTGAGATTGACTACAGCCGTGATAGCTTCCTGAAGGATGGTCAACCGTTTCGTTACATCAGCGGCAGCA
TTCACTATAGCCGTGTGCCGCGTTTCTACTGGAAAGACCGTCTGCTGAAGATGAAAATGGCGGGTCTGAACGCGATC
CAGACCTATGTTCCGTGGAACTTTCACGAGCCGTGGCCGGGTCAATATCAATTCAGCGAGGACCACGATGTGGAATA
CTTTCTGCGTCTGGCGCACGAACTGGGTCTGCTGGTTATCCTGCGTCCGGGCCCGTACATTTGCGCGGAGTGGGAAA
TGGGTGGCCTGCCGGCGTGGCTGCTGGAGAAGGAAAGCATCCTGCTGCGTAGCAGCGACCCGGATTATCTGGCGGCG
GTGGATAAATGGCTGGGTGTTCTGCTGCCGAAGATGAAACCGCTGCTGTACCAGAACGGTGGCCCGGTGATTACCGT
GCAAGTTGAGAACGAATACGGCAGCTATTTCGCGTGCGACTTTGATTACCTGCGTTTCCTGCAGAAGCGTTTTCGTC
ACCACCTGGGTGACGATGTGGTTCTGTTCACCACCGACGGCGCGCACAAGACCTTTCTGAAATGCGGTGCGCTGCAG
GGCCTGTATACCACCGTTGACTTCGGTACCGGCAGCAACATCACCGATGCGTTTCTGAGCCAGCGTAAGTGCGAGCC
GAAAGGTCCGCTGATTAACAGCGAATTCTACACCGGTTGGCTGGATCACTGGGGCCAACCGCACAGCACCATCAAGA
CCGAGGCGGTGGCGAGCAGCCTGTATGACATTCTGGCGCGTGGTGCGAGCGTTAACCTGTACATGTTCATCGGTGGC
ACCAACTTTGCGTATTGGAACGGTGCGAACAGCCCGTATGCGGCGCAGCCGACCAGCTACGACTATGATGCGCCGCT
GAGCGAGGCGGGTGACCTGACCGAAAAATATTTCGCGCTGCGTAACATCATTCAAAAGTTTGAGAAAGTTCCGGAAG
GTCCGATCCCGCCGAGCACCCCGAAGTTTGCGTACGGCAAAGTGACCCTGGAAAAGCTGAAAACCGTTGGTGCGGCG
CTGGATATCCTGTGCCCGAGCGGTCCGATTAAGAGCCTGTATCCGCTGACCTTCATTCAGGTGAAACAACACTACGG
TTTTGTTCTGTATCGTACCACCCTGCCGCAGGACTGCAGCAACCCGGCGCCGCTGAGCAGCCCGCTGAACGGCGTTC
ACGACCGTGCGTACGTGGCGGTTGATGGTATCCCGCAAGGCGTGCTGGAGCGTAACAACGTTATCACCCTGAACATT
ACCGGTAAAGCGGGTGCGACCCTGGACCTGCTGGTGGAAAACATGGGTCGTGTTAACTACGGCGCGTATATTAACGA
TTTCAAAGGTCTGGTGAGCAACCTGACCCTGAGCAGCAACATCCTGACCGACTGGACCATTTTTCCGCTGGACACCG
AGGATGCGGTTCGTAGCCACCTGGGTGGCTGGGGTCACCGTGACAGCGGTCACCACGATGAAGCGTGGGCGCACAAC
AGCAGCAACTACACCCTGCCGGCGTTCTATATGGGTAACTTTAGCATTCCGAGCGGCATTCCGGACCTGCCGCAGGA
TACCTTCATCCAATTTCCGGGTTGGACCAAAGGCCAAGTGTGGATTAACGGTTTCAACCTGGGTCGTTATTGGCCGG
CGCGTGGTCCGCAGCTGACCCTGTTTGTGCCGCAACACATCCTGATGACCAGCGCGCCGAACACCATTACCGTTCTG
GAGCTGGAATGGGCGCCGTGCAGCAGCGATGATCCGGAGCTGTGCGCGGTGACCTTCGTTGACCGTCCGGTGATCGG
CAGCAGCGTTACCTACGATCACCCGAGCAAGCCGGTGGAAAAACGTCTGATGCCGCCGCCGCCGCAGAAGAACAAAG
ACAGCTGGCTGGATCACGTTTAATGATGAAAGgtcgacAATCAACCTCTGGATTACAAAATTTGTGAAAGATTGACT
GGTATTCTTAACTATGTTGCTCCTTTTACGCTATGTGGATACGCTGCTTTAATGCCTTTGTATCATGCTATTGCTTC
CCGTATGGCTTTCATTTTCTCCTCCTTGTATAAATCCTGGTTGCTGTCTCTTTATGAGGAGTTGTGGCCCGTTGTCA
GGCAACGTGGCGTGGTGTGCACTGTGTTTGCTGACGCAACCCCCACTGGTTGGGGCATTGCCACCACCTGTCAGCTC
CTTTCCGGGACTTTCGCTTTCCCCCTCCCTATTGCCACGGCGGAACTCATCGCCGCCTGCCTTGCCCGCTGCTGGAC
AGGGGCTCGGCTGTTGGGCACTGACAATTCCGTGGTGTTGTCGGGGAAATCATCGTCCTTTCCTTGGCTGCTCGCCT
GTGTTGCCACCTGGATTCTGCGCGGGACGTCCTTCTGCTACGTCCCTTCGGCCCTCAATCCAGCGGACCTTCCTTCC
CGCGGCCTGCTGCCGGCTCTGCGGCCTCTTCCGCGTCTTCGCCTTCGCCCTCAGACGAGTCGGATCTCCCTTTGGGC
CGCCTCCCCGCCTGGAATTCGAGCTCGGTACCTTTAAGACCAATGACTTACAAGGCAGCTGTAGATCTTAGCCACTT
TTTAAAAGAAAAGGGGGGACTGGAAGGGCTAATTCACTCCCAACGAAGACAAGATCTGCTTTTTGCTTGTACTGGGT
CTCTCTGGTTAGACCAGATCTGAGCCTGGGAGCTCTCTGGCTAACTAGGGAACCCACTGCTTAAGCCTCAATAAAGC
TTGCCTTGAGTGCTTCAAGTAGTGTGTGCCCGTCTGTTGTGTGACTCTGGTAACTAGAGATCCCTCAGACCCTTTTA
GTCAGTGTGGAAAATCTCTAGCAGTAGTAGTTCATGTCATCTTATTATTCAGTATTTATAACTTGCAAAGAAATGAA
TATCAGAGAGTGAGAGGAACTTGTTTATTGCAGCTTATAATGGTTACAAATAAAGCAATAGCATCACAAATTTCACA
AATAAAGCATTTTTTTCACTGCATTCTAGTTGTGGTTTGTCCAAACTCATCAATGTATCTTATCATGTCTGGCTCTA
GCTATCCCGCCCCTAACTCCGCCCATCCCGCCCCTAACTCCGCCCAGTTCCGCCCATTCTCCGCCCCATGGCTGACT
AATTTTTTTTATTTATGCAGAGGCCGAGGCCGCCTCGGCCTCTGAGCTATTCCAGAAGTAGTGAGGAGGCTTTTTTG
GAGGCCTAGGGACGTACCCAATTCGCCCTATAGTGAGTCGTATTACGCGCGCTCACTGGCCGTCGTTTTACAACGTC
GTGACTGGGAAAACCCTGGCGTTACCCAACTTAATCGCCTTGCAGCACATCCCCCTTTCGCCAGCTGGCGTAATAGC
GAAGAGGCCCGCACCGATCGCCCTTCCCAACAGTTGCGCAGCCTGAATGGCGAATGGG
(LV mGLB1 C2C12 transfer vector)
SEQ ID NO: 30
ACGCGCCCTGTAGCGGCGCATTAAGCGCGGCGGGTGTGGTGGTTACGCGCAGCGTGACCGCTACACTTGCCAGCGCC
CTAGCGCCCGCTCCTTTCGCTTTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAACTTGATTAGGGTGATGGTTCAC
GTAGTGGGCCATCGCCCTGATAGACGGTTTTTCGCCCTTTGACGTTGGAGTCCACGTTCTTTAATAGTGGACTCTTG
TTCCAAACTGGAACAACACTCAACCCTATCTCGGTCTATTCTTTTGATTTATAAGGGATTTTGCCGATTTCGGCCTA
TTGGTTAAAAAATGAGCTGATTTAACAAAAATTTAACGCGAATTTTAACAAAATATTAACGCTTACAATTTAGGTGG
CACTTTTCGGGGAAATGTGCGCGGAACCCCTATTTGTTTATTTTTCTAAATACATTCAAATATGTATCCGCTCATGA
GACAATAACCCTGATAAATGCTTCAATAATAGCACCTAGATCAAGAGACAGGATGAGGATCGTTTCGCATGATTGAA
CAAGATGGATTGCACGCAGGTTCTCCGGCCGCTTGGGTGGAGAGGCTATTCGGCTATGACTGGGCACAACAGACAAT
CGGCTGCTCTGATGCCGCCGTGTTCCGGCTGTCAGCGCAGGGGCGCCCGGTTCTTTTTGTCAAGACCGACCTGTCCG
GTGCCCTGAATGAACTGCAAGACGAGGCAGCGCGGCTATCGTGGCTGGCCACGACGGGCGTTCCTTGCGCAGCTGTG
CTCGACGTTGTCACTGAAGCGGGAAGGGACTGGCTGCTATTGGGCGAAGTGCCGGGGCAGGATCTCCTGTCATCTCA
CCTTGCTCCTGCCGAGAAAGTATCCATCATGGCTGATGCAATGCGGCGGCTGCATACGCTTGATCCGGCTACCTGCC
CATTCGACCACCAAGCGAAACATCGCATCGAGCGAGCACGTACTCGGATGGAAGCCGGTCTTGTCGATCAGGATGAT
CTGGACGAAGAGCATCAGGGGCTCGCGCCAGCCGAACTGTTCGCCAGGCTCAAGGCGAGCATGCCCGACGGCGAGGA
TCTCGTCGTGACCCATGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCTGAAGAGCTTGGCGGC
GAATGGGCTGACCGCTTCCTCGTGCTTTACGGTATCGCCGCTCCCGATTCGCAGCGCATCGCCTTCTATCGCCTTCT
TGACGAGTTCTTCTGAATTATTAACGCTTACAATTTCCTGATGCGGTATTTTCTCCTTACGCATCTGTGCGGTATTT
CACACCGCATCAGGTGGCACTTTTCGGGGAAATGTGCGCGGAACCCCTATTTGTTTATTTTTCTAAATACATTCAAA
TATGTATCCGCTCATGACCAAAATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAATAC
TGTTCTTCTAGTGTAGCCGTAGTTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGACTCAAGACGATAGTTACCGGAT
AAGGCGCAGCGGTCGGGCTGAACGGGGGGGTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGAGCGCAGCGAGTCAGTGAGCGAG
GAAGCGGAAGAGCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAATTTCACACAGGAAACAGCTATGAC
CATGATTACGCCAAGCGCGCAATTAACCCTCACTAAAGGGAACAAAAGCTGGAGCTGCAAGCTTGGCCATTGCATAC
GTTGTATCCATATCATAATATGTACATTTATATTGGCTCATGTCCAACATTACCGCCATGTTGACATTGATTATTGA
CTAGTTATTAATAGTAATCAATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAATGACGTATGTTCCCATAGTAAC
GCCAATAGGGACTTTCCATTGACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGATGCGGTTTTGGCAGTA
CATCAATGGGCGTGGATAGCGGTTTGACTCACGGGGATTTCCAAGTCTCCACCCCATTGACGTCAATGGGAGTTTGT
TTTGGCACCAAAATCAACGGGACTTTCCAAAATGTCGTAACAACTCCGCCCCATTGACGCAAATGGGCGGTAGGCGT
GTACGGTGGGAGGTCTATATAAGCAGAGCTCGTTTAGTGAACCGGGGTCTCTCTGGTTAGACCAGATCTGAGCCTGG
GAGCTCTCTGGCTAACTAGGGAACCCACTGCTTAAGCCTCAATAAAGCTTGCCTTGAGTGCTTCAAGTAGTGTGTGC
CCGTCTGTTGTGTGACTCTGGTAACTAGAGATCCCTCAGACCCTTTTAGTCAGTGTGGAAAATCTCTAGCAGTGGCG
CCCGAACAGGGACTTGAAAGCGAAAGGGAAACCAGAGGAGCTCTCTCGACGCAGGACTCGGCTTGCTGAAGCGCGCA
CGGCAAGAGGCGAGGGGCGGCGACTGGTGAGTACGCCAAAAATTTTGACTAGCGGAGGCTAGAAGGAGAGAGATGGG
TGCGAGAGCGTCAGTATTAAGCGGGGGAGAATTAGATCGCGATGGGAAAAAATTCGGTTAAGGCCAGGGGGAAAGAA
AAAATATAAATTAAAACATATAGTATGGGCAAGCAGGGAGCTAGAACGATTCGCAGTTAATCCTGGCCTGTTAGAAA
CATCAGAAGGCTGTAGACAAATACTGGGACAGCTACAACCATCCCTTCAGACAGGATCAGAAGAACTTAGATCATTA
TATAATACAGTAGCAACCCTCTATTGTGTGCATCAAAGGATAGAGATAAAAGACACCAAGGAAGCTTTAGACAAGAT
AGAGGAAGAGCAAAACAAAAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGGAGTAGCACCCACCAAGGC
AAAGAGAAGAGTGGTGCAGAGAGAAAAAAGAGCAGTGGGAATAGGAGCTTTGTTCCTTGGGTTCTTGGGAGCAGCAG
GAAGCACTATGGGCGCAGCGTCAATGACGCTGACGGTACAGGCCAGACAATTATTGTCTGGTATAGTGCAGCAGCAG
AACAATTTGCTGAGGGCTATTGAGGCGCAACAGCATCTGTTGCAACTCACAGTCTGGGGCATCAAGCAGCTCCAGGC
AAGAATCCTGGCTGTGGAAAGATACCTAAAGGATCAACAGCTCCTGGGGATTTGGGGTTGCTCTGGAAAACTCATTT
GCACCACTGCTGTGCCTTGGAATGCTAGTTGGAGTAATAAATCTCTGGAACAGATTTGGAATCACACGACCTGGATG
GAGTGGGACAGAGAAATTAACAATTACACAAGCTTAATACACTCCTTAATTGAAGAATCGCAAAACCAGCAAGAAAA
GAATGAACAAGAATTATTGGAATTAGATAAATGGGCAAGTTTGTGGAATTGGTTTAACATAACAAATTGGCTGTGGT
ATATAAAATTATTCATAATGATAGTAGGAGGCTTGGTAGGTTTAAGAATAGTTTTTGCTGTACTTTCTATAGTGAAT
AGAGTTAGGCAGGGATATTCACCATTATCGTTTCAGACCCACCTCCCAACCCCGAGGGGACCCGACAGGCCCGAAGG
AATAGAAGAAGAAGGTGGAGAGAGAGACAGAGACAGATCCATTCGATTAGTGAACGGATCTCGACGGTATCGGTTAA
CTTTTAAAAGAAAAGGGGGGATTGGGGGGTACAGTGCAGGGGAAAGAATAGTAGACATAATAGCAACAGACATACAA
ACTAAAGAATTACAAAAACAAATTACAAAATTCAAAATTTTATCGATAAGCTTGATATCATCGATTTCCGAATTCCA
CGGGGTTGGGGTTGCGCCTTTTCCAAGGCAGCCCTGGGTTTGCGCAGGGACGCGGCTGCTCTGGGCGTGGTTCCGGG
AAACGCAGCGGCGCCGACCCTGGGTCTCGCACATTCTTCACGTCCGTTCGCAGCGTCACCCGGATCTTCGCCGCTAC
CCTTGTGGGCCCCCCGGCGACGCTTCCTGCTCCGCCCCTAAGTCGGGAAGGTTCCTTGCGGTTCGCGGCGTGCCGGA
CGTGACAAACGGAAGCCGCACGTCTCACTAGTACCCTCGCAGACGGACAGCGCCAGGGAGCAATGGCAGCGCGCCGA
CCGCGATGGGCTGTGGCCAATAGCGGCTGCTCAGCAGGGCGCGCCGAGAGCAGCGGCCGGGAAGGGGCGGTGCGGGA
GGCGGGGTGTGGGGCGGTAGTGTGGGCCCTGTTCCTGCCCGCGCGGTGTTCCGCATTCTGCAAGCCTCCGGAGCGCA
CGTCGGCAGTCGGCTCCCTCGTTGACCGAATCACCGACCTCTCTCCCCAACGCggatccAGAGCGCCCACTGCCTAA
CGGAGAGACCCCATCGTGGCGCGATCATGCTCCGGGTCCCCCTGTGTACGCCGCTCCCGCTCCTGGCACTGCTGCAA
CTGCTGGGCGCTGCGCACGGCATCTATAATGTCACCCAGAGGACATTTAAGCTCGACTACAGCCGGGACCGCTTCCT
CAAGGATGGACAGCCATTCCGATACATCTCGGGAAGCATTCATTACTTCCGGATACCCCGCTTCTACTGGGAGGACC
GGCTGCTGAAGATGAAGATGGCTGGGCTGAATGCTATCCAGATGTACGTGCCCTGGAACTTCCATGAACCCCAACCA
GGACAATATGAGTTTTCTGGGGACCGTGATGTGGAGCATTTCATCCAGCTGGCTCATGAGCTGGGACTCCTGGTGAT
CCTGAGGCCTGGGCCCTACATCTGTGCAGAGTGGGACATGGGGGGCTTACCTGCTTGGCTACTAGAGAAACAATCTA
TCGTTCTCCGGTCTTCTGACCCAGACTACCTTGTAGCTGTGGATAAATGGCTGGCAGTCCTTCTGCCCAAGATGAAG
CCCCTGCTCTACCAGAACGGAGGACCGATCATAACCGTGCAGGTTGAGAATGAGTACGGGTCCTACTTTGCCTGCGA
TTACGACTACCTACGCTTCCTGGTGCACCGCTTCCGCTACCATCTGGGTAATGACGTCATTCTCTTCACCACCGACG
GAGCAAGTGAAAAAATGCTGAAGTGTGGGACCCTGCAGGACCTGTACGCCACAGTGGATTTTGGAACAGGCAACAAT
ATCACACAAGCTTTCCTGGTCCAGAGGAAGTTTGAACCTAAAGGACCTTTGATCAATTCTGAGTTCTATACTGGCTG
GCTAGACCACTGGGGTAAACCCCATTCCACGGTGAAAACTAAAACACTGGCTACCTCCCTCTATAACCTGCTTGCCC
GTGGGGCCAACGTGAACTTGTACATGTTTATAGGTGGGACCAATTTTGCCTATTGGAATGGTGCCAACACGCCCTAT
GAGCCACAGCCCACCAGCTATGACTACGACGCCCCACTGAGCGAGGCTGGGGACCTCACTAAGAAGTATTTTGCTCT
TCGAGAAGTCATTCAGATGTTTAAAGAAGTCCCAGAAGGCCCTATCCCTCCGTCTACACCCAAATTTGCATATGGAA
AAGTTGCTCTGAGAAAGTTCAAGACAGTGGCTGAAGCTCTGGGTATCCTGTGTCCCAATGGGCCAGTGAAAAGCCTC
TATCCCCTGACATTCACTCAGGTAAAACAGTATTTTGGGTATGTGCTGTACCGAACAACGCTTCCTCAAGATTGCAG
TAACCCGAAACCCATTTTCTCTTCACCCTTCAATGGTGTCCGTGATCGGGCTTACGTCTCTGTGGACGGGGTCCCCC
AAGGAATCCTTGATCGAAACCTCATGACAGCTCTGAACATACAGGGGAAGGCTGGAGCCACGCTGGACATCCTGGTG
GAGAACATGGGGCGTGTGAACTATGGCAGATTCATCAATGACTTCAAGGGTTTGATTTCCAACATGACTATCAACTC
CACTGTCCTCACCAACTGGACGGTCTTCCCACTGGACACTGAGGCCATGGTACGCAACCATCTCTGGGGCCGGGAGG
CCAGTGATGGGGGTCACCTTGACGGACGGTCGACCTCCAATTCTTCGGACCTCATACTCCCCACCTTTTACGTGGGC
AACTTCTCCATCCCCTCGGGCATCCCAGACCTGCCACAGGACACCTTCATCCAGTTTCCTGGGTGGTCCAAGGGTCA
AGTATGGATCAATGGCTTTAACCTCGGCCGATACTGGCCCACAATGGGCCCACAAAAGACCTTGTTCGTGCCAAGGA
ACATCCTGACCACTTCAGCCCCAAACAACATCACAGTGTTGGAGCTAGAGTTTGCACCCTGCAGCGAGGGGACCCCA
GAGCTGTGTACAGTAGAGTTTGTTGACACTCCGGTCATTTCCTGACCTGACTTGACCATCGGTGGCCATTTTCCAAG
CCAGTCTGGTCAAGACTCATGGCTGAACCTCTGAGACTGAGCCTTGGGGAGCACAGCTCTACTCTGGTTACACGGAT
CACCTTTGTTGTGCTAGAATGGAAGCTACATctcgagGTCGACAATCAACCTCTGGATTACAAAATTTGTGAAAGAT
TGACTGGTATTCTTAACTATGTTGCTCCTTTTACGCTATGTGGATACGCTGCTTTAATGCCTTTGTATCATGCTATT
GCTTCCCGTATGGCTTTCATTTTCTCCTCCTTGTATAAATCCTGGTTGCTGTCTCTTTATGAGGAGTTGTGGCCCGT
TGTCAGGCAACGTGGCGTGGTGTGCACTGTGTTTGCTGACGCAACCCCCACTGGTTGGGGCATTGCCACCACCTGTC
AGCTCCTTTCCGGGACTTTCGCTTTCCCCCTCCCTATTGCCACGGCGGAACTCATCGCCGCCTGCCTTGCCCGCTGC
TGGACAGGGGCTCGGCTGTTGGGCACTGACAATTCCGTGGTGTTGTCGGGGAAATCATCGTCCTTTCCTTGGCTGCT
CGCCTGTGTTGCCACCTGGATTCTGCGCGGGACGTCCTTCTGCTACGTCCCTTCGGCCCTCAATCCAGCGGACCTTC
CTTCCCGCGGCCTGCTGCCGGCTCTGCGGCCTCTTCCGCGTCTTCGCCTTCGCCCTCAGACGAGTCGGATCTCCCTT
TGGGCCGCCTCCCCGCCTGGAATTCGAGCTCGGTACCTTTAAGACCAATGACTTACAAGGCAGCTGTAGATCTTAGC
CACTTTTTAAAAGAAAAGGGGGGACTGGAAGGGCTAATTCACTCCCAACGAAGACAAGATCTGCTTTTTGCTTGTAC
TGGGTCTCTCTGGTTAGACCAGATCTGAGCCTGGGAGCTCTCTGGCTAACTAGGGAACCCACTGCTTAAGCCTCAAT
AAAGCTTGCCTTGAGTGCTTCAAGTAGTGTGTGCCCGTCTGTTGTGTGACTCTGGTAACTAGAGATCCCTCAGACCC
TTTTAGTCAGTGTGGAAAATCTCTAGCAGTAGTAGTTCATGTCATCTTATTATTCAGTATTTATAACTTGCAAAGAA
ATGAATATCAGAGAGTGAGAGGAACTTGTTTATTGCAGCTTATAATGGTTACAAATAAAGCAATAGCATCACAAATT
TCACAAATAAAGCATTTTTTTCACTGCATTCTAGTTGTGGTTTGTCCAAACTCATCAATGTATCTTATCATGTCTGG
CTCTAGCTATCCCGCCCCTAACTCCGCCCATCCCGCCCCTAACTCCGCCCAGTTCCGCCCATTCTCCGCCCCATGGC
TGACTAATTTTTTTTATTTATGCAGAGGCCGAGGCCGCCTCGGCCTCTGAGCTATTCCAGAAGTAGTGAGGAGGCTT
TTTTGGAGGCCTAGGGACGTACCCAATTCGCCCTATAGTGAGTCGTATTACGCGCGCTCACTGGCCGTCGTTTTACA
ACGTCGTGACTGGGAAAACCCTGGCGTTACCCAACTTAATCGCCTTGCAGCACATCCCCCTTTCGCCAGCTGGCGTA
ATAGCGAAGAGGCCCGCACCGATCGCCCTTCCCAACAGTTGCGCAGCCTGAATGGCGAATGGG
(LV human MAN2B WT transfer vector)
SEQ ID NO: 31
ACGCGCCCTGTAGCGGCGCATTAAGCGCGGCGGGTGTGGTGGTTACGCGCAGCGTGACCGCTACACTTGCCAGCGCC
CTAGCGCCCGCTCCTTTCGCTTTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAACTTGATTAGGGTGATGGTTCAC
GTAGTGGGCCATCGCCCTGATAGACGGTTTTTCGCCCTTTGACGTTGGAGTCCACGTTCTTTAATAGTGGACTCTTG
TTCCAAACTGGAACAACACTCAACCCTATCTCGGTCTATTCTTTTGATTTATAAGGGATTTTGCCGATTTCGGCCTA
TTGGTTAAAAAATGAGCTGATTTAACAAAAATTTAACGCGAATTTTAACAAAATATTAACGCTTACAATTTAGGTGG
CACTTTTCGGGGAAATGTGCGCGGAACCCCTATTTGTTTATTTTTCTAAATACATTCAAATATGTATCCGCTCATGA
GACAATAACCCTGATAAATGCTTCAATAATAGCACCTAGATCAAGAGACAGGATGAGGATCGTTTCGCATGATTGAA
CAAGATGGATTGCACGCAGGTTCTCCGGCCGCTTGGGTGGAGAGGCTATTCGGCTATGACTGGGCACAACAGACAAT
CGGCTGCTCTGATGCCGCCGTGTTCCGGCTGTCAGCGCAGGGGCGCCCGGTTCTTTTTGTCAAGACCGACCTGTCCG
GTGCCCTGAATGAACTGCAAGACGAGGCAGCGCGGCTATCGTGGCTGGCCACGACGGGCGTTCCTTGCGCAGCTGTG
CTCGACGTTGTCACTGAAGCGGGAAGGGACTGGCTGCTATTGGGCGAAGTGCCGGGGCAGGATCTCCTGTCATCTCA
CCTTGCTCCTGCCGAGAAAGTATCCATCATGGCTGATGCAATGCGGCGGCTGCATACGCTTGATCCGGCTACCTGCC
CATTCGACCACCAAGCGAAACATCGCATCGAGCGAGCACGTACTCGGATGGAAGCCGGTCTTGTCGATCAGGATGAT
CTGGACGAAGAGCATCAGGGGCTCGCGCCAGCCGAACTGTTCGCCAGGCTCAAGGCGAGCATGCCCGACGGCGAGGA
TCTCGTCGTGACCCATGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCTGAAGAGCTTGGCGGC
GAATGGGCTGACCGCTTCCTCGTGCTTTACGGTATCGCCGCTCCCGATTCGCAGCGCATCGCCTTCTATCGCCTTCT
TGACGAGTTCTTCTGAATTATTAACGCTTACAATTTCCTGATGCGGTATTTTCTCCTTACGCATCTGTGCGGTATTT
CACACCGCATCAGGTGGCACTTTTCGGGGAAATGTGCGCGGAACCCCTATTTGTTTATTTTTCTAAATACATTCAAA
TATGTATCCGCTCATGACCAAAATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAATAC
TGTTCTTCTAGTGTAGCCGTAGTTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGACTCAAGACGATAGTTACCGGAT
AAGGCGCAGCGGTCGGGCTGAACGGGGGGGTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGAGCGCAGCGAGTCAGTGAGCGAG
GAAGCGGAAGAGCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAATTTCACACAGGAAACAGCTATGAC
CATGATTACGCCAAGCGCGCAATTAACCCTCACTAAAGGGAACAAAAGCTGGAGCTGCAAGCTTGGCCATTGCATAC
GTTGTATCCATATCATAATATGTACATTTATATTGGCTCATGTCCAACATTACCGCCATGTTGACATTGATTATTGA
CTAGTTATTAATAGTAATCAATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAATGACGTATGTTCCCATAGTAAC
GCCAATAGGGACTTTCCATTGACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGATGCGGTTTTGGCAGTA
CATCAATGGGCGTGGATAGCGGTTTGACTCACGGGGATTTCCAAGTCTCCACCCCATTGACGTCAATGGGAGTTTGT
TTTGGCACCAAAATCAACGGGACTTTCCAAAATGTCGTAACAACTCCGCCCCATTGACGCAAATGGGCGGTAGGCGT
GTACGGTGGGAGGTCTATATAAGCAGAGCTCGTTTAGTGAACCGGGGTCTCTCTGGTTAGACCAGATCTGAGCCTGG
GAGCTCTCTGGCTAACTAGGGAACCCACTGCTTAAGCCTCAATAAAGCTTGCCTTGAGTGCTTCAAGTAGTGTGTGC
CCGTCTGTTGTGTGACTCTGGTAACTAGAGATCCCTCAGACCCTTTTAGTCAGTGTGGAAAATCTCTAGCAGTGGCG
CCCGAACAGGGACTTGAAAGCGAAAGGGAAACCAGAGGAGCTCTCTCGACGCAGGACTCGGCTTGCTGAAGCGCGCA
CGGCAAGAGGCGAGGGGCGGCGACTGGTGAGTACGCCAAAAATTTTGACTAGCGGAGGCTAGAAGGAGAGAGATGGG
TGCGAGAGCGTCAGTATTAAGCGGGGGAGAATTAGATCGCGATGGGAAAAAATTCGGTTAAGGCCAGGGGGAAAGAA
AAAATATAAATTAAAACATATAGTATGGGCAAGCAGGGAGCTAGAACGATTCGCAGTTAATCCTGGCCTGTTAGAAA
CATCAGAAGGCTGTAGACAAATACTGGGACAGCTACAACCATCCCTTCAGACAGGATCAGAAGAACTTAGATCATTA
TATAATACAGTAGCAACCCTCTATTGTGTGCATCAAAGGATAGAGATAAAAGACACCAAGGAAGCTTTAGACAAGAT
AGAGGAAGAGCAAAACAAAAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGGAGTAGCACCCACCAAGGC
AAAGAGAAGAGTGGTGCAGAGAGAAAAAAGAGCAGTGGGAATAGGAGCTTTGTTCCTTGGGTTCTTGGGAGCAGCAG
GAAGCACTATGGGCGCAGCGTCAATGACGCTGACGGTACAGGCCAGACAATTATTGTCTGGTATAGTGCAGCAGCAG
AACAATTTGCTGAGGGCTATTGAGGCGCAACAGCATCTGTTGCAACTCACAGTCTGGGGCATCAAGCAGCTCCAGGC
AAGAATCCTGGCTGTGGAAAGATACCTAAAGGATCAACAGCTCCTGGGGATTTGGGGTTGCTCTGGAAAACTCATTT
GCACCACTGCTGTGCCTTGGAATGCTAGTTGGAGTAATAAATCTCTGGAACAGATTTGGAATCACACGACCTGGATG
GAGTGGGACAGAGAAATTAACAATTACACAAGCTTAATACACTCCTTAATTGAAGAATCGCAAAACCAGCAAGAAAA
GAATGAACAAGAATTATTGGAATTAGATAAATGGGCAAGTTTGTGGAATTGGTTTAACATAACAAATTGGCTGTGGT
ATATAAAATTATTCATAATGATAGTAGGAGGCTTGGTAGGTTTAAGAATAGTTTTTGCTGTACTTTCTATAGTGAAT
AGAGTTAGGCAGGGATATTCACCATTATCGTTTCAGACCCACCTCCCAACCCCGAGGGGACCCGACAGGCCCGAAGG
AATAGAAGAAGAAGGTGGAGAGAGAGACAGAGACAGATCCATTCGATTAGTGAACGGATCTCGACGGTATCGGTTAA
CTTTTAAAAGAAAAGGGGGGATTGGGGGGTACAGTGCAGGGGAAAGAATAGTAGACATAATAGCAACAGACATACAA
ACTAAAGAATTACAAAAACAAATTACAAAATTCAAAATTTTATCGATAAGCTTGATATCATCGATTTCCGAATTCCA
CGGGGTTGGGGTTGCGCCTTTTCCAAGGCAGCCCTGGGTTTGCGCAGGGACGCGGCTGCTCTGGGCGTGGTTCCGGG
AAACGCAGCGGCGCCGACCCTGGGTCTCGCACATTCTTCACGTCCGTTCGCAGCGTCACCCGGATCTTCGCCGCTAC
CCTTGTGGGCCCCCCGGCGACGCTTCCTGCTCCGCCCCTAAGTCGGGAAGGTTCCTTGCGGTTCGCGGCGTGCCGGA
CGTGACAAACGGAAGCCGCACGTCTCACTAGTACCCTCGCAGACGGACAGCGCCAGGGAGCAATGGCAGCGCGCCGA
CCGCGATGGGCTGTGGCCAATAGCGGCTGCTCAGCAGGGCGCGCCGAGAGCAGCGGCCGGGAAGGGGCGGTGCGGGA
GGCGGGGTGTGGGGCGGTAGTGTGGGCCCTGTTCCTGCCCGCGCGGTGTTCCGCATTCTGCAAGCCTCCGGAGCGCA
CGTCGGCAGTCGGCTCCCTCGTTGACCGAATCACCGACCTCTCTCCCCAACGCqgatccGCCGCCACCATGGGCGCC
TACGCGCGGGCTTCGGGGGTCTGCGCTCGCGGCTGCCTGGACTCAGCAGGCCCCTGGACCATGTCCCGCGCCCTGCG
GCCACCGCTCCCGCCTCTCTGCTTTTTCCTTTTGTTGCTGGCGGCTGCCGGTGCTCGGGCCGGGGGATACGAGACAT
GCCCCACAGTGCAGCCGAACATGCTGAACGTGCACCTGCTGCCTCACACACATGATGACGTGGGCTGGCTCAAAACC
GTGGACCAGTACTTTTATGGAATCAAGAATGACATCCAGCACGCCGGTGTGCAGTACATCCTGGACTCGGTCATCTC
TGCCTTGCTGGCAGATCCCACCCGTCGCTTCATTTACGTGGAGATTGCCTTCTTCTCCCGTTGGTGGCACCAGCAGA
CAAATGCCACACAGGAAGTCGTGCGAGACCTTGTGCGCCAGGGGCGCCTGGAGTTCGCCAATGGTGGCTGGGTGATG
AACGATGAGGCAGCCACCCACTACGGTGCCATCGTGGACCAGATGACACTTGGGCTGCGCTTTCTGGAGGACACATT
TGGCAATGATGGGCGACCCCGTGTGGCCTGGCACATTGACCCCTTCGGCCACTCTCGGGAGCAGGCCTCGCTGTTTG
CGCAGATGGGCTTCGACGGCTTCTTCTTTGGGCGCCTTGATTATCAAGATAAGTGGGTACGGATGCAGAAGCTGGAG
ATGGAGCAGGTGTGGCGGGCCAGCACCAGCCTGAAGCCCCCGACCGCGGACCTCTTCACTGGTGTGCTTCCCAATGG
TTACAACCCGCCAAGGAATCTGTGCTGGGATGTGCTGTGTGTCGATCAGCCGCTGGTGGAGGACCCTCGCAGCCCCG
AGTACAACGCCAAGGAGCTGGTCGATTACTTCCTAAATGTGGCCACTGCCCAGGGCCGGTATTACCGCACCAACCAC
ACTGTGATGACCATGGGCTCGGACTTCCAATATGAGAATGCCAACATGTGGTTCAAGAACCTTGACAAGCTCATCCG
GCTGGTAAATGCGCAGCAGGCAAAAGGAAGCAGTGTCCATGTTCTCTACTCCACCCCCGCTTGTTACCTCTGGGAGC
TGAACAAGGCCAACCTCACCTGGTCAGTGAAACATGACGACTTCTTCCCTTACGCGGATGGCCCCCACCAGTTCTGG
ACCGGTTACTTTTCCAGTCGGCCGGCCCTCAAACGCTACGAGCGCCTCAGCTACAACTTCCTGCAGGTGTGCAACCA
GCTGGAGGCGCTGGTGGGCCTGGCGGCCAACGTGGGACCCTATGGCTCCGGAGACAGTGCACCCCTCAATGAGGCGA
TGGCTGTGCTCCAGCATCACGACGCCGTCAGCGGCACCTCCCGCCAGCACGTGGCCAACGACTACGCGCGCCAGCTT
GCGGCAGGCTGGGGGCCTTGCGAGGTTCTTCTGAGCAACGCGCTGGCGCGGCTCAGAGGCTTCAAAGATCACTTCAC
CTTTTGCCAACAGCTAAACATCAGCATCTGCCCGCTCAGCCAGACGGCGGCGCGCTTCCAGGTCATCGTTTATAATC
CCCTGGGGCGGAAGGTGAATTGGATGGTACGGCTGCCGGTCAGCGAAGGCGTTTTCGTTGTGAAGGACCCCAATGGC
AGGACAGTGCCCAGCGATGTGGTAATATTTCCCAGCTCAGACAGCCAGGCGCACCCTCCGGAGCTGCTGTTCTCAGC
CTCACTGCCCGCCCTGGGCTTCAGCACCTATTCAGTAGCCCAGGTGCCTCGCTGGAAGCCCCAGGCCCGCGCACCAC
AGCCCATCCCCAGAAGATCCTGGTCCCCTGCTTTAACCATCGAAAATGAGCACATCCGGGCAACGTTTGATCCTGAC
ACAGGGCTGTTGATGGAGATTATGAACATGAATCAGCAACTCCTGCTGCCTGTTCGCCAGACCTTCTTCTGGTACAA
CGCCAGTATAGGTGACAACGAAAGTGACCAGGCCTCAGGTGCCTACATCTTCAGACCCAACCAACAGAAACCGCTGC
CTGTGAGCCGCTGGGCTCAGATCCACCTGGTGAAGACACCCTTGGTGCAGGAGGTGCACCAGAACTTCTCAGCTTGG
TGTTCCCAGGTGGTTCGCCTGTACCCAGGACAGCGGCACCTGGAGCTAGAGTGGTCGGTGGGGCCGATACCTGTGGG
CGACACCTGGGGGAAGGAGGTCATCAGCCGTTTTGACACACCGCTGGAGACAAAGGGACGCTTCTACACAGACAGCA
ATGGCCGGGAGATCCTGGAGAGGAGGCGGGATTATCGACCCACCTGGAAACTGAACCAGACGGAGCCCGTGGCAGGA
AACTACTATCCAGTCAACACCCGGATTTACATCACGGATGGAAACATGCAGCTGACTGTGCTGACTGACCGCTCCCA
GGGGGGCAGCAGCCTGAGAGATGGCTCGCTGGAGCTCATGGTGCACCGAAGGCTGCTGAAGGACGATGGACGCGGAG
TATCGGAGCCACTAATGGAGAACGGGTCGGGGGCGTGGGTGCGAGGGCGCCACCTGGTGCTGCTGGACACAGCCCAG
GCTGCAGCCGCCGGACACCGGCTCCTGGCGGAGCAGGAGGTCCTGGCCCCTCAGGTGGTGCTGGCCCCGGGTGGCGG
CGCCGCCTACAATCTCGGGGCTCCTCCGCGCACGCAGTTCTCAGGGCTGCGCAGGGACCTGCCGCCCTCGGTGCACC
TGCTCACGCTGGCCAGCTGGGGCCCCGAAATGGTGCTGCTGCGCTTGGAGCACCAGTTTGCCGTAGGAGAGGATTCC
GGACGTAACCTGAGCGCCCCCGTTACCTTGAACTTGAGGGACCTGTTCTCCACCTTCACCATCACCCGCCTGCAGGA
GACCACGCTGGTGGCCAACCAGCTCCGCGAGGCAGCCTCCAGGCTCAAGTGGACAACAAACACAGGCCCCACACCCC
ACCAAACTCCGTACCAGCTGGACCCGGCCAACATCACGCTGGAACCCATGGAAATCCGCACTTTCCTGGCCTCAGTT
CAATGGAAGGAGGTGGATGGTTAGGTCTGCTGGgtcgacAATCAACCTCTGGATTACAAAATTTGTGAAAGATTGAC
TGGTATTCTTAACTATGTTGCTCCTTTTACGCTATGTGGATACGCTGCTTTAATGCCTTTGTATCATGCTATTGCTT
CCCGTATGGCTTTCATTTTCTCCTCCTTGTATAAATCCTGGTTGCTGTCTCTTTATGAGGAGTTGTGGCCCGTTGTC
AGGCAACGTGGCGTGGTGTGCACTGTGTTTGCTGACGCAACCCCCACTGGTTGGGGCATTGCCACCACCTGTCAGCT
CCTTTCCGGGACTTTCGCTTTCCCCCTCCCTATTGCCACGGCGGAACTCATCGCCGCCTGCCTTGCCCGCTGCTGGA
CAGGGGCTCGGCTGTTGGGCACTGACAATTCCGTGGTGTTGTCGGGGAAATCATCGTCCTTTCCTTGGCTGCTCGCC
TGTGTTGCCACCTGGATTCTGCGCGGGACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCCCTCAGACGAGTCGGATCTCCCTTTGGG
CCGCCTCCCCGCCTGGAATTCGAGCTCGGTACCTTTAAGACCAATGACTTACAAGGCAGCTGTAGATCTTAGCCACT
TTTTAAAAGAAAAGGGGGGACTGGAAGGGCTAATTCACTCCCAACGAAGACAAGATCTGCTTTTTGCTTGTACTGGG
TCTCTCTGGTTAGACCAGATCTGAGCCTGGGAGCTCTCTGGCTAACTAGGGAACCCACTGCTTAAGCCTCAATAAAG
CTTGCCTTGAGTGCTTCAAGTAGTGTGTGCCCGTCTGTTGTGTGACTCTGGTAACTAGAGATCCCTCAGACCCTTTT
AGTCAGTGTGGAAAATCTCTAGCAGTAGTAGTTCATGTCATCTTATTATTCAGTATTTATAACTTGCAAAGAAATGA
ATATCAGAGAGTGAGAGGAACTTGTTTATTGCAGCTTATAATGGTTACAAATAAAGCAATAGCATCACAAATTTCAC
AAATAAAGCATTTTTTTCACTGCATTCTAGTTGTGGTTTGTCCAAACTCATCAATGTATCTTATCATGTCTGGCTCT
AGCTATCCCGCCCCTAACTCCGCCCATCCCGCCCCTAACTCCGCCCAGTTCCGCCCATTCTCCGCCCCATGGCTGAC
TAATTTTTTTTATTTATGCAGAGGCCGAGGCCGCCTCGGCCTCTGAGCTATTCCAGAAGTAGTGAGGAGGCTTTTTT
GGAGGCCTAGGGACGTACCCAATTCGCCCTATAGTGAGTCGTATTACGCGCGCTCACTGGCCGTCGTTTTACAACGT
CGTGACTGGGAAAACCCTGGCGTTACCCAACTTAATCGCCTTGCAGCACATCCCCCTTTCGCCAGCTGGCGTAATAG
CGAAGAGGCCCGCACCGATCGCCCTTCCCAACAGTTGCGCAGCCTGAATGGCGAATGGG
(LV human MAN2B OPT transfer vector)
SEQ ID NO: 32
ACGCGCCCTGTAGCGGCGCATTAAGCGCGGCGGGTGTGGTGGTTACGCGCAGCGTGACCGCTACACTTGCCAGCGCC
CTAGCGCCCGCTCCTTTCGCTTTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAACTTGATTAGGGTGATGGTTCAC
GTAGTGGGCCATCGCCCTGATAGACGGTTTTTCGCCCTTTGACGTTGGAGTCCACGTTCTTTAATAGTGGACTCTTG
TTCCAAACTGGAACAACACTCAACCCTATCTCGGTCTATTCTTTTGATTTATAAGGGATTTTGCCGATTTCGGCCTA
TTGGTTAAAAAATGAGCTGATTTAACAAAAATTTAACGCGAATTTTAACAAAATATTAACGCTTACAATTTAGGTGG
CACTTTTCGGGGAAATGTGCGCGGAACCCCTATTTGTTTATTTTTCTAAATACATTCAAATATGTATCCGCTCATGA
GACAATAACCCTGATAAATGCTTCAATAATAGCACCTAGATCAAGAGACAGGATGAGGATCGTTTCGCATGATTGAA
CAAGATGGATTGCACGCAGGTTCTCCGGCCGCTTGGGTGGAGAGGCTATTCGGCTATGACTGGGCACAACAGACAAT
CGGCTGCTCTGATGCCGCCGTGTTCCGGCTGTCAGCGCAGGGGCGCCCGGTTCTTTTTGTCAAGACCGACCTGTCCG
GTGCCCTGAATGAACTGCAAGACGAGGCAGCGCGGCTATCGTGGCTGGCCACGACGGGCGTTCCTTGCGCAGCTGTG
CTCGACGTTGTCACTGAAGCGGGAAGGGACTGGCTGCTATTGGGCGAAGTGCCGGGGCAGGATCTCCTGTCATCTCA
CCTTGCTCCTGCCGAGAAAGTATCCATCATGGCTGATGCAATGCGGCGGCTGCATACGCTTGATCCGGCTACCTGCC
CATTCGACCACCAAGCGAAACATCGCATCGAGCGAGCACGTACTCGGATGGAAGCCGGTCTTGTCGATCAGGATGAT
CTGGACGAAGAGCATCAGGGGCTCGCGCCAGCCGAACTGTTCGCCAGGCTCAAGGCGAGCATGCCCGACGGCGAGGA
TCTCGTCGTGACCCATGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCTGAAGAGCTTGGCGGC
GAATGGGCTGACCGCTTCCTCGTGCTTTACGGTATCGCCGCTCCCGATTCGCAGCGCATCGCCTTCTATCGCCTTCT
TGACGAGTTCTTCTGAATTATTAACGCTTACAATTTCCTGATGCGGTATTTTCTCCTTACGCATCTGTGCGGTATTT
CACACCGCATCAGGTGGCACTTTTCGGGGAAATGTGCGCGGAACCCCTATTTGTTTATTTTTCTAAATACATTCAAA
TATGTATCCGCTCATGACCAAAATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAATAC
TGTTCTTCTAGTGTAGCCGTAGTTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGACTCAAGACGATAGTTACCGGAT
AAGGCGCAGCGGTCGGGCTGAACGGGGGGGTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGAGCGCAGCGAGTCAGTGAGCGAG
GAAGCGGAAGAGCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAATTTCACACAGGAAACAGCTATGAC
CATGATTACGCCAAGCGCGCAATTAACCCTCACTAAAGGGAACAAAAGCTGGAGCTGCAAGCTTGGCCATTGCATAC
GTTGTATCCATATCATAATATGTACATTTATATTGGCTCATGTCCAACATTACCGCCATGTTGACATTGATTATTGA
CTAGTTATTAATAGTAATCAATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAATGACGTATGTTCCCATAGTAAC
GCCAATAGGGACTTTCCATTGACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGATGCGGTTTTGGCAGTA
CATCAATGGGCGTGGATAGCGGTTTGACTCACGGGGATTTCCAAGTCTCCACCCCATTGACGTCAATGGGAGTTTGT
TTTGGCACCAAAATCAACGGGACTTTCCAAAATGTCGTAACAACTCCGCCCCATTGACGCAAATGGGCGGTAGGCGT
GTACGGTGGGAGGTCTATATAAGCAGAGCTCGTTTAGTGAACCGGGGTCTCTCTGGTTAGACCAGATCTGAGCCTGG
GAGCTCTCTGGCTAACTAGGGAACCCACTGCTTAAGCCTCAATAAAGCTTGCCTTGAGTGCTTCAAGTAGTGTGTGC
CCGTCTGTTGTGTGACTCTGGTAACTAGAGATCCCTCAGACCCTTTTAGTCAGTGTGGAAAATCTCTAGCAGTGGCG
CCCGAACAGGGACTTGAAAGCGAAAGGGAAACCAGAGGAGCTCTCTCGACGCAGGACTCGGCTTGCTGAAGCGCGCA
CGGCAAGAGGCGAGGGGCGGCGACTGGTGAGTACGCCAAAAATTTTGACTAGCGGAGGCTAGAAGGAGAGAGATGGG
TGCGAGAGCGTCAGTATTAAGCGGGGGAGAATTAGATCGCGATGGGAAAAAATTCGGTTAAGGCCAGGGGGAAAGAA
AAAATATAAATTAAAACATATAGTATGGGCAAGCAGGGAGCTAGAACGATTCGCAGTTAATCCTGGCCTGTTAGAAA
CATCAGAAGGCTGTAGACAAATACTGGGACAGCTACAACCATCCCTTCAGACAGGATCAGAAGAACTTAGATCATTA
TATAATACAGTAGCAACCCTCTATTGTGTGCATCAAAGGATAGAGATAAAAGACACCAAGGAAGCTTTAGACAAGAT
AGAGGAAGAGCAAAACAAAAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGGAGTAGCACCCACCAAGGC
AAAGAGAAGAGTGGTGCAGAGAGAAAAAAGAGCAGTGGGAATAGGAGCTTTGTTCCTTGGGTTCTTGGGAGCAGCAG
GAAGCACTATGGGCGCAGCGTCAATGACGCTGACGGTACAGGCCAGACAATTATTGTCTGGTATAGTGCAGCAGCAG
AACAATTTGCTGAGGGCTATTGAGGCGCAACAGCATCTGTTGCAACTCACAGTCTGGGGCATCAAGCAGCTCCAGGC
AAGAATCCTGGCTGTGGAAAGATACCTAAAGGATCAACAGCTCCTGGGGATTTGGGGTTGCTCTGGAAAACTCATTT
GCACCACTGCTGTGCCTTGGAATGCTAGTTGGAGTAATAAATCTCTGGAACAGATTTGGAATCACACGACCTGGATG
GAGTGGGACAGAGAAATTAACAATTACACAAGCTTAATACACTCCTTAATTGAAGAATCGCAAAACCAGCAAGAAAA
GAATGAACAAGAATTATTGGAATTAGATAAATGGGCAAGTTTGTGGAATTGGTTTAACATAACAAATTGGCTGTGGT
ATATAAAATTATTCATAATGATAGTAGGAGGCTTGGTAGGTTTAAGAATAGTTTTTGCTGTACTTTCTATAGTGAAT
AGAGTTAGGCAGGGATATTCACCATTATCGTTTCAGACCCACCTCCCAACCCCGAGGGGACCCGACAGGCCCGAAGG
AATAGAAGAAGAAGGTGGAGAGAGAGACAGAGACAGATCCATTCGATTAGTGAACGGATCTCGACGGTATCGGTTAA
CTTTTAAAAGAAAAGGGGGGATTGGGGGGTACAGTGCAGGGGAAAGAATAGTAGACATAATAGCAACAGACATACAA
ACTAAAGAATTACAAAAACAAATTACAAAATTCAAAATTTTATCGATAAGCTTGATATCATCGATTTCCGAATTCCA
CGGGGTTGGGGTTGCGCCTTTTCCAAGGCAGCCCTGGGTTTGCGCAGGGACGCGGCTGCTCTGGGCGTGGTTCCGGG
AAACGCAGCGGCGCCGACCCTGGGTCTCGCACATTCTTCACGTCCGTTCGCAGCGTCACCCGGATCTTCGCCGCTAC
CCTTGTGGGCCCCCCGGCGACGCTTCCTGCTCCGCCCCTAAGTCGGGAAGGTTCCTTGCGGTTCGCGGCGTGCCGGA
CGTGACAAACGGAAGCCGCACGTCTCACTAGTACCCTCGCAGACGGACAGCGCCAGGGAGCAATGGCAGCGCGCCGA
CCGCGATGGGCTGTGGCCAATAGCGGCTGCTCAGCAGGGCGCGCCGAGAGCAGCGGCCGGGAAGGGGCGGTGCGGGA
GGCGGGGTGTGGGGCGGTAGTGTGGGCCCTGTTCCTGCCCGCGCGGTGTTCCGCATTCTGCAAGCCTCCGGAGCGCA
CGTCGGCAGTCGGCTCCCTCGTTGACCGAATCACCGACCTCTCTCCCCAACGCggatccGCCGCCACCATGGGCGCC
TACGCAAGGGCATCCGGCGTGTGCGCAAGGGGCTGTCTGGACTCTGCCGGACCATGGACCATGAGCAGAGCCCTGAG
GCCACCTCTGCCACCACTGTGCTTCTTTCTGCTGCTGCTGGCAGCAGCAGGAGCAAGGGCAGGAGGCTATGAGACAT
GTCCTACCGTGCAGCCAAACATGCTGAATGTGCACCTGCTGCCTCACACCCACGACGATGTGGGCTGGCTGAAGACA
GTGGACCAGTACTTTTATGGCATCAAGAATGATATCCAGCACGCCGGCGTGCAGTACATCCTGGACTCTGTGATCAG
CGCCCTGCTGGCCGATCCAACCCGGAGATTCATCTATGTGGAGATCGCCTTCTTTAGCAGATGGTGGCACCAGCAGA
CAAACGCCACTCAGGAGGTGGTGAGGGATCTGGTGCGCCAGGGCCGGCTGGAGTTTGCAAACGGAGGATGGGTCATG
AATGATGAGGCCGCCACACACTACGGCGCCATCGTGGACCAGATGACCCTGGGCCTGCGCTTTCTGGAGGACACATT
CGGCAACGATGGCAGACCCAGGGTGGCATGGCACATCGATCCTTTCGGCCACTCTCGGGAGCAGGCCAGCCTGTTTG
CCCAGATGGGCTTCGACGGCTTCTTTTTCGGCCGCCTGGACTACCAGGATAAGTGGGTGCGGATGCAGAAGCTGGAG
ATGGAGCAAGTGTGGAGGGCCTCTACCAGCCTGAAGCCTCCAACCGCCGACCTGTTTACAGGCGTGCTGCCTAACGG
CTATAATCCCCCTCGCAATCTGTGCTGGGACGTGCTGTGCGTGGACCAGCCACTGGTGGAGGACCCCCGGAGCCCAG
AGTACAACGCCAAGGAGCTGGTGGACTATTTCCTGAATGTGGCCACCGCCCAGGGCAGATACTATAGGACAAACCAC
ACCGTGATGACAATGGGCTCCGATTTTCAGTACGAGAACGCCAATATGTGGTTCAAGAACCTGGACAAGCTGATCCG
CCTGGTGAATGCACAGCAGGCAAAGGGCAGCTCCGTGCACGTGCTGTACAGCACCCCAGCCTGCTATCTGTGGGAGC
TGAACAAGGCCAATCTGACATGGTCCGTGAAGCACGACGACTTCTTCCCATACGCCGATGGCCCCCACCAGTTTTGG
ACCGGCTATTTCTCTAGCCGGCCCGCCCTGAAGCGGTACGAGCGGCTGTCCTATAACTTCCTGCAGGTGTGCAATCA
GCTGGAGGCCCTGGTGGGACTGGCAGCAAACGTGGGACCATACGGCTCCGGCGACTCTGCCCCCCTGAATGAGGCCA
TGGCCGTGCTGCAGCACCACGATGCCGTGAGCGGCACCTCCAGACAGCACGTGGCCAACGACTATGCAAGGCAGCTG
GCAGCAGGATGGGGACCATGCGAGGTGCTGCTGTCTAATGCCCTGGCCAGACTGAGGGGCTTTAAGGATCACTTTAC
CTTCTGCCAGCAGCTGAACATCTCCATCTGTCCTCTGTCTCAGACAGCCGCCAGATTCCAGGTCATCGTGTACAACC
CTCTGGGCAGAAAAGTGAATTGGATGGTGAGGCTGCCAGTGAGCGAGGGCGTGTTTGTGGTGAAGGACCCCAATGGC
AGGACCGTGCCTTCTGACGTGGTCATCTTCCCATCCTCTGATAGCCAGGCCCACCCACCCGAGCTGCTGTTTTCCGC
CTCTCTGCCCGCCCTGGGCTTCTCTACCTATAGCGTGGCCCAGGTGCCAAGGTGGAAGCCTCAGGCAAGGGCACCAC
AGCCAATCCCTAGGCGCAGCTGGTCCCCCGCCCTGACAATCGAGAACGAGCACATCAGAGCCACCTTCGACCCTGAT
ACAGGCCTGCTGATGGAGATCATGAACATGAATCAGCAGCTGCTGCTGCCCGTGAGGCAGACCTTTTTCTGGTACAA
CGCCTCCATCGGCGACAATGAGTCCGATCAGGCCTCTGGCGCCTATATCTTTAGACCCAATCAGCAGAAGCCACTGC
CCGTGTCTAGATGGGCCCAGATCCACCTGGTGAAGACACCTCTGGTGCAGGAGGTGCACCAGAACTTCAGCGCCTGG
TGTTCCCAGGTGGTGAGACTGTACCCTGGCCAGAGGCACCTGGAGCTGGAGTGGAGCGTGGGACCTATCCCAGTGGG
CGATACCTGGGGCAAGGAAGTGATCTCCAGATTTGACACCCCTCTGGAGACAAAGGGCCGCTTCTACACAGATTCTA
ACGGCCGGGAGATCCTGGAGCGGAGAAGGGACTATAGACCAACCTGGAAGCTGAATCAGACAGAGCCTGTGGCCGGC
AACTACTATCCAGTGAATACCAGGATCTATATCACAGACGGCAATATGCAGCTGACCGTGCTGACAGATAGATCCCA
GGGAGGCAGCTCCCTGAGGGACGGCTCTCTGGAGCTGATGGTGCACCGCCGGCTGCTGAAGGACGATGGAAGGGGCG
TGTCCGAGCCACTGATGGAGAACGGCTCTGGAGCATGGGTGCGCGGCCGGCACCTGGTGCTGCTGGATACCGCACAG
GCAGCAGCAGCAGGACACAGGCTGCTGGCCGAGCAGGAGGTGCTGGCCCCACAGGTGGTGCTGGCACCAGGAGGAGG
AGCAGCCTACAATCTGGGAGCACCTCCAAGGACCCAGTTTTCCGGACTGAGAAGGGACCTGCCACCTAGCGTGCACC
TGCTGACACTGGCATCCTGGGGACCAGAGATGGTGCTGCTGAGGCTGGAGCACCAGTTCGCAGTGGGAGAGGATAGC
GGAAGGAACCTGTCCGCCCCAGTGACCCTGAATCTGAGGGACCTGTTTTCTACCTTCACAATCACCCGGCTGCAGGA
GACCACACTGGTGGCCAACCAGCTGAGAGAGGCCGCCAGCAGGCTGAAGTGGACCACAAATACCGGCCCCACACCTC
ACCAGACACCTTACCAGCTGGACCCCGCCAACATCACCCTGGAGCCCATGGAGATCAGAACATTCCTGGCCAGCGTG
CAGTGGAAGGAGGTGGACGGCTGAGTCTGCTGGgtcgacAATCAACCTCTGGATTACAAAATTTGTGAAAGATTGAC
TGGTATTCTTAACTATGTTGCTCCTTTTACGCTATGTGGATACGCTGCTTTAATGCCTTTGTATCATGCTATTGCTT
CCCGTATGGCTTTCATTTTCTCCTCCTTGTATAAATCCTGGTTGCTGTCTCTTTATGAGGAGTTGTGGCCCGTTGTC
AGGCAACGTGGCGTGGTGTGCACTGTGTTTGCTGACGCAACCCCCACTGGTTGGGGCATTGCCACCACCTGTCAGCT
CCTTTCCGGGACTTTCGCTTTCCCCCTCCCTATTGCCACGGCGGAACTCATCGCCGCCTGCCTTGCCCGCTGCTGGA
CAGGGGCTCGGCTGTTGGGCACTGACAATTCCGTGGTGTTGTCGGGGAAATCATCGTCCTTTCCTTGGCTGCTCGCC
TGTGTTGCCACCTGGATTCTGCGCGGGACGTCCTTCTGCTACGTCCCTTCGGCCCTCAATCCAGCGGACCTTCCTTC
CCGCGGCCTGCTGCCGGCTCTGCGGCCTCTTCCGCGTCTTCGCCTTCGCCCTCAGACGAGTCGGATCTCCCTTTGGG
CCGCCTCCCCGCCTGGAATTCGAGCTCGGTACCTTTAAGACCAATGACTTACAAGGCAGCTGTAGATCTTAGCCACT
TTTTAAAAGAAAAGGGGGGACTGGAAGGGCTAATTCACTCCCAACGAAGACAAGATCTGCTTTTTGCTTGTACTGGG
TCTCTCTGGTTAGACCAGATCTGAGCCTGGGAGCTCTCTGGCTAACTAGGGAACCCACTGCTTAAGCCTCAATAAAG
CTTGCCTTGAGTGCTTCAAGTAGTGTGTGCCCGTCTGTTGTGTGACTCTGGTAACTAGAGATCCCTCAGACCCTTTT
AGTCAGTGTGGAAAATCTCTAGCAGTAGTAGTTCATGTCATCTTATTATTCAGTATTTATAACTTGCAAAGAAATGA
ATATCAGAGAGTGAGAGGAACTTGTTTATTGCAGCTTATAATGGTTACAAATAAAGCAATAGCATCACAAATTTCAC
AAATAAAGCATTTTTTTCACTGCATTCTAGTTGTGGTTTGTCCAAACTCATCAATGTATCTTATCATGTCTGGCTCT
AGCTATCCCGCCCCTAACTCCGCCCATCCCGCCCCTAACTCCGCCCAGTTCCGCCCATTCTCCGCCCCATGGCTGAC
TAATTTTTTTTATTTATGCAGAGGCCGAGGCCGCCTCGGCCTCTGAGCTATTCCAGAAGTAGTGAGGAGGCTTTTTT
GGAGGCCTAGGGACGTACCCAATTCGCCCTATAGTGAGTCGTATTACGCGCGCTCACTGGCCGTCGTTTTACAACGT
CGTGACTGGGAAAACCCTGGCGTTACCCAACTTAATCGCCTTGCAGCACATCCCCCTTTCGCCAGCTGGCGTAATAG
CGAAGAGGCCCGCACCGATCGCCCTTCCCAACAGTTGCGCAGCCTGAATGGCGAATGGG
(LV-EIF4A hGLB1 WT transfer vector)
SEQ ID NO: 33
ACGCGCCCTGTAGCGGCGCATTAAGCGCGGCGGGTGTGGTGGTTACGCGCAGCGTGACCGCTACACTTGCCAGCGCC
CTAGCGCCCGCTCCTTTCGCTTTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAACTTGATTAGGGTGATGGTTCAC
GTAGTGGGCCATCGCCCTGATAGACGGTTTTTCGCCCTTTGACGTTGGAGTCCACGTTCTTTAATAGTGGACTCTTG
TTCCAAACTGGAACAACACTCAACCCTATCTCGGTCTATTCTTTTGATTTATAAGGGATTTTGCCGATTTCGGCCTA
TTGGTTAAAAAATGAGCTGATTTAACAAAAATTTAACGCGAATTTTAACAAAATATTAACGCTTACAATTTAGGTGG
CACTTTTCGGGGAAATGTGCGCGGAACCCCTATTTGTTTATTTTTCTAAATACATTCAAATATGTATCCGCTCATGA
GACAATAACCCTGATAAATGCTTCAATAATAGCACCTAGATCAAGAGACAGGATGAGGATCGTTTCGCATGATTGAA
CAAGATGGATTGCACGCAGGTTCTCCGGCCGCTTGGGTGGAGAGGCTATTCGGCTATGACTGGGCACAACAGACAAT
CGGCTGCTCTGATGCCGCCGTGTTCCGGCTGTCAGCGCAGGGGCGCCCGGTTCTTTTTGTCAAGACCGACCTGTCCG
GTGCCCTGAATGAACTGCAAGACGAGGCAGCGCGGCTATCGTGGCTGGCCACGACGGGCGTTCCTTGCGCAGCTGTG
CTCGACGTTGTCACTGAAGCGGGAAGGGACTGGCTGCTATTGGGCGAAGTGCCGGGGCAGGATCTCCTGTCATCTCA
CCTTGCTCCTGCCGAGAAAGTATCCATCATGGCTGATGCAATGCGGCGGCTGCATACGCTTGATCCGGCTACCTGCC
CATTCGACCACCAAGCGAAACATCGCATCGAGCGAGCACGTACTCGGATGGAAGCCGGTCTTGTCGATCAGGATGAT
CTGGACGAAGAGCATCAGGGGCTCGCGCCAGCCGAACTGTTCGCCAGGCTCAAGGCGAGCATGCCCGACGGCGAGGA
TCTCGTCGTGACCCATGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCTGAAGAGCTTGGCGGC
GAATGGGCTGACCGCTTCCTCGTGCTTTACGGTATCGCCGCTCCCGATTCGCAGCGCATCGCCTTCTATCGCCTTCT
TGACGAGTTCTTCTGAATTATTAACGCTTACAATTTCCTGATGCGGTATTTTCTCCTTACGCATCTGTGCGGTATTT
CACACCGCATCAGGTGGCACTTTTCGGGGAAATGTGCGCGGAACCCCTATTTGTTTATTTTTCTAAATACATTCAAA
TATGTATCCGCTCATGACCAAAATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAATAC
TGTTCTTCTAGTGTAGCCGTAGTTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGACTCAAGACGATAGTTACCGGAT
AAGGCGCAGCGGTCGGGCTGAACGGGGGGGTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGAGCGCAGCGAGTCAGTGAGCGAG
GAAGCGGAAGAGCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAATTTCACACAGGAAACAGCTATGAC
CATGATTACGCCAAGCGCGCAATTAACCCTCACTAAAGGGAACAAAAGCTGGAGCTGCAAGCTTGGCCATTGCATAC
GTTGTATCCATATCATAATATGTACATTTATATTGGCTCATGTCCAACATTACCGCCATGTTGACATTGATTATTGA
CTAGTTATTAATAGTAATCAATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAATGACGTATGTTCCCATAGTAAC
GCCAATAGGGACTTTCCATTGACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGATGCGGTTTTGGCAGTA
CATCAATGGGCGTGGATAGCGGTTTGACTCACGGGGATTTCCAAGTCTCCACCCCATTGACGTCAATGGGAGTTTGT
TTTGGCACCAAAATCAACGGGACTTTCCAAAATGTCGTAACAACTCCGCCCCATTGACGCAAATGGGCGGTAGGCGT
GTACGGTGGGAGGTCTATATAAGCAGAGCTCGTTTAGTGAACCGGGGTCTCTCTGGTTAGACCAGATCTGAGCCTGG
GAGCTCTCTGGCTAACTAGGGAACCCACTGCTTAAGCCTCAATAAAGCTTGCCTTGAGTGCTTCAAGTAGTGTGTGC
CCGTCTGTTGTGTGACTCTGGTAACTAGAGATCCCTCAGACCCTTTTAGTCAGTGTGGAAAATCTCTAGCAGTGGCG
CCCGAACAGGGACTTGAAAGCGAAAGGGAAACCAGAGGAGCTCTCTCGACGCAGGACTCGGCTTGCTGAAGCGCGCA
CGGCAAGAGGCGAGGGGCGGCGACTGGTGAGTACGCCAAAAATTTTGACTAGCGGAGGCTAGAAGGAGAGAGATGGG
TGCGAGAGCGTCAGTATTAAGCGGGGGAGAATTAGATCGCGATGGGAAAAAATTCGGTTAAGGCCAGGGGGAAAGAA
AAAATATAAATTAAAACATATAGTATGGGCAAGCAGGGAGCTAGAACGATTCGCAGTTAATCCTGGCCTGTTAGAAA
CATCAGAAGGCTGTAGACAAATACTGGGACAGCTACAACCATCCCTTCAGACAGGATCAGAAGAACTTAGATCATTA
TATAATACAGTAGCAACCCTCTATTGTGTGCATCAAAGGATAGAGATAAAAGACACCAAGGAAGCTTTAGACAAGAT
AGAGGAAGAGCAAAACAAAAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGGAGTAGCACCCACCAAGGC
AAAGAGAAGAGTGGTGCAGAGAGAAAAAAGAGCAGTGGGAATAGGAGCTTTGTTCCTTGGGTTCTTGGGAGCAGCAG
GAAGCACTATGGGCGCAGCGTCAATGACGCTGACGGTACAGGCCAGACAATTATTGTCTGGTATAGTGCAGCAGCAG
AACAATTTGCTGAGGGCTATTGAGGCGCAACAGCATCTGTTGCAACTCACAGTCTGGGGCATCAAGCAGCTCCAGGC
AAGAATCCTGGCTGTGGAAAGATACCTAAAGGATCAACAGCTCCTGGGGATTTGGGGTTGCTCTGGAAAACTCATTT
GCACCACTGCTGTGCCTTGGAATGCTAGTTGGAGTAATAAATCTCTGGAACAGATTTGGAATCACACGACCTGGATG
GAGTGGGACAGAGAAATTAACAATTACACAAGCTTAATACACTCCTTAATTGAAGAATCGCAAAACCAGCAAGAAAA
GAATGAACAAGAATTATTGGAATTAGATAAATGGGCAAGTTTGTGGAATTGGTTTAACATAACAAATTGGCTGTGGT
ATATAAAATTATTCATAATGATAGTAGGAGGCTTGGTAGGTTTAAGAATAGTTTTTGCTGTACTTTCTATAGTGAAT
AGAGTTAGGCAGGGATATTCACCATTATCGTTTCAGACCCACCTCCCAACCCCGAGGGGACCCGACAGGCCCGAAGG
AATAGAAGAAGAAGGTGGAGAGAGAGACAGAGACAGATCCATTCGATTAGTGAACGGATCTCGACGGTATCGGTTAA
CTTTTAAAAGAAAAGGGGGGATTGGGGGGTACAGTGCAGGGGAAAGAATAGTAGACATAATAGCAACAGACATACAA
ACTAAAGAATTACAAAAACAAATTACAAAATTCAAAATTTTATCGATAAGCTTGATATCATCGATTTCCGAATTCCA
CGGGGTTGGGGTTGCGCCTTTTCCAAGGCAGCCCTGGGTTTGCGCAGGGACGCGGCTGCTCTGGGCGTGGTTCCGGG
AAACGCAGCGGCGCCGACCCTGGGTCTCGCACATTCTTCACGTCCGTTCGCAGCGTCACCCGGATCTTCGCCGCTAC
CCTTGTGGGCCCCCCGGCGACGCTTCCTGCTCCGCCCCTAAGTCGGGAAGGTTCCTTGCGGTTCGCGGCGTGCCGGA
CGTGACAAACGGAAGCCGCACGTCTCACTAGTACCCTCGCAGACGGACAGCGCCAGGGAGCAATGGCAGCGCGCCGA
CCGCGATGGGCTGTGGCCAATAGCGGCTGCTCAGCAGGGCGCGCCGAGAGCAGCGGCCGGGAAGGGGCGGTGCGGGA
GGCGGGGTGTGGGGCGGTAGTGTGGGCCCTGTTCCTGCCCGCGCGGTGTTCCGCATTCTGCAAGCCTCCGGAGCGCA
CGTCGGCAGTCGGCTCCCTCGTTGACCGAATCACCGACCTCTCTCCCCAACGCggatctGACTCACTATTTGTTTTC
GCGCCCAGTTGCAAAAAGTGTCGCCGCCACCATGCCGGGGTTCCTGGTTCGCATCCTCCCTCTGTTGCTGGTTCTGC
TGCTTCTGGGCCCTACGCGCGGCTTGCGCAATGCCACCCAGAGGATGTTTGAAATTGACTATAGCCGGGACTCCTTC
CTCAAGGATGGCCAGCCATTTCGCTACATCTCAGGAAGCATTCACTACTCCCGTGTGCCCCGCTTCTACTGGAAGGA
CCGGCTGCTGAAGATGAAGATGGCTGGGCTGAACGCCATCCAGACGTATGTGCCCTGGAACTTTCATGAGCCCTGGC
CAGGACAGTACCAGTTTTCTGAGGACCATGATGTGGAATATTTTCTTCGGCTGGCTCATGAGCTGGGACTGCTGGTT
ATCCTGAGGCCCGGGCCCTACATCTGTGCAGAGTGGGAAATGGGAGGATTACCTGCTTGGCTGCTAGAGAAAGAGTC
TATTCTTCTCCGCTCCTCCGACCCAGATTACCTGGCAGCTGTGGACAAGTGGTTGGGAGTCCTTCTGCCCAAGATGA
AGCCTCTCCTCTATCAGAATGGAGGGCCAGTTATAACAGTGCAGGTTGAAAATGAATATGGCAGCTACTTTGCCTGT
GATTTTGACTACCTGCGCTTCCTGCAGAAGCGCTTTCGCCACCATCTGGGGGATGATGTGGTTCTGTTTACCACTGA
TGGAGCACATAAAACATTCCTGAAATGTGGGGCCCTGCAGGGCCTCTACACCACGGTGGACTTTGGAACAGGCAGCA
ACATCACAGATGCTTTCCTAAGCCAGAGGAAGTGTGAGCCCAAAGGACCCTTGATCAATTCTGAATTCTATACTGGC
TGGCTAGATCACTGGGGCCAACCTCACTCCACAATCAAGACCGAAGCAGTGGCTTCCTCCCTCTATGATATACTTGC
CCGTGGGGCGAGTGTGAACTTGTACATGTTTATAGGTGGGACCAATTTTGCCTATTGGAATGGGGCCAACTCACCCT
ATGCAGCACAGCCCACCAGCTACGACTATGATGCCCCACTGAGTGAGGCTGGGGACCTCACTGAGAAGTATTTTGCT
CTGCGAAACATCATCCAGAAGTTTGAAAAAGTACCAGAAGGTCCTATCCCTCCATCTACACCAAAGTTTGCATATGG
AAAGGTCACTTTGGAAAAGTTAAAGACAGTGGGAGCAGCTCTGGACATTCTGTGTCCCTCTGGGCCCATCAAAAGCC
TTTATCCCTTGACATTTATCCAGGTGAAACAGCATTATGGGTTTGTGCTGTACCGGACAACACTTCCTCAAGATTGC
AGCAACCCAGCACCTCTCTCTTCACCCCTCAATGGAGTCCACGATCGAGCATATGTTGCTGTGGATGGGATCCCCCA
GGGAGTCCTTGAGCGAAACAATGTGATCACTCTGAACATAACAGGGAAAGCTGGAGCCACTCTGGACCTTCTGGTAG
AGAACATGGGACGTGTGAACTATGGTGCATATATCAACGATTTTAAGGGTTTGGTTTCTAACCTGACTCTCAGTTCC
AATATCCTCACGGACTGGACGATCTTTCCACTGGACACTGAGGATGCAGTGCGCAGCCACCTGGGGGGCTGGGGACA
CCGTGACAGTGGCCACCATGATGAAGCCTGGGCCCACAACTCATCCAACTACACGCTCCCGGCCTTTTATATGGGGA
ACTTCTCCATTCCCAGTGGGATCCCAGACTTGCCCCAGGACACCTTTATCCAGTTTCCTGGATGGACCAAGGGCCAG
GTCTGGATTAATGGCTTTAACCTTGGCCGCTATTGGCCAGCCCGGGGCCCTCAGTTGACCTTGTTTGTGCCCCAGCA
CATCCTGATGACCTCGGCCCCAAACACCATCACCGTGCTGGAACTGGAGTGGGCACCCTGCAGCAGTGATGATCCAG
AACTATGTGCTGTGACGTTCGTGGACAGGCCAGTTATTGGCTCATCTGTGACCTACGATCATCCCTCCAAACCTGTT
GAAAAAAGACTCATGCCCCCACCCCCGCAAAAAAACAAAGATTCATGGCTGGACCATGTATGATGATGAAAGgtcga
CAATCAACCTCTGGATTACAAAATTTGTGAAAGATTGACTGGTATTCTTAACTATGTTGCTCCTTTTACGCTATGTG
GATACGCTGCTTTAATGCCTTTGTATCATGCTATTGCTTCCCGTATGGCTTTCATTTTCTCCTCCTTGTATAAATCC
TGGTTGCTGTCTCTTTATGAGGAGTTGTGGCCCGTTGTCAGGCAACGTGGCGTGGTGTGCACTGTGTTTGCTGACGC
AACCCCCACTGGTTGGGGCATTGCCACCACCTGTCAGCTCCTTTCCGGGACTTTCGCTTTCCCCCTCCCTATTGCCA
CGGCGGAACTCATCGCCGCCTGCCTTGCCCGCTGCTGGACAGGGGCTCGGCTGTTGGGCACTGACAATTCCGTGGTG
TTGTCGGGGAAATCATCGTCCTTTCCTTGGCTGCTCGCCTGTGTTGCCACCTGGATTCTGCGCGGGACGTCCTTCTG
CTACGTCCCTTCGGCCCTCAATCCAGCGGACCTTCCTTCCCGCGGCCTGCTGCCGGCTCTGCGGCCTCTTCCGCGTC
TTCGCCTTCGCCCTCAGACGAGTCGGATCTCCCTTTGGGCCGCCTCCCCGCCTGGAATTCGAGCTCGGTACCTTTAA
GACCAATGACTTACAAGGCAGCTGTAGATCTTAGCCACTTTTTAAAAGAAAAGGGGGGACTGGAAGGGCTAATTCAC
TCCCAACGAAGACAAGATCTGCTTTTTGCTTGTACTGGGTCTCTCTGGTTAGACCAGATCTGAGCCTGGGAGCTCTC
TGGCTAACTAGGGAACCCACTGCTTAAGCCTCAATAAAGCTTGCCTTGAGTGCTTCAAGTAGTGTGTGCCCGTCTGT
TGTGTGACTCTGGTAACTAGAGATCCCTCAGACCCTTTTAGTCAGTGTGGAAAATCTCTAGCAGTAGTAGTTCATGT
CATCTTATTATTCAGTATTTATAACTTGCAAAGAAATGAATATCAGAGAGTGAGAGGAACTTGTTTATTGCAGCTTA
TAATGGTTACAAATAAAGCAATAGCATCACAAATTTCACAAATAAAGCATTTTTTTCACTGCATTCTAGTTGTGGTT
TGTCCAAACTCATCAATGTATCTTATCATGTCTGGCTCTAGCTATCCCGCCCCTAACTCCGCCCATCCCGCCCCTAA
CTCCGCCCAGTTCCGCCCATTCTCCGCCCCATGGCTGACTAATTTTTTTTATTTATGCAGAGGCCGAGGCCGCCTCG
GCCTCTGAGCTATTCCAGAAGTAGTGAGGAGGCTTTTTTGGAGGCCTAGGGACGTACCCAATTCGCCCTATAGTGAG
TCGTATTACGCGCGCTCACTGGCCGTCGTTTTACAACGTCGTGACTGGGAAAACCCTGGCGTTACCCAACTTAATCG
CCTTGCAGCACATCCCCCTTTCGCCAGCTGGCGTAATAGCGAAGAGGCCCGCACCGATCGCCCTTCCCAACAGTTGC
GCAGCCTGAATGGCGAATGGG
(LV mGLB1 WT transfer vector)
Seq ID NO: 34
ACGCGCCCTGTAGCGGCGCATTAAGCGCGGCGGGTGTGGTGGTTACGCGCAGCGTGACCGCTACACTTGCCAGCGCC
CTAGCGCCCGCTCCTTTCGCTTTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAACTTGATTAGGGTGATGGTTCAC
GTAGTGGGCCATCGCCCTGATAGACGGTTTTTCGCCCTTTGACGTTGGAGTCCACGTTCTTTAATAGTGGACTCTTG
TTCCAAACTGGAACAACACTCAACCCTATCTCGGTCTATTCTTTTGATTTATAAGGGATTTTGCCGATTTCGGCCTA
TTGGTTAAAAAATGAGCTGATTTAACAAAAATTTAACGCGAATTTTAACAAAATATTAACGCTTACAATTTAGGTGG
CACTTTTCGGGGAAATGTGCGCGGAACCCCTATTTGTTTATTTTTCTAAATACATTCAAATATGTATCCGCTCATGA
GACAATAACCCTGATAAATGCTTCAATAATAGCACCTAGATCAAGAGACAGGATGAGGATCGTTTCGCATGATTGAA
CAAGATGGATTGCACGCAGGTTCTCCGGCCGCTTGGGTGGAGAGGCTATTCGGCTATGACTGGGCACAACAGACAAT
CGGCTGCTCTGATGCCGCCGTGTTCCGGCTGTCAGCGCAGGGGCGCCCGGTTCTTTTTGTCAAGACCGACCTGTCCG
GTGCCCTGAATGAACTGCAAGACGAGGCAGCGCGGCTATCGTGGCTGGCCACGACGGGCGTTCCTTGCGCAGCTGTG
CTCGACGTTGTCACTGAAGCGGGAAGGGACTGGCTGCTATTGGGCGAAGTGCCGGGGCAGGATCTCCTGTCATCTCA
CCTTGCTCCTGCCGAGAAAGTATCCATCATGGCTGATGCAATGCGGCGGCTGCATACGCTTGATCCGGCTACCTGCC
CATTCGACCACCAAGCGAAACATCGCATCGAGCGAGCACGTACTCGGATGGAAGCCGGTCTTGTCGATCAGGATGAT
CTGGACGAAGAGCATCAGGGGCTCGCGCCAGCCGAACTGTTCGCCAGGCTCAAGGCGAGCATGCCCGACGGCGAGGA
TCTCGTCGTGACCCATGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCTGAAGAGCTTGGCGGC
GAATGGGCTGACCGCTTCCTCGTGCTTTACGGTATCGCCGCTCCCGATTCGCAGCGCATCGCCTTCTATCGCCTTCT
TGACGAGTTCTTCTGAATTATTAACGCTTACAATTTCCTGATGCGGTATTTTCTCCTTACGCATCTGTGCGGTATTT
CACACCGCATCAGGTGGCACTTTTCGGGGAAATGTGCGCGGAACCCCTATTTGTTTATTTTTCTAAATACATTCAAA
TATGTATCCGCTCATGACCAAAATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAATAC
TGTTCTTCTAGTGTAGCCGTAGTTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGACTCAAGACGATAGTTACCGGAT
AAGGCGCAGCGGTCGGGCTGAACGGGGGGGTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGAGCGCAGCGAGTCAGTGAGCGAG
GAAGCGGAAGAGCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAATTTCACACAGGAAACAGCTATGAC
CATGATTACGCCAAGCGCGCAATTAACCCTCACTAAAGGGAACAAAAGCTGGAGCTGCAAGCTTGGCCATTGCATAC
GTTGTATCCATATCATAATATGTACATTTATATTGGCTCATGTCCAACATTACCGCCATGTTGACATTGATTATTGA
CTAGTTATTAATAGTAATCAATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAATGACGTATGTTCCCATAGTAAC
GCCAATAGGGACTTTCCATTGACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGATGCGGTTTTGGCAGTA
CATCAATGGGCGTGGATAGCGGTTTGACTCACGGGGATTTCCAAGTCTCCACCCCATTGACGTCAATGGGAGTTTGT
TTTGGCACCAAAATCAACGGGACTTTCCAAAATGTCGTAACAACTCCGCCCCATTGACGCAAATGGGCGGTAGGCGT
GTACGGTGGGAGGTCTATATAAGCAGAGCTCGTTTAGTGAACCGGGGTCTCTCTGGTTAGACCAGATCTGAGCCTGG
GAGCTCTCTGGCTAACTAGGGAACCCACTGCTTAAGCCTCAATAAAGCTTGCCTTGAGTGCTTCAAGTAGTGTGTGC
CCGTCTGTTGTGTGACTCTGGTAACTAGAGATCCCTCAGACCCTTTTAGTCAGTGTGGAAAATCTCTAGCAGTGGCG
CCCGAACAGGGACTTGAAAGCGAAAGGGAAACCAGAGGAGCTCTCTCGACGCAGGACTCGGCTTGCTGAAGCGCGCA
CGGCAAGAGGCGAGGGGCGGCGACTGGTGAGTACGCCAAAAATTTTGACTAGCGGAGGCTAGAAGGAGAGAGATGGG
TGCGAGAGCGTCAGTATTAAGCGGGGGAGAATTAGATCGCGATGGGAAAAAATTCGGTTAAGGCCAGGGGGAAAGAA
AAAATATAAATTAAAACATATAGTATGGGCAAGCAGGGAGCTAGAACGATTCGCAGTTAATCCTGGCCTGTTAGAAA
CATCAGAAGGCTGTAGACAAATACTGGGACAGCTACAACCATCCCTTCAGACAGGATCAGAAGAACTTAGATCATTA
TATAATACAGTAGCAACCCTCTATTGTGTGCATCAAAGGATAGAGATAAAAGACACCAAGGAAGCTTTAGACAAGAT
AGAGGAAGAGCAAAACAAAAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGGAGTAGCACCCACCAAGGC
AAAGAGAAGAGTGGTGCAGAGAGAAAAAAGAGCAGTGGGAATAGGAGCTTTGTTCCTTGGGTTCTTGGGAGCAGCAG
GAAGCACTATGGGCGCAGCGTCAATGACGCTGACGGTACAGGCCAGACAATTATTGTCTGGTATAGTGCAGCAGCAG
AACAATTTGCTGAGGGCTATTGAGGCGCAACAGCATCTGTTGCAACTCACAGTCTGGGGCATCAAGCAGCTCCAGGC
AAGAATCCTGGCTGTGGAAAGATACCTAAAGGATCAACAGCTCCTGGGGATTTGGGGTTGCTCTGGAAAACTCATTT
GCACCACTGCTGTGCCTTGGAATGCTAGTTGGAGTAATAAATCTCTGGAACAGATTTGGAATCACACGACCTGGATG
GAGTGGGACAGAGAAATTAACAATTACACAAGCTTAATACACTCCTTAATTGAAGAATCGCAAAACCAGCAAGAAAA
GAATGAACAAGAATTATTGGAATTAGATAAATGGGCAAGTTTGTGGAATTGGTTTAACATAACAAATTGGCTGTGGT
ATATAAAATTATTCATAATGATAGTAGGAGGCTTGGTAGGTTTAAGAATAGTTTTTGCTGTACTTTCTATAGTGAAT
AGAGTTAGGCAGGGATATTCACCATTATCGTTTCAGACCCACCTCCCAACCCCGAGGGGACCCGACAGGCCCGAAGG
AATAGAAGAAGAAGGTGGAGAGAGAGACAGAGACAGATCCATTCGATTAGTGAACGGATCTCGACGGTATCGGTTAA
CTTTTAAAAGAAAAGGGGGGATTGGGGGGTACAGTGCAGGGGAAAGAATAGTAGACATAATAGCAACAGACATACAA
ACTAAAGAATTACAAAAACAAATTACAAAATTCAAAATTTTATCGATAAGCTTGATATCATCGATTTCCGAATTCCA
CGGGGTTGGGGTTGCGCCTTTTCCAAGGCAGCCCTGGGTTTGCGCAGGGACGCGGCTGCTCTGGGCGTGGTTCCGGG
AAACGCAGCGGCGCCGACCCTGGGTCTCGCACATTCTTCACGTCCGTTCGCAGCGTCACCCGGATCTTCGCCGCTAC
CCTTGTGGGCCCCCCGGCGACGCTTCCTGCTCCGCCCCTAAGTCGGGAAGGTTCCTTGCGGTTCGCGGCGTGCCGGA
CGTGACAAACGGAAGCCGCACGTCTCACTAGTACCCTCGCAGACGGACAGCGCCAGGGAGCAATGGCAGCGCGCCGA
CCGCGATGGGCTGTGGCCAATAGCGGCTGCTCAGCAGGGCGCGCCGAGAGCAGCGGCCGGGAAGGGGCGGTGCGGGA
GGCGGGGTGTGGGGCGGTAGTGTGGGCCCTGTTCCTGCCCGCGCGGTGTTCCGCATTCTGCAAGCCTCCGGAGCGCA
CGTCGGCAGTCGGCTCCCTCGTTGACCGAATCACCGACCTCTCTCCCCAACGCggatccAGAGCGCCCACTGCCTAA
CGGAGAGACCCCATCGTGGCGCGATCATGCTCCGGGTCCCCCTGTGTACGCCGCTCCCGCTCCTGGCACTGCTGCAA
CTGCTGGGCGCTGCGCACGGCATCTATAATGTCACCCAGAGGACATTTAAGCTCGACTACAGCCGGGACCGCTTCCT
CAAGGATGGACAGCCATTCCGATACATCTCGGGAAGCATTCATTACTTCCGGATACCCCGCTTCTACTGGGAGGACC
GGCTGCTGAAGATGAAGATGGCTGGGCTGAATGCTATCCAGATGTACGTGCCCTGGAACTTCCATGAACCCCAACCA
GGACAATATGAGTTTTCTGGGGACCGTGATGTGGAGCATTTCATCCAGCTGGCTCATGAGCTGGGACTCCTGGTGAT
CCTGAGGCCTGGGCCCTACATCTGTGCAGAGTGGGACATGGGGGGCTTACCTGCTTGGCTACTAGAGAAACAATCTA
TCGTTCTCCGGTCTTCTGACCCAGACTACCTTGTAGCTGTGGATAAATGGCTGGCAGTCCTTCTGCCCAAGATGAAG
CCCCTGCTCTACCAGAACGGAGGACCGATCATAACCGTGCAGGTTGAGAATGAGTACGGGTCCTACTTTGCCTGCGA
TTACGACTACCTACGCTTCCTGGTGCACCGCTTCCGCTACCATCTGGGTAATGACGTCATTCTCTTCACCACCGACG
GAGCAAGTGAAAAAATGCTGAAGTGTGGGACCCTGCAGGACCTGTACGCCACAGTGGATTTTGGAACAGGCAACAAT
ATCACACAAGCTTTCCTGGTCCAGAGGAAGTTTGAACCTAAAGGACCTTTGATCAATTCTGAGTTCTATACTGGCTG
GCTAGACCACTGGGGTAAACCCCATTCCACGGTGAAAACTAAAACACTGGCTACCTCCCTCTATAACCTGCTTGCCC
GTGGGGCCAACGTGAACTTGTACATGTTTATAGGTGGGACCAATTTTGCCTATTGGAATGGTGCCAACACGCCCTAT
GAGCCACAGCCCACCAGCTATGACTACGACGCCCCACTGAGCGAGGCTGGGGACCTCACTAAGAAGTATTTTGCTCT
TCGAGAAGTCATTCAGATGTTTAAAGAAGTCCCAGAAGGCCCTATCCCTCCGTCTACACCCAAATTTGCATATGGAA
AAGTTGCTCTGAGAAAGTTCAAGACAGTGGCTGAAGCTCTGGGTATCCTGTGTCCCAATGGGCCAGTGAAAAGCCTC
TATCCCCTGACATTCACTCAGGTAAAACAGTATTTTGGGTATGTGCTGTACCGAACAACGCTTCCTCAAGATTGCAG
TAACCCGAAACCCATTTTCTCTTCACCCTTCAATGGTGTCCGTGATCGGGCTTACGTCTCTGTGGACGGGGTCCCCC
AAGGAATCCTTGATCGAAACCTCATGACAGCTCTGAACATACGGGGGAAGGCTGGAGCCACGCTGGACATCCTGGTG
GAGAACATGGGGCGTGTGAACTATGGCAGATTCATCAATGACTTCAAGGGTTTGATTTCCAACATGACTATCAACTC
CACTGTCCTCACCAACTGGACGGTCTTCCCACTGAACACTGAGGCCATGGTACGCAACCATCTCTGGGGCCGGGAGG
CCAGTGATGAGGGTCACCTTGACGGACGGTCGACCTCCAATTCTTCGGACCTCATACTCCCCACCTTTTACGTGGGC
AACTTCTCCATCCCCTCGGGCATCCCAGACCTGCCACAGGACACCTTCATCCAGTTTCCTGGGTGGTCCAAGGGTCA
AGTATGGATCAATGGCTTTAACCTCGGCCGATACTGGCCCACAATGGGCCCACAAAAGACCTTGTTCGTGCCAAGGA
ACATCCTGACCACTTCAGCCCCAAACAACATCACAGTGTTGGAGCTAGAGTTTGCACCCTGCAGCGAGGGGACCCCA
GAGCTGTGTACAGTAGAGTTTGTTGACACTCCGGTCATTTCCTGACCTGACTTGACCATCGGTGGCCATTTTCCAAG
CCAGTCTGGTCAAGACTCATGGCTGAACCTCTGAGACTGAGCCTTGGGGAGCACAGCTCTACTCTGGTTACACGGAT
CACCTTTGTTGTGCTAGAATGGAAGCTACATctcgagGTCGACAATCAACCTCTGGATTACAAAATTTGTGAAAGAT
TGACTGGTATTCTTAACTATGTTGCTCCTTTTACGCTATGTGGATACGCTGCTTTAATGCCTTTGTATCATGCTATT
GCTTCCCGTATGGCTTTCATTTTCTCCTCCTTGTATAAATCCTGGTTGCTGTCTCTTTATGAGGAGTTGTGGCCCGT
TGTCAGGCAACGTGGCGTGGTGTGCACTGTGTTTGCTGACGCAACCCCCACTGGTTGGGGCATTGCCACCACCTGTC
AGCTCCTTTCCGGGACTTTCGCTTTCCCCCTCCCTATTGCCACGGCGGAACTCATCGCCGCCTGCCTTGCCCGCTGC
TGGACAGGGGCTCGGCTGTTGGGCACTGACAATTCCGTGGTGTTGTCGGGGAAATCATCGTCCTTTCCTTGGCTGCT
CGCCTGTGTTGCCACCTGGATTCTGCGCGGGACGTCCTTCTGCTACGTCCCTTCGGCCCTCAATCCAGCGGACCTTC
CTTCCCGCGGCCTGCTGCCGGCTCTGCGGCCTCTTCCGCGTCTTCGCCTTCGCCCTCAGACGAGTCGGATCTCCCTT
TGGGCCGCCTCCCCGCCTGGAATTCGAGCTCGGTACCTTTAAGACCAATGACTTACAAGGCAGCTGTAGATCTTAGC
CACTTTTTAAAAGAAAAGGGGGGACTGGAAGGGCTAATTCACTCCCAACGAAGACAAGATCTGCTTTTTGCTTGTAC
TGGGTCTCTCTGGTTAGACCAGATCTGAGCCTGGGAGCTCTCTGGCTAACTAGGGAACCCACTGCTTAAGCCTCAAT
AAAGCTTGCCTTGAGTGCTTCAAGTAGTGTGTGCCCGTCTGTTGTGTGACTCTGGTAACTAGAGATCCCTCAGACCC
TTTTAGTCAGTGTGGAAAATCTCTAGCAGTAGTAGTTCATGTCATCTTATTATTCAGTATTTATAACTTGCAAAGAA
ATGAATATCAGAGAGTGAGAGGAACTTGTTTATTGCAGCTTATAATGGTTACAAATAAAGCAATAGCATCACAAATT
TCACAAATAAAGCATTTTTTTCACTGCATTCTAGTTGTGGTTTGTCCAAACTCATCAATGTATCTTATCATGTCTGG
CTCTAGCTATCCCGCCCCTAACTCCGCCCATCCCGCCCCTAACTCCGCCCAGTTCCGCCCATTCTCCGCCCCATGGC
TGACTAATTTTTTTTATTTATGCAGAGGCCGAGGCCGCCTCGGCCTCTGAGCTATTCCAGAAGTAGTGAGGAGGCTT
TTTTGGAGGCCTAGGGACGTACCCAATTCGCCCTATAGTGAGTCGTATTACGCGCGCTCACTGGCCGTCGTTTTACA
ACGTCGTGACTGGGAAAACCCTGGCGTTACCCAACTTAATCGCCTTGCAGCACATCCCCCTTTCGCCAGCTGGCGTA
ATAGCGAAGAGGCCCGCACCGATCGCCCTTCCCAACAGTTGCGCAGCCTGAATGGCGAATGGG
(GALNS WT)
Seq ID NO: 35
ATGGCGGCGGTTGTCGCGGCGACGAGGTGGTGGCAGCTGTTGCTGGTGCTCAGCGCCGCGGGGATGGGGGCCTCGGG
CGCCCCGCAGCCCCCCAACATCCTGCTCCTGCTCATGGACGACATGGGATGGGGTGACCTCGGGGTGTATGGAGAGC
CCTCCAGAGAGACCCCGAATTTGGACCGGATGGCTGCAGAAGGGCTGCTTTTCCCAAACTTCTATTCTGCCAACCCT
CTGTGCTCGCCATCGAGGGCGGCACTGCTCACAGGACGGCTACCCATCCGCAATGGCTTCTACACCACCAACGCCCA
TGCCAGAAACGCCTACACACCGCAGGAGATTGTGGGCGGCATCCCAGACTCGGAGCAGCTCCTGCCGGAGCTTCTGA
AGAAGGCCGGCTACGTCAGCAAGATTGTCGGCAAGTGGCATCTGGGTCACAGGCCCCAGTTCCACCCCCTGAAGCAC
GGATTTGATGAGTGGTTTGGATCCCCCAACTGCCACTTTGGACCTTATGACAACAAGGCCAGGCCCAACATCCCTGT
GTACAGGGACTGGGAGATGGTTGGCAGATATTATGAAGAATTTCCTATTAATCTGAAGACGGGGGAAGCCAACCTCA
CCCAGATCTACCTGCAGGAAGCCCTGGACTTCATTAAGAGACAGGCACGGCACCACCCCTTTTTCCTCTACTGGGCT
GTCGACGCCACGCACGCACCCGTCTATGCCTCCAAACCCTTCTTGGGCACCAGTCAGCGAGGGCGGTATGGAGACGC
CGTCCGGGAGATTGATGACAGCATTGGGAAGATACTGGAGCTCCTCCAAGACCTGCACGTCGCGGACAACACCTTCG
TCTTCTTCACGTCGGACAACGGCGCTGCCCTCATTTCCGCCCCCGAACAAGGTGGCAGCAACGGCCCCTTTCTGTGT
GGGAAGCAGACCACGTTTGAAGGAGGGATGAGGGAGCCTGCCCTCGCATGGTGGCCAGGGCACGTCACTGCAGGCCA
GGTGAGCCACCAGCTGGGCAGCATCATGGACCTCTTCACCACCAGCCTGGCCCTTGCGGGCCTGACGCCGCCCAGCG
ACAGGGCCATTGATGGCCTCAACCTCCTCCCCACCCTCCTGCAGGGCCGGCTGATGGACAGGCCTATCTTCTATTAC
CGTGGCGACACGCTGATGGCGGCCACCCTCGGGCAGCACAAGGCTCACTTCTGGACCTGGACCAACTCCTGGGAGAA
CTTCAGACAGGGCATTGATTTCTGCCCTGGGCAGAACGTTTCAGGGGTCACAACTCACAATCTGGAAGACCACACGA
AGCTGCCCCTGATCTTCCACCTGGGACGGGACCCAGGGGAGAGGTTCCCCCTCAGCTTTGCCAGCGCCGAGTACCAG
GAGGCCCTCAGCAGGATCACCTCGGTCGTCCAGCAGCACCAGGAGGCCTTGGTCCCCGCGCAGCCCCAGCTCAACGT
GTGCAACTGGGCGGTCATGAACTGGGCACCTCCGGGCTGTGAAAAGTTAGGGAAGTGTCTGACACCTCCAGAATCCA
TTCCCAAGAAGTGCCTCTGGTCCCACTAG
(GALNS OPT)
Seq ID NO: 36
ATGGCGGCGGTTGTGGCGGCGACCCGTTGGTGGCAACTGCTGCTGGTTCTGAGCGCGGCGGGCATGGGTGCGAGCGG
TGCGCCGCAACCGCCAAACATCCTGCTGCTGCTGATGGACGATATGGGTTGGGGCGACCTGGGTGTGTATGGTGAAC
CGAGCCGTGAAACCCCGAACCTGGATCGTATGGCGGCGGAGGGTCTGCTGTTCCCGAACTTTTATAGCGCGAACCCG
CTGTGCAGCCCGAGCCGTGCGGCGCTGCTGACCGGTCGTCTGCCGATCCGTAACGGCTTCTACACCACCAACGCGCA
CGCGCGTAACGCGTATACCCCGCAGGAAATCGTTGGTGGCATTCCGGACAGCGAGCAACTGCTGCCGGAACTGCTGA
AGAAAGCGGGTTACGTGAGCAAGATTGTTGGTAAATGGCATCTGGGTCACCGTCCGCAGTTCCACCCGCTGAAGCAC
GGTTTCGATGAGTGGTTTGGCAGCCCGAACTGCCACTTTGGCCCGTACGACAACAAAGCGCGTCCGAACATCCCGGT
GTATCGTGATTGGGAAATGGTTGGTCGTTACTATGAGGAATTCCCGATTAACCTGAAGACCGGCGAGGCGAACCTGA
CCCAGATCTACCTGCAAGAAGCGCTGGACTTTATTAAACGTCAGGCGCGTCACCACCCGTTCTTTCTGTATTGGGCG
GTGGATGCGACCCATGCGCCGGTTTATGCGAGCAAGCCGTTCCTGGGTACCAGCCAGCGTGGTCGTTATGGCGACGC
GGTGCGTGAGATCGACGATAGCATCGGCAAAATTCTGGAACTGCTGCAAGACCTGCACGTGGCGGATAACACCTTTG
TTTTCTTTACCAGCGATAACGGTGCGGCGCTGATTAGCGCGCCGGAGCAGGGTGGCAGCAACGGTCCGTTCCTGTGC
GGCAAGCAAACCACCTTTGAGGGTGGCATGCGTGAACCGGCGCTGGCGTGGTGGCCGGGTCACGTGACCGCGGGCCA
GGTTAGCCACCAACTGGGTAGCATCATGGACCTGTTCACCACCAGCCTGGCGCTGGCGGGCCTGACCCCGCCGAGCG
ACCGTGCGATTGATGGTCTGAACCTGCTGCCGACCCTGCTGCAGGGCCGTCTGATGGACCGTCCGATTTTCTACTAT
CGTGGTGATACCCTGATGGCGGCGACCCTGGGTCAACACAAGGCGCACTTTTGGACCTGGACCAACAGCTGGGAAAA
CTTCCGTCAGGGTATCGATTTTTGTCCGGGTCAAAACGTGAGCGGCGTTACCACCCACAACCTGGAGGACCACACCA
AACTGCCGCTGATTTTTCACCTGGGTCGTGATCCGGGCGAACGTTTCCCGCTGAGCTTTGCGAGCGCGGAGTACCAG
GAAGCGCTGAGCCGTATTACCAGCGTGGTTCAGCAACATCAAGAGGCGCTGGTTCCGGCGCAGCCGCAACTGAACGT
GTGCAACTGGGCGGTTATGAACTGGGCGCCGCCGGGTTGCGAAAAACTGGGCAAGTGCCTGACCCCGCCGGAAAGCA
TCCCGAAAAAGTGCCTGTGGAGCCATTAA
(LV-GALNS WT cassette)
Seq ID NO: 37
ATCATCGATTTCCGAATTCCACGGGGTTGGGGTTGCGCCTTTTCCAAGGCAGCCCTGGGTTTGCGCAGGGACGCGGC
TGCTCTGGGCGTGGTTCCGGGAAACGCAGCGGCGCCGACCCTGGGTCTCGCACATTCTTCACGTCCGTTCGCAGCGT
CACCCGGATCTTCGCCGCTACCCTTGTGGGCCCCCCGGCGACGCTTCCTGCTCCGCCCCTAAGTCGGGAAGGTTCCT
TGCGGTTCGCGGCGTGCCGGACGTGACAAACGGAAGCCGCACGTCTCACTAGTACCCTCGCAGACGGACAGCGCCAG
GGAGCAATGGCAGCGCGCCGACCGCGATGGGCTGTGGCCAATAGCGGCTGCTCAGCAGGGCGCGCCGAGAGCAGCGG
CCGGGAAGGGGCGGTGCGGGAGGCGGGGTGTGGGGCGGTAGTGTGGGCCCTGTTCCTGCCCGCGCGGTGTTCCGCAT
TCTGCAAGCCTCCGGAGCGCACGTCGGCAGTCGGCTCCCTCGTTGACCGAATCACCGACCTCTCTCCCCAACGCggg
ggGCCGCCACCATGGCGGCGGTTGTCGCGGCGACGAGGTGGTGGCAGCTGTTGCTGGTGCTCAGCGCCGCGGGGATG
GGGGCCTCGGGCGCCCCGCAGCCCCCCAACATCCTGCTCCTGCTCATGGACGACATGGGATGGGGTGACCTCGGGGT
GTATGGAGAGCCCTCCAGAGAGACCCCGAATTTGGACCGGATGGCTGCAGAAGGGCTGCTTTTCCCAAACTTCTATT
CTGCCAACCCTCTGTGCTCGCCATCGAGGGCGGCACTGCTCACAGGACGGCTACCCATCCGCAATGGCTTCTACACC
ACCAACGCCCATGCCAGAAACGCCTACACACCGCAGGAGATTGTGGGCGGCATCCCAGACTCGGAGCAGCTCCTGCC
GGAGCTTCTGAAGAAGGCCGGCTACGTCAGCAAGATTGTCGGCAAGTGGCATCTGGGTCACAGGCCCCAGTTCCACC
CCCTGAAGCACGGATTTGATGAGTGGTTTGGATCCCCCAACTGCCACTTTGGACCTTATGACAACAAGGCCAGGCCC
AACATCCCTGTGTACAGGGACTGGGAGATGGTTGGCAGATATTATGAAGAATTTCCTATTAATCTGAAGACGGGGGA
AGCCAACCTCACCCAGATCTACCTGCAGGAAGCCCTGGACTTCATTAAGAGACAGGCACGGCACCACCCCTTTTTCC
TCTACTGGGCTGTCGACGCCACGCACGCACCCGTCTATGCCTCCAAACCCTTCTTGGGCACCAGTCAGCGAGGGCGG
TATGGAGACGCCGTCCGGGAGATTGATGACAGCATTGGGAAGATACTGGAGCTCCTCCAAGACCTGCACGTCGCGGA
CAACACCTTCGTCTTCTTCACGTCGGACAACGGCGCTGCCCTCATTTCCGCCCCCGAACAAGGTGGCAGCAACGGCC
CCTTTCTGTGTGGGAAGCAGACCACGTTTGAAGGAGGGATGAGGGAGCCTGCCCTCGCATGGTGGCCAGGGCACGTC
ACTGCAGGCCAGGTGAGCCACCAGCTGGGCAGCATCATGGACCTCTTCACCACCAGCCTGGCCCTTGCGGGCCTGAC
GCCGCCCAGCGACAGGGCCATTGATGGCCTCAACCTCCTCCCCACCCTCCTGCAGGGCCGGCTGATGGACAGGCCTA
TCTTCTATTACCGTGGCGACACGCTGATGGCGGCCACCCTCGGGCAGCACAAGGCTCACTTCTGGACCTGGACCAAC
TCCTGGGAGAACTTCAGACAGGGCATTGATTTCTGCCCTGGGCAGAACGTTTCAGGGGTCACAACTCACAATCTGGA
AGACCACACGAAGCTGCCCCTGATCTTCCACCTGGGACGGGACCCAGGGGAGAGGTTCCCCCTCAGCTTTGCCAGCG
CCGAGTACCAGGAGGCCCTCAGCAGGATCACCTCGGTCGTCCAGCAGCACCAGGAGGCCTTGGTCCCCGCGCAGCCC
CAGCTCAACGTGTGCAACTGGGCGGTCATGAACTGGGCACCTCCGGGCTGTGAAAAGTTAGGGAAGTGTCTGACACC
TCCAGAATCCATTCCCAAGAAGTGCCTCTGGTCCCACTAGCACCTGCGCctcgag
(LV-GALNS OPT cassette)
Seq ID NO: 38
ATCATCGATTTCCGAATTCCACGGGGTTGGGGTTGCGCCTTTTCCAAGGCAGCCCTGGGTTTGCGCAGGGACGCGGC
TGCTCTGGGCGTGGTTCCGGGAAACGCAGCGGCGCCGACCCTGGGTCTCGCACATTCTTCACGTCCGTTCGCAGCGT
CACCCGGATCTTCGCCGCTACCCTTGTGGGCCCCCCGGCGACGCTTCCTGCTCCGCCCCTAAGTCGGGAAGGTTCCT
TGCGGTTCGCGGCGTGCCGGACGTGACAAACGGAAGCCGCACGTCTCACTAGTACCCTCGCAGACGGACAGCGCCAG
GGAGCAATGGCAGCGCGCCGACCGCGATGGGCTGTGGCCAATAGCGGCTGCTCAGCAGGGCGCGCCGAGAGCAGCGG
CCGGGAAGGGGCGGTGCGGGAGGCGGGGTGTGGGGCGGTAGTGTGGGCCCTGTTCCTGCCCGCGCGGTGTTCCGCAT
TCTGCAAGCCTCCGGAGCGCACGTCGGCAGTCGGCTCCCTCGTTGACCGAATCACCGACCTCTCTCCCCAACGCgga
tccGCCGCCACCATGGCGGCGGTTGTGGCGGCGACCCGTTGGTGGCAACTGCTGCTGGTTCTGAGCGCGGCGGGCAT
GGGTGCGAGCGGTGCGCCGCAACCGCCAAACATCCTGCTGCTGCTGATGGACGATATGGGTTGGGGCGACCTGGGTG
TGTATGGTGAACCGAGCCGTGAAACCCCGAACCTGGATCGTATGGCGGCGGAGGGTCTGCTGTTCCCGAACTTTTAT
AGCGCGAACCCGCTGTGCAGCCCGAGCCGTGCGGCGCTGCTGACCGGTCGTCTGCCGATCCGTAACGGCTTCTACAC
CACCAACGCGCACGCGCGTAACGCGTATACCCCGCAGGAAATCGTTGGTGGCATTCCGGACAGCGAGCAACTGCTGC
CGGAACTGCTGAAGAAAGCGGGTTACGTGAGCAAGATTGTTGGTAAATGGCATCTGGGTCACCGTCCGCAGTTCCAC
CCGCTGAAGCACGGTTTCGATGAGTGGTTTGGCAGCCCGAACTGCCACTTTGGCCCGTACGACAACAAAGCGCGTCC
GAACATCCCGGTGTATCGTGATTGGGAAATGGTTGGTCGTTACTATGAGGAATTCCCGATTAACCTGAAGACCGGCG
AGGCGAACCTGACCCAGATCTACCTGCAAGAAGCGCTGGACTTTATTAAACGTCAGGCGCGTCACCACCCGTTCTTT
CTGTATTGGGCGGTGGATGCGACCCATGCGCCGGTTTATGCGAGCAAGCCGTTCCTGGGTACCAGCCAGCGTGGTCG
TTATGGCGACGCGGTGCGTGAGATCGACGATAGCATCGGCAAAATTCTGGAACTGCTGCAAGACCTGCACGTGGCGG
ATAACACCTTTGTTTTCTTTACCAGCGATAACGGTGCGGCGCTGATTAGCGCGCCGGAGCAGGGTGGCAGCAACGGT
CCGTTCCTGTGCGGCAAGCAAACCACCTTTGAGGGTGGCATGCGTGAACCGGCGCTGGCGTGGTGGCCGGGTCACGT
GACCGCGGGCCAGGTTAGCCACCAACTGGGTAGCATCATGGACCTGTTCACCACCAGCCTGGCGCTGGCGGGCCTGA
CCCCGCCGAGCGACCGTGCGATTGATGGTCTGAACCTGCTGCCGACCCTGCTGCAGGGCCGTCTGATGGACCGTCCG
ATTTTCTACTATCGTGGTGATACCCTGATGGCGGCGACCCTGGGTCAACACAAGGCGCACTTTTGGACCTGGACCAA
CAGCTGGGAAAACTTCCGTCAGGGTATCGATTTTTGTCCGGGTCAAAACGTGAGCGGCGTTACCACCCACAACCTGG
AGGACCACACCAAACTGCCGCTGATTTTTCACCTGGGTCGTGATCCGGGCGAACGTTTCCCGCTGAGCTTTGCGAGC
GCGGAGTACCAGGAAGCGCTGAGCCGTATTACCAGCGTGGTTCAGCAACATCAAGAGGCGCTGGTTCCGGCGCAGCC
GCAACTGAACGTGTGCAACTGGGCGGTTATGAACTGGGCGCCGCCGGGTTGCGAAAAACTGGGCAAGTGCCTGACCC
CGCCGGAAAGCATCCCGAAAAAGTGCCTGTGGAGCCATTAACACCTGCGCgtcgac
(LV human GALNS WT)
SEQ ID NO: 39
ACGCGCCCTGTAGCGGCGCATTAAGCGCGGCGGGTGTGGTGGTTACGCGCAGCGTGACCGCTACACTTGCCAGCGCC
CTAGCGCCCGCTCCTTTCGCTTTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAACTTGATTAGGGTGATGGTTCAC
GTAGTGGGCCATCGCCCTGATAGACGGTTTTTCGCCCTTTGACGTTGGAGTCCACGTTCTTTAATAGTGGACTCTTG
TTCCAAACTGGAACAACACTCAACCCTATCTCGGTCTATTCTTTTGATTTATAAGGGATTTTGCCGATTTCGGCCTA
TTGGTTAAAAAATGAGCTGATTTAACAAAAATTTAACGCGAATTTTAACAAAATATTAACGCTTACAATTTAGGTGG
CACTTTTCGGGGAAATGTGCGCGGAACCCCTATTTGTTTATTTTTCTAAATACATTCAAATATGTATCCGCTCATGA
GACAATAACCCTGATAAATGCTTCAATAATAGCACCTAGATCAAGAGACAGGATGAGGATCGTTTCGCATGATTGAA
CAAGATGGATTGCACGCAGGTTCTCCGGCCGCTTGGGTGGAGAGGCTATTCGGCTATGACTGGGCACAACAGACAAT
CGGCTGCTCTGATGCCGCCGTGTTCCGGCTGTCAGCGCAGGGGCGCCCGGTTCTTTTTGTCAAGACCGACCTGTCCG
GTGCCCTGAATGAACTGCAAGACGAGGCAGCGCGGCTATCGTGGCTGGCCACGACGGGCGTTCCTTGCGCAGCTGTG
CTCGACGTTGTCACTGAAGCGGGAAGGGACTGGCTGCTATTGGGCGAAGTGCCGGGGCAGGATCTCCTGTCATCTCA
CCTTGCTCCTGCCGAGAAAGTATCCATCATGGCTGATGCAATGCGGCGGCTGCATACGCTTGATCCGGCTACCTGCC
CATTCGACCACCAAGCGAAACATCGCATCGAGCGAGCACGTACTCGGATGGAAGCCGGTCTTGTCGATCAGGATGAT
CTGGACGAAGAGCATCAGGGGCTCGCGCCAGCCGAACTGTTCGCCAGGCTCAAGGCGAGCATGCCCGACGGCGAGGA
TCTCGTCGTGACCCATGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCTGAAGAGCTTGGCGGC
GAATGGGCTGACCGCTTCCTCGTGCTTTACGGTATCGCCGCTCCCGATTCGCAGCGCATCGCCTTCTATCGCCTTCT
TGACGAGTTCTTCTGAATTATTAACGCTTACAATTTCCTGATGCGGTATTTTCTCCTTACGCATCTGTGCGGTATTT
CACACCGCATCAGGTGGCACTTTTCGGGGAAATGTGCGCGGAACCCCTATTTGTTTATTTTTCTAAATACATTCAAA
TATGTATCCGCTCATGACCAAAATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAATAC
TGTTCTTCTAGTGTAGCCGTAGTTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGACTCAAGACGATAGTTACCGGAT
AAGGCGCAGCGGTCGGGCTGAACGGGGGGGTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGAGCGCAGCGAGTCAGTGAGCGAG
GAAGCGGAAGAGCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAATTTCACACAGGAAACAGCTATGAC
CATGATTACGCCAAGCGCGCAATTAACCCTCACTAAAGGGAACAAAAGCTGGAGCTGCAAGCTTGGCCATTGCATAC
GTTGTATCCATATCATAATATGTACATTTATATTGGCTCATGTCCAACATTACCGCCATGTTGACATTGATTATTGA
CTAGTTATTAATAGTAATCAATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAATGACGTATGTTCCCATAGTAAC
GCCAATAGGGACTTTCCATTGACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGATGCGGTTTTGGCAGTA
CATCAATGGGCGTGGATAGCGGTTTGACTCACGGGGATTTCCAAGTCTCCACCCCATTGACGTCAATGGGAGTTTGT
TTTGGCACCAAAATCAACGGGACTTTCCAAAATGTCGTAACAACTCCGCCCCATTGACGCAAATGGGCGGTAGGCGT
GTACGGTGGGAGGTCTATATAAGCAGAGCTCGTTTAGTGAACCGGGGTCTCTCTGGTTAGACCAGATCTGAGCCTGG
GAGCTCTCTGGCTAACTAGGGAACCCACTGCTTAAGCCTCAATAAAGCTTGCCTTGAGTGCTTCAAGTAGTGTGTGC
CCGTCTGTTGTGTGACTCTGGTAACTAGAGATCCCTCAGACCCTTTTAGTCAGTGTGGAAAATCTCTAGCAGTGGCG
CCCGAACAGGGACTTGAAAGCGAAAGGGAAACCAGAGGAGCTCTCTCGACGCAGGACTCGGCTTGCTGAAGCGCGCA
CGGCAAGAGGCGAGGGGCGGCGACTGGTGAGTACGCCAAAAATTTTGACTAGCGGAGGCTAGAAGGAGAGAGATGGG
TGCGAGAGCGTCAGTATTAAGCGGGGGAGAATTAGATCGCGATGGGAAAAAATTCGGTTAAGGCCAGGGGGAAAGAA
AAAATATAAATTAAAACATATAGTATGGGCAAGCAGGGAGCTAGAACGATTCGCAGTTAATCCTGGCCTGTTAGAAA
CATCAGAAGGCTGTAGACAAATACTGGGACAGCTACAACCATCCCTTCAGACAGGATCAGAAGAACTTAGATCATTA
TATAATACAGTAGCAACCCTCTATTGTGTGCATCAAAGGATAGAGATAAAAGACACCAAGGAAGCTTTAGACAAGAT
AGAGGAAGAGCAAAACAAAAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGGAGTAGCACCCACCAAGGC
AAAGAGAAGAGTGGTGCAGAGAGAAAAAAGAGCAGTGGGAATAGGAGCTTTGTTCCTTGGGTTCTTGGGAGCAGCAG
GAAGCACTATGGGCGCAGCGTCAATGACGCTGACGGTACAGGCCAGACAATTATTGTCTGGTATAGTGCAGCAGCAG
AACAATTTGCTGAGGGCTATTGAGGCGCAACAGCATCTGTTGCAACTCACAGTCTGGGGCATCAAGCAGCTCCAGGC
AAGAATCCTGGCTGTGGAAAGATACCTAAAGGATCAACAGCTCCTGGGGATTTGGGGTTGCTCTGGAAAACTCATTT
GCACCACTGCTGTGCCTTGGAATGCTAGTTGGAGTAATAAATCTCTGGAACAGATTTGGAATCACACGACCTGGATG
GAGTGGGACAGAGAAATTAACAATTACACAAGCTTAATACACTCCTTAATTGAAGAATCGCAAAACCAGCAAGAAAA
GAATGAACAAGAATTATTGGAATTAGATAAATGGGCAAGTTTGTGGAATTGGTTTAACATAACAAATTGGCTGTGGT
ATATAAAATTATTCATAATGATAGTAGGAGGCTTGGTAGGTTTAAGAATAGTTTTTGCTGTACTTTCTATAGTGAAT
AGAGTTAGGCAGGGATATTCACCATTATCGTTTCAGACCCACCTCCCAACCCCGAGGGGACCCGACAGGCCCGAAGG
AATAGAAGAAGAAGGTGGAGAGAGAGACAGAGACAGATCCATTCGATTAGTGAACGGATCTCGACGGTATCGGTTAA
CTTTTAAAAGAAAAGGGGGGATTGGGGGGTACAGTGCAGGGGAAAGAATAGTAGACATAATAGCAACAGACATACAA
ACTAAAGAATTACAAAAACAAATTACAAAATTCAAAATTTTATCGATAAGCTTGATATCATCGATTTCCGAATTCCA
CGGGGTTGGGGTTGCGCCTTTTCCAAGGCAGCCCTGGGTTTGCGCAGGGACGCGGCTGCTCTGGGCGTGGTTCCGGG
AAACGCAGCGGCGCCGACCCTGGGTCTCGCACATTCTTCACGTCCGTTCGCAGCGTCACCCGGATCTTCGCCGCTAC
CCTTGTGGGCCCCCCGGCGACGCTTCCTGCTCCGCCCCTAAGTCGGGAAGGTTCCTTGCGGTTCGCGGCGTGCCGGA
CGTGACAAACGGAAGCCGCACGTCTCACTAGTACCCTCGCAGACGGACAGCGCCAGGGAGCAATGGCAGCGCGCCGA
CCGCGATGGGCTGTGGCCAATAGCGGCTGCTCAGCAGGGCGCGCCGAGAGCAGCGGCCGGGAAGGGGCGGTGCGGGA
GGCGGGGTGTGGGGCGGTAGTGTGGGCCCTGTTCCTGCCCGCGCGGTGTTCCGCATTCTGCAAGCCTCCGGAGCGCA
CGTCGGCAGTCGGCTCCCTCGTTGACCGAATCACCGACCTCTCTCCCCAACGCGGGGGGCCGCCACCATGGCGGCGG
TTGTCGCGGCGACGAGGTGGTGGCAGCTGTTGCTGGTGCTCAGCGCCGCGGGGATGGGGGCCTCGGGCGCCCCGCAG
CCCCCCAACATCCTGCTCCTGCTCATGGACGACATGGGATGGGGTGACCTCGGGGTGTATGGAGAGCCCTCCAGAGA
GACCCCGAATTTGGACCGGATGGCTGCAGAAGGGCTGCTTTTCCCAAACTTCTATTCTGCCAACCCTCTGTGCTCGC
CATCGAGGGCGGCACTGCTCACAGGACGGCTACCCATCCGCAATGGCTTCTACACCACCAACGCCCATGCCAGAAAC
GCCTACACACCGCAGGAGATTGTGGGCGGCATCCCAGACTCGGAGCAGCTCCTGCCGGAGCTTCTGAAGAAGGCCGG
CTACGTCAGCAAGATTGTCGGCAAGTGGCATCTGGGTCACAGGCCCCAGTTCCACCCCCTGAAGCACGGATTTGATG
AGTGGTTTGGATCCCCCAACTGCCACTTTGGACCTTATGACAACAAGGCCAGGCCCAACATCCCTGTGTACAGGGAC
TGGGAGATGGTTGGCAGATATTATGAAGAATTTCCTATTAATCTGAAGACGGGGGAAGCCAACCTCACCCAGATCTA
CCTGCAGGAAGCCCTGGACTTCATTAAGAGACAGGCACGGCACCACCCCTTTTTCCTCTACTGGGCTGTCGACGCCA
CGCACGCACCCGTCTATGCCTCCAAACCCTTCTTGGGCACCAGTCAGCGAGGGCGGTATGGAGACGCCGTCCGGGAG
ATTGATGACAGCATTGGGAAGATACTGGAGCTCCTCCAAGACCTGCACGTCGCGGACAACACCTTCGTCTTCTTCAC
GTCGGACAACGGCGCTGCCCTCATTTCCGCCCCCGAACAAGGTGGCAGCAACGGCCCCTTTCTGTGTGGGAAGCAGA
CCACGTTTGAAGGAGGGATGAGGGAGCCTGCCCTCGCATGGTGGCCAGGGCACGTCACTGCAGGCCAGGTGAGCCAC
CAGCTGGGCAGCATCATGGACCTCTTCACCACCAGCCTGGCCCTTGCGGGCCTGACGCCGCCCAGCGACAGGGCCAT
TGATGGCCTCAACCTCCTCCCCACCCTCCTGCAGGGCCGGCTGATGGACAGGCCTATCTTCTATTACCGTGGCGACA
CGCTGATGGCGGCCACCCTCGGGCAGCACAAGGCTCACTTCTGGACCTGGACCAACTCCTGGGAGAACTTCAGACAG
GGCATTGATTTCTGCCCTGGGCAGAACGTTTCAGGGGTCACAACTCACAATCTGGAAGACCACACGAAGCTGCCCCT
GATCTTCCACCTGGGACGGGACCCAGGGGAGAGGTTCCCCCTCAGCTTTGCCAGCGCCGAGTACCAGGAGGCCCTCA
GCAGGATCACCTCGGTCGTCCAGCAGCACCAGGAGGCCTTGGTCCCCGCGCAGCCCCAGCTCAACGTGTGCAACTGG
GCGGTCATGAACTGGGCACCTCCGGGCTGTGAAAAGTTAGGGAAGTGTCTGACACCTCCAGAATCCATTCCCAAGAA
GTGCCTCTGGTCCCACTAGCACCTGCGCCTCGAGGTCGACAATCAACCTCTGGATTACAAAATTTGTGAAAGATTGA
CTGGTATTCTTAACTATGTTGCTCCTTTTACGCTATGTGGATACGCTGCTTTAATGCCTTTGTATCATGCTATTGCT
TCCCGTATGGCTTTCATTTTCTCCTCCTTGTATAAATCCTGGTTGCTGTCTCTTTATGAGGAGTTGTGGCCCGTTGT
CAGGCAACGTGGCGTGGTGTGCACTGTGTTTGCTGACGCAACCCCCACTGGTTGGGGCATTGCCACCACCTGTCAGC
TCCTTTCCGGGACTTTCGCTTTCCCCCTCCCTATTGCCACGGCGGAACTCATCGCCGCCTGCCTTGCCCGCTGCTGG
ACAGGGGCTCGGCTGTTGGGCACTGACAATTCCGTGGTGTTGTCGGGGAAATCATCGTCCTTTCCTTGGCTGCTCGC
CTGTGTTGCCACCTGGATTCTGCGCGGGACGTCCTTCTGCTACGTCCCTTCGGCCCTCAATCCAGCGGACCTTCCTT
CCCGCGGCCTGCTGCCGGCTCTGCGGCCTCTTCCGCGTCTTCGCCTTCGCCCTCAGACGAGTCGGATCTCCCTTTGG
GCCGCCTCCCCGCCTGGAATTCGAGCTCGGTACCTTTAAGACCAATGACTTACAAGGCAGCTGTAGATCTTAGCCAC
TTTTTAAAAGAAAAGGGGGGACTGGAAGGGCTAATTCACTCCCAACGAAGACAAGATCTGCTTTTTGCTTGTACTGG
GTCTCTCTGGTTAGACCAGATCTGAGCCTGGGAGCTCTCTGGCTAACTAGGGAACCCACTGCTTAAGCCTCAATAAA
GCTTGCCTTGAGTGCTTCAAGTAGTGTGTGCCCGTCTGTTGTGTGACTCTGGTAACTAGAGATCCCTCAGACCCTTT
TAGTCAGTGTGGAAAATCTCTAGCAGTAGTAGTTCATGTCATCTTATTATTCAGTATTTATAACTTGCAAAGAAATG
AATATCAGAGAGTGAGAGGAACTTGTTTATTGCAGCTTATAATGGTTACAAATAAAGCAATAGCATCACAAATTTCA
CAAATAAAGCATTTTTTTCACTGCATTCTAGTTGTGGTTTGTCCAAACTCATCAATGTATCTTATCATGTCTGGCTC
TAGCTATCCCGCCCCTAACTCCGCCCATCCCGCCCCTAACTCCGCCCAGTTCCGCCCATTCTCCGCCCCATGGCTGA
CTAATTTTTTTTATTTATGCAGAGGCCGAGGCCGCCTCGGCCTCTGAGCTATTCCAGAAGTAGTGAGGAGGCTTTTT
TGGAGGCCTAGGGACGTACCCAATTCGCCCTATAGTGAGTCGTATTACGCGCGCTCACTGGCCGTCGTTTTACAACG
TCGTGACTGGGAAAACCCTGGCGTTACCCAACTTAATCGCCTTGCAGCACATCCCCCTTTCGCCAGCTGGCGTAATA
GCGAAGAGGCCCGCACCGATCGCCCTTCCCAACAGTTGCGCAGCCTGAATGGCGAATGGG
(LV human GALNS OPT)
SEQ ID NO: 40
ACGCGCCCTGTAGCGGCGCATTAAGCGCGGCGGGTGTGGTGGTTACGCGCAGCGTGACCGCTACACTTGCCAGCGCC
CTAGCGCCCGCTCCTTTCGCTTTCTTCCCTTCCTTTCTCGCCACGTTCGCCGGCTTTCCCCGTCAAGCTCTAAATCG
GGGGCTCCCTTTAGGGTTCCGATTTAGTGCTTTACGGCACCTCGACCCCAAAAAACTTGATTAGGGTGATGGTTCAC
GTAGTGGGCCATCGCCCTGATAGACGGTTTTTCGCCCTTTGACGTTGGAGTCCACGTTCTTTAATAGTGGACTCTTG
TTCCAAACTGGAACAACACTCAACCCTATCTCGGTCTATTCTTTTGATTTATAAGGGATTTTGCCGATTTCGGCCTA
TTGGTTAAAAAATGAGCTGATTTAACAAAAATTTAACGCGAATTTTAACAAAATATTAACGCTTACAATTTAGGTGG
CACTTTTCGGGGAAATGTGCGCGGAACCCCTATTTGTTTATTTTTCTAAATACATTCAAATATGTATCCGCTCATGA
GACAATAACCCTGATAAATGCTTCAATAATAGCACCTAGATCAAGAGACAGGATGAGGATCGTTTCGCATGATTGAA
CAAGATGGATTGCACGCAGGTTCTCCGGCCGCTTGGGTGGAGAGGCTATTCGGCTATGACTGGGCACAACAGACAAT
CGGCTGCTCTGATGCCGCCGTGTTCCGGCTGTCAGCGCAGGGGCGCCCGGTTCTTTTTGTCAAGACCGACCTGTCCG
GTGCCCTGAATGAACTGCAAGACGAGGCAGCGCGGCTATCGTGGCTGGCCACGACGGGCGTTCCTTGCGCAGCTGTG
CTCGACGTTGTCACTGAAGCGGGAAGGGACTGGCTGCTATTGGGCGAAGTGCCGGGGCAGGATCTCCTGTCATCTCA
CCTTGCTCCTGCCGAGAAAGTATCCATCATGGCTGATGCAATGCGGCGGCTGCATACGCTTGATCCGGCTACCTGCC
CATTCGACCACCAAGCGAAACATCGCATCGAGCGAGCACGTACTCGGATGGAAGCCGGTCTTGTCGATCAGGATGAT
CTGGACGAAGAGCATCAGGGGCTCGCGCCAGCCGAACTGTTCGCCAGGCTCAAGGCGAGCATGCCCGACGGCGAGGA
TCTCGTCGTGACCCATGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCTGAAGAGCTTGGCGGC
GAATGGGCTGACCGCTTCCTCGTGCTTTACGGTATCGCCGCTCCCGATTCGCAGCGCATCGCCTTCTATCGCCTTCT
TGACGAGTTCTTCTGAATTATTAACGCTTACAATTTCCTGATGCGGTATTTTCTCCTTACGCATCTGTGCGGTATTT
CACACCGCATCAGGTGGCACTTTTCGGGGAAATGTGCGCGGAACCCCTATTTGTTTATTTTTCTAAATACATTCAAA
TATGTATCCGCTCATGACCAAAATCCCTTAACGTGAGTTTTCGTTCCACTGAGCGTCAGACCCCGTAGAAAAGATCA
AAGGATCTTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATACCAAATAC
TGTTCTTCTAGTGTAGCCGTAGTTAGGCCACCACTTCAAGAACTCTGTAGCACCGCCTACATACCTCGCTCTGCTAA
TCCTGTTACCAGTGGCTGCTGCCAGTGGCGATAAGTCGTGTCTTACCGGGTTGGACTCAAGACGATAGTTACCGGAT
AAGGCGCAGCGGTCGGGCTGAACGGGGGGGTCGTGCACACAGCCCAGCTTGGAGCGAACGACCTACACCGAACTGAG
ATACCTACAGCGTGAGCTATGAGAAAGCGCCACGCTTCCCGAAGGGAGAAAGGCGGACAGGTATCCGGTAAGCGGCA
GGGTCGGAACAGGAGAGCGCACGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGAGCGCAGCGAGTCAGTGAGCGAG
GAAGCGGAAGAGCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAATTTCACACAGGAAACAGCTATGAC
CATGATTACGCCAAGCGCGCAATTAACCCTCACTAAAGGGAACAAAAGCTGGAGCTGCAAGCTTGGCCATTGCATAC
GTTGTATCCATATCATAATATGTACATTTATATTGGCTCATGTCCAACATTACCGCCATGTTGACATTGATTATTGA
CTAGTTATTAATAGTAATCAATTACGGGGTCATTAGTTCATAGCCCATATATGGAGTTCCGCGTTACATAACTTACG
GTAAATGGCCCGCCTGGCTGACCGCCCAACGACCCCCGCCCATTGACGTCAATAATGACGTATGTTCCCATAGTAAC
GCCAATAGGGACTTTCCATTGACGTCAATGGGTGGAGTATTTACGGTAAACTGCCCACTTGGCAGTACATCAAGTGT
ATCATATGCCAAGTACGCCCCCTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATGGTGATGCGGTTTTGGCAGTA
CATCAATGGGCGTGGATAGCGGTTTGACTCACGGGGATTTCCAAGTCTCCACCCCATTGACGTCAATGGGAGTTTGT
TTTGGCACCAAAATCAACGGGACTTTCCAAAATGTCGTAACAACTCCGCCCCATTGACGCAAATGGGCGGTAGGCGT
GTACGGTGGGAGGTCTATATAAGCAGAGCTCGTTTAGTGAACCGGGGTCTCTCTGGTTAGACCAGATCTGAGCCTGG
GAGCTCTCTGGCTAACTAGGGAACCCACTGCTTAAGCCTCAATAAAGCTTGCCTTGAGTGCTTCAAGTAGTGTGTGC
CCGTCTGTTGTGTGACTCTGGTAACTAGAGATCCCTCAGACCCTTTTAGTCAGTGTGGAAAATCTCTAGCAGTGGCG
CCCGAACAGGGACTTGAAAGCGAAAGGGAAACCAGAGGAGCTCTCTCGACGCAGGACTCGGCTTGCTGAAGCGCGCA
CGGCAAGAGGCGAGGGGCGGCGACTGGTGAGTACGCCAAAAATTTTGACTAGCGGAGGCTAGAAGGAGAGAGATGGG
TGCGAGAGCGTCAGTATTAAGCGGGGGAGAATTAGATCGCGATGGGAAAAAATTCGGTTAAGGCCAGGGGGAAAGAA
AAAATATAAATTAAAACATATAGTATGGGCAAGCAGGGAGCTAGAACGATTCGCAGTTAATCCTGGCCTGTTAGAAA
CATCAGAAGGCTGTAGACAAATACTGGGACAGCTACAACCATCCCTTCAGACAGGATCAGAAGAACTTAGATCATTA
TATAATACAGTAGCAACCCTCTATTGTGTGCATCAAAGGATAGAGATAAAAGACACCAAGGAAGCTTTAGACAAGAT
AGAGGAAGAGCAAAACAAAAGTAAGACCACCGCACAGCAAGCGGCCGCTGATCTTCAGACCTGGAGGAGGAGATATG
AGGGACAATTGGAGAAGTGAATTATATAAATATAAAGTAGTAAAAATTGAACCATTAGGAGTAGCACCCACCAAGGC
AAAGAGAAGAGTGGTGCAGAGAGAAAAAAGAGCAGTGGGAATAGGAGCTTTGTTCCTTGGGTTCTTGGGAGCAGCAG
GAAGCACTATGGGCGCAGCGTCAATGACGCTGACGGTACAGGCCAGACAATTATTGTCTGGTATAGTGCAGCAGCAG
AACAATTTGCTGAGGGCTATTGAGGCGCAACAGCATCTGTTGCAACTCACAGTCTGGGGCATCAAGCAGCTCCAGGC
AAGAATCCTGGCTGTGGAAAGATACCTAAAGGATCAACAGCTCCTGGGGATTTGGGGTTGCTCTGGAAAACTCATTT
GCACCACTGCTGTGCCTTGGAATGCTAGTTGGAGTAATAAATCTCTGGAACAGATTTGGAATCACACGACCTGGATG
GAGTGGGACAGAGAAATTAACAATTACACAAGCTTAATACACTCCTTAATTGAAGAATCGCAAAACCAGCAAGAAAA
GAATGAACAAGAATTATTGGAATTAGATAAATGGGCAAGTTTGTGGAATTGGTTTAACATAACAAATTGGCTGTGGT
ATATAAAATTATTCATAATGATAGTAGGAGGCTTGGTAGGTTTAAGAATAGTTTTTGCTGTACTTTCTATAGTGAAT
AGAGTTAGGCAGGGATATTCACCATTATCGTTTCAGACCCACCTCCCAACCCCGAGGGGACCCGACAGGCCCGAAGG
AATAGAAGAAGAAGGTGGAGAGAGAGACAGAGACAGATCCATTCGATTAGTGAACGGATCTCGACGGTATCGGTTAA
CTTTTAAAAGAAAAGGGGGGATTGGGGGGTACAGTGCAGGGGAAAGAATAGTAGACATAATAGCAACAGACATACAA
ACTAAAGAATTACAAAAACAAATTACAAAATTCAAAATTTTATCGATAAGCTTGATATCATCGATTTCCGAATTCCA
CGGGGTTGGGGTTGCGCCTTTTCCAAGGCAGCCCTGGGTTTGCGCAGGGACGCGGCTGCTCTGGGCGTGGTTCCGGG
AAACGCAGCGGCGCCGACCCTGGGTCTCGCACATTCTTCACGTCCGTTCGCAGCGTCACCCGGATCTTCGCCGCTAC
CCTTGTGGGCCCCCCGGCGACGCTTCCTGCTCCGCCCCTAAGTCGGGAAGGTTCCTTGCGGTTCGCGGCGTGCCGGA
CGTGACAAACGGAAGCCGCACGTCTCACTAGTACCCTCGCAGACGGACAGCGCCAGGGAGCAATGGCAGCGCGCCGA
CCGCGATGGGCTGTGGCCAATAGCGGCTGCTCAGCAGGGCGCGCCGAGAGCAGCGGCCGGGAAGGGGCGGTGCGGGA
GGCGGGGTGTGGGGCGGTAGTGTGGGCCCTGTTCCTGCCCGCGCGGTGTTCCGCATTCTGCAAGCCTCCGGAGCGCA
CGTCGGCAGTCGGCTCCCTCGTTGACCGAATCACCGACCTCTCTCCCCAACGCGGATCCGCCGCCACCATGGCGGCG
GTTGTGGCGGCGACCCGTTGGTGGCAACTGCTGCTGGTTCTGAGCGCGGCGGGCATGGGTGCGAGCGGTGCGCCGCA
ACCGCCAAACATCCTGCTGCTGCTGATGGACGATATGGGTTGGGGCGACCTGGGTGTGTATGGTGAACCGAGCCGTG
AAACCCCGAACCTGGATCGTATGGCGGCGGAGGGTCTGCTGTTCCCGAACTTTTATAGCGCGAACCCGCTGTGCAGC
CCGAGCCGTGCGGCGCTGCTGACCGGTCGTCTGCCGATCCGTAACGGCTTCTACACCACCAACGCGCACGCGCGTAA
CGCGTATACCCCGCAGGAAATCGTTGGTGGCATTCCGGACAGCGAGCAACTGCTGCCGGAACTGCTGAAGAAAGCGG
GTTACGTGAGCAAGATTGTTGGTAAATGGCATCTGGGTCACCGTCCGCAGTTCCACCCGCTGAAGCACGGTTTCGAT
GAGTGGTTTGGCAGCCCGAACTGCCACTTTGGCCCGTACGACAACAAAGCGCGTCCGAACATCCCGGTGTATCGTGA
TTGGGAAATGGTTGGTCGTTACTATGAGGAATTCCCGATTAACCTGAAGACCGGCGAGGCGAACCTGACCCAGATCT
ACCTGCAAGAAGCGCTGGACTTTATTAAACGTCAGGCGCGTCACCACCCGTTCTTTCTGTATTGGGCGGTGGATGCG
ACCCATGCGCCGGTTTATGCGAGCAAGCCGTTCCTGGGTACCAGCCAGCGTGGTCGTTATGGCGACGCGGTGCGTGA
GATCGACGATAGCATCGGCAAAATTCTGGAACTGCTGCAAGACCTGCACGTGGCGGATAACACCTTTGTTTTCTTTA
CCAGCGATAACGGTGCGGCGCTGATTAGCGCGCCGGAGCAGGGTGGCAGCAACGGTCCGTTCCTGTGCGGCAAGCAA
ACCACCTTTGAGGGTGGCATGCGTGAACCGGCGCTGGCGTGGTGGCCGGGTCACGTGACCGCGGGCCAGGTTAGCCA
CCAACTGGGTAGCATCATGGACCTGTTCACCACCAGCCTGGCGCTGGCGGGCCTGACCCCGCCGAGCGACCGTGCGA
TTGATGGTCTGAACCTGCTGCCGACCCTGCTGCAGGGCCGTCTGATGGACCGTCCGATTTTCTACTATCGTGGTGAT
ACCCTGATGGCGGCGACCCTGGGTCAACACAAGGCGCACTTTTGGACCTGGACCAACAGCTGGGAAAACTTCCGTCA
GGGTATCGATTTTTGTCCGGGTCAAAACGTGAGCGGCGTTACCACCCACAACCTGGAGGACCACACCAAACTGCCGC
TGATTTTTCACCTGGGTCGTGATCCGGGCGAACGTTTCCCGCTGAGCTTTGCGAGCGCGGAGTACCAGGAAGCGCTG
AGCCGTATTACCAGCGTGGTTCAGCAACATCAAGAGGCGCTGGTTCCGGCGCAGCCGCAACTGAACGTGTGCAACTG
GGCGGTTATGAACTGGGCGCCGCCGGGTTGCGAAAAACTGGGCAAGTGCCTGACCCCGCCGGAAAGCATCCCGAAAA
AGTGCCTGTGGAGCCATTAACACCTGCGCGTCGACAATCAACCTCTGGATTACAAAATTTGTGAAAGATTGACTGGT
ATTCTTAACTATGTTGCTCCTTTTACGCTATGTGGATACGCTGCTTTAATGCCTTTGTATCATGCTATTGCTTCCCG
TATGGCTTTCATTTTCTCCTCCTTGTATAAATCCTGGTTGCTGTCTCTTTATGAGGAGTTGTGGCCCGTTGTCAGGC
AACGTGGCGTGGTGTGCACTGTGTTTGCTGACGCAACCCCCACTGGTTGGGGCATTGCCACCACCTGTCAGCTCCTT
TCCGGGACTTTCGCTTTCCCCCTCCCTATTGCCACGGCGGAACTCATCGCCGCCTGCCTTGCCCGCTGCTGGACAGG
GGCTCGGCTGTTGGGCACTGACAATTCCGTGGTGTTGTCGGGGAAATCATCGTCCTTTCCTTGGCTGCTCGCCTGTG
TTGCCACCTGGATTCTGCGCGGGACGTCCTTCTGCTACGTCCCTTCGGCCCTCAATCCAGCGGACCTTCCTTCCCGC
GGCCTGCTGCCGGCTCTGCGGCCTCTTCCGCGTCTTCGCCTTCGCCCTCAGACGAGTCGGATCTCCCTTTGGGCCGC
CTCCCCGCCTGGAATTCGAGCTCGGTACCTTTAAGACCAATGACTTACAAGGCAGCTGTAGATCTTAGCCACTTTTT
AAAAGAAAAGGGGGGACTGGAAGGGCTAATTCACTCCCAACGAAGACAAGATCTGCTTTTTGCTTGTACTGGGTCTC
TCTGGTTAGACCAGATCTGAGCCTGGGAGCTCTCTGGCTAACTAGGGAACCCACTGCTTAAGCCTCAATAAAGCTTG
CCTTGAGTGCTTCAAGTAGTGTGTGCCCGTCTGTTGTGTGACTCTGGTAACTAGAGATCCCTCAGACCCTTTTAGTC
AGTGTGGAAAATCTCTAGCAGTAGTAGTTCATGTCATCTTATTATTCAGTATTTATAACTTGCAAAGAAATGAATAT
CAGAGAGTGAGAGGAACTTGTTTATTGCAGCTTATAATGGTTACAAATAAAGCAATAGCATCACAAATTTCACAAAT
AAAGCATTTTTTTCACTGCATTCTAGTTGTGGTTTGTCCAAACTCATCAATGTATCTTATCATGTCTGGCTCTAGCT
ATCCCGCCCCTAACTCCGCCCATCCCGCCCCTAACTCCGCCCAGTTCCGCCCATTCTCCGCCCCATGGCTGACTAAT
TTTTTTTATTTATGCAGAGGCCGAGGCCGCCTCGGCCTCTGAGCTATTCCAGAAGTAGTGAGGAGGCTTTTTTGGAG
GCCTAGGGACGTACCCAATTCGCCCTATAGTGAGTCGTATTACGCGCGCTCACTGGCCGTCGTTTTACAACGTCGTG
ACTGGGAAAACCCTGGCGTTACCCAACTTAATCGCCTTGCAGCACATCCCCCTTTCGCCAGCTGGCGTAATAGCGAA
GAGGCCCGCACCGATCGCCCTTCCCAACAGTTGCGCAGCCTGAATGGCGAATGGG
(mGLB1 WT)
SEQ ID NO: 41
AGAGCGCCCACTGCCTAACGGAGAGACCCCATCGTGGCGCGATCATGCTCCGGGTCCCCCTGTGTACGCCGCTCCCG
CTCCTGGCACTGCTGCAACTGCTGGGCGCTGCGCACGGCATCTATAATGTCACCCAGAGGACATTTAAGCTCGACTA
CAGCCGGGACCGCTTCCTCAAGGATGGACAGCCATTCCGATACATCTCGGGAAGCATTCATTACTTCCGGATACCCC
GCTTCTACTGGGAGGACCGGCTGCTGAAGATGAAGATGGCTGGGCTGAATGCTATCCAGATGTACGTGCCCTGGAAC
TTCCATGAACCCCAACCAGGACAATATGAGTTTTCTGGGGACCGTGATGTGGAGCATTTCATCCAGCTGGCTCATGA
GCTGGGACTCCTGGTGATCCTGAGGCCTGGGCCCTACATCTGTGCAGAGTGGGACATGGGGGGCTTACCTGCTTGGC
TACTAGAGAAACAATCTATCGTTCTCCGGTCTTCTGACCCAGACTACCTTGTAGCTGTGGATAAATGGCTGGCAGTC
CTTCTGCCCAAGATGAAGCCCCTGCTCTACCAGAACGGAGGACCGATCATAACCGTGCAGGTTGAGAATGAGTACGG
GTCCTACTTTGCCTGCGATTACGACTACCTACGCTTCCTGGTGCACCGCTTCCGCTACCATCTGGGTAATGACGTCA
TTCTCTTCACCACCGACGGAGCAAGTGAAAAAATGCTGAAGTGTGGGACCCTGCAGGACCTGTACGCCACAGTGGAT
TTTGGAACAGGCAACAATATCACACAAGCTTTCCTGGTCCAGAGGAAGTTTGAACCTAAAGGACCTTTGATCAATTC
TGAGTTCTATACTGGCTGGCTAGACCACTGGGGTAAACCCCATTCCACGGTGAAAACTAAAACACTGGCTACCTCCC
TCTATAACCTGCTTGCCCGTGGGGCCAACGTGAACTTGTACATGTTTATAGGTGGGACCAATTTTGCCTATTGGAAT
GGTGCCAACACGCCCTATGAGCCACAGCCCACCAGCTATGACTACGACGCCCCACTGAGCGAGGCTGGGGACCTCAC
TAAGAAGTATTTTGCTCTTCGAGAAGTCATTCAGATGTTTAAAGAAGTCCCAGAAGGCCCTATCCCTCCGTCTACAC
CCAAATTTGCATATGGAAAAGTTGCTCTGAGAAAGTTCAAGACAGTGGCTGAAGCTCTGGGTATCCTGTGTCCCAAT
GGGCCAGTGAAAAGCCTCTATCCCCTGACATTCACTCAGGTAAAACAGTATTTTGGGTATGTGCTGTACCGAACAAC
GCTTCCTCAAGATTGCAGTAACCCGAAACCCATTTTCTCTTCACCCTTCAATGGTGTCCGTGATCGGGCTTACGTCT
CTGTGGACGGGGTCCCCCAAGGAATCCTTGATCGAAACCTCATGACAGCTCTGAACATACGGGGGAAGGCTGGAGCC
ACGCTGGACATCCTGGTGGAGAACATGGGGCGTGTGAACTATGGCAGATTCATCAATGACTTCAAGGGTTTGATTTC
CAACATGACTATCAACTCCACTGTCCTCACCAACTGGACGGTCTTCCCACTGAACACTGAGGCCATGGTACGCAACC
ATCTCTGGGGCCGGGAGGCCAGTGATGAGGGTCACCTTGACGGACGGTCGACCTCCAATTCTTCGGACCTCATACTC
CCCACCTTTTACGTGGGCAACTTCTCCATCCCCTCGGGCATCCCAGACCTGCCACAGGACACCTTCATCCAGTTTCC
TGGGTGGTCCAAGGGTCAAGTATGGATCAATGGCTTTAACCTCGGCCGATACTGGCCCACAATGGGCCCACAAAAGA
CCTTGTTCGTGCCAAGGAACATCCTGACCACTTCAGCCCCAAACAACATCACAGTGTTGGAGCTAGAGTTTGCACCC
TGCAGCGAGGGGACCCCAGAGCTGTGTACAGTAGAGTTTGTTGACACTCCGGTCATTTCCTGACCTGACTTGACCAT
CGGTGGCCATTTTCCAAGCCAGTCTGGTCAAGACTCATGGCTGAACCTCTGAGACTGAGCCTTGGGGAGCACAGCTC
TACTCTGGTTACACGGATCACCTTTGTTGTGCTAGAATGGAAGCTACAT
(LV-GLB1 WT cassette)
SEQ ID NO: 42
ATCATCGATTTCCGAATTCCACGGGGTTGGGGTTGCGCCTTTTCCAAGGCAGCCCTGGGTTTGCGCAGGGACGCGGC
TGCTCTGGGCGTGGTTCCGGGAAACGCAGCGGCGCCGACCCTGGGTCTCGCACATTCTTCACGTCCGTTCGCAGCGT
CACCCGGATCTTCGCCGCTACCCTTGTGGGCCCCCCGGCGACGCTTCCTGCTCCGCCCCTAAGTCGGGAAGGTTCCT
TGCGGTTCGCGGCGTGCCGGACGTGACAAACGGAAGCCGCACGTCTCACTAGTACCCTCGCAGACGGACAGCGCCAG
GGAGCAATGGCAGCGCGCCGACCGCGATGGGCTGTGGCCAATAGCGGCTGCTCAGCAGGGCGCGCCGAGAGCAGCGG
CCGGGAAGGGGCGGTGCGGGAGGCGGGGTGTGGGGCGGTAGTGTGGGCCCTGTTCCTGCCCGCGCGGTGTTCCGCAT
TCTGCAAGCCTCCGGAGCGCACGTCGGCAGTCGGCTCCCTCGTTGACCGAATCACCGACCTCTCTCCCCAACGCgga
tccAGAGCGCCCACTGCCTAACGGAGAGACCCCATCGTGGCGCGATCATGCTCCGGGTCCCCCTGTGTACGCCGCTC
CCGCTCCTGGCACTGCTGCAACTGCTGGGCGCTGCGCACGGCATCTATAATGTCACCCAGAGGACATTTAAGCTCGA
CTACAGCCGGGACCGCTTCCTCAAGGATGGACAGCCATTCCGATACATCTCGGGAAGCATTCATTACTTCCGGATAC
CCCGCTTCTACTGGGAGGACCGGCTGCTGAAGATGAAGATGGCTGGGCTGAATGCTATCCAGATGTACGTGCCCTGG
AACTTCCATGAACCCCAACCAGGACAATATGAGTTTTCTGGGGACCGTGATGTGGAGCATTTCATCCAGCTGGCTCA
TGAGCTGGGACTCCTGGTGATCCTGAGGCCTGGGCCCTACATCTGTGCAGAGTGGGACATGGGGGGCTTACCTGCTT
GGCTACTAGAGAAACAATCTATCGTTCTCCGGTCTTCTGACCCAGACTACCTTGTAGCTGTGGATAAATGGCTGGCA
GTCCTTCTGCCCAAGATGAAGCCCCTGCTCTACCAGAACGGAGGACCGATCATAACCGTGCAGGTTGAGAATGAGTA
CGGGTCCTACTTTGCCTGCGATTACGACTACCTACGCTTCCTGGTGCACCGCTTCCGCTACCATCTGGGTAATGACG
TCATTCTCTTCACCACCGACGGAGCAAGTGAAAAAATGCTGAAGTGTGGGACCCTGCAGGACCTGTACGCCACAGTG
GATTTTGGAACAGGCAACAATATCACACAAGCTTTCCTGGTCCAGAGGAAGTTTGAACCTAAAGGACCTTTGATCAA
TTCTGAGTTCTATACTGGCTGGCTAGACCACTGGGGTAAACCCCATTCCACGGTGAAAACTAAAACACTGGCTACCT
CCCTCTATAACCTGCTTGCCCGTGGGGCCAACGTGAACTTGTACATGTTTATAGGTGGGACCAATTTTGCCTATTGG
AATGGTGCCAACACGCCCTATGAGCCACAGCCCACCAGCTATGACTACGACGCCCCACTGAGCGAGGCTGGGGACCT
CACTAAGAAGTATTTTGCTCTTCGAGAAGTCATTCAGATGTTTAAAGAAGTCCCAGAAGGCCCTATCCCTCCGTCTA
CACCCAAATTTGCATATGGAAAAGTTGCTCTGAGAAAGTTCAAGACAGTGGCTGAAGCTCTGGGTATCCTGTGTCCC
AATGGGCCAGTGAAAAGCCTCTATCCCCTGACATTCACTCAGGTAAAACAGTATTTTGGGTATGTGCTGTACCGAAC
AACGCTTCCTCAAGATTGCAGTAACCCGAAACCCATTTTCTCTTCACCCTTCAATGGTGTCCGTGATCGGGCTTACG
TCTCTGTGGACGGGGTCCCCCAAGGAATCCTTGATCGAAACCTCATGACAGCTCTGAACATACGGGGGAAGGCTGGA
GCCACGCTGGACATCCTGGTGGAGAACATGGGGCGTGTGAACTATGGCAGATTCATCAATGACTTCAAGGGTTTGAT
TTCCAACATGACTATCAACTCCACTGTCCTCACCAACTGGACGGTCTTCCCACTGAACACTGAGGCCATGGTACGCA
ACCATCTCTGGGGCCGGGAGGCCAGTGATGAGGGTCACCTTGACGGACGGTCGACCTCCAATTCTTCGGACCTCATA
CTCCCCACCTTTTACGTGGGCAACTTCTCCATCCCCTCGGGCATCCCAGACCTGCCACAGGACACCTTCATCCAGTT
TCCTGGGTGGTCCAAGGGTCAAGTATGGATCAATGGCTTTAACCTCGGCCGATACTGGCCCACAATGGGCCCACAAA
AGACCTTGTTCGTGCCAAGGAACATCCTGACCACTTCAGCCCCAAACAACATCACAGTGTTGGAGCTAGAGTTTGCA
CCCTGCAGCGAGGGGACCCCAGAGCTGTGTACAGTAGAGTTTGTTGACACTCCGGTCATTTCCTGACCTGACTTGAC
CATCGGTGGCCATTTTCCAAGCCAGTCTGGTCAAGACTCATGGCTGAACCTCTGAGACTGAGCCTTGGGGAGCACAG
CTCTACTCTGGTTACACGGATCACCTTTGTTGTGCTAGAATGGAAGCTACAT

1. A viral vector comprising an expression cassette for expressing, in a cell, an enzyme that is deficient in a lysosomal storage disorder, preferably in a lysosomal storage disorder with skeletal involvement, said expression cassette comprising:
a) a promoter and
b) at least one polynucleotide, operably linked to said promoter, encoding an enzyme that is deficient in a lysosomal storage disorder, wherein said enzyme is selected from the group consisting of: alpha-D-mannosidase enzyme, beta-galactosidase enzyme, and N-acetylgalactosamine-6-sulfatase enzyme,
said viral vector being a lentiviral vector.

2. The viral vector of claim 1, wherein the promoter is selected from:
a.1) an isolated human PGK promoter, preferably of sequence SEQ ID NO: 3, or variants thereof;
a.2) an isolated eukaryotic Translation Elongation Factor 1 alpha 1 promoter, preferably of sequence SEQ ID NO: 6, or variants thereof;
a.3) an isolated CMV enhancer-containing promoter, preferably of sequence SEQ ID NO: 7, or variants thereof;
a.4) a CAG promoter, preferably of sequence SEQ ID NO: 8, or variants thereof,
a.5) the natural promoter of the gene encoding the enzyme that is deficient in a lysosomal storage disorder with skeletal involvement.

3. The viral vector of claim 1, being a replication-defective human immunodeficiency virus (HIV).

4. The viral vector of claim 13, further comprising one or more of: c) a 5′ long terminal repeat (5′ LTR); d) an encapsidation signal (Ψ), preferably including the 5′ portion of the gag gene (GA); e) a Rev-response element (RRE); f) a central polypurine tract (cPPT), g) a central termination sequence (CTS), h) a post-transcriptional regulatory element of woodchuck hepatitis virus (Wpre); i) a 3′ long terminal repeat region (3′LTR), preferably self-inactivating (SIN) 3′LTR; j) a polyadenylation signal; k) an SV40 origin of replication; and 1) a bacterial high copy origin of replication (f1 ori).

5. The viral vector of claim 1, wherein the at least one polynucleotide that encodes the enzyme that is deficient in a lysosomal storage disorder is a polynucleotide that encodes alpha-D-mannosidase enzyme, or a polynucleotide that encodes beta-galactosidase enzyme, or a polynucleotide that encodes N-acetylgalactosamine-6-sulfatase enzyme.

6. The viral vector of claim 5, wherein the polynucleotide that encodes alpha-D-mannosidase enzyme has sequence comprising sequence SEQ ID NO: 21, 22, or variants thereof.

7. (canceled)

8. The viral vector of claim 5, wherein the polynucleotide that encodes beta-galactosidase enzyme has sequence comprising sequence SEQ ID NO: 1, 2, 20, 41, or variants thereof.

9-10. (canceled)

11. The viral vector of claim 5, wherein the polynucleotide that encodes N-acetylgalactosamine-6-sulfatase enzyme has sequence comprising, or consisting of, sequence SEQ ID NO: 35, 36, or variants thereof.

12. The viral vector of claim 1, wherein the expression cassette has sequence comprising, or consisting of, sequence SEQ ID NO: 24, 25, or variants thereof, or sequence comprising SEQ ID NO: 4, 5, 23, 27 or variants thereof, or sequence comprising SEQ ID NO: 37, 38 or variants thereof.

13-15. (canceled)

16. The viral vector of claim 1 having sequence comprising ID NO: 31, 32, or variants thereof, or sequence comprising SEQ ID NO: 28, 29, 30, 33, 34 or variants thereof, or sequence comprising SEQ ID NO: 39, 40 or variants thereof.

17-18. (canceled)

19. An engineered cell comprising the viral vector of claim 1, preferably a hematopoietic stem and progenitor cells (HSPC) of a T cell, more preferably a CD34⁺ T.

20. The engineered cell of claim 19 integrating the expression cassette of the viral vector.

21-23. (canceled)

24. A method of producing the engineered cell of claim 19, comprising the steps of:
i. providing isolated cells; and
ii. transducing the isolated cells with the viral vector of claim 1, obtaining the engineered cells;
optionally the method further comprising:
iii. suspending the engineered cells in a freezing medium and freeze the engineered cells suspension.

25-26. (canceled)

27. The method of claim 24, further comprising the step of:
i.i stimulating the isolated cells with a mix of cytokines
before the step ii. transducing the isolated cells with the viral vector.

28. The method of claim 24, further comprising the step of:
i.ii contacting the isolated cells with one or more transduction enhancers
before the step ii. transducing the isolated cells with the viral vector.

29. The method of claim 24, wherein the viral vector integrates into the genome of the isolated cells following transduction.

30. A method of ex vivo gene therapy for treating a lysosomal storage disorder, preferably a lysosomal storage disorder with skeletal involvement, comprising the step of
administering a therapeutically effective amount of the recombinant viral vector of claim 1 or an engineered cell comprising the viral vector of claim 1, preferably a hematopoietic stem and progenitor cells (HSPC) of a T cell, more preferably a CD34⁺ T cell to a subject in need thereof.

31-33. (canceled)

34. The method of claim 30, comprising a step of chemotherapy-based conditioning regimen of the subject in need of said treatment, followed by a step of administering the lentiviral vector, or the cell to said subject.
